SEQUENCE LISTING (132 sequences)

SEQ ID NO: 1 - protein, 740 aa, synthetic construct
  REGION 1..740: note = Synthetic; Fusion construct
MGVLLTQRTLLSLVLALLFPSMASMQVQLQQSGAELVKPGASVKISCKASGYSFTGYFMNWVKQSHGKSLEWIGRIHPYDGDTFYNQNFKDKATLTVDKSSNTAHMELLSLTSEDFAVYYCTRYDGSRAMDYWGQGTTVTVSSGGGGSGGGGSGGGGSDIELTQSPASLAVSLGQRAIISCKASQSVSFAGTSLMHWYHQKPGQQPKLLIYRASNLEAGVPTRFSGSGSKTDFTLNIHPVEEEDAATYYCQQSREYPYTFGGGTKLEIKAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 2 - protein, 25 aa, synthetic construct
  REGION 1..25: note = Synthetic; Oncostatin M
MGVLLTQRTLLSLVLALLFPSMASM

SEQ ID NO: 3 - protein, 118 aa, synthetic construct
  REGION 1..118: note = Synthetic; VH
QVQLQQSGAELVKPGASVKISCKASGYSFTGYFMNWVKQSHGKSLEWIGRIHPYDGDTFYNQNFKDKATLTVDKSSNTAHMELLSLTSEDFAVYYCTRYDGSRAMDYWGQGTTVTVSS

SEQ ID NO: 4 - protein, 15 aa, synthetic construct
  REGION 1..15: note = Synthetic; Linker
GGGGSGGGGSGGGGS

SEQ ID NO: 5 - protein, 111 aa, synthetic construct
  REGION 1..111: note = Synthetic; VL
DIELTQSPASLAVSLGQRAIISCKASQSVSFAGTSLMHWYHQKPGQQPKLLIYRASNLEAGVPTRFSGSGSKTDFTLNIHPVEEEDAATYYCQQSREYPYTFGGGTKLEIK

SEQ ID NO: 6 - protein, 9 aa, synthetic construct
  REGION 1..9: note = Synthetic; Linker
AAAGSGGSG

SEQ ID NO: 7 - protein, 200 aa, synthetic construct
  REGION 1..200: note = Synthetic; CD28 fragment
ILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 8 - protein, 132 aa, synthetic construct
  REGION 1..132: note = Synthetic; CD28 EC Domain
ILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKP

SEQ ID NO: 9 - protein, 27 aa, synthetic construct
  REGION 1..27: note = Synthetic; TM Domain
FWVLVVVGGVLACYSLLVTVAFIIFWV

SEQ ID NO: 10 - protein, 41 aa, synthetic construct
  REGION 1..41: note = Synthetic; IC Domain
RSKRSRLLHSDYMNMTPRRPGPTRKHYQPYAPPRDFAAYRS

SEQ ID NO: 11 - protein, 62 aa, synthetic construct
  REGION 1..62: note = Synthetic; CD40 fragment
KKVAKKPTNKAPHPKQEPQEINFPDDLPGSNTAAPVQETLHGCQPVTQEDGKESRISVQERQ

SEQ ID NO: 12 - protein, 120 aa, synthetic construct
  REGION 1..120: note = Synthetic; VH
QVKLEQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSS

SEQ ID NO: 13 - protein, 15 aa, synthetic construct
  REGION 1..15: note = Synthetic; Linker
GGGGSGGGGSGGGGS

SEQ ID NO: 14 - protein, 106 aa, synthetic construct
  REGION 1..106: note = Synthetic; VL
ENVLTQSPSSMSASVGDRVNIACSASSSVSYMHWFQQKPGKSPKLWIYSTSNLASGVPSRFSGSGSGTDYSLTISSMQPEDAATYYCQQRSSYPLTFGGGTKLEIK

SEQ ID NO: 15 - protein, 9 aa, synthetic construct
  REGION 1..9: note = Synthetic; Linker
AAAGSGGSG

SEQ ID NO: 16 - protein, 180 aa, synthetic construct
  REGION 1..180: note = Synthetic; CD40
GEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 17 - protein, 62 aa, synthetic construct; identical to SEQ ID NO: 11
  REGION 1..62: note = Synthetic; CD40 Intracellular Domain

SEQ ID NO: 18 - protein, 107 aa, synthetic construct
  REGION 1..107: note = Synthetic; VL
ELVMTQTPSSVSAAVGGTVTITCQASQSLSNLLAWYQQKPGQPPKLLIYGASNLESGVPSRFRGSGSGTDFTLTISGMKAEDAATYYCQGGHYSGLTFGNGTNVEIK

SEQ ID NO: 19 - protein, 15 aa, synthetic construct
  REGION 1..15: note = Synthetic; Linker
GGGGSGGGGSGGGGS

SEQ ID NO: 20 - protein, 119 aa, synthetic construct
  REGION 1..119: note = Synthetic; VH
QSLEESGGRLVTPGTPLTLTCTVSGFSLSTNDMNWVRQAPGKGLEWIGVIYSDDTPDYATWAKGRFTISRTSTTVDLKITSPTTEDTATYFCARGHYDSAVYAYALNIWGPGTLVTVSS

SEQ ID NO: 21 - protein, 9 aa, synthetic construct
  REGION 1..9: note = Synthetic; Linker
AAAGSGGSG

SEQ ID NO: 22 - protein, 39 aa, synthetic construct
  REGION 1..39: note = Synthetic; CD28 Extracellular
IEVMYPPPYLDNEKSNGTIIHVKGKHLCPSPLFPGPSKP

SEQ ID NO: 23 - protein, 27 aa, synthetic construct
  REGION 1..27: note = Synthetic; CD28 Transmembrane Domain
FWVLVVVGGVLACYSLLVTVAFIIFWV

SEQ ID NO: 24 - protein, 41 aa, synthetic construct; identical to SEQ ID NO: 10
  REGION 1..41: note = Synthetic; CD28 Intracellular Domain

SEQ ID NO: 25 - protein, 62 aa, synthetic construct; identical to SEQ ID NO: 11
  REGION 1..62: note = Synthetic; CD40 Intracellular Domain

SEQ ID NO: 26 - protein, 8 aa, synthetic construct
  REGION 1..8: note = Synthetic; SH3 motif
KPTNKAPH

SEQ ID NO: 27 - protein, 4 aa, synthetic construct
  REGION 1..4: note = Synthetic; TRAF2 motif1
PKQE

SEQ ID NO: 28 - protein, 4 aa, synthetic construct
  REGION 1..4: note = Synthetic; TRAF2 motif2
PVQE

SEQ ID NO: 29 - protein, 4 aa, synthetic construct
  REGION 1..4: note = Synthetic; TRAF2 motif3
SVQE

SEQ ID NO: 30 - protein, 9 aa, synthetic construct
  REGION 1..9: note = Synthetic; TRAF6 motif
QEPQEINFP

SEQ ID NO: 31 - protein, 7 aa, synthetic construct
  REGION 1..7: note = Synthetic; PKA motif1
KKPTNKA

SEQ ID NO: 32 - protein, 7 aa, synthetic construct
  REGION 1..7: note = Synthetic; PKA motif2
SRISVQE

SEQ ID NO: 33 - protein, 440 aa, synthetic construct
MALPVTALLLPLALLLHAARPELVMTQTPSSVSAAVGGTVTITCQASQSLSNLLAWYQQKPGQPPKLLIYGASNLESGVPSRFRGSGSGTDFTLTISGMKAEDAATYYCQGGHYSGLTFGNGTNVEIKGGGGSGGGGSGGGGSQSLEESGGRLVTPGTPLTLTCTVSGFSLSTNDMNWVRQAPGKGLEWIGVIYSDDTPDYATWAKGRFTISRTSTTVDLKITSPTTEDTATYFCARGHYDSAVYAYALNIWGPGTLVTVSSAAAGSGGSGIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 34 - protein, 21 aa, synthetic construct
MALPVTALLLPLALLLHAARP

SEQ ID NO: 35 - protein, 517 aa, synthetic construct
MGVLLTQRTLLSLVLALLFPSMASMQVKLEQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSSGGGGSGGGGSGGGGSENVLTQSPSSMSASVGDRVNIACSASSSVSYMHWFQQKPGKSPKLWIYSTSNLASGVPSRFSGSGSGTDYSLTISSMQPEDAATYYCQQRSSYPLTFGGGTKLEIKAAAGSGGSGGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTLKKVAKKPTNKAPHPKQEPQEINFPDDLPGSNTAAPVQETLHGCQPVTQEDGKESRISVQERQ

SEQ ID NO: 36 - protein, 25 aa, synthetic construct
MGVLLTQRTLLSLVLALLFPSMASM

SEQ ID NO: 37 - protein, 120 aa, synthetic construct; identical to SEQ ID NO: 12

SEQ ID NO: 38 - protein, 15 aa, synthetic construct
GGGGSGGGGSGGGGS

SEQ ID NO: 39 - protein, 106 aa, synthetic construct; identical to SEQ ID NO: 14

SEQ ID NO: 40 - protein, 9 aa, synthetic construct
AAAGSGGSG

SEQ ID NO: 41 - protein, 180 aa, synthetic construct; identical to SEQ ID NO: 16

SEQ ID NO: 42 - protein, 62 aa, synthetic construct; identical to SEQ ID NO: 11

SEQ ID NO: 43 - protein, 159 aa, synthetic construct
ILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWV

SEQ ID NO: 44 - protein, 41 aa, synthetic construct; identical to SEQ ID NO: 10

SEQ ID NO: 45 - protein, 17 aa, synthetic construct
SSGGGGSGGGGSGGGGS

SEQ ID NO: 46 - protein, 536 aa, synthetic construct
MGVLLTQRTLLSLVLALLFPSMASMQVQLQQSGPELEKPGASVKLSCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGAGTKLEIKAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 47 - protein, 240 aa, synthetic construct
QVQLQQSGPELEKPGASVKLSCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGAGTKLEIK

SEQ ID NO: 48 - protein, 117 aa, synthetic construct
QVQLQQSGPELEKPGASVKLSCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTV

SEQ ID NO: 49 - protein, 106 aa, synthetic construct
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGAGTKLEIK

SEQ ID NO: 50 - protein, 532 aa, synthetic construct
MGVLLTQRTLLSLVLALLFPSMASMQVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 51 - protein, 236 aa, synthetic construct
QVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIK

SEQ ID NO: 52 - protein, 113 aa, synthetic construct
QVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTV

SEQ ID NO: 53 - protein, 106 aa, synthetic construct
DIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIK

SEQ ID NO: 54 - protein, 537 aa, synthetic construct
MGVLLTQRTLLSLVLALLFPSMASMQVQLVQSGAEVKRPGASVQVSCRASGYSINTYYMQWVRQAPGAGLEWMGVINPSGVTSYAQKFQGRVTLTNDTSTNTVYMQLNSLTSADTAVYYCARWALWGDFGMDVWGKGTLVTVSSGGGGSGGGGSGGGGSDIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIKAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 55 - protein, 241 aa, synthetic construct
QVQLVQSGAEVKRPGASVQVSCRASGYSINTYYMQWVRQAPGAGLEWMGVINPSGVTSYAQKFQGRVTLTNDTSTNTVYMQLNSLTSADTAVYYCARWALWGDFGMDVWGKGTLVTVSSGGGGSGGGGSGGGGSDIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIK

SEQ ID NO: 56 - protein, 117 aa, synthetic construct
QVQLVQSGAEVKRPGASVQVSCRASGYSINTYYMQWVRQAPGAGLEWMGVINPSGVTSYAQKFQGRVTLTNDTSTNTVYMQLNSLTSADTAVYYCARWALWGDFGMDVWGKGTLVTV

SEQ ID NO: 57 - protein, 107 aa, synthetic construct
DIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIK

SEQ ID NO: 58 - protein, 537 aa, synthetic construct
MGVLLTQRTLLSLVLALLFPSMASMQVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSGGGGSGGGGSGGGGSDIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIKAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 59 - protein, 241 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSGGGGSGGGGSGGGGSDIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIK

SEQ ID NO: 60 - protein, 117 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTV

SEQ ID NO: 61 - protein, 107 aa, synthetic construct
DIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIK

SEQ ID NO: 62 - protein, 545 aa, synthetic construct
MGVLLTQRTLLSLVLALLFPSMASMEVQLVESGGGLVQPGGSLRLSCAASGFDLGFYFYACWVRQAPGKGLEWVSCIYTAGSGSTYYASWAKGRFTISRDNSKNTLYLQMNSLRAEDTAVYYCARSTANTRSTYYLNLWGQGTLVTVSSGGGGSGGGGSGGGGSDIQMTQSPSSLSASVGDRVTITCQASQRISSYLSWYQQKPGKVPKLLIYGASTLASGVPSRFSGSGSGTDFTLTISSLQPEDVATYYCQSYAYFDSNNWHAFGGGTKVEIAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 63 - protein, 249 aa, synthetic construct
EVQLVESGGGLVQPGGSLRLSCAASGFDLGFYFYACWVRQAPGKGLEWVSCIYTAGSGSTYYASWAKGRFTISRDNSKNTLYLQMNSLRAEDTAVYYCARSTANTRSTYYLNLWGQGTLVTVSSGGGGSGGGGSGGGGSDIQMTQSPSSLSASVGDRVTITCQASQRISSYLSWYQQKPGKVPKLLIYGASTLASGVPSRFSGSGSGTDFTLTISSLQPEDVATYYCQSYAYFDSNNWHAFGGGTKVEI

SEQ ID NO: 64 - protein, 122 aa, synthetic construct
EVQLVESGGGLVQPGGSLRLSCAASGFDLGFYFYACWVRQAPGKGLEWVSCIYTAGSGSTYYASWAKGRFTISRDNSKNTLYLQMNSLRAEDTAVYYCARSTANTRSTYYLNLWGQGTLVTV

SEQ ID NO: 65 - protein, 110 aa, synthetic construct
DIQMTQSPSSLSASVGDRVTITCQASQRISSYLSWYQQKPGKVPKLLIYGASTLASGVPSRFSGSGSGTDFTLTISSLQPEDVATYYCQSYAYFDSNNWHAFGGGTKVEI

SEQ ID NO: 66 - protein, 550 aa, synthetic construct
MGVLLTQRTLLSLVLALLFPSMASMQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 67 - protein, 254 aa, synthetic construct
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLS

SEQ ID NO: 68 - protein, 121 aa, synthetic construct
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTV

SEQ ID NO: 69 - protein, 116 aa, synthetic construct
QPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLS

SEQ ID NO: 70 - protein, 715 aa, synthetic construct
QVQLQQSGAELVKPGASVKISCKASGYSFTGYFMNWVKQSHGKSLEWIGRIHPYDGDTFYNQNFKDKATLTVDKSSNTAHMELLSLTSEDFAVYYCTRYDGSRAMDYWGQGTTVTVSSGGGGSGGGGSGGGGSDIELTQSPASLAVSLGQRAIISCKASQSVSFAGTSLMHWYHQKPGQQPKLLIYRASNLEAGVPTRFSGSGSKTDFTLNIHPVEEEDAATYYCQQSREYPYTFGGGTKLEIKAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 71 - protein, 419 aa, synthetic construct
ELVMTQTPSSVSAAVGGTVTITCQASQSLSNLLAWYQQKPGQPPKLLIYGASNLESGVPSRFRGSGSGTDFTLTISGMKAEDAATYYCQGGHYSGLTFGNGTNVEIKGGGGSGGGGSGGGGSQSLEESGGRLVTPGTPLTLTCTVSGFSLSTNDMNWVRQAPGKGLEWIGVIYSDDTPDYATWAKGRFTISRTSTTVDLKITSPTTEDTATYFCARGHYDSAVYAYALNIWGPGTLVTVSSAAAGSGGSGIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 72 - protein, 492 aa, synthetic construct
QVKLEQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSSGGGGSGGGGSGGGGSENVLTQSPSSMSASVGDRVNIACSASSSVSYMHWFQQKPGKSPKLWIYSTSNLASGVPSRFSGSGSGTDYSLTISSMQPEDAATYYCQQRSSYPLTFGGGTKLEIKAAAGSGGSGGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTLKKVAKKPTNKAPHPKQEPQEINFPDDLPGSNTAAPVQETLHGCQPVTQEDGKESRISVQERQ

SEQ ID NO: 73 - protein, 511 aa, synthetic construct
QVQLQQSGPELEKPGASVKLSCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGAGTKLEIKAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 74 - protein, 507 aa, synthetic construct
QVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 75 - protein, 512 aa, synthetic construct
QVQLVQSGAEVKRPGASVQVSCRASGYSINTYYMQWVRQAPGAGLEWMGVINPSGVTSYAQKFQGRVTLTNDTSTNTVYMQLNSLTSADTAVYYCARWALWGDFGMDVWGKGTLVTVSSGGGGSGGGGSGGGGSDIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIKAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 76 - protein, 512 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSGGGGSGGGGSGGGGSDIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIKAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 77 - protein, 520 aa, synthetic construct
EVQLVESGGGLVQPGGSLRLSCAASGFDLGFYFYACWVRQAPGKGLEWVSCIYTAGSGSTYYASWAKGRFTISRDNSKNTLYLQMNSLRAEDTAVYYCARSTANTRSTYYLNLWGQGTLVTVSSGGGGSGGGGSGGGGSDIQMTQSPSSLSASVGDRVTITCQASQRISSYLSWYQQKPGKVPKLLIYGASTLASGVPSRFSGSGSGTDFTLTISSLQPEDVATYYCQSYAYFDSNNWHAFGGGTKVEIAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 78 - protein, 525 aa, synthetic construct
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSAAAGSGGSGILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKKVAKKPTNKAPHPKQEPQEINFPDDLPGSNTAAPVQETLHGCQPVTQEDGKESRISVQERQ

SEQ ID NO: 79 - protein, 8 aa, synthetic construct
GYSFTGYF

SEQ ID NO: 80 - protein, 8 aa, synthetic construct
IHPYDGDT

SEQ ID NO: 81 - protein, 10 aa, synthetic construct
RYDGSRAMDY

SEQ ID NO: 82 - protein, 10 aa, synthetic construct
QSPASLAVSL

SEQ ID NO: 83 - protein, 9 aa, synthetic construct
QQSREYPYT

SEQ ID NO: 84 - protein, 9 aa, synthetic construct
QQSREYPYT

SEQ ID NO: 85 - protein, 5 aa, synthetic construct
TNDMN

SEQ ID NO: 86 - protein, 16 aa, synthetic construct
VIYSDDTPDYATWAKG

SEQ ID NO: 87 - protein, 14 aa, synthetic construct
GHYDSAVYAYALNI

SEQ ID NO: 88 - protein, 11 aa, synthetic construct
QASQSLSNLLA

SEQ ID NO: 89 - protein, 7 aa, synthetic construct
GASNLES

SEQ ID NO: 90 - protein, 9 aa, synthetic construct
QGGHYSGLT

SEQ ID NO: 91 - protein, 7 aa, synthetic construct
GFNIKDS

SEQ ID NO: 92 - protein, 6 aa, synthetic construct
DPENGD

SEQ ID NO: 93 - protein, 9 aa, synthetic construct
TPTGPYYFD

SEQ ID NO: 94 - protein, 5 aa, synthetic construct
SSSVS

SEQ ID NO: 95 - protein, 6 aa, synthetic construct
RSSYPL

SEQ ID NO: 96 - protein, 6 aa, synthetic construct
RSSYPL

SEQ ID NO: 97 - protein, 10 aa, synthetic construct
GYSFTGYTMN

SEQ ID NO: 98 - protein, 15 aa, synthetic construct
ITPYNGASSYNQKFR

SEQ ID NO: 99 - protein, 8 aa, synthetic construct
YDGRGFDY

SEQ ID NO: 100 - protein, 10 aa, synthetic construct
SASSSVSYMH

SEQ ID NO: 101 - protein, 7 aa, synthetic construct
DTSKLAS

SEQ ID NO: 102 - protein, 9 aa, synthetic construct
QQWSKHPLT

SEQ ID NO: 103 - protein, 10 aa, synthetic construct
GYTFTDYYMH

SEQ ID NO: 104 - protein, 16 aa, synthetic construct
WINPNSGGTNYAQKFQ

SEQ ID NO: 105 - protein, 6 aa, synthetic construct
GWDFDY

SEQ ID NO: 106 - protein, 11 aa, synthetic construct
RASQSIRYYLS

SEQ ID NO: 107 - protein, 7 aa, synthetic construct
TASILQN

SEQ ID NO: 108 - protein, 8 aa, synthetic construct
LQTYTTPD

SEQ ID NO: 109 - protein, 8 aa, synthetic construct
GYSINTYY

SEQ ID NO: 110 - protein, 7 aa, synthetic construct
INPSGVT

SEQ ID NO: 111 - protein, 18 aa, synthetic construct
AVYYCARWALWGDFGMDV

SEQ ID NO: 112 - protein, 6 aa, synthetic construct
EGIYHW

SEQ ID NO: 113 - protein, 9 aa, synthetic construct
QQYSNYPLT

SEQ ID NO: 114 - protein, 9 aa, synthetic construct
QQYSNYPLT

SEQ ID NO: 115 - protein, 10 aa, synthetic construct
GGSVSSGSYY

SEQ ID NO: 116 - protein, 7 aa, synthetic construct
IYYSGST

SEQ ID NO: 117 - protein, 12 aa, synthetic construct
AREGKNGAFDIW

SEQ ID NO: 118 - protein, 6 aa, synthetic construct
QSISSY

SEQ ID NO: 119 - protein, 4 aa, synthetic construct
AASS

SEQ ID NO: 120 - protein, 10 aa, synthetic construct
QQSYSTPLTF

SEQ ID NO: 121 - protein, 8 aa, synthetic construct
FDLGFYFY

SEQ ID NO: 122 - protein, 8 aa, synthetic construct
IYTAGSGS

SEQ ID NO: 123 - protein, 15 aa, synthetic construct
ARSTANTRSTYYLNL

SEQ ID NO: 124 - protein, 6 aa, synthetic construct
QRISSY

SEQ ID NO: 125 - protein, 15 aa, synthetic construct
CQSYAYFDSNNWHAF

SEQ ID NO: 126 - protein, 15 aa, synthetic construct
CQSYAYFDSNNWHAF

SEQ ID NO: 127 - protein, 7 aa, synthetic construct
SNSATWN

SEQ ID NO: 128 - protein, 18 aa, synthetic construct
RTYYRSKWYNDYAVSVKS

SEQ ID NO: 129 - protein, 11 aa, synthetic construct
GMMTYYYGMDV

SEQ ID NO: 130 - protein, 14 aa, synthetic construct
TLRSGINVGPYRIY

SEQ ID NO: 131 - protein, 11 aa, synthetic construct
YKSDSDKQQGS

SEQ ID NO: 132 - protein, 9 aa, synthetic construct
MIWHSSAAV

(19) United States
(12) Patent Application Publication
(10) Pub. No.: US 2024/0058447 A1
(43) Pub. Date: Feb. 22, 2024

(54) USE OF FUSION CONSTRUCTS FOR IL-2 INDEPENDENT T CELL THERAPY

(71) Applicant: Instil Bio (UK) Limited, Manchester (GB)

(72) Inventors: Owen R. Moon, Manchester (GB); Rubén Alvarez-Rodríguez, Dallas, TX (US); John Bridgeman, Manchester (GB)

(21) Appl. No.: 18/323,342

(22) Filed: May 24, 2023

Related U.S. Application Data
(60) Provisional application No. 63/345,821, filed on May 25, 2022.
(60) Provisional application No. 63/369,719, filed on Jul. 28, 2022.
(60) Provisional application No. 63/379,534, filed on Oct. 14, 2022.
(60) Provisional application No. 63/491,709, filed on Mar. 22, 2023.

(51) Int. Cl. A61K 39/00; A61K 38/17; A61K 39/395; C07K 14/705; A61P 35/00

(52) U.S. Cl. CPC A61K 39/464482; A61K 38/1774; A61K 39/39558; A61K 39/4611; A61K 39/4631; A61K 39/464468; C07K 14/70521; C07K 14/70578; A61K 38/1793; A61P 35/00; C07K 2319/03; C07K 2317/622; A61K 2039/505

(57) ABSTRACT

Provided herein are methods employing various fusion constructs in T cell therapy. The fusion constructs allow for one to reduce, to the point of full removal if desired, the use of IL-2 that would otherwise accompany an in vivo T cell therapy.

### INCORPORATION BY REFERENCE TO ANY PRIORITY APPLICATIONS

This application claims the benefit of U.S. Provisional Ser. No. 63/345,821, filed May 25, 2022; U.S. Provisional Ser. No. 63/369,719, filed Jul. 28, 2022; U.S. Provisional Ser. No. 63/379,534, filed Oct. 14, 2022; and U.S. Provisional Ser. No. 63/491,709 filed Mar. 22, 2023, each of which is hereby incorporated by reference in their entireties.

### RELATED APPLICATIONS AND INCORPORATION BY REFERENCE

This application is related to U.S. Provisional Application Ser. No. 63/290,939, filed Dec. 17, 2021, U.S. Provisional Application Ser. No. 63/366,891, filed Jun. 23, 2022, and PCT Application Serial No. PCT/US2022/034606, filed on Jun. 22, 2022, all of which are hereby expressly incorporated herein by reference in their entireties. This application is also related to U.S. provisional application filed on Oct. 14, 2022, entitled “Pre-Stimulation of Therapeutic Immune Cells”, which is hereby expressly incorporated herein by reference in its entirety. This application is also related to PCT Application Serial No. PCT/US2023/060937, filed on Jan. 19, 2023, which is hereby expressly incorporated herein by reference in its entirety.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The present application is being filed along with a Sequence Listing in electronic format. The Sequence Listing is provided as a file entitled SeqListINSTB010A.xml, which was created on May 23, 2023, which is 141,863 bytes in size. The information in the electronic Sequence Listing is hereby incorporated by reference in its entirety.

### BACKGROUND

### Field

Provided herein are fusion proteins, such as chimeric costimulatory antigen receptors, that can be used in cell therapy (such as an adoptive cell therapy). The presence of these fusion proteins in the cell therapy allows for one to provide the cell therapy in an IL-2 independent manner.

### Description of the Related Art

Adoptive cell therapy (ACT) using autologous T-cells to mediate cancer regression has shown much promise in early clinical trials. Several general approaches have been taken such as the use of naturally occurring tumor reactive or tumor infiltrating lymphocytes (TILs) expanded ex vivo. Additionally, T-cells can be genetically modified to retarget them towards defined tumor antigens. This can be done via the gene transfer of peptide (p)-major histocompatibility complex (MHC) specific T-cell Receptors (TCRs) or synthetic fusions between tumor specific single chain antibody fragment (scFv) and T-cell signaling domains (e.g. CD3z), the latter being termed chimeric antigen receptors (CARs).

TIL and TCR transfer has proven particularly good when targeting melanoma (Rosenberg et al. 2011; Morgan 2006), whereas CAR therapy has shown much promise in the treatment of certain B-cell malignancies (Grupp et al. 2013).

Costimulatory signals are useful to achieve robust CAR T cell expansion, function, persistence and antitumor activity. The success of CAR therapy in leukemia has been partly attributed to the incorporation of costimulatory domains (e.g. CD28 or CD137) into the CAR construct, signals from which synergize with the signal provided by CD3z to enhance anti-tumor activity. The basis of this observation relates to the classical signal 1/signal 2 paradigm of T-cell activation. Here signal 1, provided by the TCR complex, synergizes with signal 2 provided by costimulatory receptors such as CD28, CD137 or CD134 to permit the cells to undergo clonal expansion, IL-2 production and long-term survival without the activation induced cell death (AICD) associated with signal 1 alone. Furthermore, the involvement of signal 2 enhances the signal generated through signal 1 allowing the cells to respond better to low avidity interactions such as those encountered during anti-tumor responses.

Citation or identification of any document in this application is not an admission that such document is available as prior art to the present invention.

### SUMMARY

In some embodiments, a method of treating cancer in a subject that expresses a tumor associated antigen (TAA) is provided. In some embodiments, the method comprises: a. identifying a subject, wherein the subject has cancer that expresses a TAA; and b. administering to the subject a cell comprising a fusion protein. In some embodiments, the fusion protein comprises: i) a binding domain specific for the TAA linked to; ii) a transmembrane domain that is linked to; iii) a CD28 signaling domain that is linked to; iv) a CD40 signaling domain. In some embodiments, the subject does not receive exogenous IL-2 in a manner that is adequate for cell stimulation of TILs in vivo. In some embodiments, the TAA is folate receptor α (FRα). In some embodiments, the TAA is mesothelin (MSLN). In some embodiments, the TAA is cancer antigen 125 (CA125). In some embodiments, the TAA is CD228. In some embodiments, the TAA is melanoma chondroitin sulfate proteoglycan (MCSP). In some embodiments, the TAA is carcinoembryonic antigen (CEA).

In some embodiments, a method of treating cancer in a subject that expresses FOLR1 (FRα) is provided. The method comprises: a) identifying a subject, wherein the subject has cancer that expresses FRα; and b) administering to the subject a cell comprising a fusion protein, wherein the fusion protein comprises: i) a binding domain specific for FRα linked to; ii) a transmembrane domain that is linked to; iii) a CD28 signaling domain that is linked to; iv) a CD40 signaling domain. The subject does not receive exogenous IL-2 in a manner that is adequate for cell stimulation of TILs in vivo.

In some embodiments, a method of cell therapy is provided. The method comprises: a) identifying a subject in need of tumor infiltrating lymphocyte (“TIL”) cell therapy; and b) administering to the subject a TIL cell therapy. The TIL cell therapy: i) comprises a fusion protein that comprises: a) a binding domain specific for FRα linked to; b) a transmembrane domain that is linked to; c) a CD28 signaling domain that is linked to; d) a CD40 signaling domain. The TIL cell therapy does not include a level of IL-2 administered to the subject, wherein the level is one that is sufficient to provide for IL-2 stimulated TIL cell therapy.

In some embodiments, a method of administering a cell therapy is provided. The method comprises: a) administering to a subject a TIL cell therapy, wherein the TIL cell therapy comprises a fusion protein that comprises: i) a binding domain specific for FRα linked to; ii) a transmembrane domain that is linked to; iii) a CD28 signaling domain that is linked to; iv) a CD40 signaling domain. The method excludes a step of administering IL-2 to the subject to promote stimulation of the TILs in vivo, wherein stimulation of the TILs in vivo is achieved via the fusion protein.

In some embodiments, a method of administering a cell therapy is provided. The method comprises: administering a costimulatory antigen receptor (“CoStAR”) to a subject in the absence of a level of IL-2, wherein the level of IL-2 is one sufficient to cause TIL stimulation in vivo when the CoStAR is absent. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

In some embodiments, a method of administering a cell therapy for a cancer treatment is provided. The method comprises administering a T cell comprising a costimulatory antigen receptor (“CoStAR”) to a subject, wherein IL-2 is not used in the therapy at a level sufficient to promote TIL stimulation in the absence of the CoStAR.

In some embodiments, a method of in vivo T cell expansion is provided. The method comprises administering a T cell comprising a fusion protein to a subject. IL-2 is not used to promote TIL stimulation. The fusion protein comprises: a) a binding domain specific for FRα linked to; b) a transmembrane domain that is linked to; c) a CD28 signaling domain that is linked to; d) a CD40 signaling domain.

In some embodiments, a population of genetically engineered immune cells is provided. Each immune cell comprises a fusion protein that comprises: a) a binding domain specific for FRα linked to; b) a transmembrane domain linked to; c) a CD28 signaling domain linked to; d) a CD40 signaling domain. The population of genetically engineered immune cells has been administered to a subject who has not received an amount of IL-2 that is adequate to promote proliferation in vivo without the fusion protein, and wherein the population of immune cells has been expanded in the absence of IL-2 in vivo.

In some embodiments, promoting TIL stimulation denotes a level of stimulation sufficient to achieve a therapeutically effective level of stimulation for a treatment of cancer in the subject. In some embodiments, stimulation is achieved via a TCR dependent mechanism that binds to a peptide that binds to an MHC.

In some embodiments, a population of genetically engineered immune cells is provided. In some embodiments, each immune cell comprises a fusion protein that comprises: a) a binding domain specific for folate receptor α (FRα) linked to; b) a transmembrane domain linked to; c) a CD28 signaling domain linked to; d) a CD40 signaling domain. In some embodiments, the population of genetically engineered immune cells has been administered to a subject who has not received an amount of IL-2 that is adequate to promote proliferation in vivo without the fusion protein. In some embodiments, the population of immune cells has been expanded in the absence of IL-2 in vivo.

In some embodiments, the cells are T cells. In some embodiments, the cells are donor T cells, from the subject. In some embodiments, the cells are tumor infiltrating lymphocytes.

In some embodiments, the fusion protein comprises the polypeptide of SEQ ID NO: 1, comprising the polypeptides of SEQ ID NO: 2-11. In some embodiments, a cancer specific CAR or TCR is present in the cell that contains the fusion protein or CoStAR. In some embodiments, prior to administration to the subject, the cells comprising the fusion protein or CoStAR are incubated with irradiated feeder cells and supplemented with IL-2/mL. In some embodiments, there is no expansion-effective amount IL-2 remaining when the cells are administered to the subject. In some embodiments, the cell is isolated from human PBMCs. In some embodiments, the cell comprises a human tumor infiltrating lymphocyte (TIL), an αβ T cell, a γδ T cell, or an NK T cell.

In some embodiments, the binding domain of the fusion protein comprises an scFv, a peptide, an antibody heavy-chain, a natural ligand or a receptor specific for FRα.

In some embodiments, the binding domain of the fusion protein comprises an scFv specific for FRα with a VH comprising SEQ ID NO: 3 and a VL comprising SEQ ID NO: 5.

In some embodiments, the binding domain is linked to the transmembrane domain by a linker and/or a spacer. In some embodiments, the fusion protein comprises a linker and/or a spacer. In some embodiments, the linker comprises SEQ ID NO: 6.

In some embodiments, the transmembrane domain comprises the transmembrane domain of CD28. In some embodiments, the fusion protein comprises a transmembrane domain of CD28 comprising SEQ ID NO: 9.

In some embodiments, the CD40 domain consists of, consists essentially of, or comprises an SH3 motif (KPTNKAPH, SEQ ID NO:26), TRAF2 motif (PKQE, SEQ ID NO:27, PVQE, SEQ ID NO:28, SVQE, SEQ ID NO:29), TRAF6 motif (QEPQEINFP, SEQ ID NO:30), PKA motif (KKPTNKA, SEQ ID NO:31, SRISVQE, SEQ ID NO:32), a combination thereof, or is a full length CD40 intracellular domain. In some embodiments, the fusion protein comprises an SH3 motif (KPTNKAPH, SEQ ID NO:26), a TRAF2 motif (PKQE, SEQ ID NO:27, PVQE, SEQ ID NO:28, SVQE, SEQ ID NO:29), a TRAF6 motif (QEPQEINFP, SEQ ID NO:30), a PKA motif (KKPTNKA, SEQ ID NO:31, SRISVQE, SEQ ID NO:32), a combination thereof, or a full length CD40 intracellular domain.

In some embodiments, the first signaling domain comprises a full length CD28 signaling domain or where the fusion protein comprises a full length CD28 signaling domain comprising SEQ ID NO: 7.

In some embodiments, the fusion protein or CoStAR enhances antitumor activity by providing costimulatory signaling. In some embodiments, exogenous IL-2 is not needed to support engineered immune cell engraftment within the subject. In some embodiments, the CoStAR expressing cells exhibit increased engraftment. In some embodiment, the CoStAR expressing cells exhibit increased persistence. In some embodiments, the increased engraftment and/or increased persistence of the CoStAR expressing cells is in the peripheral blood and/or in the tumor tissue. In some embodiments, a presence of FRα expressing cells induces engineered cell survival and proliferation.

In some embodiments, the cells are capable of sustained survival in the presence of FRα cells and the absence of IL-2 in vivo. In some embodiments, the engineered cells are capable of surviving at least 60 days post injection in the presence of FRα cells without exogenous IL-2 in vivo.

In some embodiments, FRα stimulated engineered immune cells have reduced PD-1 expression following sustained proliferation.

In some embodiments, the cell is derived from the subject's PBMCs.

In some embodiments, the cancer comprises at least one of: solid tumors, renal cancer, lung cancer, or ovarian cancer.

In some embodiments, the method further comprises: a) providing a cell expressing a fusion protein and a target cell; b) co-culturing the cell expressing the fusion protein with the target cell; c) permeabilizing the cell expressing the fusion protein; and d) evaluating at least one intracellular T cell activation markers. In some embodiments, the method further comprises flow cytometry for evaluating the at least one intracellular T cell activation marker. In some embodiments, the at least one intracellular T cell activation markers comprise CD107a, IFN-γ, CD137, and/or TNFα. In some embodiments, the fusion protein comprises: i) a binding domain specific for FRα, CEA, MSLN, CA125, CD19, CD228, or pembrolizumab linked to; ii) a transmembrane domain that is linked to; iii) a CD28 signaling domain that is linked to; iv) a CD40 signaling domain. In some embodiments, the cell expressing the fusion protein is capable of survival and proliferation in the absence of exogenous IL-2 both in vitro and in vivo. In some embodiments, the fusion protein comprises i) a binding domain specific for CEA linked to; ii) a transmembrane domain that is linked to; iii) a CD28 signaling domain linked to; iv) a CD40 signaling domain.

In some embodiments, a method of providing treatment to a subject that expresses CEA, MSLN or has pembrolizumab in their system is provided. In some embodiments, the method comprises: a) identifying a subject. In some embodiments, the subject has cancer that expresses CEA, MSLN, or has pembrolizumab in their system; and b) administering to the subject a cell comprising a fusion protein. In some embodiments, the fusion protein comprises: i) a binding domain specific for the corresponding CEA, MSLN or pembrolizumab linked to; ii) a transmembrane domain that is linked to; iii) a CD28 signaling domain for the MSLN or pembrolizumab binding domain or ICOS for the CEA binding domain that is linked to; iv) a CD40 signaling domain. In some embodiments, the subject does not receive exogenous IL-2 in a manner that is adequate for cell stimulation of TILs in vivo. In some embodiments, the fusion protein comprises i) a binding domain specific for CEA linked to; ii) a transmembrane domain that is linked to; iii) a CD28 signaling domain linked to; iv) a CD40 signaling domain.

In some embodiments, a method of cell therapy is provided, comprising: a) identifying a subject in need of tumor infiltrating lymphocyte (“TIL”) cell therapy; and b) administering to the subject a TIL cell therapy. In some embodiments, the TIL cell therapy comprises a fusion protein that comprises: a) a binding domain specific for CEA, MSLN or pembrolizumab linked to; b) a transmembrane domain that is linked to; c) a CD28 signaling domain (for the MSLN or pembrolizumab binding domain) or an ICOS domain (for the CEA binding domain) that is linked to; d) a CD40 signaling domain. In some embodiments, the TIL cell therapy does not include a level of IL-2 administered to the subject. In some embodiments, the level is one that is sufficient to provide for IL-2 stimulated TIL cell therapy. In some embodiments, the fusion protein comprises a) a binding domain specific for CEA linked to; b) a transmembrane domain that is linked to; c) a CD28 signaling domain linked to; d) a CD40 signaling domain.

In some embodiments, a method of administering a cell therapy is provided, the method comprising: administering to a subject a TIL cell therapy. In some embodiments, the TIL cell therapy comprises a CoStAR that comprises: a) a binding domain specific for CEA, MSLN or pembrolizumab linked to; b) a transmembrane domain that is linked to; c) a CD28 signaling domain (for the MSLN or pembrolizumab binding domain) or an ICOS domain (for the CEA binding domain) that is linked to; d) a CD40 signaling domain. In some embodiments, the method excludes a step of administering IL-2 to the subject to promote stimulation of the TILs in vivo. In some embodiments, stimulation of the TILs in vivo is achieved via the CoStAR. In some embodiments, the fusion protein comprises a) a binding domain specific for CEA linked to; b) a transmembrane domain that is linked to; c) a CD28 signaling domain linked to; d) a CD40 signaling domain.

In some embodiments, a method of in vivo T cell expansion is provided, the method comprising administering a T cell comprising a fusion protein to a subject. In some embodiments, IL-2 is not used to promote TIL stimulation. In some embodiments, the fusion protein comprises: a) a binding domain specific for CEA, MSLN or pembrolizumab linked to; b) a transmembrane domain that is linked to; c) a CD28 signaling domain (for the MSLN or pembrolizumab binding domain) or an ICOS domain (for the CEA binding domain) that is linked to; d) a CD40 signaling domain. In some embodiments, the fusion protein comprises a) a binding domain specific for CEA linked to; b) a transmembrane domain that is linked to; c) a CD28 signaling domain linked to; d) a CD40 signaling domain. In some embodiments, the fusion protein comprises any one or more of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

In some embodiments, the fusion protein comprises the CDRs as depicted in any one of FIG. 20C, 21C, 22C, or 23D.

In some embodiments, the fusion protein comprises the VH as depicted in any one of FIG. 20C, 21C, 22C, or 23D or a binding fragment thereof.

In some embodiments, the fusion protein comprises the VL as depicted in any one of FIG. 20C, 21C, 22C, or 23D or a binding fragment thereof.

In some embodiments, the fusion protein comprises the CD40 as depicted in any one of FIG. 20C, 21C, 22C, or 23D or a binding fragment thereof.

In some embodiments, the fusion protein comprises the CD28 or ICOS domain as depicted in any one of FIG. 20C, 21C, 22C, or 23C.

In some embodiments, the fusion protein comprises the sequence as depicted in any one of FIG. 20D, 21D, 22D, or 23D or a sequence at least 85, 86, 87, 88, 89, 90, 91, 92, 93, 94, 95, 96, 97, 98, 99 or 100% identical thereto.

In some embodiments, the fusion protein lacks the signal peptide sequence.

In some embodiments, the fusion protein lacks one of SEQ ID NO: 36, 34, or 2.

In some embodiments, the signal sequence has been cleaved from the fusion protein.

In some embodiments, the cell therapy administered comprises a dosage of 5×10\hat{ }8 CoStAR-positive (CoStAR+) T cells, 1×10\hat{ }9 CoStAR+ viable T cells, 3×10\hat{ }9 CoStAR+ viable T cells, or 6×10\hat{ }9 CoStAR+ viable T cells. In some embodiments, the cell therapy administered comprises a dosage of at least 5×10\hat{ }8 CoStAR+ T cells. In some embodiments, the cell therapy administered comprises a dosage of at least 1×10\hat{ }9 CoStAR+viable T cells.

In some embodiments, the cell therapy administered comprises a dosage of at least 3×10\hat{ }9 CoStAR+ viable T cells. In some embodiments, the cell therapy administered comprises a dosage of at least 6×10\hat{ }9 CoStAR+ viable T cells. In some embodiments, the cell therapy administered comprises a dosage of any number of cells between 5×10\hat{ }8 and 6×10\hat{ }9 CoStAR+ viable T cells. In some embodiments, the cell therapy administered initially comprises a dosage of 5×10\hat{ }8 CoStAR+ T cells and is subsequently increased to 1×10\hat{ }9 CoStAR+ viable T cells, increased to 3×10\hat{ }9 CoStAR+ viable T cells, or increased to 6×10\hat{ }9 CoStAR+ viable T cells during the course of treatment.

In some embodiments, the cell therapy administered enhances duration of response, objective response rate, progression free survival, and/or overall survival of the subject receiving the administration.

In some embodiments, the cell therapy administered reduces tumor volume in a subject.

In some embodiments, a method of cell therapy comprising administering a population of cells engineered to express a FRα targeting CoStAR is provided. In some embodiments, FRα expression by target cells enhances engineered T cell activation in a dose dependent manner.

In some embodiments, the dose dependent response of CoStAR engineered cells is to membrane bound FRα.

In some embodiments, the dose dependent response of CoStAR engineered cells to membrane bound FRα requires engagement of TCR signal 1.

In some embodiments, the CoStAR engineered cells do not exhibit a dose dependent T cell activation response to soluble FRα.

In some embodiments, a method of selecting a subject for CoStAR therapy is provided, comprising: assessing expression of FRα. In some embodiments, expression of FRα confers a sensitivity to FRα targeting fusion proteins or CoStARs, in a biological sample obtained from said subject; and selecting said subject as one having a sensitivity to FRα targeting fusion proteins or CoStARs, when said expression of FRα is identified.

In some embodiments, a method of administering a cell therapy in a subject is provided, the method comprising: assessing expression of FRα. In some embodiments, expression of FRα confers a sensitivity to FRα targeting fusion proteins or CoStARs, in a biological sample obtained from said subject, selecting said subject as one having a sensitivity to FRα targeting fusion proteins or CoStARs, when said expression of FRα is identified; and administering to a subject a TIL cell therapy. In some embodiments, the TIL cell therapy comprises a CoStAR.

In some embodiments, FRα expression in a tumor enhances CoStAR antitumor activity via supplemental costimulatory signaling (signal 2).

In some embodiments, based on the mechanism of action of the CoStAR and lack of cytotoxicity in the absence of TCR-pMHC (signal 1), FRα expression in normal tissue is not expected to cause off-tumor toxicity.

In some embodiments, reduction in FRα expression is evaluated following CoStAR cell therapy as a clinical endpoint.

In some embodiments, FRα expression levels are assessed by immunohistochemistry (IHC), polymerase chain reaction (PCR), next generation sequencing (NGS), antibody detection, or companion diagnostic (cDx) assays.

In some embodiments, the cell therapy is administered intravenously in a cell suspension. In some embodiments, the cell therapy is provided as a single infusion.

In some embodiments, the method further comprises pre-stimulation of CoStAR expressing cells. In some embodiments, Signal 2 activation is performed before Signal 1 activation.

In some embodiments, pre-stimulation of the CoStAR expressing cells enhances subsequent stimulation to Signal 1.

In some embodiments, pre-stimulation of the CoStAR expressing cells with Signal 2 is completed before exposure of the cells to Signal 1.

In some embodiments, pre-stimulation of the CoStAR expressing cells with Signal 2 overlaps with exposure of the cells to Signal 1.

### BRIEF DESCRIPTION OF THE DRAWINGS

FIG. 1 illustrates a schematic showing a structure of some of the CoStAR embodiments provided herein. In some embodiments, the FOLR1 (FRα) scFv can be replaced with a pembrolizumab scFv, MSLN scFv, or a CEA scFv (or other binding domain). In some embodiments, the scFv can be any other desired scFv. In some embodiments, the scFv can be any other desired binding domain.

FIG. 2 is a series of graphs illustrating an assessment of all T cell groups for (panel A) their viable number before and after a rapid expansion protocol and (panel B) their expression of transgenic anti-FRα CoStAR molecule and anti-CEA TCR after recovery from cryopreservation.

FIG. 3 is a series of graphs illustrating an assessment of all healthy donor T cell groups for the frequency of different CD3+ T cell sub-populations (panel A) The frequency of αβTCR, γδTCR and T reg cells was assessed (panel B) Within the αβTCR T cell population the expression of αβTCR and transgenic anti-CEA TCR was broken down.

FIG. 4 is a series of graphs illustrating an assessment of all T cell groups for the frequency of different CD3+ T cell sub-populations (panel A) The CD4:CD8 T cell ratio is shown by donor (panel B) The CD4:CD8 ratio is shown by transduction status for each donor (panel C) The CD3 T cell phenotypes are shown by transduction status for each donor.

FIG. 5 is a series of graphs illustrating an assessment of all T cell groups for the frequency co-inhibitory and co-stimulatory markers. (panel A) Donor 41179 CD4 T cell marker expression (panel B) Donor 41179 CD8 T cell marker expression (panel C) Donor 37636 CD4 T cell marker expression (panel D) Donor 37636 CD8 T cell marker expression.

FIG. 6 is a series of graphs illustrating an assessment of all T cell groups for IFNγ, TNFα, IL-17 or IL-22 expression following maximal stimulation by PMA and Ionomycin. (panel A) Donor 41179 CD4 T cell cytokine production (panel B) Donor 41179 CD8 T cell cytokine production (panel C) Donor 37636 CD4 T cell cytokine production (panel D) Donor 37636 CD8 T cell cytokine production.

FIG. 7 is a series of graphs illustrating an assessment of all T cell groups for cytotoxicity against H508.Luc.Puro.FRα target cells at a 10:1 T cell to target ratio over 48 hours. The area under the curve of normalized cell index over time stratified by treatment group.

FIG. 8 is a series of graphs illustrating an experiment where H508.Luc.GFP.FRα were injected into mice 21 days prior to ACT at Day 0 (A) The individual and mean tumor volumes (cm³) of all mice used in the study (B) The individual and mean tumor volumes (cm³) of mice following randomization into 14 groups for treatment.

FIG. 9 illustrates a schema outlining the engraftment of NSG mice with H508.Luc.GFP.FRα tumors, adoptive cell transfer of non-Td, TCR-Td, CoStAR-Td, and TCR.CoStAR-Td T cells, administration of supportive IL-2, the days of tail vein bleed collection, and the study endpoint. Caliper measurements and mouse weight were assessed twice weekly from days −1 to 99.

FIG. 10 is a series of graphs illustrating the TCR expression of non-Td, TCR-Td, CoStAR-Td, and TCR.CoStAR-Td T cells following cryopreservation and their viability following adoptive cell transfer (panel A) The TCR frequency of CD3 T cells for donor 41179 and 37636 (panel B) The percentage of viable cells following recovery from the needle used for adoptive cell transfer into mice.

FIG. 11 is a series of graphs illustrating a flow cytometric assessment of CD3 T cells/mL in mouse tail-vein bleeds in all T-cell groups from donor 41179 (left) or 37636 (middle) with supportive IL-2, or mice receiving T cells from donor 37636 without supportive IL-2 (right) on (panel A) day 14 and (panel B) day 21. N=6 except on day 21 for donor 37636 TCR-Td treatment group without supportive IL-2, which was N=5. Mean and individual data points shown. Detection of T cells in mice that received no treatment is used to define the detection limit of the assay and is shown as lines on the y axis, solid line=mean and dotted line=SD.

FIG. 12 is a series of graphs illustrating a direct post-analysis comparison of TCR.CoStAR-Td T cells from donor 37636 with and without exogenous IL-2. Flow cytometric assessment of CD3 T cells/mL in mouse tail-vein bleeds on (panel A) day 14 and (panel B) day 21.N=6, mean and individual data points shown, detection of T cells in mice that received. Statistical test: 1-way ANOVA with Tukey's test for multiple comparisons. Growth of established H508.Luc.GFP.FRα tumors was monitored from day −1 to 58 by regular digital caliper measurement. (panel C) The average tumor growth of the TCR.CoStAR-Td treatment group N=6, mean±SD, statistical test: Mixed-effects model with Tukey's multiple comparisons test. (panel D) Survival of mice with established subcutaneous H508.Luc.GFP.FRα tumors up to experimental endpoints. The Kaplan-Meier curve for TCR.CoStAR-Td treatment group.

FIG. 13 is a series of graphs illustrating growth of established H508.Luc.GFP.FRα tumors as monitored from days −1 to 58 by regular digital caliper measurement. (panel A) Individual mouse growth curves for tumor-bearing mice either untreated or treated with non-Td, CoStAR-Td, TCR-Td, or TCR.CoStAR-Td T cells from donor 41179. (panel B) The average tumor growth of each treatment group.

FIG. 14 is a series of graphs illustrating growth of established H508.Luc.GFP.FRα tumors as monitored from days −1 to 58 by regular digital caliper measurement. (panel A) Individual mouse growth curves for tumor-bearing mice either untreated or treated with non-Td, CoStAR-Td, TCR-Td, or TCR.CoStAR-Td T cells from donor 37636. (panel B) The average tumor growth of each treatment group (panel C) Individual mouse growth curves for tumor-bearing mice either untreated or treated with non-Td, CoStAR-Td, TCR-Td, or TCR.CoStAR-Td T cells from donor 37636 without IL-2 support. (panel D) The average tumor growth of each treatment group without IL-2 support.

FIG. 15 is a series of graphs illustrating assessment of the survival of mice with established subcutaneous H508.Luc.GFP.FRα tumors after adoptive cell transfer of non-Td and Td T cells, and mice were sacrificed at experimental endpoints. The Kaplan-Meier curve for donor (panel A) 41179, (panel B) 37636 with and (panel C) without exogenous IL-2 support.

FIG. 16 provides some embodiments of various sequences that can be in part or in whole used in some of the embodiments provided herein. The sequences include some FRα protein embodiments, some CEA embodiments, some MSLN embodiments, and some pembrolizumab embodiments.

FIG. 17 illustrates a schematic for some embodiments of administering some FRα CoStARs. The schematic in FIG. 17 depicts a process for a ITIL 306-206 study, which is a multicenter, first in human, single arm phase 1a/1b dose escalation and expansion study evaluating the safety and feasibility of ITIL-306 in adult patients with solid tumors whose disease has relapsed or is refractory to standard therapies.

FIG. 18 illustrates the CoStAR platform engineered to enhance TIL functional activity. Similar to unmodified T cells, CoStAR TILs require specific antigen recognition via the native TCR for activation. CoStAR ligand engagement also provides T cells with synthetic costimulatory signals critical for mounting effective antitumor responses. Therefore, CoStAR-expressing T cells supplement tumor-specific antigen recognition via TCR with a robust costimulatory signal delivered via CoStAR.

FIG. 19 depicts a schematic for some embodiments of administering some FRα CoStARs. FIG. 19 describes ITIL-306-201, a phase 1a/1b dose escalation and expansion study evaluating the safety and feasibility of ITIL-306 in adult patients with advanced EOC, NSCLC, and RCC who relapsed from or are refractory to ≥1 prior line of systemic standard-of-care therapy.

FIGS. 20A-20D illustrates a schematic showing some embodiments of a FRα CoStAR and/or a fusion protein. Some general embodiments are depicted in FIG. 20A. FIG. 20B depicts some embodiments of a FRα CoStAR or a fusion protein. FIG. 20C depicts some embodiments of a CoStAR or a fusion protein. FIG. 20D depicts some embodiments of a CoStAR or a fusion protein. In some embodiments, the sequences describe the structure in its entirety and no further functional aspects are required to describe the CoStAR or fusion protein.

FIGS. 21A-21D illustrates a schematic showing some embodiments of an anti-pembrolizumab CoStAR and/or a fusion protein. Some general embodiments are depicted in FIG. 20A. FIG. 20B depicts some embodiments of an anti-pembrolizumab CoStAR or a fusion protein. FIG. 20C depicts some embodiments of a CoStAR or a fusion protein. FIG. 20D depicts some embodiments of a CoStAR or a fusion protein. In some embodiments, the sequences describe the structure in its entirety and no further functional aspects are required to describe the CoStAR or fusion protein.

FIGS. 22A-22D illustrates a schematic showing some embodiments of a CEA CoStAR and/or a fusion protein. Some general embodiments are depicted in FIG. 22A. FIG. 22B depicts some embodiments of a CEA CoStAR or a fusion protein. FIG. 22C depicts some embodiments of a CoStAR or a fusion protein. FIG. 22D depicts some embodiments of a CoStAR or a fusion protein. In some embodiments, the sequences describe the structure in its entirety and no further functional aspects are required to describe the CoStAR or fusion protein.

FIGS. 23A-23D illustrates a schematic showing some embodiments of a CEA CoStAR and/or a fusion protein. Some general embodiments are depicted in FIG. 23A. FIG. 23B depicts some embodiments of a MSLN CoStAR or a fusion protein. FIG. 23C depicts some embodiments of a CoStAR or a fusion protein. FIG. 23D depicts some embodiments of a CoStAR or a fusion protein. In some embodiments, the sequences describe the structure in its entirety and no further functional aspects are required to describe the CoStAR or fusion protein.

FIG. 24 depicts a schematic of the CD40 signaling domain and indicates regions bound by TRAF and Jak proteins.

FIG. 25 depicts the results of a proliferation assay where the clinical CoStAR construct MFE23.CD28.CD40 was compared to various TRAF binding domain mutants. In the assay, fold expansion was assessed over a 15 day period, with tumor challenges occurring at days 0 and 7 at an E:T ratio of 8:1.

FIG. 26 depicts schematics for the clinical anti-FRα construct CTP205, TRAF binding mutants of the clinical anti-FRα construct CTP338-CTP341, anti-FRα CD40 control CTP342, anti-FRα CD28 control CTP343, anti-CD19 CD28.CD40 control CTP357, anti-CD19 HLA-A2 control CTP358, and anti-FRα HLA-A2 control CTP359.

FIG. 27 depicts a schematic for a method of manufacturing CoStAR expressing cells from frozen healthy donor peripheral blood pan-T cells.

FIG. 28 depicts the results of an assessment of transduction rate in CoStAR expressing cells on day 12 post activation, prior to enrichment. Transduction rate in CD3, CD4, and CD8 T cells was measured for the constructs in FIG. 26

FIG. 29 depicts the results of an assessment of transduction rate in CoStAR expressing cells on day 14 post T cell activation, 24 hours after protein-Fc enrichment for positive/unsorted and negative fractions. Transduction rate in CD3 T cells was measured for the constructs in FIG. 3.

FIG. 30 depicts vitality and absolute cell counts of T cells transduced with the CoStAR constructs of FIG. 3 on day 12 after positive/negative enrichment and rapid expansion protocol (REP).

FIG. 31 depicts transduction rates of T cells transduced with the CoStAR constructs of FIG. 3 on day 12 after positive/negative enrichment and rapid expansion protocol (REP).

FIG. 32 depicts transduction rates of T cells transduced with the CTP 342, CTP357, and CTP358 CoStAR constructs of FIG. 3 on day 9 following REP of the negative fractions.

FIG. 33A-33D depict the results of a serial stimulation assay where the clinical anti-FRα construct CTP205 was compared to CD19 controls. An effector to target (E:T) ratio of 8:1 was used where the targets were BAF3.0KT3.FRα cells. No exogenous IL-2 was added in the experiment, and targets were added every 6-7 days. FIG. 33A depicts a schematic of the CoStAR constructs evaluated in this experiment and includes CD3 cell transduction rate at day 0 for cells from four donors. Fold expansion and CD4/CD8 ratios for CoStAR expressing cells from donor 02 and 1C are shown in FIG. 33B, and results for donor 05 and 2C are shown in FIG. 33C. FIG. 33D shows the results for T cell phenotype for donor 02, 1C, 05, 2C.

FIG. 34A-34D depict the results of a serial stimulation assay where the clinical anti-FRα construct CTP205 was compared to CD28 and HLA-A2 controls. An effector to target (E:T) ratio of 8:1 was used where the targets were BAF3.0KT3.FRα cells. No exogenous IL-2 was added in the experiment, and targets were added every 6-7 days. FIG. 34A depicts a schematic of the CoStAR constructs evaluated in this experiment. Fold expansion and CD4/CD8 ratios for CoStAR expressing cells from donor 02 and 1C are shown in FIG. 34B, and results for donor 05 and 2C are shown in FIG. 34C. FIG. 34D shows the results for T cell phenotype for donor 02, 1C, 05, 2C.

FIG. 35A-35D depict the results of a serial stimulation assay where the clinical anti-FRα construct CTP205 was compared to CD40 variants with TRAF binding mutations. An effector to target (E:T) ratio of 8:1 was used where the targets were BAF3.0KT3.FRα cells. No exogenous IL-2 was added in the experiment, and targets were added every 6-7 days. FIG. 35A depicts a schematic of the CoStAR constructs evaluated in this experiment. Fold expansion and CD4/CD8 ratios for CoStAR expressing cells from donor 02 and 1C are shown in FIG. 35B, and results for donor 05 and 2C are shown in FIG. 35C.

FIG. 35D shows the results for T cell phenotype for donor 02, 1C, 05, 2C.

FIG. 36A-36D depict the exhaustion profile of T cells transduced with the CoStARs shown in FIG. 36A. PD-1 (FIG. 36B), LAG3 (FIG. 36C), TIM3 (FIG. 36D) expression are shown at day 0, 14, and 21.

FIG. 37 depicts expression of MART-1 TCR and FRα CoStAR in healthy donor T cells from three donors compared to non-transduced T cells (top panel). The percentage of CD4/CD8 subsets of MART-1 TCR, FRα CoStAR, and non-transduced cells is depicted for three T cell donors (bottom panel).

FIG. 38 depicts assays of T2 target cells loaded with exogenous peptides (FAT, ELA, ELT, ALG) with or without FRα expression and incubated with donor T cells transduced with MART-1 TCR and/or FRα CoStAR. Magnitude of T cell response was determined by interferon gamma (IFNγ) secretion.

FIG. 39 depicts a comparison of the EC50 values between TCR.CoStAR-Td and TCR-Td T cells for experiments similar to those conducted in FIG. 38 where IFNγ, IL-2, and TNFα were the measured analytes.

FIG. 40A-40B depict assessment of the FRα CoStAR on T cell cytokine secretion and cytotoxicity. FIG. 40A depicts a schematic of the FRα CoStAR and secretion of IFNγ, TNFα, and IL-2 from T cells+/−FRα expression following incubation with BAF/3 target cells+/−expression of OKT3 and FRα. FIG. 40B depicts an experiment with similar conditions to FIG. 40A where cytotoxicity of T cells+/−FRα expression is assessed against BAF/3 target cells+/−expression of OKT3 and FRα. Fold expansion of T cells with or without FRα expression was also assessed over a 100 day period

FIG. 41 depicts fold expansion of T cells+/−FRα CoStAR expression upon single (30 day period) or serial (100 day period) stimulation. FIG. 41 also depicts the CD4/CD8 composition and the immune phenotype of CD4 and CD8 T cells in T cells+/−FRα CoStAR expression at days 0 and 10. Additionally, FIG. 41 depicts the expression of PD1 in CD4 and CD8 subsets in T cells+/−FRα CoStAR expression at days 0 and 10.

FIG. 42A-42D depict FRα expression among K562 cell lines and IL-2 expression by T cells+/−FRα CoStAR when cultured with target cells+/−OKT3 expression or soluble FRα. FIG. 42B-42D depict an assay where T cells with or without CoStAR transduction were incubated with K562 cell lines expressing variable levels of FRα and +/−OKT3 expression. Secretion of IFNγ (42B), IL-2 (42C), TNFα (42D) were assessed to evaluate T cell functional avidity.

FIG. 43A-43B depict a study to evaluate the effect of CoStAR expression on survival, T cell persistence and tumor control in a murine xenograft model even in the absence of exogenous IL2 infusions. FIG. 43A depicts the experimental plan, and results for the tumor volume and % survival assessments conducted over an 80-day period post T cell injection. FIG. 43B depicts the assessments of tumor volume, percent survival, and expansion at day 14 for nontransduced, TCR td, CoStAR td, and TCR.CoStAR td+/−IL-2.

FIG. 44A-44B depict evaluation of CoStAR expression in TILs and assessment of effect on effector T cell functions against autologous tumors. FIG. 44A depicts assessment of CoStARs on CD3 cells and CD8 subtypes of ovarian cancer, renal cancer, and non-small cell lung cancer infiltrating lymphocytes. Cytokine secretion by TILs and CoStAR expressing TILs was evaluated for TILs+/−BAF/3 cells (FIG. 44A-44B) for IFNγ (FIG. 47A) and IL-2 (FIG. 44B). Additionally, FIG. 44B depicts TIL CoStAR IFNγ response to autologous tumor digest cells.

FIG. 45 depicts a flow diagram of starting material procurement for ITIL-306 manufacturing.

FIG. 46 depicts a flow diagram of ITIL-306 manufacturing processes.

FIG. 47 depicts a flow diagram for the lentivirus manufacturing processes and genetic elements.

FIG. 48 depicts the lentivirus release testing procedures and additional characterization tests.

FIG. 49 depicts the steps of the lentivirus stability program.

FIG. 50 depicts a summary of the transduction, purity, viability, dose, VCN, and potency data of the ITIL-306 product across four different production runs.

FIG. 51 depicts a summary of potency data for ITIL-306 across three different production runs, where potency was assessed by detection of a degranulation marker, CD107a, and activation marker, interferon-gamma (IFN-γ), by flow cytometry.

FIG. 52 depicts transduction results of NK cells using lentivirus from four different lots of INIL-306.

FIG. 53 depicts a flow chart of the processes undertaken to reduce impurities during the ITIL-306 manufacturing process.

FIG. 54 depicts binding and fitting curves between MOV19 anti-FRα antibody and human histidine-tagged FRα.

FIG. 55 depicts measurement of FRα expression across various tumor types as measured by IHC and reported as H score or % positive cells.

FIG. 56 depicts T cell infiltration in tumor and adjacent normal tissue samples as measured by detection CD3E expression.

FIG. 57 depicts the results of a prescreen to determine the level of background binding of the test antibody to non-transfected and FRα overexpressing HEK293 cells. The test antibody was screened for binding against fixed HEK293 cells overexpressing the protein library to identify hits. All library hits were re-expressed, and probed with the test antibody or control treatments, to determine which hit(s), if any, were repeatable and specific to the test antibody.

FIG. 58 depicts the results of surface expression and vector copy number assessment of anti-FRα CoStAR-transduced healthy donor T cells and ovarian TILs. Where anti-FRα CoStAR expression levels were measured via flow cytometry utilizing soluble FRα fused to Fc tag (sFRα-Fc) followed by a secondary antibody staining and vector copy number was measured via ddPCR using primers specific against the anti-FRα CoStAR transgene.

FIG. 59 depicts cytolytic activity of anti-FRα CoStAR-transduced healthy donor T cells and ovarian TILs against BA/F3 target cells. Anti-FRα CoStAR-transduced T cells and TILs were cocultured with either wildtype BA/F3 (no stimulation), BA/F3-OKT3 (signal 1 alone), BA/F3-FRα (signal 2 alone), or BA/F3-OKT3-FRα (signal 1+2) target cells. Twenty-four hours post coculture, supernatants were analyzed by flow cytometry for cellular cytotoxicity and proliferation.

FIG. 60 depicts IL-2 secretion activity of anti-FRα CoStAR-transduced healthy donor T cells and ovarian TILs against BA/F3 target cells. Anti-FRα CoStAR-transduced T cells and TILs were cocultured with either wildtype BA/F3 (no stimulation), BA/F3-OKT3 (signal 1 alone), BA/F3-FRα (signal 2 alone), or BA/F3-OKT3-FRα (signal 1+2) target cells. Twenty-four hours post coculture, supernatants were analyzed by V-PLEX Proinflammatory Panel 1 Human Kit from MesoScale Discovery (MSD) for cytokine production.

FIG. 61 depicts measurement of IFN-γ and TNF-α secretion in nontransduced and anti-FRα CoStAR ovarian cancer TILs against autologous tumor coculture (n=5) (panel A), and relative IFN-γ secretion in the presence of MHC Class I and Class II blocking antibodies (panel B)

FIG. 62 depicts the percentage of intracellular TNF-α within Anti-FRα CoStAR-transduced or nontransduced TIL populations (n=5).

FIG. 63 depicts comparative analysis of IFN-γ secretion levels of nontransduced and anti-FRα CoStAR ovarian TILs (n=5) against autologous tumor coculture or BA/F3 and BA/F3 OKT3-FRα stable cell lines.

FIG. 64A-64E depict an experiment where CoStAR is intimately tuned to synergise with signal 1 agonists even at low levels of FRα. FIG. 64A depicts analysis of FRα expression in tumor tissue, normal tissue, and K562 cells. FIG. 64B-64E depict the results of an experiment where healthy donor T cells were engineered with CoStAR and cocultured with the target lines before assessment of remaining target cells (FIG. 64B), and IFNγ (FIG. 64C), IL2 (FIG. 64D), and TNFα (FIG. 64E) in the supernatant.

FIG. 65 depicts an experiment where CoStAR engagement enhances subsequent stimulation to signal 1 agonism. FIG. 65 depicts a schematic of a serial stimulation assay using Ba/F3 cells engineered with either OKT3 (Signal 1) and/or FRα (Signal 2), to recapitulate scenarios in which CoStAR cells may encounter tumour and normal tissue in sequence.

FIG. 66 depicts an experiment where CoStAR engagement enhances subsequent stimulation to signal 1. FIG. 66 depicts the experiment of FIG. 65, where Healthy donor T cells engineered with CoStAR were cocultured with the indicated Ba/F3 cells presenting either signal 1, alone, signal 2 alone, both or neither. After 7 days the T cells were restimulated with additional Ba/F3 cells before analysis of cytokines.

FIG. 67A-67C depict an experiment where CoStAR enhances the maximum responsiveness of T cells to any given defined pMHC agonist in a dose dependent manner. Healthy donor T cells were singly or co-transduced with an HLA-A*02 Melan-A/MART-1 specific TCR and FRα specific CoStAR. HLA-A*02+T2 were transduced with FRα or left non-transduced (FIG. 67A-67B). FIG. 67C depicts an experiment where T2-FRα were then pulsed with Melan-A/MART-1 heteroclitic (ELAGIGILTV 17 μM) or altered peptide ligands of varying antigenicity FATGIGIITV (3 μM), ELTGIGILTV (82 μM) and ALGIGILTV (very low affinity) (10,11) and cytokine secretion measured after 20 h coculture.

FIG. 68 depicts an experiment where CoStAR does not affect the overall antigen threshold (EC50) of T cells stimulated through pMHC. FIG. 68 depicts the results of FIG. 67A-C where EC50 values nonlinear regression curves were fitted with a log(agonist) versus response (3 parameters) model to calculate LogEC50 fits. Comparisons of best fit LogEC50 values were calculated. Log EC50 values from 3 donors were analyzed by Friedman statistical test with Dunn's multiple comparisons.

FIG. 69 depicts an experiment where CoStAR-TILs transduced with ITIL-306 were incubated with matching autologous tumor from NSCLC, RCC, and ovarian patients and anti-tumor activity was evaluated by assessing IFNγ secretion.

FIG. 70A-70F depict a proliferation assay of 6 different CoStAR constructs, where healthy donor (HD) T cells from four different donors were modified with the CoStAR constructs and cocultured with target cells+/−OKT3, and an E:T ratio of less than 8 was maintained. Cells were cultured+/−IL-2 for a period of 21 days and proliferation was assessed by measuring CD2 live cell counts at days 0, 7, 14, and 21 compared to nontransduced controls. FIG. 70A depicts the results for FRα CoStAR (CTP205). FIG. 70B depicts the results for CEA CoStAR (CTP194). FIG. 70C depicts the results for MSLN CoStAR (CTP224). FIG. 70D depicts the results for FRα CoStAR (C7, CTP132). FIG. 70E depicts the results for CA125 CoStAR (CTP111). FIG. 70F depicts the results for CD228 CoStAR (CTP175).

FIG. 71A-71F depict a proliferation assay of 6 different CoStAR constructs, where healthy donor (HD) T cells from four different donors were modified with the CoStAR constructs and cocultured with target cells+OKT3, and an E:T ratio of less than 8 was maintained. Cells were cultured+/−IL-2 for a period of 21 days and proliferation was assessed by measuring CD2 live cell counts at days 0, 7, 14, and 21 compared to nontransduced controls. FIG. 71A depicts the results for FRα CoStAR (CTP205). FIG. 71B depicts the results for CEA CoStAR (CTP194). FIG. 71C depicts the results for MSLN CoStAR (CTP224). FIG. 71D depicts the results for FRα CoStAR (C7, CTP132). FIG. 71E depicts the results for CA125 CoStAR (CTP111). FIG. 71F depicts the results for CD228 CoStAR (CTP175).

FIG. 72 illustrates a schematic for some embodiments of administering FRα CoStAR expressing cells. The schematic in FIG. 72 depicts a process for an ITIL-306-201 study, which includes Dose Escalation and Expansion phases and Screening, Enrollment/Tumor Resection, Lymphodepleting Chemotherapy, ITIL-306 Infusion without IL-2, and Post Treatment Assessment steps.

FIG. 73 depicts the oncology diagnosis history, systemic oncology therapy history, and oncology radiation therapy history of Patient 1 enrolled in the ITIL-306-201 study.

FIG. 74 depicts the viability and product overview of CoStAR transduced T cells ITIL-306-201 (30622001) generated from Patient 1.

FIG. 75A depicts the leukocyte composition of the final 30622001 product in CoStAR+ and CoStAR− populations. FIG. 75B depicts the gamma delta (γδ) TCR distribution in the final product and the percent of CoStAR positive cells in the γδ+, γδ-, and CD3+ cell populations.

FIG. 76 depicts cytokine production analyzed following autologous coculture by V-PLEX Proinflammatory Panel 1 Human Kit from MesoScale Discovery (MSD) for TILs alone, transduced TILs alone (TD), and TIL+TD derived from Patient 1.

FIG. 77 depicts results from blood testing of Patient 1 the ITIL-306-201 study from initial screening to Day 28.

FIG. 78A depicts lymphocyte count for Patient 1 during the ITIL-306-201 study from seven days prior to infusion out to 91 days post infusion. FIG. 78B depicts peripheral blood cell count for Patient 1 during the ITIL-306 201 study from eight days prior to infusion to nine days post infusion.

FIG. 79A depicts an assessment of CoStAR transgene copy number for Patient 1 during the ITIL-306-201 study from five days prior to infusion out to 28 days post infusion as measured by droplet digital (dd)PCR. FIG. 79B depicts an assessment of the number of CoStAR positive cells per microliter of blood for Patient 1 during the ITIL-306-201 study from five days prior to infusion out to 28 days post infusion.

FIG. 80 depicts an assessment of serum levels of IL-15 during the ITIL-306-201 study from enrollment prior to infusion out to 28 days post infusion.

FIG. 81 depicts an assessment of the concentration of IL-7 (left panel) and IL-15 (right panel) for Patient 1 in ITIL-306-201 compared to the IL-7 and IL-15 levels of six patients in ITIL-168-101.

FIG. 82 depicts an assessment of the persistence of product related clones in ITIL-306 201 and ITIL-168-101 as evidenced by measurement of the fraction of PBMCs TCR beta clones observed.

FIG. 83 depicts an assessment of changes in tumor size from baseline for Patient 1 during the ITIL-306-201 study from Day 0 out to approximately 180 days post infusion.

FIG. 84 illustrates CT scan images of the mediastinal lymph node of Patient 1 showing a 17% reduction in size following the ITIL-306-201 study.

FIG. 85 depicts an assessment of healthy donor T cell expression of CoStAR with common CD28.CD40 intracellular signalling domains targeted toward several antigens via different scFv regions. Flow cytometric analysis of CoStAR construct expression was performed upon live CD3+ T cells, n=4 individual donors.

FIG. 86A depicts an experimental schema evaluating the dependence on intracellular signalling domains, rather than scFv region and tumour associated antigen target, of CoStAR enhancement of cytokine secretion by T cells.

FIG. 86B depicts an assessment of the dependency of CoStAR enhancement of T cell TNFα secretion on intracellular signalling domains. Enhancement was observed against several distinct tumour associated antigen targets and was not dependent on IL-2 supplementation. Assessment was performed by assessed by MSD immunoassay, n=4 individual donors; statistical test, two-way ANOVA with Šidák's test for multiple comparisons.

FIG. 86C depicts an assessment of the dependency of CoStAR enhancement of T cell IL-2 secretion on intracellular signalling domains. Enhancement was observed against several distinct tumour associated antigen targets and was not dependent on IL-2 supplementation. Assessment was performed by assessed by MSD immunoassay, n=4 individual donors; statistical test, two-way ANOVA with Šidák's test for multiple comparisons.

FIG. 86D depicts an assessment of the dependency of CoStAR enhancement of T cell IFNγ secretion on intracellular signalling domains. Enhancement was observed against several distinct tumour associated antigen targets and was not dependent on IL-2 supplementation. Assessment was performed by assessed by MSD immunoassay, n=4 individual donors; statistical test, two-way ANOVA with Šidák's test for multiple comparisons.

FIG. 87A depicts an experimental schema evaluating the dependence on intracellular signalling domains, rather than scFv region and tumour associated antigen target, of CoStAR enhancement of T cell proliferation.

FIG. 87B-87C depict an assessment of the dependency of CoStAR enhancement of T cell proliferation on intracellular signalling domains. Enhancement was observed against several distinct tumour associated antigen targets and was not dependent on IL-2 supplementation. Assessment was performed by flow cytometric cell counting of live CD2+ cell counts. In FIG. 87C, CoStAR transduced T cells had their live CD2+ counts normalised to their CoStAR construct frequency on CD3+ T cells as determined by flow cytometric analysis, n=4 individual donors.

### DETAILED DESCRIPTION

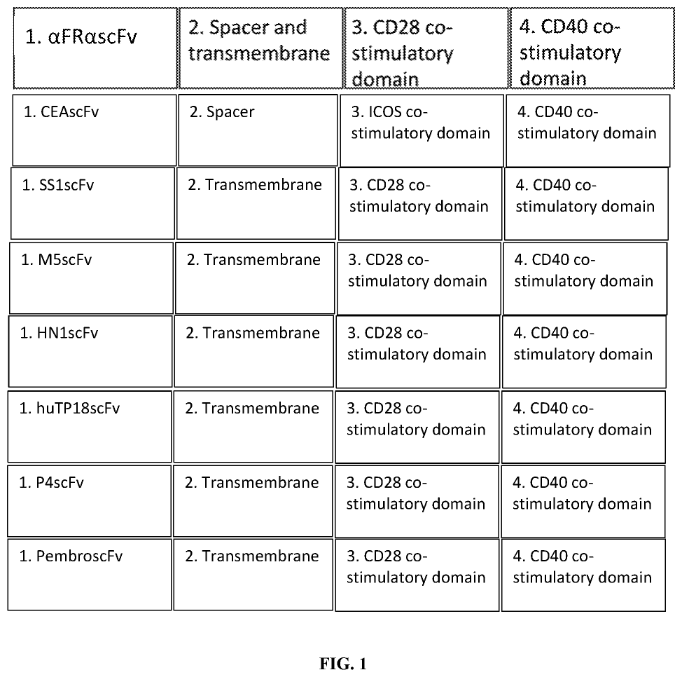

Provided herein are methods of cell therapy treatment that involve no or reduced levels of IL-2 being administered to a subject for the in vivo component of the cell therapy treatment. It has been discovered that the use of various fusion constructs, such as CoSTaRs, surprisingly allows for one to avoid or minimize the administration of IL-2 to the subject. That is, the use of various fusion constructs (such as that depicted in FIG. 1), allows for cell stimulation during cell therapy, independent of IL-2. For any of the appropriate methods provided herein, the FOLR1 (FRα) scFv (in FIG. 1) can be replaced with a pembrolizumab scFv or a CEA scFv (or other binding domain, such as those binding domains described herein that bind to various TAAs).

In some embodiments, a method of treating cancer in a subject that expresses folate receptor alpha 1 (aka “FOLR1”), alternatively referred to as “FR-alpha”, or FRα is provided. The method comprises identifying a subject. The subject has a cancer that expresses FRα and administering to the subject a cell comprising a fusion protein. The fusion protein comprises a binding domain specific for FRα linked to a transmembrane domain that is linked to a CD28 signaling domain that is linked to a CD40 signaling domain. The subject does not receive exogenous IL-2 in a manner that is adequate for cell stimulation of TILs in vivo. In the present disclosure, reference to a “FRα CoStAR” or similar phrase denotes a CoStAR that binds to folate receptor alpha 1. In the present disclosure, reference to a “FRα CoStAR” or similar phrase denotes a CoStAR that binds to FRα and/or a CoStAR construct that contains a sequence of a binding domain for FRα.

In some embodiments, the method comprises a cell expressing a fusion protein. In some embodiments, the cell can possess cytotoxic ability. In some embodiments, the cell can be provided co-stimulation (Signal 2) by the fusion protein upon recognition of FRα. In some embodiments, the cell receives proliferation and survival signals from the fusion protein upon activation of the fusion protein. In some embodiments, the cell is an immune cell. In some embodiments, the cell is a T cell including an αβ T cell, a γδ T cell, or an NK T cell. In some embodiments, the cell is a tumor infiltrating lymphocyte (TIL). In some embodiments, the cell is or has been isolated from PBMCs. In some embodiments, the cell is an immune cell, T cell, PBMC, or TIL from an autologous donor.

In some embodiments, the fusion protein comprises: (i) an antigen binding domain (e.g., a tumor associated antigen binding domain), (ii) a first intracellular segment comprising signaling domain of a CD28 receptor protein or signal transducing fragment thereof, and (iii) a second intracellular signaling domain of a CD40 receptor protein or signal transducing fragment thereof. In some embodiments, the extracellular segment of the stimulatory receptor protein is capable of binding a ligand. In some embodiments, the ligand is folate receptor alpha 1 (aka FRα protein). In some embodiments, the fusion protein comprises an intervening transmembrane domain between the disease or tumor antigen binding domain and the first intracellular domain. In some embodiments, the primary costimulatory receptor can be less than a full-length protein but is sufficient to bind cognate ligand and transduce a signal. In some embodiments, selection of one or more costimulatory domain signaling component or motif is guided by the cell in which the fusion protein is to be expressed and/or a desired costimulatory activity more closely identified with a signaling component or motif, or avoidance of a costimulatory activity more closely identified with a signaling component or motif.

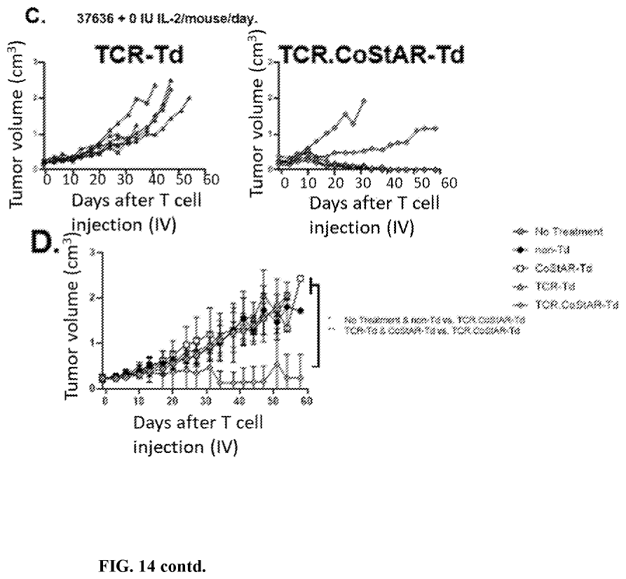

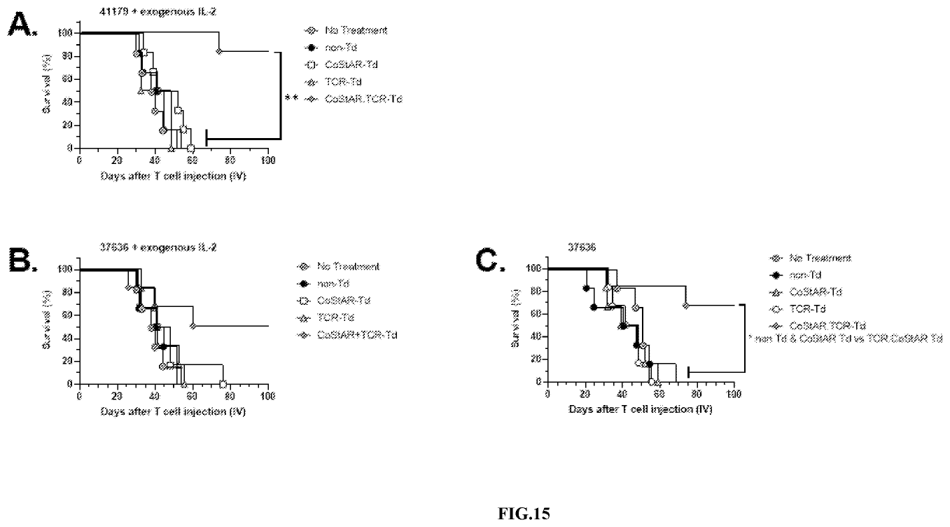

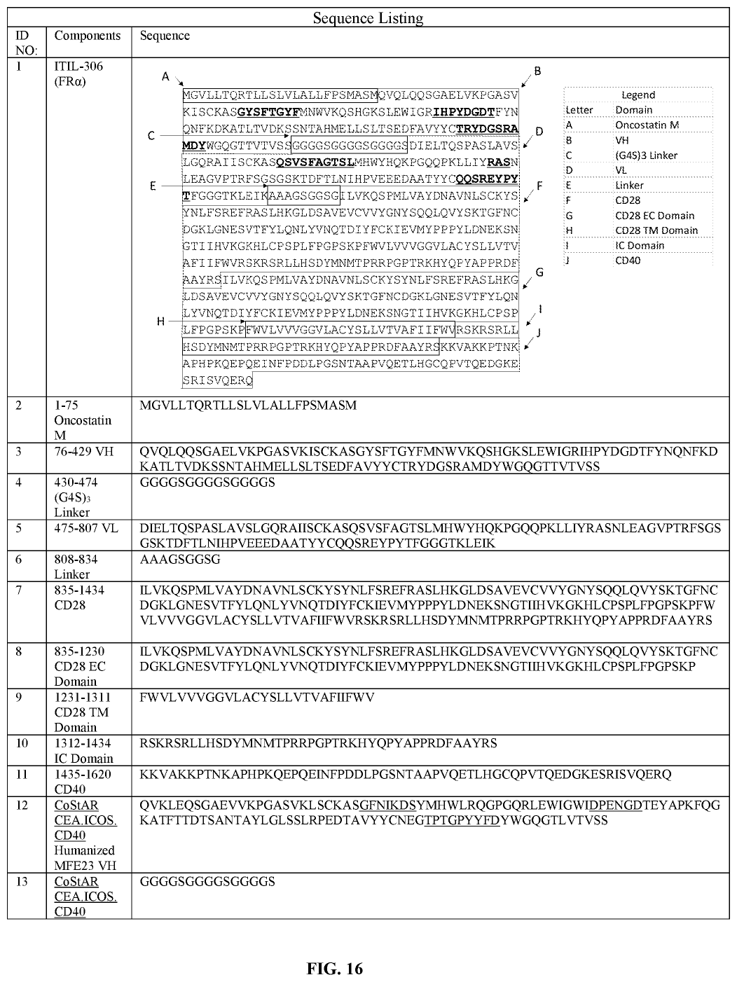

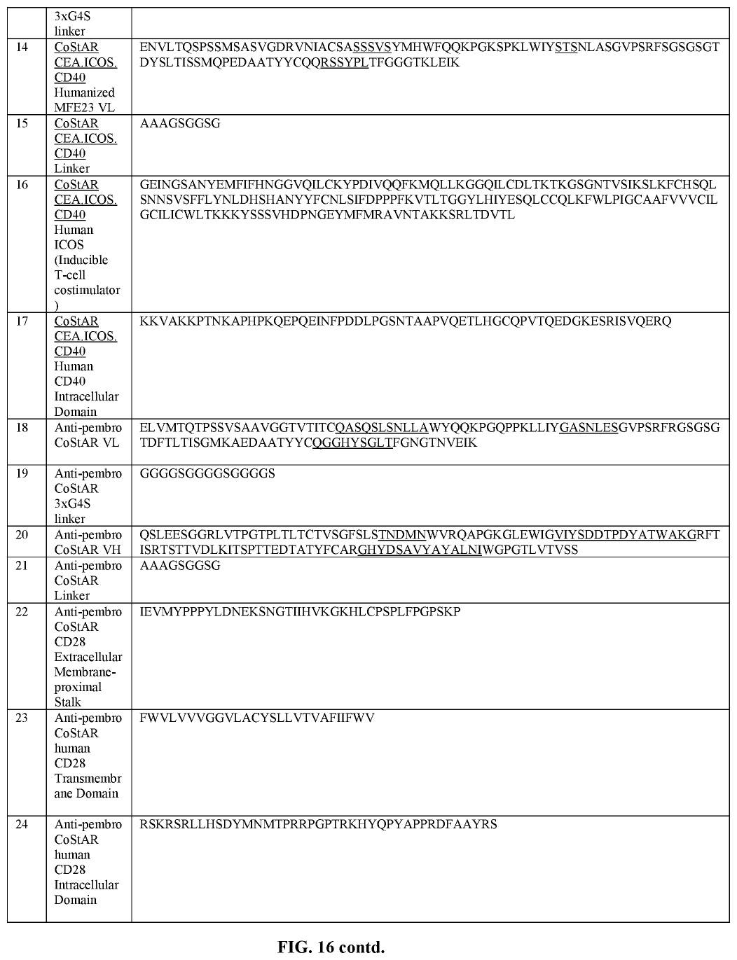

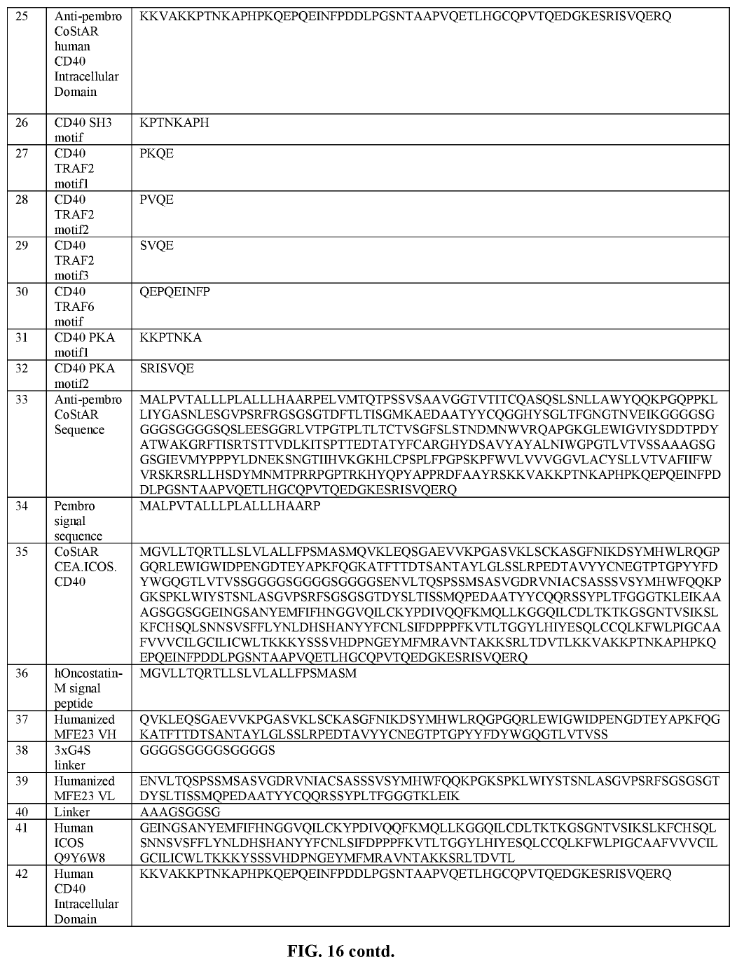

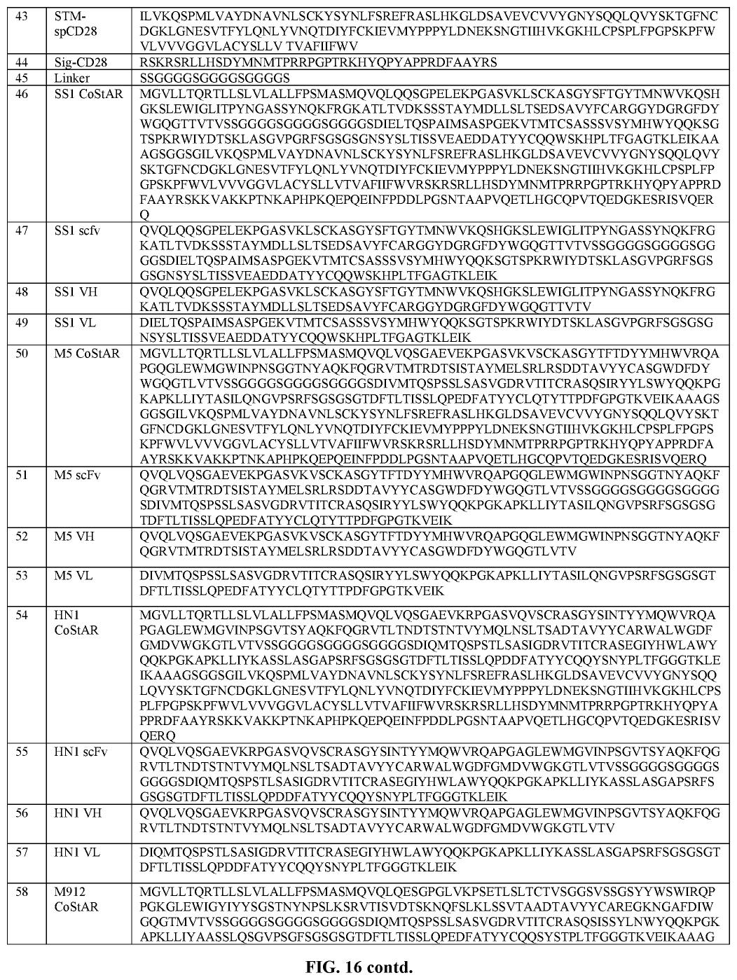

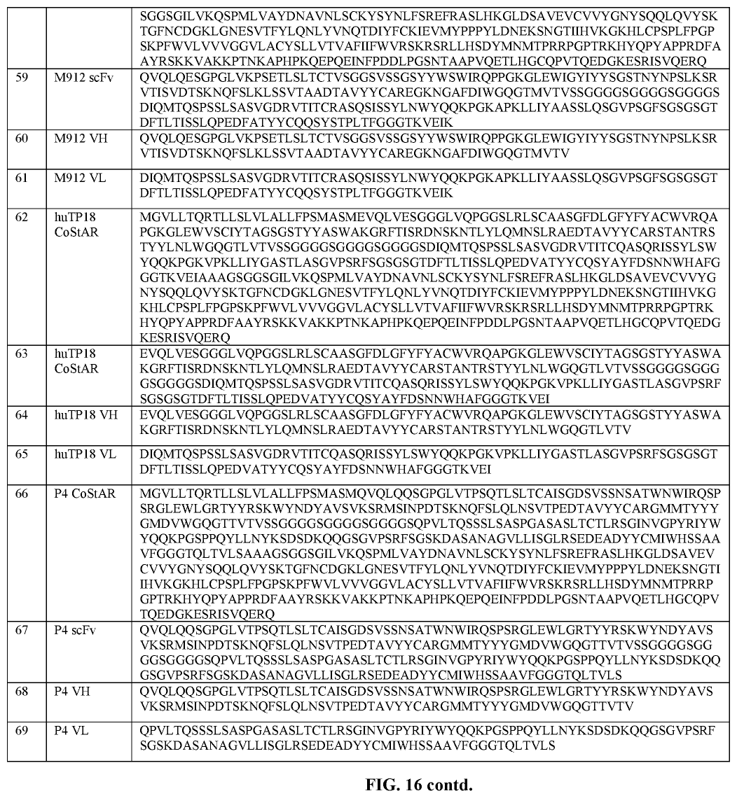

In some embodiments, the fusion protein extracellular domain comprises a linker. In some embodiments, linkers comprise short runs of amino acids used to connect domains, for example a binding domain with a spacer or transmembrane domain. In some embodiments, in order for there to be flexibility to bind ligand, a ligand binding domain will usually be connected to a spacer or a transmembrane domain by flexible linker comprising from about 5 to 25 amino acids, such as, for example, AAAGSGGSG (SEQ ID NO:), GGGGSGGGGSGGGGS (SEQ ID NO:4) where the sequences are shown in FIG. 16. In some embodiments, the fusion protein comprises a binding domain joined directly to a transmembrane domain by a linker, and without a spacer. In some embodiments, a fusion protein comprises a binding domain joined directly to a transmembrane by a spacer and without a linker. In some embodiments, the linker comprises one or more serine or glycine and/or alanine. In some embodiments, the linker is at least 50, 60, 70, 80, 90, 95, 98, 99 or 100% serine, glycine, and/or alanine.

In some embodiments the binding domain and transmembrane domain are linked directly. In some embodiments the binding domain and transmembrane domain are linked indirectly. In some embodiments the binding domain and transmembrane domain are linked covalently. In some embodiments the covalent linkage between binding domain and transmembrane domain is through the amino acid backbone. In some embodiments the covalent linkage between binding domain and transmembrane domain is through a disulfide bond. In some embodiments the covalent linkage between binding domain and transmembrane domain is through an amino acid backbone with an optional linker.

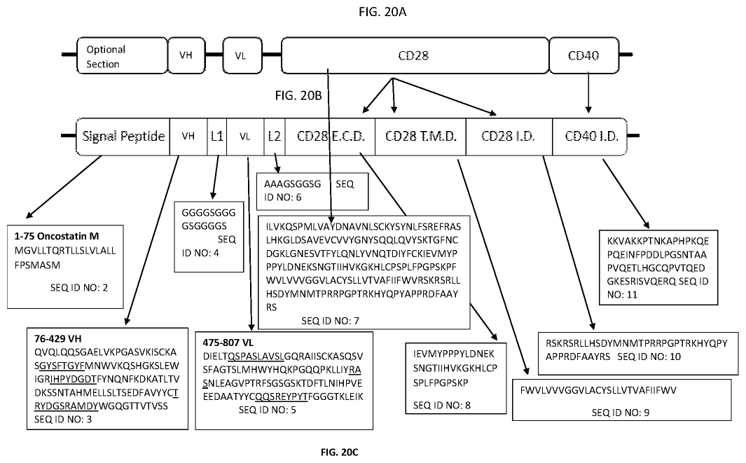

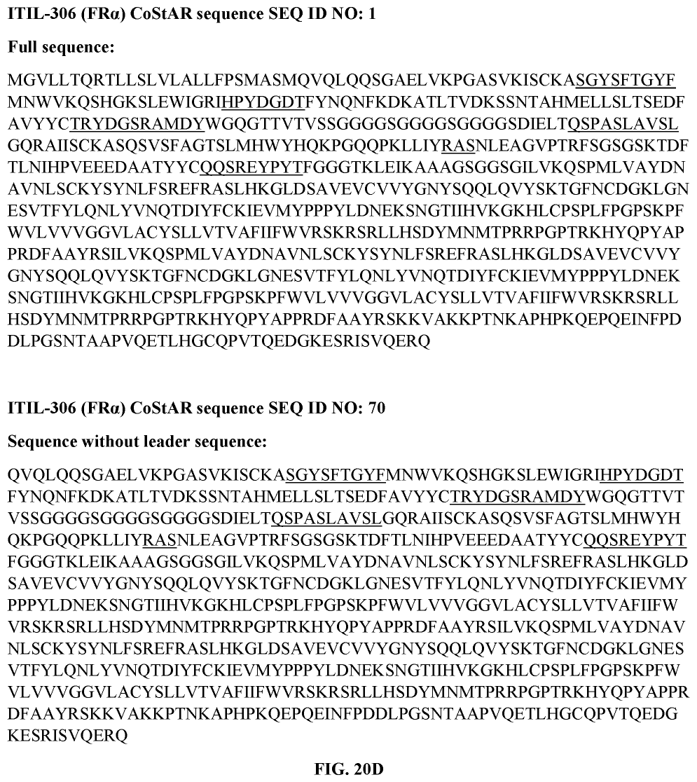

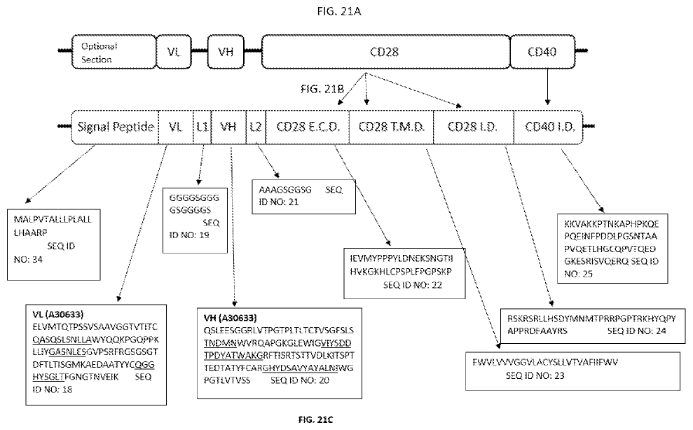

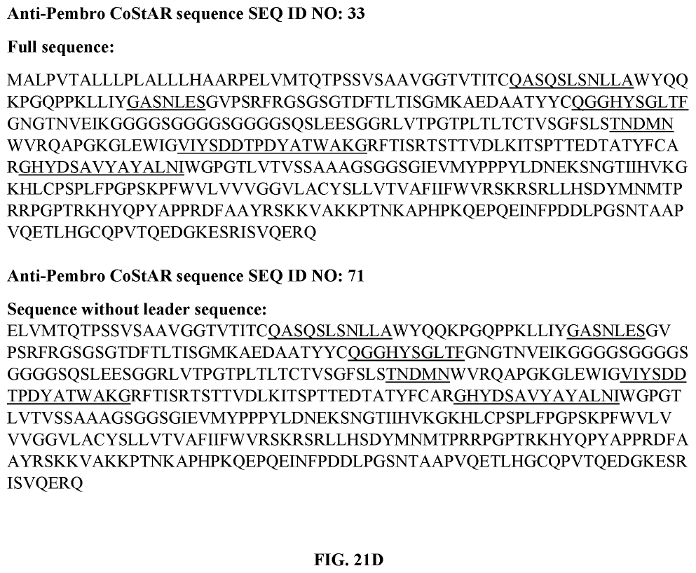

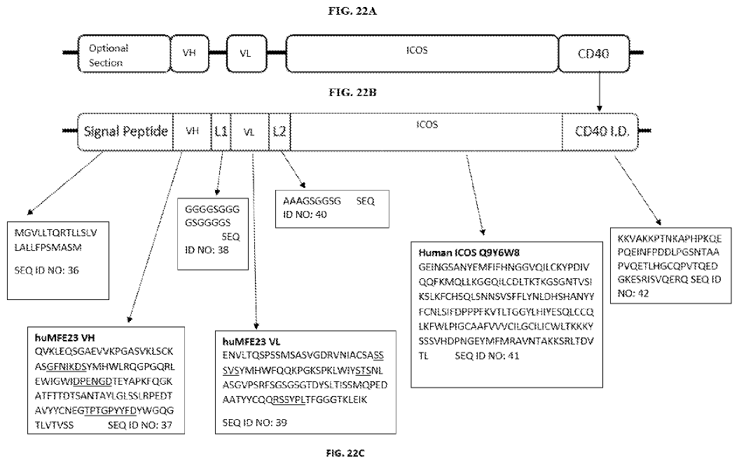

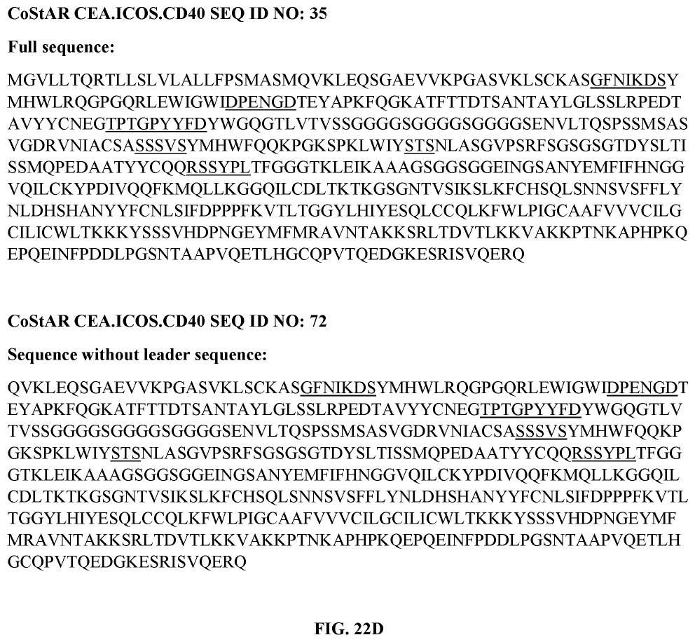

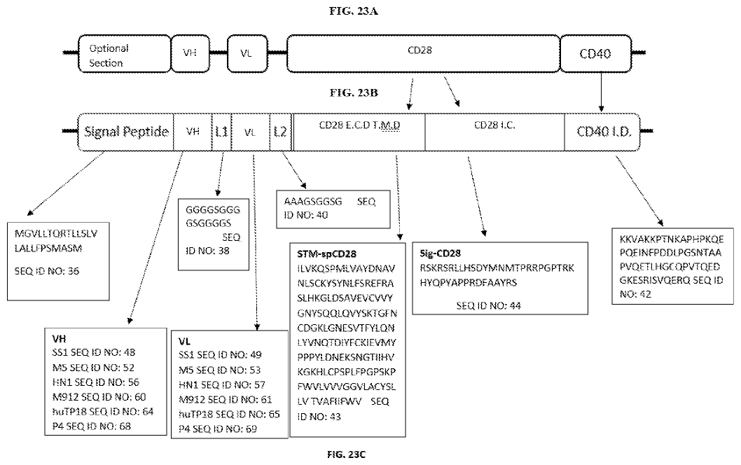

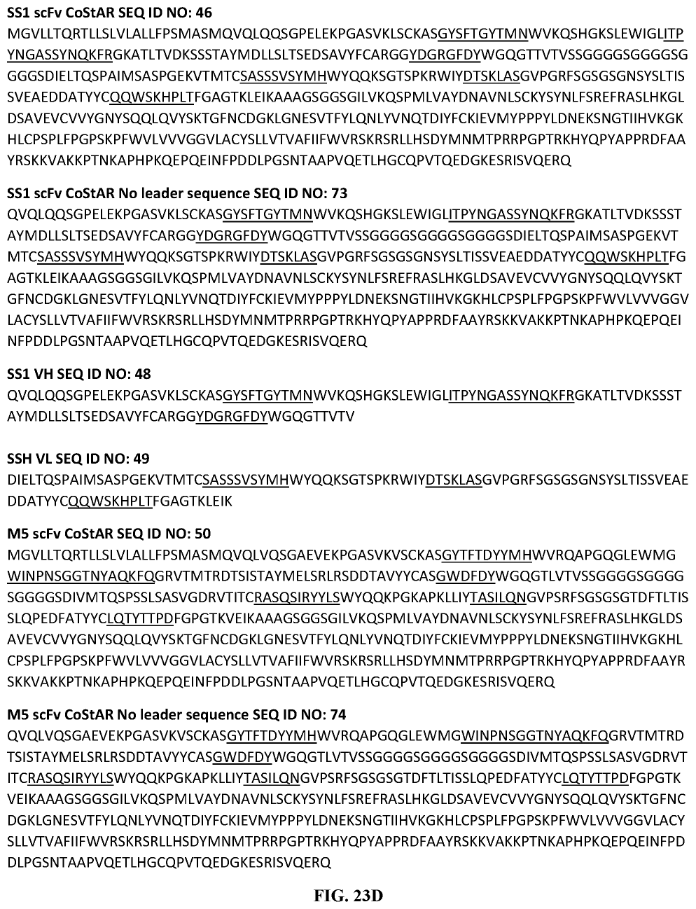

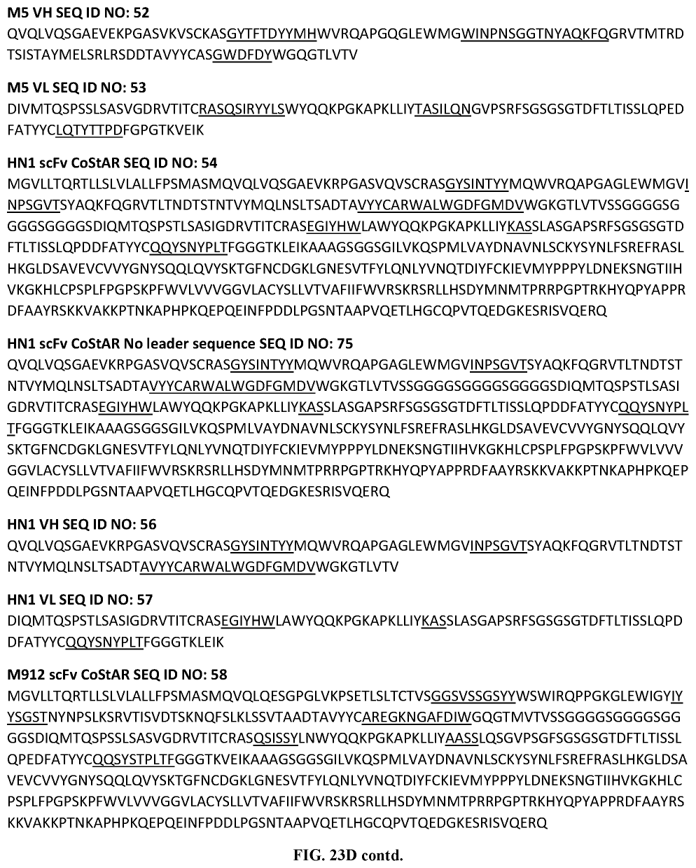

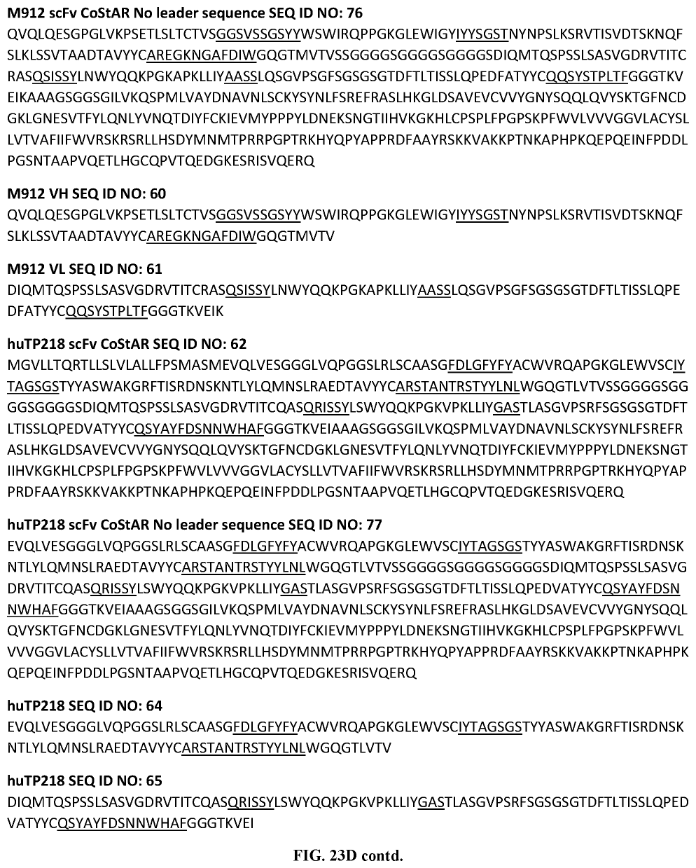

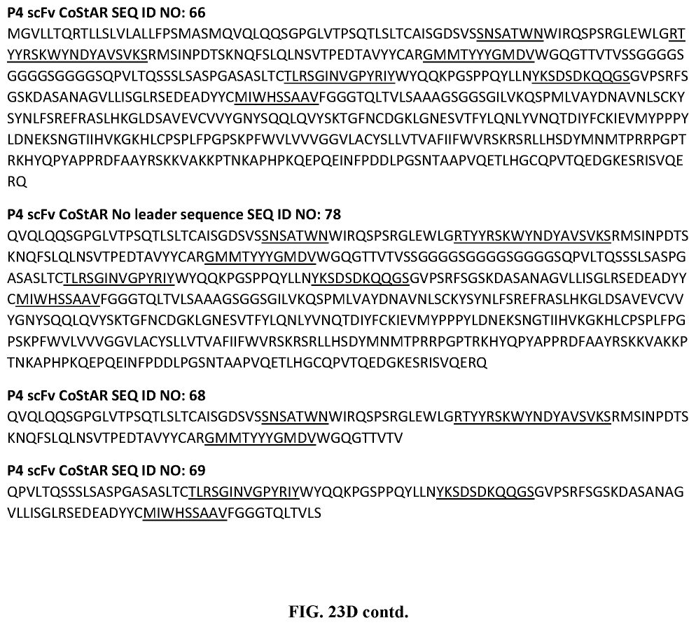

In some embodiments, the transmembrane anchors the CoStAR in the T cell membrane. In some embodiments, the transmembrane domain influences CoStAR function. In some embodiments, the transmembrane domain is comprised by the full length primary costimulatory receptor domain. In some embodiments wherein the CoStAR construct comprises an extracellular domain of one receptor and an intracellular signaling domain of a second receptor, the transmembrane domain can be that of the extracellular domain or the intracellular domain. In some embodiments, the transmembrane domain is from CD4, CD8a, CD28, or ICOS. Gueden et al. associated use of the ICOS transmembrane domain with increased CAR T cell persistence and overall anti-tumor efficacy (Guedan S. et al., Enhancing CAR T cell persistence through ICOS and 4-1BB costimulation. JCI Insight. 2018; 3:96976). In some embodiments, the transmembrane domain comprises a hydrophobic a helix that spans the cell membrane. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

In some embodiments, the chimeric costimulatory antigen receptor, and/or fusion protein provided herein can include or exclude a signal peptide (which may be cleaved upon processing within the cell), and thus, any of the embodiments including a signal peptide in any one or more of, for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN) are envisioned as including and also envisioned in an arrangement excluding the signal peptide, for all of the other embodiments and disclosures provided herein. Thus, an arrangement of FIG. 20D, lacking SEQ ID NO: 2, an arrangement of FIG. 21D, lacking SEQ ID NO: 34, an arrangement of FIG. 22D, lacking SEQ ID NO: 36, and an arrangement of FIG. 23D (SEQ ID NOs: 46, 50, 54, 58, 62, and 66), lacking SEQ ID NO: 36, are all envisioned as optional constructs for all of the embodiments and arrangements provided herein. Exemplary fusion protein sequences lacking the optional signal peptide are included in FIG. 20D (anti-FRα), FIG. 21D (anti-pembrolizumab), FIG. 22D (anti-CEA), and FIG. 23D (anti-MSLN). In some embodiments, the fusion protein comprises a) a binding domain specific for CEA linked to; b) a transmembrane domain that is linked to; c) a CD28 signaling domain linked to; d) a CD40 signaling domain.

In some embodiments, the transmembrane domain comprises amino acids of the CD28 transmembrane domain from about amino acid 153 to about amino acid 179. In some embodiments, the transmembrane domain comprises amino acids of the CD8 transmembrane domain from about amino acid 183 to about amino acid 203. In some embodiments, the CoStARs can include several amino acids between the transmembrane domain and signaling domain. For example, in some constructs described herein the link from a CD8 transmembrane domain to a signaling domain comprises several amino acids of the CD8 cytoplasmic domain (e.g., amino acids 204-210 of CD8).

In some embodiments, the CoStAR extracellular domain comprises a linker. Linkers comprise short runs of amino acids used to connect domains, for example a binding domain with a spacer or transmembrane domain. In order for there to be flexibility to bind ligand, a ligand binding domain will usually be connected to a spacer or a transmembrane domain by flexible linker comprising from about 5 to 25 amino acids, such as, for example, AAAGSGGSG (SEQ ID NO:7), GGGGSGGGGSGGGGS (SEQ ID NO:62). In some embodiments, a CoStAR comprises a binding domain joined directly to a transmembrane domain by a linker, and without a spacer. In some embodiments, a CoStAR comprises a binding domain joined directly to a transmembrane by a spacer and without a linker.

A CoStAR optionally comprises a spacer region between the antigen binding domain and the costimulatory receptor. As used herein, the term “spacer” refers to the extracellular structural region of a CoStAR that separates the antigen binding domain from the external ligand binding domain of the costimulatory protein. The spacer provides flexibility to access the targeted antigen and receptor ligand. In some embodiments long spacers are employed, for example to target membrane-proximal epitopes or glycosylated antigens (see Guest R. D. et al. The role of extracellular spacer regions in the optimal design of chimeric immune receptors: evaluation of four different scFvs and antigens. J. Immunother. 2005; 28:203-211; Wilkie S. et al., Retargeting of human T cells to tumor-associated MUC1: the evolution of a chimeric antigen receptor. J. Immunol. 2008; 180:4901-4909). In some embodiments, CoStARs bear short spacers, for example to target membrane distal epitopes (see Hudecek M. et al., Receptor affinity and extracellular domain modifications affect tumor recognition by ROR1-specific chimeric antigen receptor T cells. Clin. Cancer Res. 2013; 19:3153-3164; Hudecek M. et al., The nonsignalling extracellular spacer domain of chimeric antigen receptors is decisive for in vivo antitumor activity. Cancer Immunol. Res. 2015; 3:125-135). In some embodiments, the spacer comprises all or part of or is derived from an IgG hinge, including but not limited to IgG1, IgG2, or IgG4. By “derived from an Ig hinge” is meant a spacer comprising insertions, deletions, or mutations in an IgG hinge. In some embodiments, a spacer can comprise all or part of one or more antibody constant domains, such as but not limited to CH2 and/or CH3 domains. In some embodiments, in a spacer comprising all or part of a CH2 domain, the CH2 domain is modified so as not to bind to an Fc receptor. For example, Fc receptor binding in myeloid cells has been found to impair CAR T cell functionality. In some embodiments, the spacer comprises all or part of an Ig-like hinge from CD28, CD8, or other protein comprising a hinge region. In some embodiments, that comprise a spacer, the spacer is from 1 and 50 amino acids in length.

In some embodiments, the spacer comprises essentially all of an extracellular domain, for example a CD28 extracellular domain (e.g., from about amino acid 19, 20, 21, or 22 to about amino acid 152) or an extracellular domain of another protein, including but not limited to another TNFR superfamily member. In some embodiments, the spacer comprises a portion of an extracellular domain, for example a portion of a CD28 extracellular domain, and can lack all or most of the Ig domain. In some embodiments, the spacer includes amino acids of CD28 from about 141 to about 152 but not other portions of the CD28 extracellular domain. In some embodiments, the spacer includes amino acids of CD8 from about 128 to about 182 but not other portions of the CD8 extracellular domain.

In some embodiments, a CoStAR comprises a full length primary costimulatory receptor which can comprise an extracellular ligand binding and intracellular signaling portion of, without limitation, CD2, CD9, CD26, CD27, CD28, CD29, CD38, CD40, CD43, CD46, CD49d, CD55, CD73, CD81, CD82, CD99, CD100, CD134 (OX40), CD137 (4 IBB), CD 150 (SLAM), CD270 (HVEM), CD278 (ICOS), CD357 (GITR), or EphB6. In some embodiments, the costimulatory receptor comprises a chimeric protein, for instance comprising an extracellular ligand binding domain of one of the aforementioned proteins and an intracellular signaling domain of another of the aforementioned proteins. In some embodiments, the signaling portion of the CoStAR comprises a single signaling domain. In some embodiments, the signaling portion of the CoStAR comprises a second intracellular signaling domain such as but not limited to: CD2, CD27, CD28, CD40, CD134 (OX40), CD137 (4-1BB), CD150 (SLAM). In some embodiments, the first and second intracellular signaling domains are the same. In some embodiments, the first and second intracellular signaling domains are different. In some embodiments, the costimulatory receptor is capable of dimerization. Without being bound by theory, it is thought that CoStARs dimerize or associate with other accessory molecules for signal initiation. In some embodiments, CoStARs dimerize or associate with accessory molecules through transmembrane domain interactions. In some embodiments, dimerization or association with accessory molecules is assisted by costimulatory receptor interactions in the intracellular portion, and/or the extracellular portion of the costimulatory receptor.

In some embodiments, the binding domain allows targeting of the cancer treatment specifically to folate receptor alpha 1 expressing cancer cells (e.g., cells that have the FRα gene expressing). In some embodiments, the binding domain can comprise an scFv, a peptide, an antibody heavy-chain, a natural ligand, or a receptor specific for folate receptor alpha 1. In some embodiments, the binding domain can comprise a polypeptide comprising an scFv with the VH polypeptide comprising SEQ ID NO: 3, and the VL polypeptide sequence comprising SEQ ID NO: 5, where the sequence is shown in FIG. 16. In some embodiments, the binding domain can be linked to the transmembrane domain by a linker and/or a spacer. In some embodiments, the binding domain is that in SEQ ID NO: 1. In some embodiments, the binding domain is at least 70, 80, 90, 95, 96, 97, 98, 99 or 100% identical to that in SEQ ID NO: 1 (or any of the corresponding sequences for a different target in FIG. 16). In some embodiments, the binding domain comprises a VH and/or VL that is at least 70, 80, 90, 95, 96, 97, 98, 99 or 100% identical to the VH, and/or VL in SEQ ID NOs: 3 and 5 (or any of the corresponding sequences for a different target in FIG. 16, such as for pembrolizumab or CEA). In some embodiments, the binding domain comprises a HCDR1, HCDR2, HCDR3, LCDR1, LCDR2, and/or LCDR3 that is at least 70, 80, 90, or 100% identical to the HCDR1, HCDR2, HCDR3, LCDR1, LCDR2, and/or LCDR3 in SEQ ID NOs: 3 and 5 (or any of the corresponding sequences for a different target in FIG. 16, such as for pembrolizumab or CEA).

In some embodiments, the term “antigen binding domain” as used herein refers to an antibody fragment including, but not limited to, a diabody, a Fab, a Fab′, a F(ab′)2, an Fv fragment, a disulfide stabilized Fv fragment (dsFv), a (dsFv)2, a bispecific dsFv (dsFv-dsFv 1), a disulfide stabilized diabody (ds diabody), a single-chain antibody molecule (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen. In some embodiments, an antigen binding domain is capable of binding to the same antigen to which the parent antibody or a parent antibody fragment (e.g., a parent scFv) binds. In some embodiments, an antigen-binding fragment can comprise one or more complementarity determining regions (CDRs) from a particular human antibody grafted to frameworks (FRs) from one or more different human antibodies.

In some embodiments, the scFV comprises a VH and/or VL with at 70% identity to the polypeptides in SEQ ID NOs: 3 and 5. In some embodiments, the scFV comprises a VH and/or VL with at 75% identity to the polypeptides in SEQ ID NOs: 3 and 5. In some embodiments, the scFV comprises a VH and/or VL with at 80% identity to the polypeptides in SEQ ID NOs: 3 and 5. In some embodiments, the scFV comprises a VH and/or VL with at 85% identity to the polypeptides in SEQ ID NOs: 3 and 5. In some embodiments, the scFV comprises a VH and/or VL with at 90% identity to the polypeptides in SEQ ID NOs: 3 and 5. In some embodiments, the CDRs of SEQ ID NOs: 3 and 5 have 1 point mutation. In some embodiments, the CDRs of SEQ ID NOs: 3 and 5 have 2 point mutations. In some embodiments, the CDRs of SEQ ID NOs: 3 and 5 have 3, 4 or 5 point mutations. In some embodiments, the sequence(s) are those shown in FIG. 16 (e.g., 3 and 5 for FRα; 12 and 14 for CEA, and 18 and 20 for pembrolizumab). In some embodiments, the binding domain is defined by the amino acid structure alone, and can be any one of those sequences provided herein regarding such amino acid structures. It shall be appreciated that all embodiments disclosed herein regarding FRα also apply for the corresponding CEA and pembrolizumab arrangements in FIG. 16.

In some embodiments, the scFV comprises a VH and/or VL with at 70% identity to the polypeptides in SEQ ID NOs: 12 and 14. In some embodiments, the scFV comprises a VH and/or VL with at 75% identity to the polypeptides in SEQ ID NOs: 12 and 14. In some embodiments, the scFV comprises a VH and/or VL with at 80% identity to the polypeptides in SEQ ID NOs: 12 and 14. In some embodiments, the scFV comprises a VH and/or VL with at 85% identity to the polypeptides in SEQ ID NOs: 12 and 14. In some embodiments, the scFV comprises a VH and/or VL with at 90% identity to the polypeptides in SEQ ID NOs: 12 and 14. In some embodiments, the CDRs of SEQ ID NOs: 12 and 14 have 1 point mutation. In some embodiments, the CDRs of SEQ ID NOs: 12 and 14 have 2 point mutations. In some embodiments, the CDRs of SEQ ID NOs: 12 and 14 have 3, 4 or 5 point mutations. In some embodiments, the sequence(s) are those shown in FIG. 16. In some embodiments, the binding domain is defined by the amino acid structure alone, and can be any one of those sequences provided herein regarding such amino acid structures.

In some embodiments, the scFV comprises a VH and/or VL with at 70% identity to the polypeptides in SEQ ID NOs: 20 and 18. In some embodiments, the scFV comprises a VH and/or VL with at 75% identity to the polypeptides in SEQ ID NOs: 20 and 18. In some embodiments, the scFV comprises a VH and/or VL with at 80% identity to the polypeptides in SEQ ID NOs: 20 and 18. In some embodiments, the scFV comprises a VH and/or VL with at 85% identity to the polypeptides in SEQ ID NOs: 20 and 18. In some embodiments, the scFV comprises a VH and/or VL with at 90% identity to the polypeptides in SEQ ID NOs: 20 and 18. In some embodiments, the CDRs of SEQ ID NOs: 20 and 18 have 1 point mutation. In some embodiments, the CDRs of SEQ ID NOs: 20 and 18 have 2 point mutations. In some embodiments, the CDRs of SEQ ID NOs: 20 and 18 have 3, 4 or 5 point mutations. In some embodiments, the sequence(s) are those shown in FIG. 16. In some embodiments, the binding domain is defined by the amino acid structure alone, and can be any one of those sequences provided herein regarding such amino acid structures.

In some embodiments, the antigen binding domain can be made specific for any disease-associated antigen, including but not limited to tumor-associated antigens (TAAs) and infectious disease-associated antigens. In some embodiments, the ligand binding domain is bispecific. Antigens have been identified in most of the human cancers, including Burkitt lymphoma, neuroblastoma, melanoma, osteosarcoma, renal cell carcinoma, breast cancer, prostate cancer, lung carcinoma, and colon cancer. TAA's include, without limitation, CD 19, CD20, CD22, CD24, CD33, CD38, CD 123, CD228, CD138, BCMA, GPC3, CEA, folate receptor (FRα), mesothelin, CD276, gp100, 5T4, GD2, EGFR, MUC-1, PSMA, EpCAM, melanoma chondroitin sulfate proteoglycan (MCSP), SM5-1, MICA, MICB, ULBP and HER-2. TAAs further include neoantigens, peptide/MHC complexes, and HSP/peptide complexes.

In some embodiments, the antigen binding domain comprises a T-cell receptor or binding fragment thereof that binds to a defined tumor specific peptide-MHC complex. The term “T cell receptor,” or “TCR,” refers to a heterodimeric receptor composed of ab or gd chains that pair on the surface of a T cell. Each a, b, g, and d chain is composed of two Ig-like domains: a variable domain (V) that confers antigen recognition through the complementarity determining regions (CDR), followed by a constant domain (C) that is anchored to cell membrane by a connecting peptide and a transmembrane (TM) region. The TM region associates with the invariant subunits of the CD3 signaling apparatus. Each of the V domains has three CDRs. These CDRs interact with a complex between an antigenic peptide bound to a protein encoded by the major histocompatibility complex (pMHC) (Davis and Bjorkman (1988) Nature, 334, 395-402; Davis et al. (1998) Annu Rev Immunol, 16, 523-544; Murphy (2012), xix, 868 p.).

In some embodiments, the antigen binding domain comprises a natural ligand of a tumor expressed protein or tumor-binding fragment thereof. A non-limiting example is PD1 which binds to PDL1. Another example is the transferrin receptor 1 (TfR1), also known as CD71, a homodimeric protein that is a key regulator of cellular iron homeostasis and proliferation. Although TfR1 is expressed at a low level in a broad variety of cells, it is expressed at higher levels in rapidly proliferating cells, including malignant cells in which overexpression has been associated with poor prognosis. In some embodiments, the antigen binding domain comprises transferrin or a transferrin receptor-binding fragment thereof.

In some embodiments, the antigen binding domain is specific to a defined tumor associated antigen, such as but not limited to FRα, CEA, 5T4, CA125, SM5-1 or CD71. In some embodiments, the binding domain binds to pembrolizumab. In some embodiments, the tumor associated antigen can be a tumor-specific peptide-MHC complex. In some such embodiments, the peptide is a neoantigen. In other embodiments, the tumor associated antigen it a peptide-heat shock protein complex.

As use herein, the term “specifically binds” or “is specific for” refers to measurable and reproducible interactions, such as binding between a target and an antibody or antibody moiety that is determinative of the presence of the target in the presence of a heterogeneous population of molecules, including biological molecules. For example, an antibody moiety that specifically binds to a target (which can be an epitope) is an antibody moiety that binds the target with greater affinity, avidity, more readily, and/or with greater duration than its bindings to other targets. In some embodiments, an antibody moiety that specifically binds to an antigen reacts with one or more antigenic determinants of the antigen (for example a cell surface antigen or a peptide/MHC protein complex) with a binding affinity that is at least about 10 times its binding affinity for other targets.

In some embodiments, the fusion protein comprises a transmembrane domain linked to the binding domain and CD28 signaling domain. In some embodiments, the transmembrane domain influences fusion protein function. In some embodiments, the transmembrane domain is comprised of the full length primary costimulatory receptor domain. In some embodiments, the transmembrane domain can comprise the transmembrane domain of CD28. In some embodiments, the transmembrane domain comprises amino acids of the CD28 transmembrane domain from about amino acid 153 to about amino acid 179. In some embodiments, the CD28 domain is simply the amino acid structure shown in FIG. 16, or one at least 60, 70, 80, 90, 95, 96, 97, 98, 99, or 100% identical thereto.

In some embodiments, the fusion protein comprises a CD28 signaling domain linked to a transmembrane domain and CD40 costimulatory domain. In some embodiments, the CD28 signaling domain can provide costimulatory signal to the cell upon recognition of folate receptor alpha 1 by the scFV. In some embodiments, the co-stimulatory signal provided by the CD28 signaling domain can enhance cell survival and proliferation. The co-stimulatory signal provided from the CD28 and CD40 signaling domains upon folate receptor alpha 1 recognition by the binding domain can be sufficient to promote desired T-cell function, including stimulation, survival and proliferation of fusion protein expressing cells in the absence of IL-2. In some embodiments, the CD28 signaling domain can comprise a full length signaling domain.

For reference, in some embodiments, the human CD28 protein sequence is set forth in GenBank accession No. NP 006130.1, including signal peptide (amino acids 1-18), extracellular domain (amino acids 19-152), transmembrane domain (amino acids 153-179) and cytoplasmic domain (amino acids 180-200). In some embodiments, the extracellular domain includes an immunoglobulin type domain (amino acids 21-136) which contains amino acids with compose the antigen binding site and amino acids that form the homodimer interface. In some embodiments, the extracellular domain includes several asparagine residues which can be glycosylated, and the intracellular domain comprises serine and tyrosine residues, which can be phosphorylated, where the sequence is shown in FIG. 16.

In some embodiments, the fusion protein comprises a CD40 signaling domain linked to the CD28 signaling domain. The CD40 signaling domain can provide co-stimulatory signal to the cell upon recognition of folate receptor alpha 1 by the scFV. In some embodiments, the co-stimulatory signal provided by the CD40 signaling domain can enhance cell survival and proliferation. the co-stimulatory signal provided from the CD28 and CD40 signaling domains upon folate receptor alpha 1 recognition by the binding domain can be sufficient to promote survival and proliferation of fusion protein expressing cells in the absence of IL-2. In some embodiments, the CD40 signaling domain can comprise SEQ ID NO: 11. In some embodiments, the CD40 signaling domain can comprise an SH3 motif (SEQ ID NO:26), TRAF2 motif (SEQ ID NO:27, 28, or 29), TRAF6 motif (SEQ ID NO: 30), PKA (SEQ ID NO: 31 or 32), or a combination thereof, where the sequence list is shown in FIG. 16. In some embodiments, the CD40 domain is simply the amino acid structure shown in FIG. 16, or one at least 60. 70. 80, 90, 95, 96, 97, 98, 99, or 100% identical thereto.

CD40 is a member of the tumor necrosis factor receptor (TNFR) superfamily and several isoforms are generated by alternative splicing. Its ligand, CD 154 (also called CD40L) is a protein that is primarily expressed on activated T cells. For reference, the human CD40 isoform 1 protein sequence is set forth in GenBank accession No. NP 001241.1, including signal peptide (amino acids 1-20), transmembrane domain (amino acids 194-215), and cytoplasmic domain (amino acids 216-277)(SEQ ID NO:33, RRRGKTNHYQ TTVEKKSLTI YAQVQKPGPL QKKLDSFPAQ DPCTTIYVAA TEPVPESVQE TNSITVYASV TLPES). CD40 receptor signaling involves adaptor proteins including but not limited to TNF receptor-associated factors (TRAF), and the CD40 cytoplasmic domain comprises signaling components, including amino acid sequences fitting an SH3 motif (KPTNKAPH or PTNKAPHP or PTNKAPH), TRAF 2 motif (PKQE, PKQET, PVQE, PVQET, SVQE, SVQET), TRAF 6 motif (QEPQEINF or QEPQEINFP) and PKA motif (KKPTNKA, SRISVQE). Embodiments can further include engineered signaling domains, such as engineered CD40 signaling domains, comprising TRAF-binding amino acid sequences. Engineered signaling domains that bind to TRAF1, TRAF2, TRAF3, and TRAFS can comprise the major consensus sequence (P/S/A/T)X(Q/E)E or minor consensus sequence PXQXXD and can be identified in or obtained from, without limitation, TNFR family members such as CD30, 0x40, 4-1BB, and the EBV oncoprotein LMP1. (See, e.g., Ye, H et al, The Structural Basis for the Recognition of Diverse Receptor Sequences by TRAF2. Molecular Cell, 1999; 4(3):321-30. doi: 10.1016/SI 097-2765(00)80334-2; Park H H, Structure of TRAF Family: Current Understanding of Receptor Recognition. Front. Immunol. 2018; 9:1999. doi: 10.3389/fimmu.2018.01999; Chung, J. Y. et al., All TRAF s are not created equal: common and distinct molecular mechanisms of TRAF-mediated signal transduction. Journal of Cell Science 2002; 115:679-688).

In some embodiments, selection of one or more costimulatory domain signaling component or motif is guided by the cell in which the CoStAR is to be expressed and/or a desired costimulatory activity more closely identified with a signaling component or motif, or avoidance of a costimulatory activity more closely identified with a signaling component or motif.

In some embodiments, amino acid sequence variants of the antibody moieties or other moieties provided herein are contemplated. For example, it can be desirable to improve the binding affinity and/or other biological properties of the antibody moiety. Amino acid sequence variants of an antibody moiety can be prepared by introducing appropriate modifications into the nucleotide sequence encoding the antibody moiety, or by peptide synthesis. Such modifications include, for example, deletions from, and/or insertions into and/or substitutions of residues within the amino acid sequences of the antibody moiety. Any combination of deletion, insertion, and substitution can be made to arrive at the final construct, provided that the final construct possesses the desired characteristics, e.g., antigen-binding.

In some embodiments, antibody binding domain moieties comprising one or more amino acid substitutions, deletions, or insertions are provided. Sites of interest for mutational changes include the antibody binding domain heavy and light chain variable regions (VRs) and frameworks (FRs). Amino acid substitutions can be introduced into a binding domain of interest and the products screened for a desired activity, e.g., retained/improved antigen binding or decreased immunogenicity. In some embodiments, amino acid substitutions can be introduced into one or more of the primary co-stimulatory receptor domain (extracellular or intracellular), secondary costimulatory receptor domain, or extracellular co-receptor domain. Accordingly, some embodiments encompass CoStAR proteins and component parts particularly disclosed herein as well as variants thereof, e.g., CoStAR proteins and component parts having at least 75%, at least 80%, at least 85%, at least 87%, at least 90%, at least 91%, at least 92%, at least 93%, at least 94%, at least 95%, at least 96%, at least 97%, at least 98%, at least 99% sequence identity to the amino acid sequences particularly disclosed herein. The terms “percent similarity,” “percent identity,” and “percent homology” when referring to a particular sequence are used as set forth in the University of Wisconsin GCG software program BestFit. Other algorithms can be used, e.g. BLAST, psiBLAST or TBLASTN (which use the method of Altschul et al. (1990) J. Mol. Biol. 215: 405-410), FASTA (which uses the method of Pearson and Lipman (1988) PNAS USA 85: 2444-2448).

Particular amino acid sequence variants can differ from a reference sequence by insertion, addition, substitution or deletion of 1 amino acid, 2, 3, 4, 5-10, 10-20 or 20-30 amino acids. In some embodiments, a variant sequence can comprise the reference sequence with 1, 2, 3, 4, 5, 6, 7, 8, 9, 10 or more residues inserted, deleted or substituted. For example, 5, 10, 15, up to 20, up to 30 or up to 40 residues can be inserted, deleted or substituted.

In some embodiments, a variant can differ from a reference sequence by 1, 2, 3, 4, 5, 6, 7, 8, 9, 10 or more conservative substitutions. Conservative substitutions involve the replacement of an amino acid with a different amino acid having similar properties. For example, an aliphatic residue can be replaced by another aliphatic residue, a non-polar residue can be replaced by another non-polar residue, an acidic residue can be replaced by another acidic residue, a basic residue can be replaced by another basic residue, a polar residue can be replaced by another polar residue or an aromatic residue can be replaced by another aromatic residue. Conservative substitutions can, for example, be between amino acids within the following groups:

Conservative substitutions are as follows: Amino acids can be grouped into different classes according to common side-chain properties: a. hydrophobic: Norleucine, Met, Ala, Val, Leu, lie; b. neutral hydrophilic: Cys, Ser, Thr, Asn, Gin; c. acidic: Asp, Glu; d. basic: His, Lys, Arg; e. residues that influence chain orientation: Gly, Pro; aromatic: Trp, Tyr, Phe. Non-conservative substitutions will entail exchanging a member of one of these classes for another class.

### Cells

The cells used can be any lymphocyte that is useful in adoptive cell therapy, such as a T-cell or a natural killer (NK) cell, an NKT cell, a gamma/delta T-cell or T regulatory cell. The cells can be allogeneic or autologous to the patient.

T cells or T lymphocytes are a type of lymphocyte that have a central role in cell-mediated immunity. They can be distinguished from other lymphocytes, such as B cells and natural killer cells (NK cells), by the presence of a T-cell receptor (TCR) on the cell surface. There are various types of T cell, as summarized below. Cytotoxic T cells (TC cells, or CTLs) destroy virally infected cells and tumor cells, and are also implicated in transplant rejection. CTLs express the CD8 molecule at their surface.

These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells can be either CD4+ or CD8+. Memory T cells typically express the cell surface protein CD45RO. Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4+ Treg cells have been described—naturally occurring Treg cells and adaptive Treg cells. Naturally occurring Treg cells (also known as CD4+ CD25+ FoxP3+ Treg cells) arise in the thymus and have been linked to interactions between developing T cells with both myeloid (CD1 1c+) and plasmacytoid (CD123+) dendritic cells that have been activated with TSLP. Naturally occurring Treg cells can be distinguished from other T cells by the presence of an intracellular molecule called FoxP3. Adaptive Treg cells (also known as Tr1 cells or Th3 cells) can originate during a normal immune response.

Natural Killer Cells (or NK cells) are a type of cytolytic cell which form part of the innate immune system. NK cells provide rapid responses to innate signals from virally infected cells in an MHC independent manner. NK cells (belonging to the group of innate lymphoid cells) are defined as large granular lymphocytes (LGL) and constitute the third kind of cells differentiated from the common lymphoid progenitor generating B and T lymphocytes.

In some embodiments, therapeutic cells comprise autologous cells engineered to express a CoStAR. In some embodiments, therapeutic cells comprise allogeneic cells engineered to express a CoStAR. Autologous cells expressing CoStARs can be advantageous in avoiding graft-versus-host disease (GVHD) due to TCR-mediated recognition of recipient alloantigens. Also, the immune system of a CoStAR recipient could attack the infused CoStAR cells, causing rejection. In some embodiments, to prevent GVHD, and to reduce rejection, endogenous TcR is removed from allogeneic CoStAR cells by genome editing. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

### Nucleic Acids

In some embodiments, a nucleic acid sequence as provided encodes any of the CoStARs, polypeptides, or proteins described herein (including functional portions and functional variants thereof). As used herein, the terms “polynucleotide”, “nucleotide”, and “nucleic acid” are intended to be synonymous with each other. It will be understood by a skilled person that numerous different polynucleotides and nucleic acids can encode the same polypeptide as a result of the degeneracy of the genetic code. In addition, it is to be understood that skilled persons can, using routine techniques, make nucleotide substitutions that do not affect the polypeptide sequence encoded by the polynucleotides described here to reflect the codon usage of any particular host organism in which the polypeptides are to be expressed, e.g. codon optimisation. Nucleic acids can comprise DNA or RNA. They can be single stranded or double-stranded. They can also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. It is to be understood that the polynucleotides can be modified by any method available in the art. Such modifications can be carried out in order to enhance the in vivo activity or life span of polynucleotides of interest. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

The terms “variant”, “homologue” or “derivative” in relation to a nucleotide sequence include any substitution of, variation of, modification of, replacement of, deletion of or addition of one (or more) nucleic acid from or to the sequence.

The nucleic acid sequence can encode the protein sequence shown in any of the figures provided herein or a variant thereof. The nucleotide sequence can comprise a codon optimized nucleic acid sequence shown engineered for expression in human cells.

A nucleic acid sequence which comprises a nucleic acid sequence encoding a CoStAR and a further nucleic acid sequence encoding a T-cell receptor (TCR) and/or chimeric antigen receptor (CAR) is also contemplated.

The nucleic acid sequences can be joined by a sequence allowing coexpression of the two or more nucleic acid sequences. For example, the construct can comprise an internal promoter, an internal ribosome entry sequence (IRES) sequence or a sequence encoding a cleavage site. The cleavage site can be self-cleaving, such that when the polypeptide is produced, it is immediately cleaved into the discrete proteins without the need for any external cleavage activity. Various self-cleaving sites are known, including the Foot- and Mouth disease virus (FMDV) and the 2A self-cleaving peptide. The co-expressing sequence can be an internal ribosome entry sequence (IRES). The co-expressing sequence can be an internal promoter.

### Vectors

In some embodiments, a vector is provided which comprises a nucleic acid sequence or nucleic acid construct as provided herein.

Such a vector can be used to introduce the nucleic acid sequence(s) or nucleic acid construct(s) into a host cell so that it expresses one or more CoStAR(s) according to some embodiments and, optionally, one or more other proteins of interest (POI), for example a TCR or a CAR. The vector can, for example, be a plasmid or a viral vector, such as a retroviral vector or a lentiviral vector, or a transposon-based vector or synthetic mRNA. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

The nucleic acids can also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties.

Vectors derived from retroviruses, such as the lentivirus, are suitable tools to achieve long-term gene transfer since they allow long-term, stable integration of a transgene or transgenes and its propagation in daughter cells. The vector can be capable of transfecting or transducing a lymphocyte including a T cell or an NK cell. Provided herein are vectors in which a nucleic acid is inserted. The expression of natural or synthetic nucleic acids encoding a CoStAR, and optionally a TCR or CAR is typically achieved by operably linking a nucleic acid encoding the CoStAR and TCR/CAR polypeptide or portions thereof to one or more promoters, and incorporating the construct into an expression vector.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1a (EF-1a). However, other constitutive promoter sequences can also be used, including, but not limited to the simian virus 40 (SV40) early promoter, mouse mammary tumor virus (MMTV), human immunodeficiency virus (HIV) long terminal repeat (LTR) promoter, MoMuLV promoter, MSCV promoter, MND promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

The vectors can be suitable for replication and integration in eukaryotic cells. Typical cloning vectors contain transcription and translation terminators, initiation sequences, and promoters useful for regulation of the expression of the desired nucleic acid sequence. Viral vector technology is well known in the art and is described, for example, in Sambrook et al. (2001, Molecular Cloning: A Laboratory Manual, Cold Spring Harbor Laboratory, New York), and in other virology and molecular biology manuals, see also, WO 01/96584; WO 01/29058; and U.S. Pat. No. 6,326,193). In some embodiments, the constructs expressed are as shown in SEQ ID NOS:32-65 and 67-79. In some embodiments the nucleic acids are multi-cistronic constructs that permit the expression of multiple transgenes (e.g., CoStAR and a TCR and/or CAR etc.) under the control of a single promoter. In some embodiments, the transgenes (e.g., CoStAR and a TCR and/or CAR etc.) are separated by a self-cleaving 2A peptide. Examples of 2A peptides useful in the nucleic acid constructs include F2A, P2A, T2A and E2A. In other embodiments, the nucleic acid construct is a multi-cistronic construct comprising two promoters; one promoter driving the expression of CoStAR and the other promoter driving the expression of the TCR or CAR. In some embodiments, the dual promoter constructs are uni-directional. In some embodiments, the dual promoter constructs are bi-directional. In order to assess the expression of the CoStAR polypeptide or portions thereof, the expression vector to be introduced into a cell can also contain either a selectable marker gene or a reporter gene or both to facilitate identification and selection of expressing cells from the population of cells sought to be transfected or transduced through viral vectors.

As noted herein, the use of the fusion protein comprising a first domain, a transmembrane domain, a CD28 domain and a CD40 domain (such as a CoStAR) in an appropriate manner allows for one to reduce and/or eliminate the use of IL-2 in a subject during cell therapy.

In some embodiments, subject undergoing cancer treatment with fusion protein expressing cells does not require repeated doses of exogenous IL-2 in amounts adequate for stimulation of cell survival during the course of treatment. In some embodiments, subject undergoing cancer treatment with fusion protein expressing cells does not require co-administration of IL-2 in amounts adequate for stimulation of cell survival and proliferation in vivo. In some embodiments, subject undergoing cancer treatment with fusion protein expressing cells is not exposed to toxicity associated with exogenous IL-2 administration. In some embodiments, survival of fusion protein expressing cells is stimulated by the CD28 and CD40 signaling domains activated by the presence of folate receptor alpha 1 expressing cells. In some embodiments, the IL-2 administered to the subject will be less than 600,000 IU/kg to 720,000 IU/kg/day. In some embodiments, the IL-2 administered to the subject will be less than 125,000 IU/kg/day. In some embodiments, the IL-2 administered to the subject will be less than 112,500 IU/kg/day. In some embodiments, the IL-2 administered to the subject will be less than 100,000 IU/kg/day. In some embodiments, the IL-2 administered to the subject will be less than 87,500 IU/kg/day. In some embodiments, the IL-2 administered to the subject will be less than 75,000 IU/kg/day. In some embodiments, the IL-2 administered to the subject will be less than 62,500 IU/kg/day. In some embodiments, the IL-2 administered to the subject will be less than 50,000 IU/kg/day. In some embodiments, the IL-2 administered to the subject will be less than 37,500 IU/kg/day. In some embodiments, the IL-2 administered to the subject will be less than 25,000 IU/kg/day. In some embodiments, the IL-2 administered to the subject will be less than 12,500 IU/kg/day. In some embodiments, the IL-2 administered to the subject will be less than 6,250 IU/kg/day. In some embodiments, the IL-2 administered to the subject will be less than 2,500 IU/kg/day. In some embodiments, the IL-2 administered to the subject will be less than 1,250 IU/kg/day. In some embodiments, the amount of IL-2 administered to a subject is reduced by 10%. In some embodiments, the amount of IL-2 administered to a subject is reduced by 20%. In some embodiments, the amount of IL-2 administered to a subject is reduced by 30%. In some embodiments, the amount of IL-2 administered to a subject is reduced by 40%. In some embodiments, the amount of IL-2 administered to a subject is reduced by 50%. In some embodiments, the amount of IL-2 administered to a subject is reduced by 60%. In some embodiments, the amount of IL-2 administered to a subject is reduced by 70%. In some embodiments, the amount of IL-2 administered to a subject is reduced by 80%. In some embodiments, the amount of IL-2 administered to a subject is reduced by 90%. In some embodiments, the amount of IL-2 administered to a subject is reduced by 95%. In some embodiments, the amount of IL-2 administered to a subject is reduced by 98%. In some embodiments, the amount of IL-2 administered to a subject is reduced by 99%. In some embodiments, any IL-2 administered to a subject is de minimis. In some embodiments, the any IL-2 administered to a subject during cell therapy is not to assist with the stimulation of the cells for the cell therapy in vivo, as it is not needed. In some embodiments, reduction in IL-2 dose will not lead to decreased efficacy of the cell therapy.

In some embodiments less than 10 doses, or less than 9 doses, or less than 8 doses, or less than 7 doses, or less than 6 doses, or less than 5 doses, or less than 4 doses, or less than 3 doses, or less than 2 doses, or less than 1 dose of IL-2 will be administered to the subject during the cellular (or for the cellular) therapy.

In some embodiments, IL-2 will be administered less often than every hour, or less often than every 2 hours, or less often than every 3 hours, or less often than every 4 hours, or less often than every 6 hours, or less often than every 8 hours, or less often than every 12 hours, or less often than every 16 hours, or less often than every 20 hours, or less often than every day, or less often than every 2 days, or less often than every 3 days, or less often than every week, or less often than every 2 weeks, or less often than every month, or less often than every 6 months, or less often than every year.

In some embodiments, a subject undergoing cancer treatment with fusion protein expressing cells is not exposed to one or more toxicity associated with exogenous IL-2 administration including at least one of: capillary leak syndrome, impaired neurophil function; hypothermia; shock; bradycardia; ventricular extrasystoles; myocardial ischemia; syncope; hemorrhage; atrial arrhythmia; phlebitis; AV block second degree; endocarditis; pericardial effusion; peripheral gangrene; thrombosis; coronary artery disorder; stomatitis; nausea and vomiting; liver function tests abnormal; gastrointestinal hemorrhage; hematemesis; bloody diarrhea; gastrointestinal disorder; intestinal perforation; pancreatitis; anemia; leukopenia; leukocytosis; hypocalcemia; alkaline phosphatase increase; BUN increase; NPN increase; respiratory acidosis; somnolence; agitation; neuropathy; paranoid reaction; convulsion; grand mal convulsion; delirium; asthma, lung edema; hyperventilation; hypoxia; hemoptysis; hypoventilation; pneumothorax; mydriasis; pupillary disorder; kidney function abnormal; kidney failure; acute tubular necrosis; duodenal ulceration; bowel necrosis; myocarditis; supraventricular tachycardia; permanent or transient blindness secondary to optic neuritis; transient ischemic attacks; meningitis; cerebral edema; pericarditis; allergic interstitial nephritis; tracheo-esophageal fistula; malignant hyperthermia; cardiac arrest; myocardial infarction; pulmonary emboli; stroke; intestinal perforation; liver or renal failure; severe depression leading to suicide; pulmonary edema; respiratory arrest; and/or respiratory failure.

In some embodiments, the cells are used to treat cancers and neoplastic diseases associated with a target antigen. In some embodiments, cancers and neoplastic diseases that can be treated using any of the methods described herein include tumors that are not vascularized, or not yet substantially vascularized, as well as vascularized tumors. In some embodiments, the cancers can comprise non-solid tumors (such as hematological tumors, for example, leukemias and lymphomas) or can comprise solid tumors. In some embodiments, types of cancers to be treated with the fusion protein expressing cells of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included. In some embodiments, the cancers overexpress FRα. FRα expression been reported in the literature to be restricted mostly to the apical surfaces of epithelial tissues such as the kidney, lungs, choroid plexus, ovary, uterus, fallopian tubes, epididymis, submandibular salivary and bronchial glands, and placental trophoblasts (Weitman et al, 1992). FRα levels are often elevated in cancers of epithelial origin compared with normal tissue, and overexpression has been reported in many solid tumors, including NSCLC, epithelial ovarian, fallopian tube and peritoneal carcinomas (EOC), renal cell carcinoma (RCC), cervical, endometrial, breast, brain, kidney, colon, pancreatic, and bladder cancer (Ross et al, 1994; Parker et al, 2005; Assaraf et al, 2014). Studies suggest that the expression of FRα may contribute to cancer development by both cell growth regulation and signaling functions (Bagnoli et al, 2000; Kelemen, 2006). FRα expression has been associated with poor prognosis in various cancers including breast, ovarian, and endometrial cancers (Kurosaki et al, 2016; Liu et al, 2020).

In some embodiments, hematologic cancers of the blood or bone marrow can be treated with the fusion protein expressing cells. In some embodiments, examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, plasmacytoma, Waldenstrom's macroglobulinemia, heavy chain disease, myelodysplastic syndrome, hairy cell leukemia and myelodysplasia. In some embodiments, any cancer expressing the FRα protein product can be targeted.

In some embodiments, solid tumors can be treated with the fusion protein expressing cells. In some embodiments, examples of solid tumors are sarcomas and carcinomas, include adrenocortical carcinoma, cholangiocarcinoma, fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, stomach cancer, lymphoid malignancy, pancreatic cancer, breast cancer (e.g., triple negative breast cancer “TNBC”), lung cancers (e.g., lung adenocarcinomas or non-small cell lung cancer), ovarian cancer, prostate cancer, hepatocellular carcinoma, squamous cell carcinoma, basal cell carcinoma, adenocarcinoma, sweat gland carcinoma, thyroid cancer (e.g., medullary thyroid carcinoma and papillary thyroid carcinoma), pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer (e.g., cervical carcinoma and pre-invasive cervical dysplasia), colorectal cancer, cancer of the anus, anal canal, or anorectum, vaginal cancer, cancer of the vulva (e.g., squamous cell carcinoma, intraepithelial carcinoma, adenocarcinoma, and fibrosarcoma), penile cancer, oropharyngeal cancer, esophageal cancer, head cancers (e.g., squamous cell carcinoma), neck cancers (e.g., squamous cell carcinoma), testicular cancer (e.g., seminoma, teratoma, embryonal carcinoma, teratocarcinoma, choriocarcinoma, sarcoma, Ley dig cell tumor, fibroma, fibroadenoma, adenomatoid tumors, and lipoma), bladder carcinoma, kidney cancer, melanoma, cancer of the uterus (e.g., endometrial carcinoma), urothelial cancers (e.g., squamous cell carcinoma, transitional cell carcinoma, adenocarcinoma, ureter cancer, and urinary bladder cancer), and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

In some embodiments, the subject in need of TIL therapy can be suffering from a type of cancer where folate receptor alpha 1 is upregulated by cancer cells within the tumor. In some embodiments, the folate receptor alpha 1 expressing cancer cells can be targeted by fusion protein expressing cells. folate receptor alpha 1 expression can drive survival and proliferation of the fusion protein expressing cells. In some embodiments, the subject can be suffering from types of cancer comprising solid tumors, renal cancer, lung cancer, or ovarian cancer.

In some embodiments, an individual suitable for treatment as described above can be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutan, gibbon), or a human. In some embodiments, the individual is a human. In some embodiments, non-human mammals, especially mammals that are conventionally used as models for demonstrating therapeutic efficacy in humans (e.g. murine, primate, porcine, canine, or rabbit animals) can be employed.

In some embodiments, prior to expansion and genetic modification, a source of cells (e.g., immune effector cells, e.g., T cells or NK cells) is obtained from a subject. In some embodiments, the term “subject” is intended to include living organisms in which an immune response can be elicited (e.g., mammals). In some embodiments, examples of subjects include humans, dogs, cats, mice, rats, and transgenic species thereof. In some embodiments, T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, the cells are returned to the same subject for cell therapy.

In some embodiments, the subject receiving fusion protein expressing cells as cancer treatment does not receive exogenous IL-2 in a manner that is adequate for cell stimulation of TILs in vivo (absent the presence of the fusion protein). In some embodiments, the fusion protein expressing cells can receive co-stimulatory signals from the CD28 and CD40 signaling domains of the fusion receptor following scFV recognition of folate receptor alpha 1. In some embodiments, the co-stimulatory signals provided by the signaling domains of the fusion receptor can provide sufficient survival and proliferation signals to prevent the requirement of exogenous IL-2. In some embodiments, cells expressing the fusion protein can be capable of sustained survival and proliferation in the absence of IL-2 and the presence of folate receptor alpha 1 in vivo. In some embodiments, cells expressing the fusion can be capable of surviving at least 60, 80, 100, or more days post injection in the absence of IL-2 and presence of folate receptor alpha 1.

In some embodiments, a method of cell therapy comprising identifying a subject in need of tumor infiltrating lymphocyte (“TIL”) cell therapy and administering to the subject a TIL cell therapy is provided. The TIL cell therapy comprises a fusion protein that comprises a binding domain specific for folate receptor alpha 1 linked to a transmembrane domain that is linked to a CD28 signaling domain that is linked to a CD40 signaling domain. The TIL cell therapy does not include a level of IL-2 administered to the subject. The level of IL-2 is one that is sufficient to provide for IL-2 stimulated TIL cell therapy. Any of the elements of the fusion protein can be any of those provided herein.

In some embodiments, the method of cell therapy can comprise collecting subject TILs and transducing them with the fusion protein as provided herein. In some embodiments, the transduction method can comprise a viral vector. In some embodiments, expression of fusion protein on TILs can be verified before administration to the subject. In some embodiments, the cell therapy method can comprise administration of fusion protein expressing TILs to the subject in the absence of IL-2 during the engraftment process. In some embodiments, the cell therapy method can comprise no co-administration of exogenous IL-2 during the course of cell therapy to support survival of the TILs. In some embodiments, the method of cell therapy can comprise fusion protein expressing TILs, where the survival of the fusion peptide expressing TILs is stimulated by folate receptor alpha 1 expressing cells, rather than by the addition of exogenous IL-2 when the cells are administered to the subject (or thereafter).

In some embodiments, the in vitro expansion of fusion protein expressing TILs can be conducted in a supplemented cell culture medium comprising IL-2, OKT-3, and antigen-presenting feeder cells. In some embodiments, the in vitro expansion cell culture medium comprises IL-2. In some embodiments, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL, or between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or between 8000 IU/mL of IL-2. In some embodiments, fusion protein expressing cells are not treated with IL-2 following in vitro expansion.

In some embodiments, the IL-2 administered to the subject will be less than 125,000 IU/kg/day, less than 112,500 IU/kg/day, less than 100,000 IU/kg/day, less than 87,500 IU/kg/day, less than 75,000 IU/kg/day, less than 62,500 IU/kg/day, less than 50,000 IU/kg/day, less than 37,500 IU/kg/day, less than 25,000 IU/kg/day, less than 12,500 IU/kg/day, less than 6,250 IU/kg/day, 2,500 IU/kg/day, less than 1,250 IU/kg/day, or any IL-2 administered to a subject is de minimis.

In some embodiments, a method of administering a cell therapy is provided. The method comprises administering to a subject a TIL cell therapy. The TIL cell therapy comprises a fusion protein that comprises a binding domain specific for folate receptor alpha 1 linked to a transmembrane domain that is linked to a CD28 signaling domain that is linked to a CD40 signaling domain. The method excludes a step of administering IL-2 to the subject to promote stimulation of the TILs in vivo. Instead, stimulation of the TILs in vivo is achieved via the fusion protein. In some embodiments, any of the elements of the fusion protein can be any of those provided herein.

In some embodiments, Tumor-infiltrating lymphocytes (TILs) are a polyclonal cell product that encompasses broad diversity of antitumor reactivity with an unrestricted T-cell receptor (TCR) repertoire, thereby offering the broadest diversity of antitumor reactivity. In some embodiments, in vivo stimulation of fusion protein expressing TILs is accomplished by co-stimulatory signaling provided by the fusion protein recognizing folate receptor alpha 1. In some embodiments IL-2 administration can be excluded during engraftment. In some embodiments, administration of IL-2 can be excluded following the engraftment phase. In some embodiments IL-2 administration can be excluded at any phase during the course of treatment with TILs expressing the fusion protein. In some embodiments the stimulation provided to the TILs by the fusion receptor is sufficient to support survival of the TILs throughout the treatment process. In some embodiments, the IL-2 administered to the subject will be less than 125,000 IU/kg/day, less than 112,500 IU/kg/day, less than 100,000 IU/kg/day, less than 87,500 IU/kg/day, less than 75,000 IU/kg/day, less than 62,500 IU/kg/day, less than 50,000 IU/kg/day, less than 37,500 IU/kg/day, less than 25,000 IU/kg/day, less than 12,500 IU/kg/day, less than 6,250 IU/kg/day, 2,500 IU/kg/day, less than 1,250 IU/kg/day, or any IL-2 administered to a subject is de minimis.

In some embodiments a method of administering a cell therapy is provided. The method comprises administering a costimulatory antigen receptor (“CoStAR”) to a subject in the absence of a level of IL-2. The level of IL-2 is one sufficient to cause TIL stimulation in vivo when the CoStAR is absent. Any of the elements of the CoStAR can be any of those provided herein. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

In some embodiments, a recombinant costimulatory antigen receptors (CoStARs) is provided, comprising: (i) a disease- or tumor-associated antigen binding domain, (ii) a first intracellular segment comprising an intracellular signaling domain of CD28, and (iii) a second intracellular signaling domain of a CD40 receptor protein or signal transducing fragment thereof. In some embodiments, the antigen binding domain is specific for folate receptor alpha 1. In some embodiments, the CoStAR provides Signal 2 upon recognition of folate receptor alpha 1. In some embodiments, Signal 1 is provided by the native receptor expressed by the immune cell.

In some embodiments, the CoStAR is capable of providing sufficient Signal 2 co-stimulation upon folate receptor alpha 1 recognition to allow for the absence of IL-2 during treatment. In some embodiments, stimulation provided by the CoStAR upon recognition of folate receptor alpha 1 provides sufficient TIL stimulation, such that IL-2 levels normally required for the stimulation of TILs not expressing the CoStAR can be absent in vivo during the course of treatment.

In some embodiments, a method of administering a cell therapy for a cancer treatment is provided. The method comprises administering a costimulatory antigen receptor (“CoStAR”) to a subject. IL-2 is not used in the therapy at a level sufficient to promote TIL stimulation in the absence of the CoStAR. Any of the elements of the CoStAR can be any of those provided herein. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

In some embodiments, IL-2 is not used during cell therapy at a level sufficient to promote TIL stimulation in vivo in the absence of CoStAR expression. In some embodiments, co-stimulatory signal provided by the CoStAR upon folate receptor alpha 1 recognition is sufficient to promote TIL stimulation in vivo during the course of cell therapy for cancer treatment. In some embodiments, levels of IL-2 generally required for supporting TIL stimulation in vivo can be absent from the treatment in vivo due to the co-stimulation provided from the CoStAR. In some embodiments, the IL-2 administered to the subject will be less than 125,000 IU/kg/day, less than 112,500 IU/kg/day, less than 100,000 IU/kg/day, less than 87,500 IU/kg/day, less than 75,000 IU/kg/day, less than 62,500 IU/kg/day, less than 50,000 IU/kg/day, less than 37,500 IU/kg/day, less than 25,000 IU/kg/day, less than 12,500 IU/kg/day, less than 6,250 IU/kg/day, 2,500 IU/kg/day, less than 1,250 IU/kg/day, or any IL-2 administered to a subject is de minimis. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

In some embodiments, a method of in vivo T cell expansion is provided. The method comprises administering a T cell comprising a fusion protein to a subject. IL-2 is not used to promote TIL stimulation, and the fusion protein comprises a binding domain specific for folate receptor alpha 1 linked to a transmembrane domain that is linked to a CD28 signaling domain that is linked to a CD40 signaling domain. Any of the elements of the fusion protein can be any of those provided herein.

In some embodiments, T cell expansion involves signal 1, provided by the TCR complex, which synergizes with signal 2 provided by costimulatory receptors such as CD28, CD137 or CD134 to permit the cells to undergo clonal expansion, IL-2 production and long term survival without the activation induced cell death (AICD) associated with signal 1 alone. In some embodiments, the involvement of signal 2 enhances the signal generated through signal 1 allowing the cells to respond better to low avidity interactions such as those encountered during anti-tumor responses. This can be used to reduce the need or completely eliminate the need for IL-2. In some embodiments, the IL-2 administered to the subject will be less than 125,000 IU/kg/day, less than 112,500 IU/kg/day, less than 100,000 IU/kg/day, less than 87,500 IU/kg/day, less than 75,000 IU/kg/day, less than 62,500 IU/kg/day, less than 50,000 IU/kg/day, less than 37,500 IU/kg/day, less than 25,000 IU/kg/day, less than 12,500 IU/kg/day, less than 6,250 IU/kg/day, 2,500 IU/kg/day, less than 1,250 IU/kg/day, or any IL-2 administered to a subject is de minimis.

In some embodiments, Signal 1 is provided by the native TCR. In some embodiments, Signal 1 is provided by an non-native TCR. In some embodiments, Signal 2 is provided by the fusion protein upon folate receptor alpha 1 binding. In some embodiments, Signal 2 is provided from CD28 and CD40 co-stimulatory domains. In some embodiments, Signal 2 is not provided by the fusion protein without signal 1 from the TCR/peptide/MHC interaction.

In some embodiments, promoting TIL stimulation denotes a level of stimulation sufficient to achieve a therapeutically effective level of stimulation for a treatment of cancer in the subject.

In some embodiments, the term “therapeutically effective amount” refers to an amount of a fusion protein as provided herein, a CoStAR or composition comprising a CoStAR as disclosed herein, effective to “treat” a disease or disorder in an individual. In some embodiments, in the case of cancer, the therapeutically effective amount of a CoStAR or composition comprising a CoStAR as disclosed herein can reduce the number of cancer cells; reduce the tumor size or weight; inhibit (e.g., slow to some extent and preferably stop) cancer cell infiltration into peripheral organs; inhibit (e.g., slow to some extent and preferably stop) tumor metastasis; inhibit, to some extent, tumor growth; and/or relieve to some extent one or more of the symptoms associated with the cancer. In some embodiments, to the extent a CoStAR or composition comprising a CoStAR as disclosed herein can prevent growth and/or kill existing cancer cells, it can be cytostatic and/or cytotoxic. In some embodiments, the therapeutically effective amount is a growth inhibitory amount sufficient for a therapeutic benefit. In some embodiments, the therapeutically effective amount is an amount that improves progression free survival of a patient. In some embodiments, in the case of infectious disease, such as viral infection, the therapeutically effective amount of a CoStAR or composition comprising a CoStAR as disclosed herein can reduce the number of cells infected by the pathogen; reduce the production or release of pathogen-derived antigens; inhibit (i.e., slow to some extent and preferably stop) spread of the pathogen to uninfected cells; and/or relieve to some extent one or more symptoms associated with the infection. In some embodiments, the therapeutically effective amount is an amount that extends the survival of a patient. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

In some embodiments, cellular stimulation is achieved via a TCR dependent mechanism that binds to a peptide that binds to an MHC.

In some embodiments, stimulation of the fusion protein expressing cells involves recognition of cognate peptide presented on an MHC by a TCR (signal 1). In some embodiments a CoStAR of the invention is engineered not to provide signal 1. In some embodiments, a CoStAR of the invention does not comprise a signal 1 signaling domain. In some embodiments, a CoStAR of the invention does not comprise a CD3z signaling domain.

In some embodiments, a CoStAR of the invention is configured to provide signal 2 in a cell that is capable of providing signal 1 upon antigen binding (e.g., a T cell receptor provides signal 1 upon antigen engagement). In some embodiments, a CoStAR is configured to provide signal 2 in a cell in response to antigen-specific binding by the CoStAR when the antigen is on the surface of a target cell. In some embodiments, a CoStAR is engineered not to provide signal 2 in a cell in response to antigen-specific binding by the CoStAR when the antigen is soluble and not attached to the surface of a target cell.

In some embodiments, when combined with TCR-specific peptide:MHC binding, the CoStAR significantly enhances T-cell proliferation, persistence, and antitumor activity in vivo versus TCR alone, resulting in tumor control and prolonged survival, even in the absence of IL-2, as shown in the examples. As shown in the examples, prosurvival effects were not observed with CoStAR alone. In some embodiments, signaling through the CoStAR delivers a strict costimulatory signal and, without accompanying TCR-dependent signaling, does not induce T-cell effector function. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

In some embodiments, a population of genetically engineered immune cells is provided. Each immune cell in the population comprises a fusion protein that comprises a binding domain specific for folate receptor alpha 1 linked to a transmembrane domain linked to a CD28 signaling domain linked to a CD40 signaling domain. The population of genetically engineered immune cells has been administered to a subject who has not received an amount of IL-2 that is adequate to promote proliferation in vivo without the fusion protein, and wherein the population of immune cells has been expanded in the absence of IL-2 in vivo.

In some embodiments, the immune cells are engineered to express a CoStAR. In some embodiments, the immune cells are engineered using a viral vector. The cells used in the present invention can be any lymphocyte that is useful in adoptive cell therapy, such as a T-cell or a natural killer (NK) cell, an NKT cell, a gamma/delta T-cell or T regulatory cell. The cells can be allogeneic or autologous to the patient. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

In some embodiments, the subject has not and/or will not receive an amount of IL-2 that is adequate to promote immune cell proliferation in vivo prior to immune cell administration. In some embodiments, the subject does not receive an amount of IL-2 that is adequate to promote proliferation in vivo concomitantly to immune cell administration. In some embodiments, the subject does not receive an amount of IL-2 that is adequate to promote proliferation in vivo at any point during treatment with immune cells. In some embodiments, the IL-2 administered to the subject will be less than 125,000 IU/kg/day, less than 112,500 IU/kg/day, less than 100,000 IU/kg/day, less than 87,500 IU/kg/day, less than 75,000 IU/kg/day, less than 62,500 IU/kg/day, less than 50,000 IU/kg/day, less than 37,500 IU/kg/day, less than 25,000 IU/kg/day, less than 12,500 IU/kg/day, less than 6,250 IU/kg/day, 2,500 IU/kg/day, less than 1,250 IU/kg/day, or any IL-2 administered to a subject is de minimis.

In some embodiments, the engineered immune cells are capable of proliferation and survival in vivo without exogenous IL-2. In some embodiments, the engineered immune cells receive proliferation signals from the fusion protein upon recognition of folate receptor alpha 1. In some embodiments, the proliferation signals form the fusion receptor are sufficient for survival and proliferation of the immune cells in vivo without exogenous IL-2.

In some embodiments, the cells are T cells. In some embodiments, T cells or T lymphocytes are a type of lymphocyte that have a central role in cell-mediated immunity. In some embodiments, they can be distinguished from other lymphocytes, such as B cells and natural killer cells (NK cells), by the presence of a T-cell receptor (TCR) on the cell surface. In some embodiments, cytotoxic T cells (TC cells, or CTLs) destroy virally infected cells and tumor cells, and are also implicated in transplant rejection. In some embodiments, CTLs express the CD8 molecule at their surface.

In some embodiments, these cells recognize their targets by binding to antigen associated with MEW class I, which is present on the surface of all nucleated cells. In some embodiments, through IL-10, adenosine and other molecules secreted by regulatory T cells, the CD8+ cells can be inactivated to an anergic state, which prevent autoimmune diseases such as experimental autoimmune encephalomyelitis.

In some embodiments, the cells are donor T cells, from the subject.

In some embodiments, therapeutic cells comprise autologous cells engineered to express a fusion protein as provided herein or a CoStAR. In some embodiments, therapeutic cells of the invention comprise allogeneic cells engineered to express a fusion protein as provided herein or a CoStAR. In some embodiments, autologous cells expressing a fusion protein as provided herein or CoStARs can be advantageous in avoiding graft-versus-host disease (GVHD) due to TCR-mediated recognition of recipient alloantigens. In some embodiments, the immune system of a fusion protein as provided herein or CoStAR recipient could attack the infused CoStAR cells, causing rejection. In some embodiments, to prevent GVHD, and to reduce rejection, endogenous TcR is removed from allogeneic fusion protein as provided herein or CoStAR cells by genome editing. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

In some embodiments, the method or population the cells are tumor infiltrating lymphocytes.

In some embodiments, Tumor infiltrating cells (TILs) are isolated and/or expanded from a tumor, for example by a fragmented, dissected, or enzyme digested tumor biopsy or mass. In some embodiments, the TILs can be produced in a two-stage process using a tumor biopsy as the starting material:

Stage 1 (generally performed over 2-3 hours) initial collection and processing of tumor material using dissection, enzymatic digestion and homogenization to produce a single cell suspension which can be directly cryopreserved to stabilize the starting material for subsequent manufacture and Stage 2 which can occur days or years later.

Stage 2 can be performed over 4 weeks, which can be a continuous process starting with thawing of the product of Stage 1 and growth of the TIL out of the tumor starting material (about 2 weeks) followed by a rapid expansion process of the TIL cells (about 2 weeks) to increase the amount of cells and therefore dose. The TILs can be concentrated and washed prior to formulation as a liquid suspension of cells.

Prior to expansion and genetic modification, a source of cells (e.g., immune effector cells, e.g., T cells or NK cells) is obtained from a subject. The term “subject” is intended to include living organisms in which an immune response can be elicited (e.g., mammals). Examples of subjects include humans, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

In some embodiments, T cells are isolated from peripheral blood lymphocytes by lysing the red blood cells and depleting the monocytes, for example, by centrifugation through a PERCOLL™ gradient or by counterflow centrifugal elutriation. T cell can be collected at an apheresis center and cell storage facility where T cells can be harvested, maintained, and easily transferred. The T cells can be cryopreserved and stored for later use. An acceptable duration of storage can be determined and validated and can be up to 6 months, up to a year, or longer.

The TIL population can be transduced at any point following collection. In some embodiments, a cryopreserved TIL population is transduced to express a CoStAR following thawing. In some embodiments, a TIL population is transduced to express a CoStAR during outgrowth or initial expansion from tumor starting material. In some embodiments, a TIL population is transduced to express a CoStAR during REP, for example but not limited to from about day 8 to about day 10 of REP. An exemplary TIL preparation is described in Applicant's U.S. patent application Ser. No. 62/951,559, filed Dec. 20, 2019. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

A specific subpopulation of T cells, such as CD3+, CD28+, CD4+, CD8+, CD45RA+, and CD45RO+ T cells, can be further isolated by positive or negative selection techniques. For example, in one aspect, T cells are isolated by incubation with anti-CD 3/anti-CD28-conjugated beads, such as DYNABEADS® M-450 CD3/CD28 T, for a time period sufficient for positive selection of the desired T cells. In some embodiments, the time period is about 30 minutes. In some embodiments, the time period ranges from 30 minutes to 36 hours or longer and all integer values there between. In some embodiments, the time period is at least 1, 2, 3, 4, 5, or 6 hours. In some embodiments, the time period is 10 to 24 hours. In one aspect, the incubation time period is 24 hours. Longer incubation times can be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used. In certain aspects, it can be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD 14, CD20, CD 16, HLA-DR, and CD8. In certain aspects, it can be desirable to enrich for or positively select for regulatory T cells which typically express CD4+, CD25+, CD62Lhi, GITR+, CD137, PD1, TIM3, LAG-3, CD150 and FoxP3+. Alternatively, in certain aspects, T regulatory cells are depleted by anti-CD25 conjugated beads or other similar method of selection.

The methods described herein can include, e.g., selection of a specific subpopulation of immune effector cells, e.g., T cells, that are a T regulatory cell-depleted population, CD25+ depleted cells, using, e.g., a negative selection technique, e.g., described herein. Preferably, the population of T regulatory depleted cells contains less than 30%, 25%, 20%, 15%, 10%, 5%, 4%, 3%, 2%, 1% of CD25+ cells.

A specific subpopulation of CoStAR effector cells that specifically bind to a target antigen can be enriched for by positive selection techniques. For example, in some embodiments, effector cells are enriched for by incubation with target antigen-conjugated beads for a time period sufficient for positive selection of the desired abTCR effector cells. In some embodiments, the time period is about 30 minutes. In some embodiments, the time period ranges from 30 minutes to 36 hours or longer (including all ranges between these values). In some embodiments, the time period is at least one, 2, 3, 4, 5, or 6 hours. In some embodiments, the time period is 10 to 24 hours. In some embodiments, the incubation time period is 24 hours. For isolation of effector cells present at low levels in the heterogeneous cell population, use of longer incubation times, such as 24 hours, can increase cell yield. Longer incubation times can be used to isolate effector cells in any situation where there are few effector cells as compared to other cell types. The skilled artisan would recognize that multiple rounds of selection can also be used.

T cells for stimulation can also be frozen after a washing step. After the washing step that removes plasma and platelets, the cells can be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing can be used as well as uncontrolled freezing immediately at −20° C. or in liquid nitrogen.

### Allogenic CoStAR

In some embodiments described herein, the immune effector cell can be an allogeneic immune effector cell, e.g., T cell or NK cell. For example, the cell can be an allogeneic T cell, e.g., an allogeneic T cell lacking expression of endogenous T cell receptor (TCR) and/or human leukocyte antigen (HLA), e.g., HLA class I and/or HLA class II.

A T cell lacking a functional endogenous TCR can be, e.g., engineered such that it does not express any functional TCR on its surface, engineered such that it does not express one or more subunits that comprise a functional TCR (e.g., engineered such that it does not express (or exhibits reduced expression) of TCR alpha, TCR beta, TCR gamma, TCR delta, TCR epsilon, and/or TCR zeta) or engineered such that it produces very little functional TCR on its surface. In some embodiments, the T cell can express a substantially impaired TCR, e.g., by expression of mutated or truncated forms of one or more of the subunits of the TCR. The term “substantially impaired TCR” means that this TCR will not elicit an adverse immune reaction in a host.

A T cell described herein can be, e.g., engineered such that it does not express a functional HLA on its surface. For example, a T cell described herein, can be engineered such that cell surface expression HLA, e.g., HLA class 1 and/or HLA class II, is downregulated. In some aspects, downregulation of HLA can be accomplished by reducing or eliminating expression of beta-2 microglobulin (B2M).

In some embodiments, the T cell can lack a functional TCR and a functional HLA, e.g., HLA class I and/or HLA class II. Modified T cells that lack expression of a functional TCR and/or HLA can be obtained by any suitable means, including a knock out or knock down of one or more subunit of TCR or HLA. For example, the T cell can include a knock down of TCR and/or HLA using siRNA, shRNA, clustered regularly interspaced short palindromic repeats (CRISPR) transcription-activator like effector nuclease (TALEN), or zinc finger endonuclease (ZFN).

In some embodiments, the allogeneic cell can be a cell which does not expresses or expresses at low levels an inhibitory molecule, e.g. a cell engineered by any method described herein. For example, the cell can be a cell that does not express or expresses at low levels an inhibitory molecule, e.g., that can decrease the ability of a CoStAR-expressing cell to mount an immune effector response. Examples of inhibitory molecules include PD1, PD-L1, PD-L2, CTLA4, TIM3, CEACAM (e.g, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, Gal9, adenosine, and TGFR beta. Inhibition of an inhibitory molecule, e.g, by inhibition at the DNA, RNA or protein level, can optimize a CAR-expressing cell performance. In embodiments, an inhibitory nucleic acid, e.g, an inhibitory nucleic acid, e.g, a dsRNA, e.g, an siRNA or shRNA, a clustered regularly interspaced short palindromic repeats (CRISPR), a transcription-activator like effector nuclease (TALEN), or a zinc finger endonuclease (ZFN), e.g, as described herein, can be used. In some embodiments, the fusion protein or CoSTaR comprises polypeptides of SEQ ID NO: 1, SEQ ID NO: 2, and/or any one of SEQ ID NO: 3-5, where the sequences are shown in FIG. 16. In some embodiments, this includes some part of SEQ ID NO: 1 and/or parts of SEQ ID NO: 2-11, and/or variants thereof. In some embodiments, the fusion protein or CoSTaR comprises the CEA construct components provided in FIG. 16 (all or in part or variants thereof). In some embodiments, this includes SEQ ID Nos: 12-17 (all or in part or variants thereof). In some embodiments, the fusion protein or CoSTaR comprises the pembrolizumab construct components provided in FIG. 16 (all or in part or variants thereof). In some embodiments, this includes SEQ ID Nos: 18-25 (all or in part or variants thereof). In some embodiments, the fusion protein or CoStAR will be the same as shown in FIG. 1, but with the first component being a binding domain (such as an scFv) to pembrolizumab or CEA. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

In some embodiments, a cancer specific CAR or TCR is present in the cell that contains the fusion protein or CoStAR. In some embodiments, a fusion protein or CoStAR can be expressed alone under the control of a promoter in a therapeutic population of cells that have therapeutic activity, for example, Tumor Infiltrating Lymphocytes (TILs). In some embodiments, the fusion protein or CoStAR can be expressed along with a therapeutic transgene such as a chimeric antigen receptor (CAR) and/or T-cell Receptor (TCR). In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

In some embodiments, suitable TCRs and CARs can be those that are well known in the literature, for example HLA-A*02-NYESO-1 specific TCRs (Rapoport et al. Nat Med 2015) or anti-CD19scFv.CD3z fusion CARs (Kochenderfer et al. J Clin Oncol 2015) which have been successfully used to treat Myeloma or B-cell malignancies respectively. In some embodiments, the CoStARs described herein can be expressed with any known CAR or TCR thus providing the cell with a regulatable growth switch to allow cell expansion in-vitro or in-vivo, and a conventional activation mechanism in the form of the TCR or CAR for anti-cancer activity. In some embodiments, a cell for use in adoptive cell therapy is provided and comprises a CoStAR as described herein and a TCR and/or CAR that specifically binds to a tumor associated antigen. In some embodiments, an exemplary CoStAR comprising CD28 includes an extracellular antigen binding domain and an extracellular, transmembrane and intracellular signaling domain. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

In some embodiments, prior to administration to the subject, the cells comprising the fusion protein or the CoStAR are incubated with irradiated feeder cells and supplemented with IL-2/mL, and wherein there is no expansion-effective amount IL-2 remaining when the cells are administered to the subject. This process of exposure to 11-2 is distinct from the absence or reduction of IL-2 noted herein, that instead occurs in vivo. Instead, this process of adding IL-2 happens in vitro, and the 11-2 remaining is inadequate to provide any significant in vivo benefit to the subject once the cells are administered to the subject.

In some embodiments, a combination of IL-2, IL-7, IL-15, and/or IL-21 are employed as a combination during the expansion. In some embodiments, IL-2, IL-7, IL-15, and/or IL-21 as well as any combinations thereof can be included during the expansion. In some embodiments, a combination of IL-2, IL-15, and IL-21 are employed as a combination during the expansion. In some embodiments, IL-2, IL-15, and IL-21 as well as any combinations thereof can be included. In some embodiments, the expansion can be conducted in a supplemented cell culture medium comprising IL-2, OKT-3, and antigen-presenting feeder cells.

In some embodiments, the cell culture medium comprises IL-2. In some embodiments, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL, or between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or between 8000 IU/mL of IL-2. As noted above, this is distinct from the in vivo process, which occurs with no or reduced levels of IL-2. In some embodiments the antigen-presenting feeder cells (APCs) are PBMCs. In some embodiments, the ratio of CoStAR cells to PBMCs and/or antigen-presenting cells in the expansion is about 1 to 25, about 1 to 50, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500, or between 1 to 50 and 1 to 300, or between 1 to 100 and 1 to 200.

### Activation and Expansion of T Cells

T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. As provided herein, this use of IL-2 is pre-in vivo use, and thus is consistent with the methods provided herein where low or no amounts of IL-2 are used during the in vivo stimulation of the cells.

Generally, the T cells can be expanded by contact with a surface having attached thereto an agent that stimulates a CD3/TCR complex associated signal and a ligand that stimulates a costimulatory molecule on the surface of the T cells. In particular, T cell populations can be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besancon, France) can be used as can other methods commonly known in the art (Berg et al, Transplant Proc. 30(8):3975-3977, 1998; Haanen et ah, J. Exp. Med. 190(9): 13191328, 1999; Garland et al, J. Immunol Meth. 227(1-2):53-63, 1999). In some embodiments, expansion can be performed using flasks or containers, or gas-permeable containers known by those of skill in the art and can proceed for 7 days, 8 days, 9 days, 10 days, 11 days, 12 days, 13 days, or 14 days, about 7 days to about 14 days, about 8 days to about 14 days, about 9 days to about 14 days, about 10 days to about 14 days, about 11 days to about 14 days, about 12 days to about 14 days, or about 13 days to about 14 days. In some embodiments, the second TIL expansion can proceed for about 14 days.

In some embodiments, the expansion can be performed using non-specific T-cell receptor stimulation in the presence of interleukin-2 (IL-2) or interleukin-15 (IL-15). The non specific T-cell receptor stimulus can include, for example, an anti-CD3 antibody, such as about 30 ng/ml of OKT3, a mouse monoclonal anti-CD3 antibody (commercially available from Ortho-McNeil, Raritan, N.J. or Miltenyi Biotech, Auburn, Calif.) or UHCT-1 (commercially available from BioLegend, San Diego, Calif., USA). CoStAR cells can be expanded in vitro by including one or more antigens, including antigenic portions thereof, such as epitope(s), of a cancer, which can be optionally expressed from a vector, such as a human leukocyte antigen A2 (HLA-A2) binding peptide, e.g., 0.3 .mu.M MART-E26-35 (27L) or gp100:209-217 (210M), optionally in the presence of a T-cell growth factor, such as 300 IU/mL IL-2 or IL-15. Other suitable antigens can include, e.g., NY-ESO-1, TRP-1, TRP-2, tyrosinase cancer antigen, MAGE-A3, SSX-2, and VEGFR2, or antigenic portions thereof. CoStAR cells can also be rapidly expanded by re stimulation with the same antigen(s) of the cancer pulsed onto HLA-A2-expressing antigen-presenting cells. In some embodiments, the CoStAR cells can be further stimulated with, e.g., example, irradiated, autologous lymphocytes or with irradiated HLA-A2+ allogeneic lymphocytes and IL-2. In some embodiments, the stimulation occurs as part of the expansion. In some embodiments, the expansion occurs in the presence of irradiated, autologous lymphocytes or with irradiated HLA-A2+ allogeneic lymphocytes and IL-2. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

In some embodiments, the cell culture medium comprises OKT3 antibody. In some embodiments, the cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, about 1 pg/mL or between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, or between 50 ng/mL and 100 ng/mL of OKT3 antibody.

In some embodiments, the expansion culture media comprises about 500 IU/mL of IL-15, about 400 IU/mL of IL-15, about 300 IU/mL of IL-15, about 200 IU/mL of IL-15, about 180 IU/mL of IL-15, about 160 IU/mL of IL-15, about 140 IU/mL of IL-15, about 120 IU/mL of IL-15, or about 100 IU/mL of IL-15, or about 500 IU/mL of IL-15 to about 100 IU/mL of IL-15, or about 400 IU/mL of IL-15 to about 100 IU/mL of IL-15 or about 300 IU/mL of IL-15 to about 100 IU/mL of IL-15 or about 200 IU/mL of IL-15, or about 180 IU/mL of IL-15.

In some embodiments, the expansion culture media comprises about 20 IU/mL of IL-21, about 15 IU/mL of IL-21, about 12 IU/mL of IL-21, about 10 IU/mL of IL-21, about 5 IU/mL of IL-21, about 4 IU/mL of IL-21, about 3 IU/mL of IL-21, about 2 IU/mL of IL-21, about 1 IU/mL of IL-21, or about 0.5 IU/mL of IL-21, or about 20 IU/mL of IL-21 to about 0.5 IU/mL of IL-21, or about 15 IU/mL of IL-21 to about 0.5 IU/mL of IL-21, or about 12 IU/mL of IL-21 to about 0.5 IU/mL of IL-21, or about 10 IU/mL of IL-21 to about 0.5 IU/mL of IL-21, or about 5 IU/mL of IL-21 to about 1 IU/mL of IL-21, or about 2 IU/mL of IL-21. In some embodiments, the cell culture medium comprises about 1 IU/mL of IL-21, or about 0.5 IU/mL of IL-21.

In some embodiments, the primary stimulatory signal and the costimulatory signal for the T cell can be provided by different protocols. For example, the agents providing each signal can be in solution or coupled to a surface. When coupled to a surface, the agents can be coupled to the same surface (i.e., in “cis” formation) or to separate surfaces (i.e., in “trans” formation). In some embodiments, one agent can be coupled to a surface and the other agent in solution. In one aspect, the agent providing the costimulatory signal is bound to a cell surface and the agent providing the primary activation signal is in solution or coupled to a surface. In some embodiments, both agents can be in solution. In some embodiments, the agents can be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells.

In some embodiments, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof; and both agents are co-immobilized to the same bead in equivalent molecular amounts. In one aspect, a 1:1 ratio of each antibody bound to the beads for CD4+ T cell expansion and T cell growth is used. In some embodiments, a ratio of anti CD3:CD28 antibodies bound to the beads is used such that an increase in T cell expansion is observed as compared to the expansion observed using a ratio of 1:1. In one particular aspect an increase of from about 1 to about 3 fold is observed as compared to the expansion observed using a ratio of 1:1. In one aspect, the ratio of CD3:CD28 antibody bound to the beads ranges from 100:1 to 1:100 and all integer values there between. In some embodiments, more anti-CD28 antibody is bound to the particles than anti-CD3 antibody, i.e., the ratio of CD3:CD28 is less than one. In some embodiments, the ratio of anti CD28 antibody to anti CD3 antibody bound to the beads is greater than 2:1. In some embodiments, a 1:100 CD3:CD28 ratio of antibody bound to beads is used. In one aspect, a 1:75 CD3:CD28 ratio of antibody bound to beads is used. In some embodiments, a 1:50 CD3:CD28 ratio of antibody bound to beads is used. In one aspect, a 1:30 CD3:CD28 ratio of antibody bound to beads is used. In one preferred aspect, a 1:10 CD3:CD28 ratio of antibody bound to beads is used. In some embodiments, a 1:3 CD3:CD28 ratio of antibody bound to the beads is used. In yet one aspect, a 3:1 CD3:CD28 ratio of antibody bound to the beads is used.

Ratios of particles to cells from 1:500 to 500:1 and any integer values in between can be used to stimulate T cells or other target cells. As those of ordinary skill in the art can readily appreciate, the ratio of particles to cells can depend on particle size relative to the target cell. For example, small sized beads could only bind a few cells, while larger beads could bind many. In some embodiments, the ratio of cells to particles ranges from 1:100 to 100:1 and any integer values in-between and in further aspects the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In one aspect, a ratio of particles to cells of 1:1 or less is used. In some embodiments, a preferred particle: cell ratio is 1:5. In some embodiments, the ratio of particles to cells can be varied depending on the day of stimulation. For example, in one aspect, the ratio of particles to cells is from 1:1 to 10:1 on the first day and additional particles are added to the cells every day or every other day thereafter for up to 10 days, at final ratios of from 1:1 to 1:10 (based on cell counts on the day of addition In some embodiments, the ratio of particles to cells is 1:1 on the first day of stimulation and adjusted to 1:5 on the third and fifth days of stimulation. In some embodiments, particles are added on a daily or every other day basis to a final ratio of 1:1 on the first day, and 1:5 on the third and fifth days of stimulation. In some embodiments, the ratio of particles to cells is 2:1 on the first day of stimulation and adjusted to 1:10 on the third and fifth days of stimulation. In some embodiments, particles are added on a daily or every other day basis to a final ratio of 1:1 on the first day, and 1:10 on the third and fifth days of stimulation. One of skill in the art will appreciate that a variety of other ratios can be suitable. In particular, ratios will vary depending on particle size and on cell size and type. In one aspect, the most typical ratios for use are in the neighborhood of 1:1, 2:1 and 3:1 on the first day.

In some embodiments, the cells, such as T cells, are combined with agent-coated beads, the beads and the cells are subsequently separated, and then the cells are cultured. In an alternative aspect, prior to culture, the agent-coated beads and cells are not separated but are cultured together. In some embodiments, the beads and cells are first concentrated by application of a force, such as a magnetic force, resulting in increased ligation of cell surface markers, thereby inducing cell stimulation.

In some embodiments, following in vitro expansion, CoStAR cells are no longer supplemented with exogenous IL-2. In some embodiments, the residual IL-2 present during administration of CoStAR cells is not an expansion effective amount for in vivo proliferation. In some embodiments, there is no residual IL-2 present during administration of CoStAR cells. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

In some embodiments, the cell is isolated from human PBMCs.

In some embodiments, T cells are isolated from peripheral blood lymphocytes by lysing the red blood cells and depleting the monocytes, for example, by centrifugation through a PERCOLL′ gradient or by counterflow centrifugal elutriation. In some embodiments, T cell can be collected at an apheresis center and cell storage facility where T cells can be harvested, maintained, and easily transferred. In some embodiments, the T cells can be cryopreserved and stored for later use. In some embodiments, an acceptable duration of storage can be determined and validated and can be up to 6 months, up to a year, or longer.

In some embodiments, the fusion protein or CoStAR enhances antitumor activity by providing costimulatory signaling.

In some embodiments, enhanced antitumor activity comprises reduction in tumor size or weight. In some embodiments, enhanced antitumor activity comprises inhibition of tumor metastasis. In some embodiments, enhanced antitumor activity comprises inhibition of tumor growth. In some embodiments, enhanced antitumor activity comprises relieving symptoms of one or more cancer symptoms. In some embodiments, enhanced antitumor activity comprises an increase in cytotoxic or cytostatic activity against cancer cells. In some embodiments, enhanced antitumor activity comprises reduction in the number of cancer cells.

In some embodiments, exogenous IL-2 is not needed to support engineered immune cell engraftment within the subject.

In some embodiments, a therapeutically effective dose of engineered immune cells can be administered to the patient without subsequent intravenous administration of IL-2 to support initial expansion and engraftment of the engineered immune cells in the host. In some embodiments, co-stimulatory signaling from the CD40 and CD28 domains provides sufficient co-stimulation to support initial expansion and engraftment.

In some embodiments, a presence of folate receptor alpha 1 expressing cells induces engineered cell survival and proliferation.

In some embodiments, folate receptor alpha 1 activates the CoStAR. In some embodiments, the activated CoStAR provides Signal 2. In some embodiments the native TCR provides Signal 1. In some embodiments the presence of folate receptor alpha 1 is provided by cancer cells. In some embodiments, folate receptor alpha 1 activates the CD28 and CD40 domains of the CoStAR. In some embodiments the activated CD28 and CD40 domains provide survival and proliferation signals to the cells. In some embodiments, the survival and proliferation signals provided by folate receptor alpha 1 are capable of supporting cell survival and proliferation in vivo without exogenous IL-2. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

In some embodiments, the cells are capable of sustained survival in the presence of folate receptor alpha 1 expressing cells and the absence of IL-2 in vivo.

In some embodiments, the survival and proliferation signals provided by folate receptor alpha 1 can support cell survival and proliferation in vivo without exogenous IL-2. In some embodiments, the presence of folate receptor alpha 1 expressing cells can stimulate survival of engineered cells through the engraftment process. In some embodiments, the presence of folate receptor alpha 1 expressing cells can stimulate survival of engineered cells throughout the course of treatment. In some embodiments, the IL-2 administered to the subject will be less than 125,000 IU/kg/day, less than 112,500 IU/kg/day, less than 100,000 IU/kg/day, less than 87,500 IU/kg/day, less than 75,000 IU/kg/day, less than 62,500 IU/kg/day, less than 50,000 IU/kg/day, less than 37,500 IU/kg/day, less than 25,000 IU/kg/day, less than 12,500 IU/kg/day, less than 6,250 IU/kg/day, 2,500 IU/kg/day, less than 1,250 IU/kg/day, or any IL-2 administered to a subject is de minimis.

In some embodiments, the engineered cells are capable of surviving at least 60 days post injection in the presence of folate receptor alpha 1 expressing cells without exogenous IL-2 in vivo. In some embodiments, the engineered cells are capable of surviving at least 2, 5, 7, 10, 14, 20, 21, 28, 30, 60, 90, or 120 days without exogenous IL-2 in vivo. In some embodiments, the engineered cells are capable of surviving at least 1, 2, 3, 4, 5 or more years without exogenous IL-2 in vivo.

In some embodiments, folate receptor alpha 1 stimulated engineered immune cells have reduced PD-1 expression following sustained proliferation.

In some embodiments, CoStAR expressing cells provide reduced PD-1 expression (e.g., as shown in the examples) following repeated stimulation with folate receptor alpha 1. In some embodiments, CoStAR expressing cells demonstrate reduced PD-1 expression following sustained proliferation stimulated with folate receptor alpha 1. In some embodiments, CoStAR expressing cells demonstrate reduced T cell exhaustion marker expression following repeated stimulation with folate receptor alpha 1. In some embodiments, CoStAR expressing cells do not show significant upregulation of PD-1 following repeated stimulation. In some embodiments, stimulated CoStAR expressing cells demonstrate delayed development of a T cell exhaustion phenotype compared to stimulated T cells not expressing a CoStAR. In some embodiments, this can be measured via the use of a percent marker comparison. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

In some embodiments, the cell therapy administered comprises a dosage of at least 5×10\hat{ }8 CoStAR-positive (CoStAR+) T cells, 1×10\hat{ }9 CoStAR+ viable T cells, 3×10\hat{ }9 CoStAR+ viable T cells, or 6×10\hat{ }9 CoStAR+ viable T cells.

In some embodiments, the cell therapy administered comprises a dosage of at least 5×10\hat{ }8 CoStAR+ T cells.

In some embodiments, the cell therapy administered comprises a dosage of at least 1×10\hat{ }9 CoStAR+ viable T cells.

In some embodiments, the cell therapy administered comprises a dosage of at least 3×10\hat{ }9 CoStAR+ viable T cells.

In some embodiments, the cell therapy administered comprises a dosage of at least 6×10\hat{ }9 CoStAR+ viable T cells.

In some embodiments, the cell therapy administered comprises a dosage of any number between 5×10\hat{ }8 and 6×10\hat{ }9 CoStAR+ viable T cells.

In some embodiments, the cell therapy administered initially comprises a dosage of 5×10\hat{ }8 CoStAR+ T cells and is subsequently increased to 1×10\hat{ }9 CoStAR+ viable T cells, increased to 3×10\hat{ }9 CoStAR+ viable T cells, or increased to 6×10\hat{ }9 CoStAR+ viable T cells during the course of treatment.

In some embodiments, the T cells CoStAR+ viable T cells can be transduced TILs. In some embodiments the TILs can be derived from EOC, NSCLC, or RCC tumors. In some embodiments, the T cells can be autologous. In some embodiments, dosing can be based on a target number of viable, CoStAR+ T cells. In some embodiments, TILs that have been transduced with a CoStAR can exhibit enhanced the activity of TILs and overcome the limitations posed by the tumor microenvironment on unmodified TILs.

In some embodiments, the CoStAR+ viable T cells can comprise a FRα targeting CoStAR, a CEA targeting CoStAR, a pembrolizumab targeting CoStAR, or a MSLN targeting CoStAR. In some embodiments, the final CoStAR product can consist of both nontransduced and transduced T cells. In some embodiments, dose levels can be increased by half logs.

In some embodiments, the cell therapy administered enhances duration of response (DOR), objective response rate (ORR), progression free survival (PFS), and/or overall survival (OS) of the subject receiving the administration.

In some embodiments, for participants who experience an objective response, DOR is defined as the time from their first objective response to disease progression or death. In some embodiments, ORR is defined as the incidence of a complete response (CR) or a partial response (PR) per a modified response. In some embodiments, PFS is defined as the time from the CoStAR infusion date to the date of disease progression or death from any cause. In some embodiments, PFS is defined as the time from the CoStAR infusion date to the date of disease progression or death from any cause. In some embodiments, the CoStAR can delay disease progression in a subject.

In some embodiments, the cell therapy administered reduces tumor volume in a subject.

In some embodiments, the solid tumor can be a RCC, NSCLC, or EOC tumor. In some embodiments, the cell therapy can reduce tumor volume in a subject by 5%, 10%, 20%, 30%, 40%, 50%, 60%, 60%. 70%, 80%, 90%, or 100%.

In some embodiments, the cell therapy can reduce tumor volume in the presence of exogenously provided IL-2. In some embodiments, the cell therapy can reduce tumor volume without the presence of exogenous IL-2. In some embodiments, the cell therapy enables enhanced T cell infiltration of the tumor in a subject. In some embodiments, the cell therapy enables enhanced T cell survival within the tumor of a subject.

In some embodiments, a method is provided comprising administering a population of cells engineered to express a FRα targeting CoStAR, wherein FRα expression by target cells enhances engineered T cell activation in a dose dependent manner.

In some embodiments, the FRα targeting CoStAR expressing cells are T cells. In some embodiments, the FRα targeting CoStAR expressing cells are TILs. In some embodiments, the FRα targeting CoStAR expressing cells are autologous to a subject. In some embodiments, the enhanced engineered T cell activation comprises increased secretion of effector cytokines comprising IFNγ, TNFα, and/or IL-2 upon recognition of signal 1 and signal 2. In some embodiments, the enhanced engineered T cell activation comprises expression of activation markers 4-1BB and CD69, or proliferation. In some embodiments, the enhanced engineered T cell activation comprises increased cytotoxicity against target cells. In some embodiments, the target cells are RCC, NSCLC, or EOC tumor cells. In some embodiments, a dose dependent manner indicates an enhanced T cell response to cells that express higher levels of FRα.

In some embodiments, the FRα targeting CoStAR expressing cells are intimately tuned to respond to even low levels of target antigen. In some embodiments, high levels of FRα expression alone are insufficient to activate FRα targeting CoStAR expressing cells without signal 1. In some embodiments, a dose dependent response manner of T cell activation may also be seen in T cells engineered to express other CoStAR constructs. In some embodiments, administration of the FRα targeting CoStAR expressing cells will not result in off tumor toxicity in the absence of signal 1.

In some embodiments, the dose dependent response of CoStAR engineered cells is to membrane bound FRα.

In some embodiments, the dose dependent response of CoStAR engineered cells to membrane bound FRα requires engagement of TCR signal 1.

In some embodiments, the CoStAR engineered cells do not exhibit a dose dependent T cell activation response to soluble FRα.

FRα is known to be released from the cells via membrane-associated protease or phospholipases. Soluble FRα in serum is significantly higher in malignant ovarian cancer patients compared to early stage.

In some embodiments, recombinant soluble FRα binds anti-FRα CoStAR expressed on the T cell surface. In some embodiments, soluble FRα does not inhibit the co-stimulatory signal provided by the CoStAR. In some embodiments, soluble FRα does not inhibit cytotoxicity of CoStAR transduced cells. In some embodiments, soluble FRα does not inhibit cytokine secretion from CoStAR transduced cells. In some embodiments increasing amounts of soluble FRα from at least Ong/mL to 200 ng/mL fails inhibit the co-stimulatory signal provided by the CoStAR.

In some embodiments, a method of selecting a subject for CoStAR therapy is provided. In some embodiments, the method comprises assessing expression of FRα. In some embodiments, expression of FRα confers a sensitivity to FRα targeting CoStARs, in a biological sample obtained from said subject. In some embodiments, the method comprises selecting said subject as one having a sensitivity to FRα targeting CoStARs, when said expression of FRα is identified.

In some embodiments the subject has been diagnosed with a cancer characterized by solid tumors with FRα expression. In some embodiments, the cancer can be EOC, NSCLC, or RCC. In some embodiments, expression of FRα within the tumor provides the CoStAR expressing T cell with signal 2 co-stimulation. In some embodiments, elevated expression of FRα in solid tumors can provide increased sensitivity to FRα targeting CoStARs. In some embodiments, assessment of tumor expression of FRα can be measured in a patient biological sample.

In some embodiments, the patient biological sample may include, but is not limited to: blood, saliva, tumor biopsy, tissue biopsy, and urine. In some embodiments, low levels of FRα expression within the tumor may be sufficient to sensitize a tumor to FRα targeting CoStARs.

In some embodiments, the subject selected to receive CoStAR therapy can also receive supplemental cancer therapy including but limited to: chemotherapy, radiation treatment, anticancer antibodies, CAR T therapy, CAR NK cell therapy, tumor resection, and other immunotherapy treatments.

In some embodiments, a method is provided for assessing expression of FRα. In some embodiments, expression of FRα confers a sensitivity to FRα targeting CoStARs, in a biological sample obtained from said subject. In some embodiments, a method is provided for selecting said subject as one having a sensitivity to FRα targeting CoStARs, when said expression of FRα is identified. In some embodiments, a method is provided for administering to a subject a TIL cell therapy, wherein the TIL cell therapy comprises a CoStAR.

In some embodiments, the TIL is specific for a tumor associated antigen. In some embodiments, signal 1 is provided by the TIL TCR and signal 2 is provided by the CoStAR. In some embodiments, signal 2 through the CoStAR drives enhanced cytokine production, clonal expansion, and upregulation of anti-apoptotic proteins in TILs. In some embodiments the TIL is derived from an EOC, NSCLC, or RCC tumor.

In some embodiments, FRα expression in a tumor enhances CoStAR antitumor activity via supplemental costimulatory signaling (signal 2).

In some embodiments, based on the mechanism of action of the CoStAR and lack of cytotoxicity in the absence of TCR-pMHC (signal 1), FRα expression in normal tissue is not expected to cause off-tumor toxicity.

In some embodiments, reduction in FRα expression is evaluated following CoStAR cell therapy as a clinical endpoint.

In some embodiments, FRα expression in subject tumor tissue as a secondary endpoint utilizing an analytically validated IHC assay. In some embodiments, administration of FRα targeting CoStARs can reduce FRα expression at tumor site. In some embodiments, FRα expression in tumor tissue is evaluated before and after CoStAR therapy.

In some embodiments, the CoStAR therapy can reduce FRα expression at tumor site in a subject by 5%, 10%, 20%, 30%, 40%, 50%, 60%, 60%. 70%, 80%, 90%, or 100%. In some embodiments, the CoStAR therapy can reduce the expression of other tumor markers at the tumor site in a subject by 5%, 10%, 20%, 30%, 40%, 50%, 60%, 60%. 70%, 80%, 90%, or 100%.

In some embodiments, FRα expression levels are assessed by immunohistochemistry (IHC), polymerase chain reaction (PCR), next generation sequencing (NGS), antibody detection, or companion diagnostic (cDx) assays.

IHC comprises the process of selectively identifying antigens (proteins) in cells of a tissue section by exploiting the principle of antibodies binding specifically to antigens in biological tissues. PCR comprises identification of FRα expression levels by detecting amplified DNA or RNA. Antibody detection comprises the use of a recombinant antibody to detect its cognate antigen in a sample. NGS comprises performing sequencing of millions of small fragments of DNA in parallel and mapping the reads to the human reference genome using bioinformatics. cDx assays comprise a medical device, often an in vitro device, which provides information that is essential for the safe and effective use of a corresponding drug or biological product.

In some embodiments, the cell therapy is administered intravenously in a cell suspension, wherein the cell therapy is provided as a single infusion.

In some embodiments, the cell therapy can be administered to the subject parenterally. In some embodiments the cell therapy can be administered in an inpatient setting. In some embodiments, subjects will remain hospitalized through day 7 post-infusion period. In some embodiments, the cell therapy is provided in multiple infusions.

In some embodiments, IL-2 is not co-administered to the subject with the cell therapy infusion.

In some embodiments, any one of the above steps can have further steps added between them. In some embodiments, any one or more of the above steps can be repeated. In some embodiments, any one or more of the above steps can be performed concurrently or out of the order provided herein.

In some embodiments, the CoStAR can maintain a “younger” T-cell phenotype. In some embodiments, the CoStAR can have a low or lower PD-1 expression. The low or lower PD-1 expression can be 1) lower than the corresponding cell that lacks the CoSTAR construct, but has otherwise been treated the same, and/or 2) equal to or lower than the corresponding cell that have been treated the same, but has received IL-2 during the in vivo therapy component (and optionally can lack the CoSTaR construct). In some embodiments, the CoStAR can have a low or lower fraction of Temra. The low or lower fraction of Temra can be 1) lower than the corresponding cell that lacks the CoSTAR construct, but has otherwise been treated the same, and/or 2) equal to or lower than the corresponding cell that has been treated the same, but has received IL-2 during the in vivo therapy component (and optionally can lack the CoSTaR construct). In some embodiments, the CoStAR can have a high proliferation potential. This can be 1) higher than the corresponding cell that lacks the CoSTAR construct, but has otherwise been treated the same, and/or 2) equal to or higher than the corresponding cell that have been treated the same, but has received IL-2 during the in vivo therapy component (and optionally can lack the CoSTaR construct). In some embodiments, the PD-1 expression or lower fraction of Temra can be decreased by 10, 20, 30, 40, 50, 60, 70, 80, 90, 95, 96, 97, 98, 99, or 100%. In some embodiments, the increase can be at least 10, 50, 100, 200, 300, 400, 500% or more. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

In some embodiments, ITIL-306 is an engineered autologous tumor-infiltrating lymphocyte (TIL) cell therapy product for the treatment of advanced solid tumors associated with expression of folate receptor α (FRα). In some embodiments, ITIL-306 is comprised of TILs engineered using a self-inactivating third-generation lentiviral vector (LVV) to express a plasma-membrane-bound, costimulatory antigen receptor (CoStAR) consisting of an extracellular, antibody derived, single-chain variable fragment (scFv) that recognizes FRα and an intracellular region containing both CD28 and CD40 costimulatory domains. In some embodiments, the anti-FRα CoStAR molecule is designed to enhance TIL activity upon engagement of FRα while in the presence of concurrent T-cell receptor (TCR)-mediated peptide-major histocompatibility complex (pMHC) recognition on the tumor cell.

In some embodiments, he anti-FRα CoStAR molecule is designed to enhance TIL activity upon engagement of FRα while in the presence of concurrent T-cell receptor (TCR)-mediated peptide-major histocompatibility complex (pMHC) recognition on the tumor cell.

In some embodiments, resected tumor tissue is first digested by automated mechanical compression in the presence of media with digestion enzymes and then cryopreserved. In some embodiments, the tumor digest is then processed in a manufacturing facility to both transduce TILs with the anti-FRα CoStAR LVV and increase the number of TILs for infusion. In some embodiments, ITIL-306 undergoes conventional ex vivo expansion in cell culture supported by the addition of irradiated allogeneic peripheral-blood mononuclear cells (PBMCs) and recombinant interleukin (IL)-2. In some embodiments, the TILs are propagated in culture for up to 23 days until a sufficient number of anti-FRα CoStAR+ TILs have been produced for administration.

In some embodiments, the method further comprises pre-stimulation of fusion protein expressing cells; wherein Signal 2 activation is performed before Signal 1 activation.

In some embodiments, “pre-stimulation” constitutes exposure of the fusion protein expressing cell to Signal 2 before exposure to Signal 1.

In some embodiments, pre-stimulation of the fusion protein expressing cells enhances subsequent stimulation to Signal 1.

In some embodiments, enhanced stimulation can be determined by methods including but not limited to: release of effector cytokines at elevated levels, elevated expression of activation markers, or increased cytotoxic activity.

In some embodiments, pre-stimulation of the fusion protein expressing cells with Signal 2 is completed before exposure of the cells to Signal 1.

In some embodiments, pre-stimulation of the fusion protein expressing cells with Signal 2 overlaps with exposure of the cells to Signal 1.

### Preparation of Fusion and CoStAR Cells

Viral- and non-viral-based genetic engineering tools can be used to generate CoStAR cells, including without limitation T cells, NK cells resulting in permanent or transient expression of therapeutic genes. Retrovirus-based gene delivery is a mature, well-characterized technology, which has been used to permanently integrate CARs into the host cell genome (Scholler J., e.g. Decade-long safety and function of retroviral-modified chimeric antigen receptor T cells. Sci. Transl. Med. 2012; 4:132ra53; Rosenberg S. A. et al, Gene transfer into humans—immunotherapy of patients with advanced melanoma, using tumor-infiltrating lymphocytes modified by retroviral gene transduction. N. Engl. J. Med. 1990; 323:570-578). In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

Non-viral DNA transfection methods can also be used. For example, Singh et al describes use of the Sleeping Beauty (SB) transposon system developed to engineer CAR T cells (Singh H., et al., Redirecting specificity of T-cell populations for CD19 using the Sleeping Beauty system. Cancer Res. 2008; 68:2961-2971) and is being used in clinical trials (see e.g., ClinicalTrials.gov: NCT00968760 and NCT01653717). The same technology is applicable to engineer CoStARs cells.

Multiple SB enzymes have been used to deliver transgenes. Mates describes a hyperactive transposase (SB100×) with approximately 100-fold enhancement in efficiency when compared to the first-generation transposase. SB100× supported 35-50% stable gene transfer in human CD34(+) cells enriched in hematopoietic stem or progenitor cells. (Mates L. et al., Molecular evolution of a novel hyperactive Sleeping Beauty transposase enables robust stable gene transfer in vertebrates. Nat. Genet. 2009; 41:753-761) and multiple transgenes can be delivered from multi cistronic single plasmids (e.g., Thokala R. et al., Redirecting specificity of T cells using the Sleeping Beauty system to express chimeric antigen receptors by mix-and-matching of VL and VH domains targeting CD123+ tumors. PLoS ONE. 2016; 1 1:e0159477) or multiple plasmids (e.g., Hurton L. V. et al., Tethered IL-15 augments antitumor activity and promotes a stem-cell memory subset in tumor-specific T cells. Proc. Natl. Acad. Sci. EISA. 2016; 113:E7788-E7797). Such systems are used with CoStARs.

Morita et al, describes the piggyBac transposon system to integrate larger transgenes (Morita D. et al., Enhanced expression of anti-CD19 chimeric antigen receptor in piggyBac transposon-engineered T cells. Mol. Ther. Methods Clin. Dev. 2017; 8: 131-140) Nakazawa et al. describes use of the system to generate EBV-specific cytotoxic T-cells expressing HER2-specific chimeric antigen receptor (Nakazawa Y et al, PiggyBac-mediated cancer immunotherapy using EBV-specific cytotoxic T-cells expressing HER2-specific chimeric antigen receptor. Mol. Ther. 2011; 19:2133-2143). Manuri et al used the system to generate CD-19 specific T cells (Manuri P. V. R. et al., piggyBac transposon/transposase system to generate CD19-specific T cells for the treatment of B-lineage malignancies. Hum. Gene Ther. 2010; 21:427-437).

Transposon technology is easy and economical. One potential drawback is the longer expansion protocols currently employed can result in T cell differentiation, impaired activity and poor persistence of the infused cells. Monjezi et al describe development mini circle vectors that minimize these difficulties through higher efficiency integrations (Monjezi R. et al., Enhanced CAR T-cell engineering using non-viral Sleeping Beauty transposition from mini circle vectors. Leukemia. 2017; 31:186-194). These transposon technologies can be used for CoStARs.

### Pharmaceutical Compositions

Some embodiments also relate to a pharmaceutical composition containing a vector or a CoStAR expressing cell together with a pharmaceutically acceptable carrier, diluent or excipient, and optionally one or more further pharmaceutically active polypeptides and/or compounds. Such compositions need not include IL-2 or any significant amount of Il-2 in some embodiments.

In some embodiments, a pharmaceutical composition is provided comprising a CoStAR described above and a pharmaceutically acceptable carrier. In some embodiments, a pharmaceutical composition is provided comprising a nucleic acid encoding a CoStAR according to any of the embodiments described above and a pharmaceutically acceptable carrier. In some embodiments, a pharmaceutical composition is provided comprising an effector cell expressing a CoStAR described above and a pharmaceutically acceptable carrier. Such a formulation can, for example, be in a form suitable for intravenous infusion. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

As used herein, by “pharmaceutically acceptable” or “pharmacologically compatible” is meant a material that is not biologically or otherwise undesirable, e.g., the material can be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

In some embodiments, provided is a population of modified T cells expressing a recombinant CoStAR. A suitable population can be produced by a method described above. The population of modified T cells can be for use as a medicament. For example, a population of modified T cells as described herein can be used in cancer immunotherapy therapy, for example adoptive T cell therapy. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

Other embodiments provide the use of a population of modified T cells as described herein for the manufacture of a medicament for the treatment of cancer, a population of modified T cells as described herein for the treatment of cancer, and a method of treatment of cancer can comprise administering a population of modified T cells as described herein to an individual in need thereof.

The population of modified T cells can be autologous i.e. the modified T cells were originally obtained from the same individual to whom they are subsequently administered (i.e. the donor and recipient individual are the same). A suitable population of modified T cells for administration to the individual can be produced by a method comprising providing an initial population of T cells obtained from the individual, modifying the T cells to express a cAMP PDE or fragment thereof and an antigen receptor which binds specifically to cancer cells in the individual, and culturing the modified T cells.

The population of modified T cells can be allogeneic i.e. the modified T cells were originally obtained from a different individual to the individual to whom they are subsequently administered (i.e. the donor and recipient individual are different). The donor and recipient individuals can be HLA matched to avoid GVHD and other undesirable immune effects. A suitable population of modified T cells for administration to a recipient individual can be produced by a method comprising providing an initial population of T cells obtained from a donor individual, modifying the T cells to express a CoStAR which binds specifically to cancer cells in the recipient individual, and culturing the modified T cells. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

Following administration of the modified T cells, the recipient individual can exhibit a T cell mediated immune response against cancer cells in the recipient individual. This can have a beneficial effect on the cancer condition in the individual.

Cancer conditions can be characterised by the abnormal proliferation of malignant cancer cells and can include leukaemias, such as AML, CIVIL, ALL and CLL, lymphomas, such as Hodgkin lymphoma, non-Hodgkin lymphoma and multiple myeloma, and solid cancers such as sarcomas, skin cancer, melanoma, bladder cancer, brain cancer, breast cancer, uterus cancer, ovary cancer, prostate cancer, lung cancer, colorectal cancer, cervical cancer, liver cancer, head and neck cancer, oesophageal cancer, pancreas cancer, renal cancer, adrenal cancer, stomach cancer, testicular cancer, cancer of the gall bladder and biliary tracts, thyroid cancer, thymus cancer, cancer of bone, and cerebral cancer, as well as cancer of unknown primary (CUP). Cancer cells within an individual can be immunologically distinct from normal somatic cells in the individual (i.e. the cancerous tumor can be immunogenic). For example, the cancer cells can be capable of eliciting a systemic immune response in the individual against one or more antigens expressed by the cancer cells. The tumor antigens that elicit the immune response can be specific to cancer cells or can be shared by one or more normal cells in the individual.

An individual suitable for treatment as described above can be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutan, gibbon), or a human.

In some embodiments, the individual is a human. In some embodiments, non-human mammals, especially mammals that are conventionally used as models for demonstrating therapeutic efficacy in humans (e.g. murine, primate, porcine, canine, or rabbit animals) can be employed.

### Method of Treatment

Cells, including T and NK cells, expressing CoStARs can either be created ex vivo either from a patient's own peripheral blood (autologous), or in the setting of a haematopoietic stem cell transplant from donor peripheral blood (allogenic), or peripheral blood from an unconnected donor (allogenic). In some embodiments, T-cells or NK cells can be derived from ex-vivo differentiation of inducible progenitor cells or embryonic progenitor cells to T-cells or NK cells. In these instances, T-cells expressing a CoStAR and, optionally, a CAR and/or TCR, are generated by introducing DNA or RNA coding for the CoStAR and, optionally, a CAR and/or TCR, by one of many means including transduction with a viral vector, transfection with DNA or RNA. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

T or NK cells expressing a CoStAR and, optionally, expressing a TCR and/or CAR can be used for the treatment of haematological cancers or solid tumors.

A method for the treatment of disease relates to the therapeutic use of a vector or cell, including a T or NK cell. In this respect, the vector, or T or NK cell can be administered to a subject having an existing disease or condition in order to lessen, reduce or improve at least one symptom associated with the disease and/or to slow down, reduce or block the progression of the disease. The method can cause or promote T-cell mediated killing of cancer cells. The vector, or T or NK cell can be administered to a patient with one or more additional therapeutic agents. The one or more additional therapeutic agents can be co-administered to the patient. By “co-administering” is meant administering one or more additional therapeutic agents and the vector, or T or NK cell sufficiently close in time such that the vector, or T or NK cell can enhance the effect of one or more additional therapeutic agents, or vice versa. In this regard, the vectors or cells can be administered first and the one or more additional therapeutic agents can be administered second, or vice versa. Alternatively, the vectors or cells and the one or more additional therapeutic agents can be administered simultaneously. One co-administered therapeutic agent that can be useful is IL-2, as this is currently used in existing cell therapies to boost the activity of administered cells. However, IL-2 treatment is associated with toxicity and tolerability issues.

As mentioned, for administration to a patient, the CoStAR effector cells can be allogeneic or autologous to the patient. In some embodiments, allogeneic cells are further genetically modified, for example by gene editing, so as to minimize or prevent GVHD and/or a patient's immune response against the CoStAR cells. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

The CoStAR effector cells are used to treat cancers and neoplastic diseases associated with a target antigen. Cancers and neoplastic diseases that can be treated using any of the methods described herein include tumors that are not vascularized, or not yet substantially vascularized, as well as vascularized tumors. The cancers can comprise non-solid tumors (such as hematological tumors, for example, leukemias and lymphomas) or can comprise solid tumors. Types of cancers to be treated with the CoStAR effector cells include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, plasmacytoma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include adrenocortical carcinoma, cholangiocarcinoma, fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, stomach cancer, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, thyroid cancer (e.g., medullary thyroid carcinoma and papillary thyroid carcinoma), pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer (e.g., cervical carcinoma and pre-invasive cervical dysplasia), colorectal cancer, cancer of the anus, anal canal, or anorectum, vaginal cancer, cancer of the vulva (e.g., squamous cell carcinoma, intraepithelial carcinoma, adenocarcinoma, and fibrosarcoma), penile cancer, oropharyngeal cancer, esophageal cancer, head cancers (e.g., squamous cell carcinoma), neck cancers (e.g., squamous cell carcinoma), testicular cancer (e.g., seminoma, teratoma, embryonal carcinoma, teratocarcinoma, choriocarcinoma, sarcoma, Ley dig cell tumor, fibroma, fibroadenoma, adenomatoid tumors, and lipoma), bladder carcinoma, kidney cancer, melanoma, cancer of the uterus (e.g., endometrial carcinoma), urothelial cancers (e.g., squamous cell carcinoma, transitional cell carcinoma, adenocarcinoma, ureter cancer, and urinary bladder cancer), and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

When “an immunologically effective amount,” “an anti-tumor effective amount,” “a tumor-inhibiting effective amount,” or “therapeutic amount” is indicated, the precise amount of the compositions to be administered can be determined by a physician with consideration of individual differences in age, weight, tumor size, extent of infection or metastasis, and condition of the patient (subject). It can generally be stated that a pharmaceutical composition comprising the T cells described herein can be administered at a dosage of 104 to 109 cells/kg body weight, in some instances 105 to 106 cells/kg body weight, including all integer values within those ranges. T cell compositions can also be administered multiple times at these dosages. The cells can be administered by using infusion techniques that are commonly known in immunotherapy (see, e.g., Rosenberg et ah, New Eng. J. of Med. 319:1676, 1988).

As provided herein, in the present methods of treatment, no or low levels of 11-2 are administered to the subject during the in vivo process of the therapy.

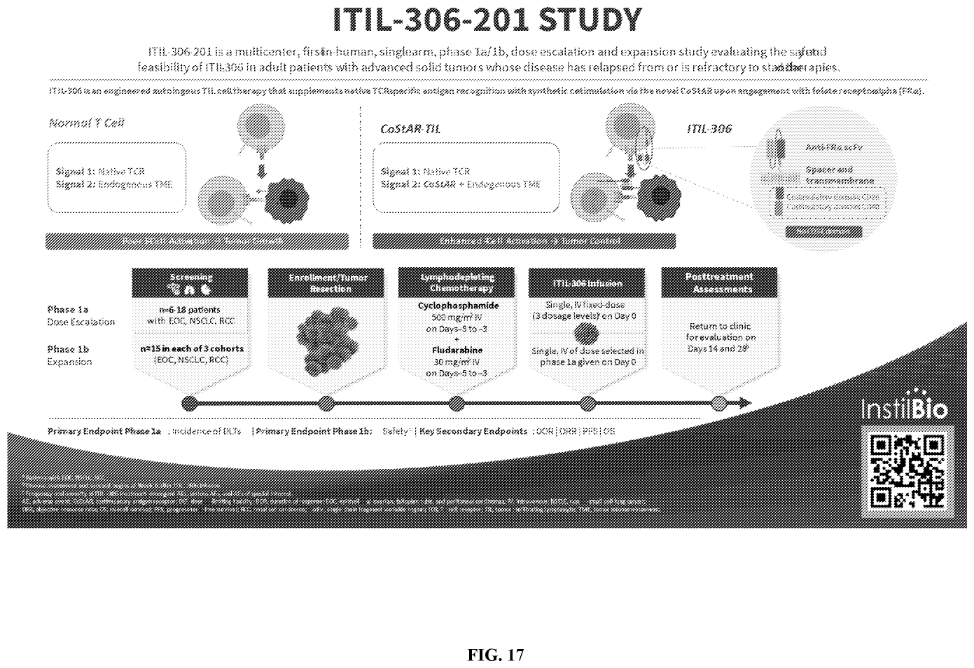

In some embodiments, additional aspects of the method are shown in part or whole in FIG. 17 and/or TABLES 1-3.

In some embodiments, the dosage can be 1×10\hat{ }9 CoStAR-positive (CoStAR+) viable T cells (±20% target dose). In some embodiments, the dosage can be, or be increased to 5×10\hat{ }8 CoStAR+ viable T cells (±20% target dose). In some embodiments, the dosage can be, or be increased to 3×10\hat{ }9 CoStAR+ viable T cells (±20% target dose). In some embodiments, the dosage can be, or be increased to, 6×10\hat{ }9 CoStAR+ viable T cells (+20% target dose). In some embodiments, the dosage is at least any one of the preceding values. In some embodiments, the dosage is between any two of the preceding values. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

### Combination Therapies

A CoStAR-expressing cell described herein can be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A CoStAR-expressing cell described herein and the at least one additional therapeutic agent can be administered simultaneously, in the same or in separate compositions, or sequentially. For sequential administration, the CAR-expressing cell described herein can be administered first, and the additional agent can be administered second, or the order of administration can be reversed. The CoStAR therapy and/or other therapeutic agents, procedures or modalities can be administered during periods of active disorder, or during a period of remission or less active disease. The CoStAR therapy can be administered before the other treatment, concurrently with the treatment, post-treatment, or during remission of the disorder. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

When administered in combination, the therapy and the additional agent (e.g., second or third agent), or all, can be administered in an amount or dose that is higher, lower or the same than the amount or dosage of each agent used individually, e.g., as a monotherapy. In some embodiments, the administered amount or dosage of the CoStAR therapy, the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the CoStAR therapy, the additional agent (e.g., second or third agent), or all, that results in a desired effect (e.g., treatment of cancer) is lower (e.g., at least 20%, at least 30%, at least 40%, or at least 50% lower) than the amount or dosage of each agent used individually, e.g., as a monotherapy, required to achieve the same therapeutic effect.

In further aspects, a CoStAR-expressing cell described herein can be used in a treatment regimen in combination with surgery, chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, cyclosporin, FK506, rapamycin, mycophenolic acid, steroids, FR901228, cytokines, and irradiation, peptide vaccine, such as that described in Izumoto et al. 2008 J Neurosurg 108:963-971. In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

In some embodiments, compounds are combined with other therapeutic agents, such as other anti-cancer agents, anti-allergic agents, anti-nausea agents (or anti-emetics), pain relievers, cytoprotective agents, and combinations thereof.

In some embodiments, a CoStAR-expressing cell described herein can be used in combination with a chemotherapeutic agent. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). General Chemotherapeutic agents considered for use in combination therapies include busulfan (Myleran®), busulfan injection (Busulfex®), cladribine (Leustatin®), cyclophosphamide (Cytoxan® or Neosar®), cytarabine, cytosine arabinoside (Cytosar-U®), cytarabine liposome injection (DepoCyt®), daunorubicin hydrochloride (Cerubidine®), daunorubicin citrate liposome injection (DaunoXome®), dexamethasone, doxorubicin hydrochloride (Adriamycin®, Rubex®), etoposide (Vepesid®), fludarabine phosphate (Fludara®), hydroxyurea (Hydrea®), Idarubicin (Idamycin®), mitoxantrone (Novantrone®), Gemtuzumab Ozogamicin (Mylotarg®). In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

In some embodiments, general chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Treatments can be evaluated, for example, by tumor regression, tumor weight or size shrinkage, time to progression, duration of survival, progression free survival, overall response rate, duration of response, quality of life, protein expression and/or activity. Approaches to determining efficacy of the therapy can be employed, including for example, measurement of response through radiological imaging.

As provided herein, some such therapies, no or low levels of 11-2 are administered to the subject during the in vivo section of the therapy, as the fusion protein allows for stimulation without the need for IL-2 administration to the subject.

In some embodiments, any one of the sequences used or provided in FIG. 16 can be employed in the methods provided herein. In FIG. 16, the underlined regions comprise CDRs. In some embodiments, the antigen binding domain of the fusion protein can target FRα. In some embodiments, the antigen binding domain of the fusion protein can target CEA. In some embodiments the antigen binding domain of the fusion protein can target Pembrolizumab. In some embodiments, this includes some part of SEQ ID NO: 1 and/or parts of SEQ ID NO: 2-11, and/or variants thereof. In some embodiments, the fusion protein or CoSTaR comprises the CEA construct components provided in FIG. 16 (all or in part or variants thereof). In some embodiments, this includes SEQ ID Nos: 12-17 (all or in part or variants thereof). In some embodiments, the fusion protein or CoSTaR comprises the pembrolizumab construct components provided in FIG. 16 (all or in part or variants thereof). In some embodiments, this includes SEQ ID Nos: 18-25 (all or in part or variants thereof). In some embodiments, the chimeric costimulatory antigen receptor, and/or fusion protein is any one or more of those disclosed herein, including for example, any of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN). In some embodiments, the fusion protein comprises a) a binding domain specific for CEA linked to; b) a transmembrane domain that is linked to; c) a CD28 signaling domain linked to; d) a CD40 signaling domain.

In some embodiments, a method of evaluating the potency of a fusion protein expressing cells is provided. The method comprises thawing and recovering fusion protein expressing cells and target cells overnight on Day 1, co-culturing fusion protein expressing cells with target cells for 5 hours on Day 2, and permeabilizing fusion protein expressing cells and evaluating intracellular T cell activation markers via flow cytometry on Day 3. The intracellular T cell activation markers to be evaluated comprise CD107a, IFNγ, CD137, and TNFα. The fusion protein comprises a binding domain specific for FRα, linked to a transmembrane domain, that is linked to a CD28 signaling domain, that is linked to a CD40 signaling domain. The fusion protein expressing cell is capable of survival and proliferation in the absence of exogenous IL-2 both in vitro and in vivo. In some embodiments, the assay is performed at a single cell level to evaluate the potency of a fusion protein expressing cell. In some embodiments, the assay is a one, two, or three or more day assay. In some embodiments, the potency analysis method is used to determine potency of a TIL population co-cultured with tumor cells or cells engineered to express a tumor associated antigen. In some embodiments, the potency analysis method is used to determine potency of a TIL population co-cultured with tumor cells from the same patient as the source of the TILs.

In some embodiments, Day 1 of the assay can comprise a thaw and overnight recovery. In some embodiments, controls include FMO (fluorescence minus one)-guided gating, a TIL positive control for system suitability and sample acceptance criteria for technical triplicates.

In some embodiments, Day 2 involves a 2, 3, 4, 5, 6, 7, or 8 hour co-culture to capture what happens inside the cell and shows a T cell producing a potency marker. In some embodiments, the target cells are K562 cells that are engineered to activate T cells via CD3, the signaling component of the T-cell receptor (TCR). In some embodiments, K562-OKT3 are the clonal target cells derived from K562 cells that were stably transduced to express the single-chain variable fragment (ScFv) from the CD3 agonist antibody OKT3. In some embodiments, the co-culture ratio is performed at 1:1. In some embodiments, co-culture of fusion protein expressing cells with target cells allows for T cell activation via TCR. In some embodiments, there is provided a negative control, for example, without limitation, non-transduced clonal K562 cells, K562-NT. The ratio of TILs to activating cells can be adjusted as needed. In some embodiments, the ratio of TILs to activating cells is from 10:1 to 1:10. In some embodiments, non-limiting examples include co-culture of TILs with stimulatory K562-OKT3 cells in ratios such as 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10.

In some embodiments, Day 3 involves permeabilizing the cell and evaluating intracellular stain markers. In some embodiments, tested analytes include CD107a (degranulation of T cell indicated activated cytotoxic T cells), IFN-γ (proinflammatory cytokine made by activated T cells), CD137 (4-1BB) (costimulatory activation marker for T cells) and TNFα (proinflammatory cytokine made by activated T cells). In some embodiments, the potency analysis is calculated using two of the analytes specific for T-cell mechanism of action, IFN-γ and CD107a. In some embodiment, to calculate potency, the total number of cells which express one (or both) of these analytes is quantified in each sample group.

In some embodiments, a method of evaluating the potency of a fusion protein expressing cells comprises: co-culturing fusion protein expressing cells with target cells, permeabilizing fusion protein expressing cells, and evaluating intracellular T cell activation markers. In some embodiments, the intracellular T cell activation markers to be evaluated comprise one or more of CD107a, IFNγ, CD137, and TNFα. In some embodiments, the fusion protein can comprise a binding domain specific for FRα (or, e.g., CEA or Pembrolizumab), linked to a transmembrane domain, that is linked to a CD28 signaling domain, that is linked to a CD40 signaling domain. The fusion protein expressing cell is capable of survival and proliferation in the absence of exogenous IL-2 both in vitro and in vivo.

In some embodiments non-limiting examples of analytes indicative of TIL activation and potency include IFN-γ, CD107a, CD137 (4-1BB). In some embodiments, other markers indicative or TIL activation or beneficial anti-tumor characteristics include, but are not limited to, IL-1beta, IL-2, IL-4, IL-6, IL-8, IL-10, IL-12p70, granzyme A/B, perforin, caspase 3 and other chemokine markers. In some embodiments, potency may be calculated as the frequency of all viable CD2+ cells that are positive for one or more of CD137, CD107a, TNF-α and IFN-γ, preferably CD107a and IFN-γ.

In some embodiments non-limiting examples of analytes indicative of TIL activation and potency include IFN-γ, CD107a, CD137 (4-1BB). In some embodiments, other markers indicative or TIL activation or beneficial anti-tumor characteristics include, but are not limited to, IL-1beta, IL-2, IL-4, IL-6, IL-8, IL-10, IL-12p70, granzyme A/B, perforin, caspase 3 and other chemokine markers. In some embodiments, potency may be calculated as the frequency of all viable CD2+ cells that are positive for one or more of CD137, CD107a, TNF-α and IFN-γ, preferably CD107a and IFN-γ.

In some embodiments, other potency mechanisms of action involving killing of tumor cells, secretion of cytokines and proliferation are evaluated. In some embodiments, tumor cell killing potency is characterized by flow cytometry to enumerate T cells and target cells and plate-based fluorescence or luminescence to measure percent killing. In some embodiments, cytokine secretion potency is characterized at the single cell level by flow cytometry and ELISA/MSD to characterize the population. In some embodiments, proliferation potency is determined by flow cytometry to characterize the population. In some embodiments, TIL potency may be determined by additional analytes, memory phenotype, cytotoxicity using cell lines, cytotoxicity using a patient specific tumor, a cytokine panel, cell proliferation and/or cellular composition. Additional information about potency assays and uses thereof can be found in PCT App. PCT/US2022/034606, filed on Jun. 22, 2022 with the title “Methods Of Isolating Of Tumor Infiltrating Lymphocytes And Use Thereof”, hereby expressly incorporated by reference in its entirety.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of and “consists essentially of have the meaning ascribed to them in U.S. Patent law, e.g., they allow for elements not explicitly recited, but exclude elements that are found in the prior art or that affect a basic or novel characteristic of the invention.

FIG. 17 provides some embodiments for the administration of various fusion proteins, such as a FRα CoSTaR to subjects. As shown in FIG. 17, the CoStAR-TIL (ITIL-306) can be administered to a subject, following lymphodepleting therapy (which can, in some embodiments, be accomplished by cyclophosphamide 500 mg/m 2 IV and fludarabine 30 mg/m 2 IV, both provided, (optionally) on Days −5 to −3). In some embodiments, the lymphodepleting regimen is reduced when administering TILs comprising a CoStAR. The ITIL-306 can be administered via infusion of, for example, a single IV fixed dose on Day 0. In some embodiments, subjects can be assessed posttreatment on Days 14 and 28. Some endpoints include duration of response, objective response rate, progression free survival, and overall survival. In some embodiments, the subjects are those outlined in TABLES 1-3. TABLES 1-3 include a description of ITIL-306-201. This outlines a phase 1a/1b, multicenter, clinical trial evaluating the safety and feasibility of ITIL-306 in adult participants with advanced solid tumors whose disease has progressed after standard therapy. As noted herein, ITIL-306 is a cell therapy derived from a participant's own tumor-infiltrating immune cells (lymphocytes; TILs) and contains a CoStAR that binds to folate receptor α (FRα) on the tumor. In some embodiments, any of the methods provided herein can employ any one or more of the aspects depicted in FIG. 17 and/or TABLES 1-3, especially with respect to: subjects to be treated, end goals to achieve, amount of administration, additional steps before or after the use of the cell therapy; and/or timing of administration. In some embodiments, no (or only low levels as provided herein) IL-2 is administered to the subject when or after the cell therapy is administered to the subject. In some embodiments, the dosage escalation can be 1×10\hat{ }9 CoStAR-positive (CoStAR+) viable T cells (±20% target dose), then to 5×10\hat{ }8 CoStAR+ viable T cells (±20% target dose), then to 3×10\hat{ }9 CoStAR+ viable T cells (±20% target dose), then to 6×10\hat{ }9 CoStAR+ viable T cells (±20% target dose). In some embodiments, the dosage is at least any one of the preceding values. In some embodiments, the dosage is between any two of the preceding values.

In some embodiments, a portion of the participant's tumor is surgically removed to make a personalized ITIL-306 product (or any CoStAR containing cell therapy). In some embodiments, once ITIL-306 (or any corresponding CoStAR containing cell therapy) has been made, the participant is treated with 3 days of lymphodepleting chemotherapy including cyclophosphamide and fludarabine, followed by 2 days of rest then a single infusion of ITIL-306. In some embodiments, no (or only low levels as provided herein) IL-2 is administered to the subject when or after the cell therapy is administered to the subject. In some embodiments, the DL1 for FIG. 17/TABLES 1-3 is 1×10\hat{ }9 CoStAR-positive (CoStAR+) viable T cells (±20% target dose), the DL-1 (optional) for FIG. 17/TABLES 1-3 is 5×10\hat{ }8 CoStAR+ viable T cells (±20% target dose), the DL2 for FIG. 17/TABLES 1-3 is 3×10\hat{ }9 CoStAR+ viable T cells (±20% target dose), and the DL3 for FIG. 17/TABLES 1-3 is 6×10\hat{ }9 CoStAR+ viable T cells (±20% target dose).

TABLE 1
Arms and Interventions of ITIL-306-201

| Arms | Interventions |
|---|---|
| Experimental: Phase 1a: | Biological/Vaccine: ITIL-306 |
| Dose Escalation | ITIL-306 is a cell therapy product derived from a |
| Various doses will be | participant's own TILs and contains a unique |
| tested in participants | molecule designed to increase TIL activity when it |
| with EOC, NSCLC and RCC. | encounters FRα on the tumor. A portion of the |
| | participant's tumor is surgically removed to make a |
| | personalized ITIL-306 product. Once ITIL-306 has |
| | been made, the participant is treated with 3 days of |
| | lymphodepleting chemotherapy including |
| | cyclophosphamide and fludarabine, followed by 2 |
| | days of rest then a single infusion of ITIL-306. |
| Experimental: Phase 1b: Expansion | Biological/Vaccine: ITIL-306 |
| Cohort 1: Participants with | ITIL-306 is a cell therapy product derived from a |
| epithelial ovarian cancer (EOC) | participant's own TILs and contains a unique |
| Cohort 2: Participants with | molecule designed to increase TIL activity when it |
| non-small cell lung cancer | encounters FRα on the tumor. A portion of the |
| (NSCLC) | participant's tumor is surgically removed to make a |
| Cohort 3: Participants with | personalized ITIL-306 product. Once ITIL-306 has |
| renal cell carcinoma (RCC) | been made, the participant is treated with 3 days of |
| | lymphodepleting chemotherapy including |
| | cyclophosphamide and fludarabine, followed by 2 |
| | days of rest then a single infusion of ITIL-306. |

TABLE 2
Outcome Measures of ITIL-306-201

| | |
|---|---|
| Primary Outcome Measure 1 | 1. Frequency and severity of ITIL-306 treatment-emergent adverse |
| | events (AEs), serious AEs, and AEs of special interest (AESI) [Time |
| | Frame: Up to 24 months] |
| Secondary Outcome Measure 2 | 2. Objective response rate (ORR) |
| | ORR defined as the incidence of a complete response (CR) or a |
| | partial response (PR) per a modified Response Evaluation Criteria in |
| | Solid Tumors (RECIST v1.1) criteria, as assessed by investigator |
| | review. |
| | [Time Frame: Up to 60 months] |
| Secondary Outcome Measure 3 | 3. Duration of response (DOR) |
| | For participants who experience an objective response, DOR is |
| | defined as the time from their first objective response to disease |
| | progression or death. |
| | [Time Frame: Up to 60 months] |
| Secondary Outcome Measure 4 | 4. Progression-free survival (PFS) |
| | PFS is defined as the time from the ITIL-306 infusion date to the date |
| | of disease progression or death from any cause. |
| | [Time Frame: Up to 60 months] |
| Secondary Outcome Measure 5 | 5. Overall Survival (OS) |
| | OS is defined as the time from the ITIL-306 infusion date to the date |
| | of death from any cause. |
| | [Time Frame: Up to 60 months] |

TABLE 3
Eligibility

| | |
|---|---|
| Key Inclusion Criteria | Histologically documented advanced (metastatic and/or unresectable) disease |
| | as appropriate per cohort. |
| | Phase 1a Dose Escalation: High-grade serous epithelial carcinoma of |
| | the ovary, fallopian tube, or peritoneum, adenocarcinoma of the lung, or clear- |
| | cell renal cell carcinoma. |
| | Phase 1b Expansion: |
| | Cohort 1: High grade serous, endometrioid, or clear cell epithelial |
| | carcinoma of the ovary, fallopian tube, or peritoneum. |
| | Cohort 2: Squamous-cell carcinoma or adenocarcinoma of the lung. |
| | Cohort 3: Clear cell or papillary RCC. |
| | Disease must have unequivocally progressed during or after at least 1 prior line |
| | of systemic therapy that must include the following parameters (by indication): |
| | Phase 1a dose escalation and Phase 1b Cohort 1: Participants with |
| | EOC whose disease has progressed during or after 1 prior line (at least 4 cycles) |
| | of platinum-based chemotherapy and had disease progression within 6 months |
| | from the last dose of the platinum agent. Participants who received 2 or more |
| | lines of platinum therapy must have disease which has progressed on or within |
| | 6 months after the date of the last dose of the platinum agent. Participants |
| | with BRCA-mutated EOC must have received previous PARP inhibitor therapy. |
| | Phase 1a dose escalation and Phase 1b Cohort 2: Participants with |
| | NSCLC whose disease has progressed after 1 prior line of platinum-based |
| | doublet chemotherapy and a CPI. Participants with targetable mutations (e.g. |
| | EGFR/ALK/KRAS) are required to have progressed on targeted therapy in |
| | addition to a platinum-based doublet chemotherapy |
| | Phase 1a dose escalation and Phase 1b Cohort 3: Participants with RCC |
| | whose disease has progressed after 1 prior line of antiangiogenic therapy and |
| | a PD-1-axis inhibitor. |
| | Medically suitable for surgical resection of tumor tissue. |
| | Following tumor resection for TIL harvest, will have, at minimum, 1 |
| | remaining measurable lesion as identified by CT or MRI per Response. |
| | Evaluation Criteria in Solid Tumors (RECIST) v1.1. |
| | Eastern Cooperative Oncology Group (ECOG) performance status 0 or |
| | 1. Adequate bone marrow and organ function. |
| Key Exclusion Criteria | History of another primary malignancy within the previous 3 years |
| | Phase 1a: |
| | EOC of the following subtypes: low-grade, endometrioid, clear cell, |
| | mucinous, sarcomatous, or mixed. |
| | NSCLC of the following subtypes: squamous, neuroendocrine |
| | differentiation. |
| | RCC of the following subtypes: nonclear-cell RCC |
| | Phase 1b: |
| | Cohort 1: Subjects with mucinous, sarcomatous, and low-grade EOC. |
| | Cohort 2: Subjects with small cell lung cancer, or NSCLC with |
| | neuroendocrine differentiation. |
| | Cohort 3: Subjects with nonclear-cell RCC, except papillary RCC. |
| | Previously received an allogeneic stem cell transplant or organ |
| | allograft. |
| | Previously received TIL or engineered cell therapy (eg, CAR T-cell). |
| | Significant cardiac disease. |
| | Stroke or transient ischemic attack within 12 months of enrollment. |
| | History of significant central nervous system (CNS) disorder. |
| | Symptomatic and/or untreated CNS metastases. |
| | History of significant autoimmune disease within 2 years prior to |
| | enrollment. |
| | Known history of severe, immediate hypersensitivity reaction |
| | attributed to cyclophosphamide, fludarabine, dimethyl sulfoxide (DMSO), |
| | human serum albumin (HAS), phosphate buffer or gentamycin. |

In some embodiments, the CoStAR constructs comprise the CDRs listed in TABLE 4.

TABLE 4
CoStAR Constructs CDR Sequences

| CoStAR | | |
|---|---|---|
| Construct | CDR | Sequence |
| ITIL-306 FRα | H-CDR1 | GYSFTGYF SEQ IS NO: 79 |
| | H-CDR2 | IHPYDGDT SEQ IS NO: 80 |
| | H-CDR3 | RYDGSRAMDY SEQ IS NO: 81 |
| | L-CDR1 | QSPASLAVSL SEQ IS NO: 82 |
| | L-CDR2 | RAS SEQ IS NO: 83 |
| | L-CDR3 | QQSREYPYT SEQ IS NO: 84 |
| Pembro | H-CDR1 | TNDMN SEQ IS NO: 85 |
| | H-CDR2 | VIYSDDTPDYATWAKG |
| | | SEQ IS NO: 86 |
| | H-CDR3 | GHYDSAVYAYALNI SEQ IS NO: 87 |
| | L-CDR1 | QASQSLSNLLA SEQ IS NO: 88 |
| | L-CDR2 | GASNLES SEQ IS NO: 89 |
| | L-CDR3 | QGGHYSGLT SEQ IS NO: 90 |
| CEA | H-CDR1 | GFNIKDS SEQ IS NO: 91 |
| | H-CDR2 | DPENGD SEQ IS NO: 92 |
| | H-CDR3 | TPTGPYYFD SEQ IS NO: 93 |
| | L-CDR1 | SSSVS SEQ IS NO: 94 |
| | L-CDR2 | STS SEQ IS NO: 95 |
| | L-CDR3 | RSSYPL SEQ IS NO: 96 |
| MSLN SS1 | H-CDR1 | GYSFTGYTMN SEQ IS NO: 97 |
| | H-CDR2 | ITPYNGASSYNQKFR |
| | | SEQ IS NO: 98 |
| | H-CDR3 | YDGRGFDY SEQ IS NO: 99 |
| | L-CDR1 | SASSSVSYMH SEQ IS NO: 100 |
| | L-CDR2 | DTSKLAS SEQ IS NO: 101 |
| | L-CDR3 | QQWSKHPLT SEQ IS NO: 102 |
| MSLN M5 | H-CDR1 | GYTFTDYYMH SEQ IS NO: 103 |
| | H-CDR2 | WINPNSGGTNYAQKFQ |
| | | SEQ IS NO: 104 |
| | H-CDR3 | GWDFDY SEQ IS NO: 105 |
| | L-CDR1 | RASQSIRYYLS SEQ IS NO: 106 |
| | L-CDR2 | TASILQN SEQ IS NO: 107 |
| | L-CDR3 | LQTYTTPD SEQ IS NO: 108 |
| MSLN HN1 | H-CDR1 | GYSINTYY SEQ IS NO: 109 |
| | H-CDR2 | INPSGVT SEQ IS NO: 110 |
| | H-CDR3 | AVYYCARWALWGDFGMDV |
| | | SEQ IS NO: 111 |
| | L-CDR1 | EGIYHW SEQ IS NO: 112 |
| | L-CDR2 | KAS SEQ IS NO: 113 |
| | L-CDR3 | QQYSNYPLT SEQ IS NO: 114 |
| MSLN M912 | H-CDR1 | GGSVSSGSYY SEQ IS NO: 115 |
| | H-CDR2 | IYYSGST SEQ IS NO: 116 |
| | H-CDR3 | AREGKNGAFDIW SEQ IS NO: 117 |
| | L-CDR1 | QSISSY SEQ IS NO: 118 |
| | L-CDR2 | AASS SEQ IS NO: 119 |
| | L-CDR3 | QQSYSTPLTF SEQ IS NO: 120 |
| MSLN huTP218 | H-CDR1 | FDLGFYFY SEQ IS NO: 121 |
| | H-CDR2 | IYTAGSGS SEQ IS NO: 122 |
| | H-CDR3 | ARSTANTRSTYYLNL |
| | | SEQ IS NO: 123 |
| | L-CDR1 | QRISSY SEQ IS NO: 124 |
| | L-CDR2 | GAS SEQ IS NO: 125 |
| | L-CDR3 | CQSYAYFDSNNWHAF |
| | | SEQ IS NO: 126 |
| MSLN P4 | H-CDR1 | SNSATWN SEQ IS NO: 127 |
| | H-CDR2 | RTYYRSKWYNDYAVSVKS |
| | | SEQ IS NO: 128 |
| | H-CDR3 | GMMTYYYGMDV SEQ IS NO: 129 |
| | L-CDR1 | TLRSGINVGPYRIY |
| | | SEQ IS NO: 130 |
| | L-CDR2 | YKSDSDKQQGS SEQ IS NO: 131 |
| | L-CDR3 | MIWHSSAAV SEQ IS NO: 132 |

### Arrangements

The following arrangements provide further options envisioned herein.
1. A method of treating cancer in a subject that expresses a tumor associated antigen (TAA), the method comprising:
   a. identifying a subject, wherein the subject has cancer that expresses a TAA; and
   b. administering to the subject a cell comprising a fusion protein, wherein the fusion protein comprises:
      i) a binding domain specific for the TAA linked to;
      ii) a transmembrane domain that is linked to;
      iii) a CD28 signaling domain that is linked to;
      iv) a CD40 signaling domain;
   c. wherein the subject does not receive exogenous IL-2 in a manner that is adequate for cell stimulation of TILs in vivo.
2. The method of arrangement 1, wherein the TAA is folate receptor α (FRα).
3. The method of arrangement 1, wherein the TAA is mesothelin (MSLN).
4. The method of arrangement 1, wherein the TAA is cancer antigen 125 (CA125).
5. The method of arrangement 1, wherein the TAA is CD228.
6. The method of arrangement 1, wherein the TAA is melanoma chondroitin sulfate proteoglycan (MCSP).
7. The method of arrangement 1, wherein the TAA is carcinoembryonic antigen (CEA).
8. A method of treating cancer in a subject that expresses folate receptor α (FRα), the method comprising:
   d. identifying a subject, wherein the subject has cancer that expresses FRα; and
   e. administering to the subject a cell comprising a fusion protein, wherein the fusion protein comprises:
      i) a binding domain specific for FRα linked to;
      ii) a transmembrane domain that is linked to;
      iii) a CD28 signaling domain that is linked to;
      iv) a CD40 signaling domain;
   c. wherein the subject does not receive exogenous IL-2 in a manner that is adequate for cell stimulation of TILs in vivo.
9. A method of cell therapy comprising:
   a. identifying a subject in need of tumor infiltrating lymphocyte (“TIL”) cell therapy; and
   b. administering to the subject a TIL cell therapy, wherein the TIL cell therapy:
      i. comprises a fusion protein that comprises:
         a) a binding domain specific for folate receptor α (FRα) linked to;
         b) a transmembrane domain that is linked to;
         c) a CD28 signaling domain that is linked to;
         d) a CD40 signaling domain;
         and
      ii. wherein the TIL cell therapy does not include a level of IL-2 administered to the subject, wherein the level is one that is sufficient to provide for IL-2 stimulated TIL cell therapy.
10. A method of administering a cell therapy, the method comprising:
   a. administering to a subject a TIL cell therapy, wherein the TIL cell therapy comprises a fusion protein that comprises:
      a) a binding domain specific for folate receptor α (FRα) linked to;
      b) a transmembrane domain that is linked to;
      c) a CD28 signaling domain that is linked to;
      d) a CD40 signaling domain;
         and
   b. wherein the method excludes a step of administering IL-2 to the subject to promote stimulation of the TILs in vivo, wherein stimulation of the TILs in vivo is achieved via the fusion protein.
11. A method of administering a cell therapy, the method comprising: administering a costimulatory antigen receptor (“CoStAR”) to a subject in the absence of a level of IL-2, wherein the level of IL-2 is one sufficient to cause TIL stimulation in vivo when the CoStAR is absent.
12. A method of administering a cell therapy for a cancer treatment, the method comprising administering a costimulatory antigen receptor (“CoStAR”) to a subject, wherein IL-2 is not used in the therapy at a level sufficient to promote TIL stimulation in the absence of the CoStAR.
13. A method of in vivo T cell expansion, the method comprising administering a T cell comprising a fusion protein to a subject, wherein IL-2 is not used to promote TIL stimulation, and wherein the fusion protein comprises:
   a) a binding domain specific for folate receptor α (FRα) linked to;
   b) a transmembrane domain that is linked to;
   c) a CD28 signaling domain that is linked to;
   d) a CD40 signaling domain.
14. The method of arrangement 13, wherein promoting TIL stimulation denotes a level of stimulation sufficient to achieve a therapeutically effective level of stimulation for a treatment of cancer in the subject.
15. The method of any one of arrangements 1-14, wherein stimulation is achieved via a TCR dependent mechanism that binds to a peptide that binds to an MHC.
16. A population of genetically engineered immune cells, wherein each immune cell comprises a fusion protein that comprises:
a) a binding domain specific for folate receptor α (FRα) linked to;
b) a transmembrane domain linked to;
c) a CD28 signaling domain linked to;
d) a CD40 signaling domain; and
wherein the population of genetically engineered immune cells has been administered to a subject who has not received an amount of IL-2 that is adequate to promote proliferation in vivo without the fusion protein, and wherein the population of immune cells has been expanded in the absence of IL-2 in vivo.
17. The method or population of any one of arrangements 1-16, wherein the cells are T cells.
18. The method or population of any one of arrangements 1-16, wherein the cells are donor T cells, from the subject.
19. The method or population of any one of arrangements 1-16, wherein the cells are tumor infiltrating lymphocytes.
20. The method or population of any one of arrangements 1-16, wherein the fusion protein or CoSTaR comprises the polypeptide of SEQ ID NO: 1, comprising the polypeptides of SEQ ID NO: 2-11.
21. The method of any one of the preceding method arrangements, wherein a cancer specific CAR or TCR is present in the cell that contains the fusion protein or CoStAR.
22. The method of any one of the preceding method arrangements, wherein prior to administration to the subject, the cells comprising the fusion protein or the CoStAR are incubated with irradiated feeder cells and supplemented with IL-2/mL, and wherein there is no expansion-effective amount IL-2 remaining when the cells are administered to the subject.
23. The method of any one of the preceding method arrangements, wherein the cell is isolated from human PBMCs.
24. The method of any one of the preceding method arrangements, wherein the cell comprises a human tumor infiltrating lymphocyte (TIL), an αβ T cell, a γδ T cell, or an NK T cell.
25. The method of any one of the preceding method arrangements, wherein the binding domain of the fusion protein or the CoSTaR comprises an scFv, a peptide, an antibody heavy-chain, a natural ligand or a receptor specific for FRα.
26. The method of any one of the preceding method arrangements, wherein the binding domain of the fusion protein or the CoSTaR comprises an scFv specific for FRα with a VH comprising SEQ ID NO: 3 and a VL comprising SEQ ID NO: 5.
27. The method of any one of the preceding method arrangements, wherein the binding domain is linked to the transmembrane domain by a linker and/or a spacer or wherein the fusion protein or CoSTaR comprises a linker and/or a spacer, wherein the linker comprises SEQ ID NO: 6.
28. The method of any one of the preceding method arrangements, wherein the transmembrane domain comprises the transmembrane domain of CD28, or wherein the fusion protein or CoSTaR comprises a transmembrane domain of CD28 comprising SEQ ID NO: 9.
29. The method of any one of the preceding method arrangements, wherein the CD40 domain consists of, consists essentially of, or comprises an SH3 motif (KPTNKAPH, SEQ ID NO:26), TRAF2 motif (PKQE, SEQ ID NO:27, PVQE, SEQ ID NO:28, SVQE, SEQ ID NO:29), TRAF6 motif (QEPQEINFP, SEQ ID NO:30), PKA motif (KKPTNKA, SEQ ID NO:31, SRISVQE, SEQ ID NO:32), a combination thereof, or is a full length CD40 intracellular domain, or wherein the fusion protein or CoSTaR comprises an SH3 motif (KPTNKAPH, SEQ ID NO:26), a TRAF2 motif (PKQE, SEQ ID NO:27, PVQE, SEQ ID NO:28, SVQE, SEQ ID NO:29), a TRAF6 motif (QEPQEINFP, SEQ ID NO:30), a PKA motif (KKPTNKA, SEQ ID NO:31, SRISVQE, SEQ ID NO:32), a combination thereof, or a full length CD40 intracellular domain.
30. The method of any of the preceding method arrangements, wherein the first signaling domain comprises a full length CD28 signaling domain or where the fusion protein or CoSTaR comprises a full length CD28 signaling domain comprising SEQ ID NO: 7.
31. The method of any one of the preceding method arrangements, wherein the fusion protein or CoStAR enhances antitumor activity by providing costimulatory signaling.
32. The method of any one of the preceding method arrangements, wherein exogenous IL-2 is not needed to support engineered immune cell engraftment within the subject.
33. The method of any one of the preceding method arrangements, wherein a presence of FRα expressing cells induces engineered cell survival and proliferation.
34. The method of any one of the preceding method arrangements, wherein the cells are capable of sustained survival in the presence of FRα cells and the absence of IL-2 in vivo.
35. The method of any one of the preceding method arrangements, wherein the engineered cells are capable of surviving at least 60 days post injection in the presence of FRα cells without exogenous IL-2 in vivo.
36. The method of any one of the preceding method arrangements, wherein FRα stimulated engineered immune cells have reduced PD-1 expression following sustained proliferation.
37. The method of any one of the preceding method arrangements, wherein the cell is derived from the subject's PBMCs.
38. The method of any one of the preceding arrangements, wherein the cancer comprises at least one of: solid tumors, renal cancer, lung cancer, or ovarian cancer.
39. The method of arrangement 14, further comprising:
   a) providing a cell expressing a fusion protein and a target cell;
   b) co-culturing the cell expressing the fusion protein with the target cell;
   c) permeabilizing the cell expressing the fusion protein; and
   d) evaluating at least one intracellular T cell activation markers.
40. The method of arrangement 39, further comprising flow cytometry for evaluating the at least one intracellular T cell activation marker.
41. The method of arrangement 39, wherein the at least one intracellular T cell activation markers comprise CD107a, IFN-γ, CD137, and/or TNFα.
42. The method of arrangement 39, wherein the fusion protein comprises:
   i) a binding domain specific for FRα, CEA MSLN, CA125, CD19, CD228, or pembrolizumab linked to;
   ii) a transmembrane domain that is linked to;
   iii) a CD28 signaling domain that is linked to;
   iv) a CD40 signaling domain. 43. The method of arrangement 42, wherein the cell expressing the fusion protein is capable of survival and proliferation in the absence of exogenous IL-2 both in vitro and in vivo.
44. A method of providing treatment to a subject that expresses CEA, MSLN or has pembrolizumab in their system, the method comprising:
   a. identifying a subject, wherein the subject has cancer that expresses CEA, MSLN, or has pembrolizumab in their system; and
   b. administering to the subject a cell comprising a fusion protein, wherein the fusion protein comprises:
      i) a binding domain specific for the corresponding CEA, MSLN or pembrolizumab linked to;
      ii) a transmembrane domain that is linked to;
      iii) a CD28 signaling domain for the MSLN or pembrolizumab binding domain or ICOS for the CEA binding domain that is linked to;
      iv) a CD40 signaling domain;
   c. wherein the subject does not receive exogenous IL-2 in a manner that is adequate for cell stimulation of TILs in vivo.
45. A method of cell therapy comprising:
   a. identifying a subject in need of tumor infiltrating lymphocyte (“TIL”) cell therapy; and
   b. administering to the subject a TIL cell therapy, wherein the TIL cell therapy:
      i. comprises a fusion protein that comprises:
         a) a binding domain specific for CEA, MSLN or pembrolizumab linked to;
         b) a transmembrane domain that is linked to;
         c) a CD28 signaling domain (for the MSLN or pembrolizumab binding domain) or an ICOS domain (for the CEA binding domain) that is linked to;
         d) a CD40 signaling domain;
         and
      ii. wherein the TIL cell therapy does not include a level of IL-2 administered to the subject, wherein the level is one that is sufficient to provide for IL-2 stimulated TIL cell therapy.
46. A method of administering a cell therapy, the method comprising:
   c. administering to a subject a TIL cell therapy, wherein the TIL cell therapy comprises a fusion protein that comprises:
      a) a binding domain specific for CEA, MSLN or pembrolizumab linked to;
      b) a transmembrane domain that is linked to;
      c) a CD28 signaling domain (for the MSLN or pembrolizumab binding domain) or an ICOS domain (for the CEA binding domain) that is linked to;
      d) a CD40 signaling domain;
         and
   d. wherein the method excludes a step of administering IL-2 to the subject to promote stimulation of the TILs in vivo, wherein stimulation of the TILs in vivo is achieved via the fusion protein.
47. A method of in vivo T cell expansion, the method comprising administering a T cell comprising a fusion protein to a subject, wherein IL-2 is not used to promote TIL stimulation, and wherein the fusion protein comprises:
   a) a binding domain specific for CEA, MSLN or pembrolizumab linked to;
   b) a transmembrane domain that is linked to;
   c) a CD28 signaling domain (for the MSLN or pembrolizumab binding domain) or an ICOS domain (for the CEA binding domain) that is linked to;
   d) a CD40 signaling domain.
48. The method of any one of the preceding arrangements, wherein the fusion protein comprises any one or more of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN).
49. The method of any one of the preceding arrangements, wherein the fusion protein comprises the CDRs as depicted in any one of FIG. 20C, 21C, 22C, or 23D.
50. The method of any one of the preceding arrangements, wherein the fusion protein comprises the VH as depicted in any one of FIG. 20C, 21C, 22C, or 23D or a binding fragment thereof.
51. The method of any one of the preceding arrangements, wherein the fusion protein comprises the VL as depicted in any one of FIG. 20C, 21C, 22C, or 23D or a binding fragment thereof.
52. The method of any one of the preceding arrangements, wherein the fusion protein comprises the CD40 as depicted in any one of FIG. 20C, 21C, 22C, or 23D or a binding fragment thereof.
53. The method of any one of the preceding arrangements, wherein the fusion protein comprises the CD28 or ICOS domain as depicted in any one of FIG. 20C, 21C, 22C, or 23C.
54. The method of any one of the preceding arrangements, wherein the fusion protein comprises the sequence as depicted in any one of FIG. 20D, 21D, 22D, or 23D or a sequence at least 85, 86, 87, 88, 89, 90, 91, 92, 93, 94, 95, 96, 97, 98, 99 or 100% identical thereto.
55. The method of any one of the preceding arrangements, wherein the fusion protein lacks the signal peptide sequence.
56. The method of any one of the preceding arrangements, wherein the fusion protein lacks one of SEQ ID NO: 36, 34, or 2.
57. The method of any one of the preceding arrangements, wherein the signal sequence has been cleaved from the fusion protein.
58. The method of any one of the preceding arrangements, wherein the cell therapy administered comprises a dosage of 5×10\hat{ }8 CoStAR-positive (CoStAR+) T cells, 1×10\hat{ }9 CoStAR+ viable T cells, 3×10\hat{ }9 CoStAR+ viable T cells, or 6×10\hat{ }9 CoStAR+ viable T cells.
59. The method of any one of the preceding arrangements, wherein the cell therapy administered comprises a dosage of at least 5×10\hat{ }8 CoStAR+ T cells.
60. The method of any one of the preceding arrangements, wherein the cell therapy administered comprises a dosage of at least 1×10\hat{ }9 CoStAR+ viable T cells.
61. The method of any one of the preceding arrangements, wherein the cell therapy administered comprises a dosage of at least 3×10\hat{ }9 CoStAR+ viable T cells.
62. The method of any one of the preceding arrangements, wherein the cell therapy administered comprises a dosage of at least 6×10\hat{ }9 CoStAR+ viable T cells.
63. The method of any one of the preceding arrangements, wherein the cell therapy administered comprises a dosage of any number of cells between 5×10\hat{ }8 and 6×10\hat{ }9 CoStAR+ viable T cells.
64. The method of any one of the preceding arrangements, wherein the cell therapy administered initially comprises a dosage of 5×10\hat{ }8 CoStAR+ T cells and is subsequently increased to 1×10\hat{ }9 CoStAR+ viable T cells, increased to 3×10\hat{ }9 CoStAR+ viable T cells, or increased to 6×10\hat{ }9 CoStAR+ viable T cells during the course of treatment.
65. The method of any one of the preceding arrangements, wherein the cell therapy administered enhances duration of response, objective response rate, progression free survival, and/or overall survival of the subject receiving the administration.
66. The method of any one of the preceding arrangements, wherein the cell therapy administered reduces tumor volume in a subject.
67. A method of cell therapy comprising administering a population of cells engineered to express a FRα targeting CoStAR, wherein FRα expression by target cells enhances engineered T cell activation in a dose dependent manner.
68. The method of any one of the preceding arrangements, wherein the dose dependent response of CoStAR engineered cells is to membrane bound FRα.
69. The method of any one of the preceding arrangements, wherein the dose dependent response of CoStAR engineered cells to membrane bound FRα requires engagement of TCR signal 1.
70. The method of any one of the preceding arrangements, wherein the CoStAR engineered cells do not exhibit a dose dependent T cell activation response to soluble FRα.
71. A method of selecting a subject for CoStAR therapy comprising:
   assessing expression of FRα, wherein expression of FRα confers a sensitivity to FRα targeting CoStARs, in a biological sample obtained from said subject; and
   selecting said subject as one having a sensitivity to FRα targeting CoStARs, when said expression of FRα is identified.
72. A method of administering a cell therapy in a subject, the method comprising:
   a) assessing expression of FRα, wherein expression of FRα confers a sensitivity to FRα targeting CoStARs, in a biological sample obtained from said subject,
   b) selecting said subject as one having a sensitivity to FRα targeting CoStARs, when said expression of FRα is identified; and
   c) administering to a subject a TIL cell therapy, wherein the TIL cell therapy comprises a CoStAR.
73. A method of any one of the preceding arrangements, wherein FRα expression in a tumor enhances CoStAR antitumor activity via supplemental costimulatory signaling (signal 2).
74. A method of any one of the preceding arrangements, wherein, based on the mechanism of action of the CoStAR and lack of cytotoxicity in the absence of TCR-pMHC (signal 1), FRα expression in normal tissue is not expected to cause off-tumor toxicity.
75. A method of any one of the preceding arrangements, wherein reduction in FRα expression is evaluated following CoStAR cell therapy as a clinical endpoint.
76. The method of any one of the preceding arrangements, wherein FRα expression levels are assessed by immunohistochemistry (IHC), polymerase chain reaction (PCR), next generation sequencing (NGS), antibody detection, or companion diagnostic (cDx) assays.
77. The method of any one of the preceding arrangements where the cell therapy is administered intravenously in a cell suspension, wherein the cell therapy is provided as a single infusion.
78. The method of any one of the preceding arrangements wherein the method further comprises pre-stimulation of fusion protein expressing cells; wherein Signal 2 activation is performed before Signal 1 activation.
79. The method of arrangement 78, wherein pre-stimulation of the fusion protein expressing cells enhances subsequent stimulation to Signal 1.
80. The method of arrangement 78, wherein pre-stimulation of the fusion protein expressing cells with Signal 2 is completed before exposure of the cells to Signal 1.
81. The method of arrangement 78, wherein pre-stimulation of the fusion protein expressing cells with Signal 2 overlaps with exposure of the cells to Signal 1.

### EXAMPLES

Additional embodiments are disclosed in further detail in the following examples, which are not in any way intended to limit the scope of the claims.

### Example 1

### Production of Non-Td, TCR-Td, CoStAR-Td, and TCR.CoStAR-Td Healthy Donor T Cells Materials and Methods

Human T cells were isolated from peripheral blood mononuclear cells (PBMCs) from healthy donors (HD) using a STEMCELL CD3 T cell isolation kit according to the manufacturers protocol. Cells were counted using the ViCell BLU automated cell counter and plated in complete T cell media (TCM; RPMI 1640 Medium GlutaMAX™ Supplement, HEPES, 10% FBS, 1× penicillin-streptomycin and 50 μM β-mercaptoethanol) supplemented with 200 IU IL-2/mL at 1×106 T cells/mL. Activating CTS Dynabeads were added to T cell cultures at a bead to T cell ratio of 1:3 prior to incubation for 48 hours in a humidified incubator (37° C., 5% CO2). Cryopreserved lentivirus were thawed at room temperature before transfer to a class II safety cabinet. Activated human CD3 T cells were counted and resuspended at 1×106 live cells/mL. Transduction solutions were made with 5 transforming units (TU) per T cell in TCM supplemented with 0.4 mg/mL polybrene and 200 IU IL-2/mL in a volume equivalent to the activated T cell suspension. All T cell groups were centrifuged (400 g, 5 min, RT) and the supernatant discarded before being resuspended in LV transduction solutions, and centrifuged (1200 g, 1.5h, 32° C.). HD T cells were either not transduced (Non-Td T cells), transduced with lentivirus encoding anti-FRα CoStAR molecule (CoStAR-Td T cells) or anti CEA TCR (TCR-Td T cells). To produce TCR.CoStAR-Td T cells, HD T cells were dual transduced with two separate lentiviruses encoding anti-FRα CoStAR molecule and anti-CEA TCR. An equal volume of TCM supplemented with 200 IU IL-2/mL was added to activated T cell LV cell suspensions before to incubation for 72 hours in a humidified incubator (37° C., 5% CO2). CTS Dynabeads were removed from all T cell groups using a STEMCELL magnet. All T cell groups were then counted and resuspended in TCM supplemented with 200 IU IL-2/mL. Cells were treated with 50 nM dasatinib and stained for FACS. The staining panel consisted of a viability stain, a stain with FRα-Fc and antibodies against FRα-Fc and murine TCRββ. CoStAR-Td T cells were sorted for CoStARpos T cells, TCR-Td T cells were sorted for TCRpos T cells and dual transduced T cells were sorted for CoStARpos TCRpos Td T cells. CoStAR-Td, TCR-Td and TCR.CoStAR-Td T cells from donor 37636 underwent FACS sorting 24 hours after bead removal, while the same groups from donor 41179 went through FACS sorting 48 hours after beads removal. Following FACS, all groups of T cells were counted, resuspended at 1×106 live cells/mL in TCM supplemented with 200 IU IL-2 and incubated in a humidified incubator (37° C., 5% CO2). 72 hours after CTS Dynabead removal, irradiated feeders, all groups of T cells were counted and T cells resuspended in TCM supplemented with 200 IU IL-2/mL and irradiated feeders at a ratio of 1:200. Feeder cell suspensions were seeded in 6-well G-REX plates at 30 mL/well before incubation in a humidified incubator for (37° C., 5% CO2) for 12 days. Cultures were supplemented with 200 IU IL-2/mL every 2 to 3 days and 5/7 of media replaced when required to maintain a neutral pH. All T cell groups were harvested, and their number and viability determined prior to cryopreservation. To determine T cell viability, a 50 μL aliquot was taken per sample for dead (DRAQ7+Annexin V+) and apoptotic (DRAQ7−Annexin V+) cell staining. The live (DRAQ7−Annevin V−) cell number was determined using a Novocyte 3005 Flow Cytometer System. Cells were centrifuged (400 g, 5 min, RT) and the supernatant discarded before being resuspended in Sigma-Aldrich CryoStor CS10 and aliquoted into cryovials at 1×107 or 1×108 T cells per vial for in vitro characterization or in vivo use, respectively.

### Example 2

### Recovery of Non-Td, TCR-Td, CoStAR-Td, and TCR.CoStAR-Td Healthy Donor T Cells from Cryopreservation

### Materials and Methods

All cryopreserved T cell groups were thawed in a 37° C. bead bath before transfer to a class II safety cabinet. Samples were immediately transferred into a 10× volume of TCM. To remove residual cryopreservation medium, all groups of T cells were centrifuged (400 g, 5 min, room temperature), the supernatant discarded and cells were then resuspended in 10× volume of TCM. To determine T cell viability, a 50 μL aliquot was taken per sample for dead (DRAQ7+Annexin V+) and apoptotic (DRAQ7−Annexin V+) cell staining. The viable (DRAQ7−Annevin V−) cell number was determined using a Novocyte 3005 Flow Cytometer System.

### Example 3

### Non-Td, TCR-Td, CoStAR-Td, and TCR.CoStAR-Td Healthy Donor T Cell Fold Expansion and Quantification of Transduction Efficiency Via Flow Cytometry

### Results

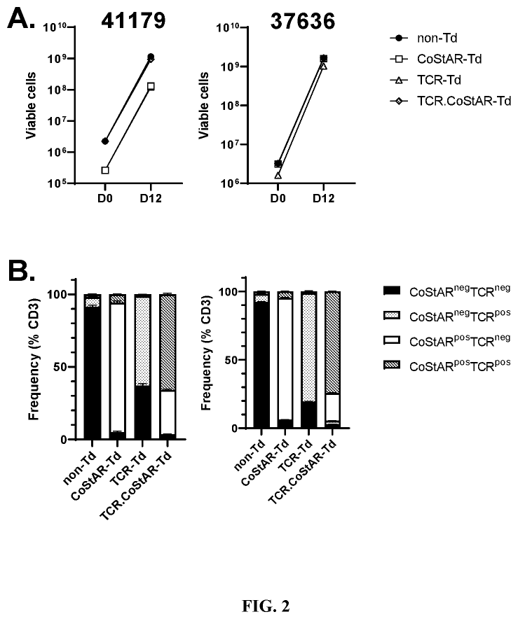

HD T cells of sufficient number from two independent donors were successfully produced as in Example 1 for in vitro characterization and in vivo studies. For donor 41179, an average (±standard deviation [SD]) of 5.9±5.37×10⁸ cells across all conditions were produced with Non-Td, CoStAR-Td, TCR-Td and TCR.CoStAR-Td cells having similar fold increases of 516-, 503-, 460-, and 419-fold, respectively (FIG. 2 panel A). Similarly, for donor 37636, an average of 1.5±0.31×10⁹ cells across all conditions were produced with Non-Td, CoStAR-Td, TCR-Td and TCR.CoStAR-Td having similar fold increases of 513-, 513-, 646-, and 509-fold, respectively (FIG. 2 panel A). Product cells were stimulated with a single (Day 0; IL-2) or serial (Day 0, 7, 14, 21; no IL-2) addition of target cells expressing membrane-anchored OKT3 (muromonab-CD3; to allow for TCR/CD3 complex crosslinking) and FRα (to provide signaling through anti-FRα CoStAR).

Expanded T cells efficiently expressed transgenic high-affinity anti-CEA TCR and/or anti-FRα CoStAR molecule following transduction and expansion. For donor 41179, 89.1% (SD±1.27%) of CoStAR-Td T cells were CoStARP^{pos}TCR^{neg} and for TCR-Td T cells 61.6% (SD±1.27%) were CoStAR^{neg}TCR^{pos}. TCR.CoStAR-Td T cells were 66.1% (SD±0.82%) CoStARP^{pos}TCRβ^{pos} whilst 30.3% (SD±0.76%) were CoStARP^{pos}TCR^{neg} (FIG. 2 panel B). For donor 37636, 88.9% (SD±0.51%) of CoStAR-Td T cells were CoStARP^{pos}TCR^{neg} and TCR-Td T cells were 79.5% (SD±0.59%) CoStAR^{neg}TCR^{pos}. In the TCR.CoStAR-Td T cells, 74.3% (SD±0.35%) were CoStARP^{pos}TCRβ^{pos} whilst 20.2% (SD±0.37%) were CoStARP^{pos}TCR^{neg} (FIG. 2 panel B).

### Example 4

### Phenotypic Characterization of Non-Td, TCR-Td, CoStAR-Td, and TCR.CoStAR-Td Healthy Donor T Cells

### Materials and Methods

All T cell groups recovered from donors 37636 and 41179 were resuspended in TCM without IL-2 at 1×106 live T cells/mL and rested overnight in a humidified incubator (37° C., 5% CO2). Viable cell counts were determined using the ViCell BLU automated cell counter and 1×10⁵ cells per well taken for flow cytometric staining with several antibody panels to assess: (1) T cell phenotype, (2) T cell expression of co-stimulatory and inhibitory markers, (3) maximal T cell cytokine intracellular expression (4) and cellular subsets in the HD T cells. Prior to staining for maximal cytokine detection, T cells were treated with 200 ng/mL of PMA, 1 μg/mL ionomycin and 1× brefeldin A in TCM at 1×106 T cells/mL in a humidified incubator (37° C., 5% CO2). All cytometric panels included a viability stain and human Fc receptor block. The T cell phenotype panel contained antibodies against murine TCRβ, anti-FRα CoStAR, CD3, CD4, CD8, CD27, CD45RA, CD45RO, CD95 and CCR7. The T cell costimulatory and inhibitory marker panel contained antibodies against murine TCRβ, anti-FRα CoStAR molecule, CD4, CD8, CD137, CTLA-4, PD-1, SLAM, TIM-3, and LAG-3. The maximal cytokine intracellular expression panel contained antibodies against murine TCRβ, anti-FRα CoStAR molecule, CD3, CD4, CD8, IL-17, IL-22, TNFα and IFNγ. The cellular subset panel contained antibodies against murine TCRβ, anti FRα CoStAR molecule, CD3, CD4, CD25, CD56, CD127, TCRαβ, TCRγδ and FOXP3. Staining for anti-FRα CoStAR with recombinant FRα-Fc was performed in 1% BSA-PEF (PBS, 0.4% EDTA, 1% BSA and 0.5% FBS) and extracellular antibody stains were conducted in a 50:50 mixture of BD Brilliant stain buffer and PEF (PBS, 0.4% EDTA and 0.5 FBS). Cells were fixed and permeabilized using BD Cytofix/Cytoperm according to the manufacturers protocol. Intracellular antibody stains were conducted in BD Perm/Wash buffer. Following staining, cells were washed and resuspended in PEF for analysis using a Novocyte 3005 Flow Cytometer System.

All groups of healthy donor T cells were approximately 95% αβTCR T cells with no contaminating Treg populations and little to no γδ T cells were detected. Transduction of HD T cells did not impact upon the αβ T cell frequency, although exclusion in the expression between endogenous and transgenic anti-CEA TCR surface expression was observed in donor 37636.

### Results

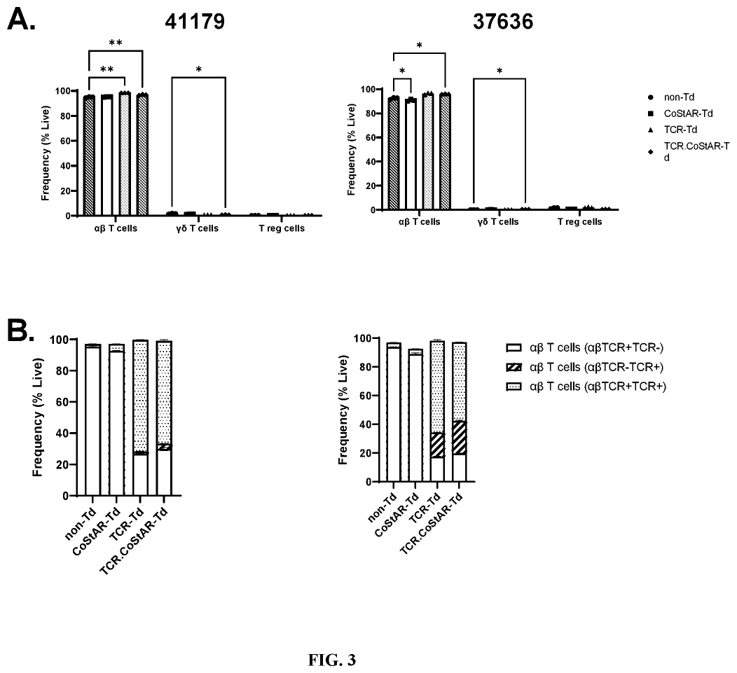

For donor 41179, 94.9% (SD±0.46%), 95.25% (SD±0.27%), 98.8% (SD±0.02%) and 96.8% (SD±0.45%) of the cell product for Non-Td, CoStAR-Td, TCR-Td and TCR.CoStAR-Td cells was αβTCR T cells, respectively (FIG. 3 panel A). Similarly, for donor 37636, αβ TCR T cells made up 92.5% (SD±0.79%), 90.82% (SD±0.76%), 96.3% (SD±0.67%) and 95.8% (SD±0.17%) of the expanded cell population, respectively (FIG. 3 panel A).

For donor 41179, there were small increases in αβ T cell frequencies in TCR-Td and TCR+CoStAR-Td conditions relative to Non-Td T cells (**P<0.01) but this was minimal, ranging from 2% to 4%. For donor 37636, a significant 3.3% increase in αβ T cell frequency was observed for TCR.CoStAR-Td T cells relative to Non-Td T cells (*P<0.05). However, for CoStAR-Td T cells, a minor 1.7% decrease in αβ T cell frequency relative to Non-Td T cells was observed. Therefore, the small but statistically significant fluctuations in αβ T cell frequency were due to experimental variation and not lentiviral modification or transgene expression.

The αβ T cell population was solely comprised of endogenous αβTCR+ populations in Non-Td and CoStAR-Td populations. In TCR-Td group, the αβ T cell population was divided into αβTCR+TCR−, αβTCR−TCR+ and αβTCR+TCR+. For TCR-Td and TCR.CoStAR-Td αβ T cells from donor 41179, 26.9% (SD±0.38%) and 29.8% (SD±0.98%) were αβTCR+TCR− whilst 71.4% (SD±0.18%) and 65.8% (SD±0.99%) were αβTCR+TCR+, respectively (FIG. 3 panel B). In both TCR-Td and TCR.CoStAR-Td αβ T cells from donor 41179, <4% were αβTCR−TCR+(FIG. 3 panel B). TCR-Td and TCR.CoStAR-Td αβ T cells from donor 37636 were 17.3% (SD±0.43%) and 19.5% (SD±0.49%) αβTCR+TCR− whilst 63.85% (SD±0.87%) and 54.6% (SD±0.16%) were αβTCR+TCR+, respectively (FIG. 3 panel B). T cells from donor 37636 in TCR-Td and TCR.CoStAR-Td conditions were 16.8% (SD±0.18%) and 22.9% (SD±0.58%) αβTCR−TCR+, respectively (FIG. 3 panel B).

Across all donors and conditions, γδ T cell and Treg cell frequency made up <2%. When comparing Non-Td and TCR.CoStAR-Td T cells, for donor 41179 a minor but significant 0.8% decrease in γδ T cell frequency was observed whilst for donor 37636 a significant 0.4% increase was measured (FIG. 3 panel A). Further, no differences in Treg frequency were measured among all T cell conditions (FIG. 3 panel A). When taken together, these data demonstrate that produced HD T-cell subtype frequencies were not impacted by the lentiviral transduction or transgene expression.

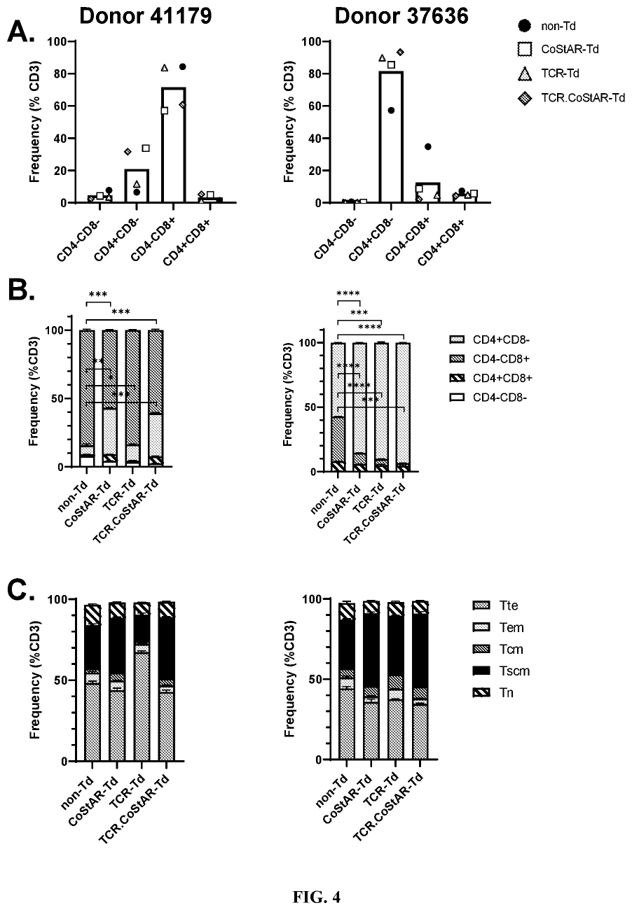

All T cell groups from donors 41179 and 37636 had contrasting CD4 to CD8 ratios (CD4:CD8), enabling evaluation of cells that cover potential product CD4:CD8 heterogeneity by in vitro characterization, and downstream in vivo measurement of anti-FRα CoStAR molecule efficacy when transduced in T cells along with a TCR. Overall, donor 41179 all T cell groups had 20.9% (SD±13.8%) CD4 T cells and 71.5% (SD±14.6%) CD8 T cells. Conversely, donor 37636 had 81.5% (SD±16.5%) CD4 T cells and 12.6% (SD±15.1%) CD8 T cells (FIG. 4 panel A). Between both donors, <5% T cells were CD4−CD8−, and <6% were CD4+ and CD8+ (FIG. 4 panel A).

During the production process, a bias toward CD4 T cells in transduced populations was observed which resulted in a lower CD8 T cell frequency within the transduced populations, regardless the transgene.

For donor 41179, the Non-Td condition had 6.57% (SD±1.16%) CD4 and 84.4% (SD±0.57%) CD8 T cells. CoStAR-Td T cells had 33.8% (SD±0.42%) CD4 and 57.2% (SD±0.52%) CD8 T cells, TCR-Td T cells had 11.6% (SD±0.43%) CD4 and 83.8% (SD±0.41%) CD8 T cells whilst TCR.CoStAR-Td T cells had 31.7% (SD±0.40%) CD4 and 60.8% (SD±0.66%) CD8 T cells (FIG. 4 panel B). Significant increases in CD4 T cell frequency were observed for CoStAR-Td (**P<0.01), TCR-Td (*P<0.05) and TCR.CoStAR-Td groups (***P<0.001, FIG. 4 panel B). Conversely, significant decreases in CD8 T cell frequency were observed between Non-Td T cells and CoStAR-Td and TCR.CoStAR-Td groups (***P<0.001, FIG. 4 panel B).

For donor 37636, Non-Td T cells had 57.4% (SD±0.11%) CD4 and 34.8% (SD±0.26%) CD8 T cells (FIG. 4 panel B).CoStAR-Td T cells had 85.5% (SD±0.17%) CD4 and 8.65% (SD±0.28%) CD8 T cells, TCR-Td T cells had 90.4% (SD±0.49%) CD4 and 4.69% (SD±0.28%) CD8 T cells whilst TCR.CoStAR-Td T cells had 93.5% (SD±0.13%) CD4 and 2.14% (SD±0.20%) CD8 T cells (FIG. 4 panel B). Increases in CD4 T cell frequencies were significant for CoStAR (****P<0.0001), TCR-Td (***P<0.001) and TCR.CoStAR-Td groups (****P<0.0001) compared to Non-Td T cells (FIG. 4 panel B). Conversely, significant decreases in CD8 T cell frequency were observed between Non-Td T cells and CoStAR-Td (****P<0.0001), TCR-Td (****P<0.0001) and TCR.CoStAR-Td groups (***P<0.001, FIG. 4 panel B).

Between donors and all T cell groups, T cell phenotypes were predominantly Tscm (16.4%-45.6%) and Tte (34.6%-67.3%) with small, comparable, frequencies of Tn (7.47%-2.7%), Tcm (1.62%-9.08%) and Tem (3.37%-6.83%) T cells (FIG. 4 panel C). Across donors and all T cell groups, the processes of T cell manufacture was observed to have variable impact on the relative frequencies of T cell phenotypes with a trend toward Tscm being associated with anti-FRα CoStAR molecule expression in CoStAR-Td and TCR.CoStAR-Td groups (TABLE 5; FIG. 4 panel C). For other phenotypes, small significant differences are likely due to experimental and donor variability rather than the production process and all T cell groups were comparable. One outlier condition was observed for donor 41179, where the Tscm population decreased from 26.6% (SD±0.99%) in the Non-Td T cell group to 16.6% (SD±1.00%) in the TCR-Td T cell group (TABLE 5; FIG. 4 panel C). Conversely, the Tte population increased from 48.2% (SD±1.22%) to 67.3% (SD±0.91%) (TABLE 5; FIG. 4 panel C). However, this skew toward Tte from Tscm was not observed in TCR-Td T cells from donor 37636 indicating that the production process or transgene expression were unlikely to be the cause. Moreover, donor 41179 frequencies of Tn, Tcm and Tem were of similar magnitude to other groups. The exact proportions of each phenotype and statistical comparisons between each donor across all T cell groups are listed in TABLE 5.

TABLE 5
Non-Transduced and Transduced Healthy Donor T Cell Phenotypes

| | Td | Tn | | | Tscm | | | Tcm | | | Tem | | | Tte | | |
|---|---|---|---|---|---|---|---|---|---|---|---|---|---|---|---|---|
| Donor | status | Av. | SD | P | Av. | SD | P | Av. | SD | P | Av. | SD | P | Av. | SD | P |
| 41179 | Non | 12.7 | 0.47 | — | 26.6 | 0.99 | — | 2.58 | 0.06 | — | 6.40 | 0.77 | — | 48.2 | 1.22 | — |
| | CoStAR | 9.56 | 0.49 | * | 33.7 | 0.59 | * | 5.02 | 0.21 | ** | 5.85 | 0.41 | ns | 43.8 | 1.33 | ns |
| | TCR | 7.47 | 0.10 | ** | 16.4 | 1.00 | * | 1.62 | 0.10 | * | 5.18 | 0.23 | ns | 67.3 | 0.91 | ns |
| | TCR + | 9.38 | 0.43 | * | 37.8 | 0.53 | ** | 4.30 | 0.47 | ns | 4.11 | 0.37 | ns | 42.8 | 1.26 | ns |
| | CoStAR | | | | | | | | | | | | | | | |
| 37636 | Non | 10.3 | 1.13 | — | 30.4 | 0.54 | — | 5.65 | 0.01 | — | 6.83 | 0.89 | — | 44.3 | 1.18 | — |
| | CoStAR | 7.61 | 7.61 | ns | 45.3 | 0.82 | ** | 6.39 | 0.57 | ns | 3.55 | 0.30 | ns | 35.8 | 1.77 | ns |
| | TCR | 8.44 | 8.44 | ns | 36.5 | 0.42 | ** | 9.08 | 0.34 | ** | 6.52 | 0.21 | ns | 37.5 | 0.28 | * |
| | TCR + | 7.81 | 7.81 | ns | 45.6 | 1.22 | ** | 7.37 | 0.36 | * | 3.37 | 0.26 | * | 34.6 | 0.56 | ** |
| | CoStAR | | | | | | | | | | | | | | | |

P indicates level of significance relative to Non-Td T cells in the indicated donor.
Abbreviations:
Tn, T naïve;
Tscm, T stem cell memory;
Tcm, T central memory;
Tem, T effector memory;
Tte, T terminal effector;
Td, transduced;
TCR, anti-carcinoembryonic antigen T cell receptor;
Av, the mean average;
SD, standard deviation;
statistical analysis was performed using a matched two-way ANOVA;
P, the level of significance;
* 0.05;
** 0.01;
ns, non-significant.

Between donors, and across CD4 and CD8 T cell subsets, marker expression had small but significant fluctuations. No consistent trends were observed between donors in comparable subsets and so the expression of costimulatory and coinhibitory markers was overall unaffected. Additionally, the magnitude of the observed variations are unlikely to impact functional cell characteristics or in vivo efficacy.

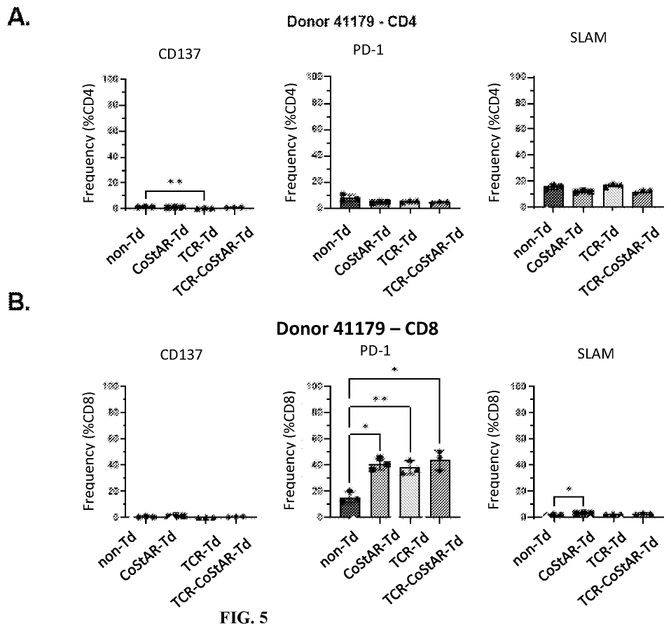

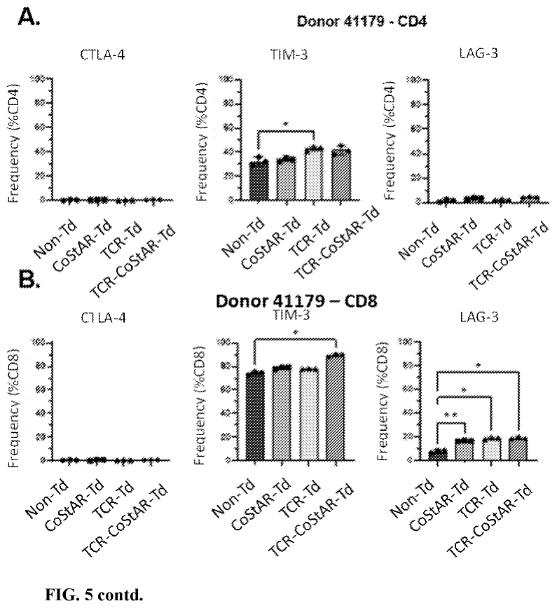

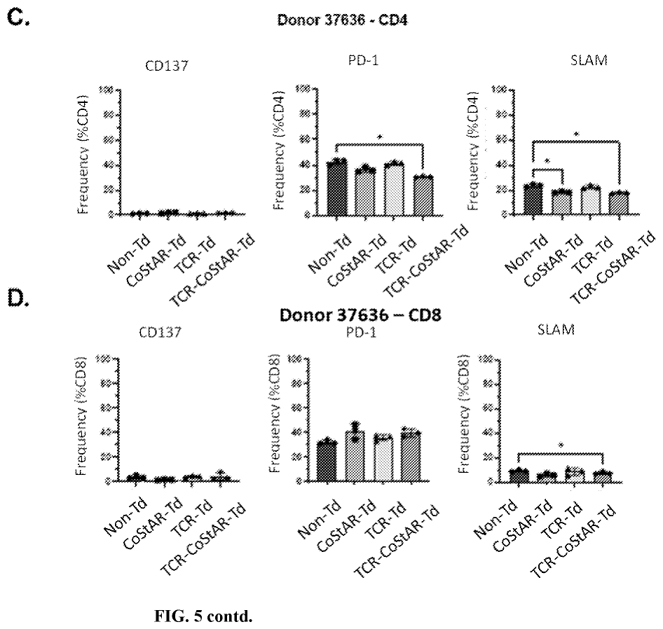

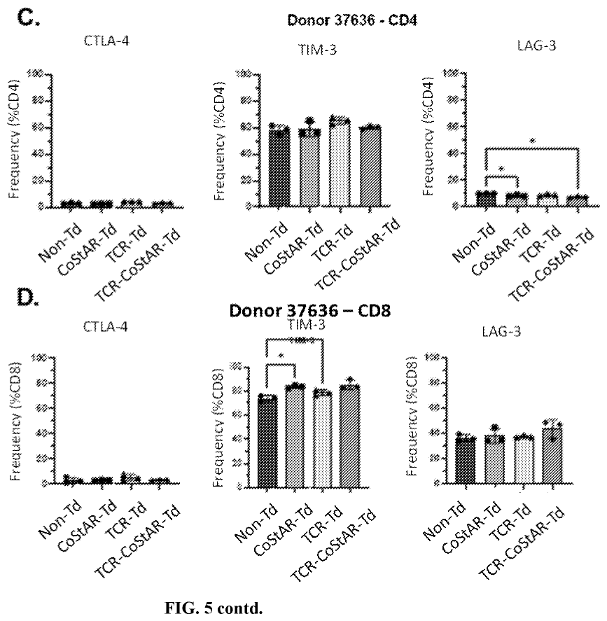

For donor 41179, the frequencies of CD137 or PD1 expression by both CD4 and CD8 T cells was <2% (FIG. 5 panels A, B). Moreover, there was no significant difference between all T cells groups for CD4 T cell expression of CTLA-4, PD-1 (4%-8%), SLAM (12%-18%) or LAG-3 (1%-5%; FIG. 5 panel A). However, in TCR-Td, CoStAR-Td and TCR.CoStAR-Td groups was observed to increase expression of the co-inhibitory markers PD-1 and LAG-3 in CD8 T cells for donor 41179 (FIG. 5 panels A, B). TIM-3 was expressed by 32.6% (SD±3.53%) of Non-Td CD4 T cells which was significantly increased in TCR-Td T cells to 42.9% (SD±1.82%; *P<0.05) but not in other conditions. The number of CD8 T cells expressing TIM-3 was 74%-90%, and 74.2% (SD±1.42%) of Non-Td T cells expressed TIM-3 which was significantly less (*P<0.05) that the 89.4% (SD±1.13%) TCR.CoStAR-Td T cells which also expressed the coinhibitory molecule (FIG. 5 panel B). In TCR-Td, CoStAR-Td and TCR.CoStAR-Td groups, within the CD8 T cells subset, PD-1 and LAG-3 were expressed at significantly greater frequencies than by Non-Td T cells from donor 41179 (FIG. 5 panel B). The frequency of Non-Td T cells expressing PD-1 was 15.7% (SD±4.2%) whilst for CoStAR-Td, TCR-Td and TCR.CoStAR-Td T cell groups this was 41.0% (SD±4.4%; *P<0.05), 38.5% (SD±5.1%; **P<0.01)% and 44.0% (SD±7.40%; *P<0.05), respectively (FIG. 5 panel B). Similarly for LAG-3, the frequency of Non-Td T cells was 7.61% (SD±1.3%) whilst for CoStAR-Td, TCR-Td and TCR.CoStAR-Td populations this was 16.7% (SD±0.24%; **P<0.01), 18.9% (SD±0.81; *P<0.05) and 18.8% (SD±1.00%; *P<0.05), respectively (FIG. 5 panel B). Within T cell groups from donor 41179, the increased of these coinhibitory markers in TCR-Td, CoStAR-Td and TCR.CoStAR-Td groups relative to Non-Td T cells indicates a that the process of lentiviral transduction may modulate certain phenotypic markers under the conditions tested. However, these increases are small and unlikely to have a significant impact in T cell function.

Similarly for donor 37636, less than <6% of both CD4 and CD8 T cells expressed CD137 or CTLA-4 with no significant differences between Non-Td and Td T cell groups (FIG. 5 panels C, D). Additionally, there were no significant differences in CD4 T cell populations which expressed TIM-3 (58%-66%; FIG. 5 panel C) nor in CD8 T cells were there differences for PD-1 (32%-42%) or LAG-3 (36%-44%; FIG. 5 panel D). Unlike in donor 41179, T cell expression of coinhibitory markers was not observed to be in CoStAR-Td, TCR-Td or TCR.CoStAR-Td groups compared to Non-Td in donor 37636 derived cells (FIG. 5 panels C, D). Furthermore, in CD4 T cell populations the frequency of T cell populations expressing PD-1, SLAM and LAG-3 was significantly decreased in TCR-Td, CoStAR-Td and TCR.CoStAR-Td relative to Non-Td T cells (*P<0.05; FIG. 5 panel C). Most likely, the observed differences were due to experimental and donor variation. Specifically, 42. 5% (SD±2.11%) of CD4 T cells in Non-Td groups express PD-1 whilst 30.8% (SD±0.39%) of TCR.CoStAR-Td T cells express it (*P<0.05; FIG. 5 panel C). Non-Td CD4 T cells express SLAM and LAG-3 at a frequency of 23.8% (SD±1.04%) and 10.0% (SD±0.22%)%, respectively (FIG. 5 panel C). In CoStAR-Td CD4 T cells, these markers were expressed at significantly reduced frequencies of 18.4% (SD±0.57%; *P<0.05;) and 8.18% (SD±0.64%; *P<0.05) %, respectively (FIG. 5 panel C). Similarly, in TCR.CoStAR-Td CD4 T cells, these markers were expressed at significantly reduced frequencies of 17.8% (SD±0.57%; *P<0.05) and 7.18% (SD±0.50%; *P<0.05;), respectively (FIG. 5 panel C). For SLAM, a small but significant reduction in the frequency of SLAM expressing CD8 T cells was observed for TCR.CoStAR-Td populations relative to Non-Td populations, from 9.1% (SD±0.86%) to 7.7% (SD±1.20%; *P<0.05; FIG. 5 panel D).

Conversely, 73.9% (SD±2.5%) TIM-3 positive CD8 T cells in Non-Td T cells was observed, which was significantly elevated to 83.6% (SD±1.33%) and 85.5% (SD±4.06%) for CoStAR-Td and TCR.CoStAR-Td T cell populations, respectively (*P<0.05; FIG. 5 panel D). Differences measured among all groups of T cells from donor 37636 were evidenced to be small, and unlikely to have a functional impact on T-cell biology.

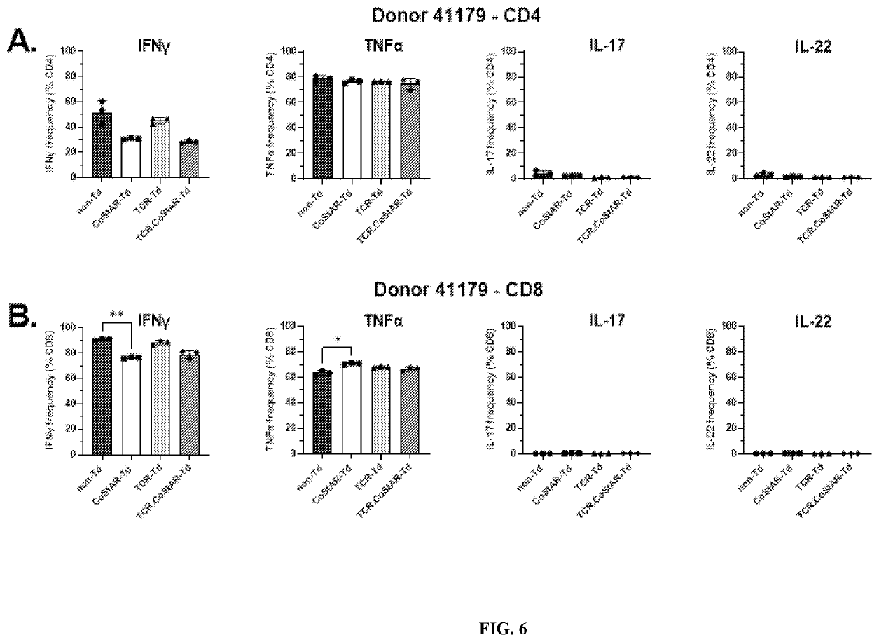

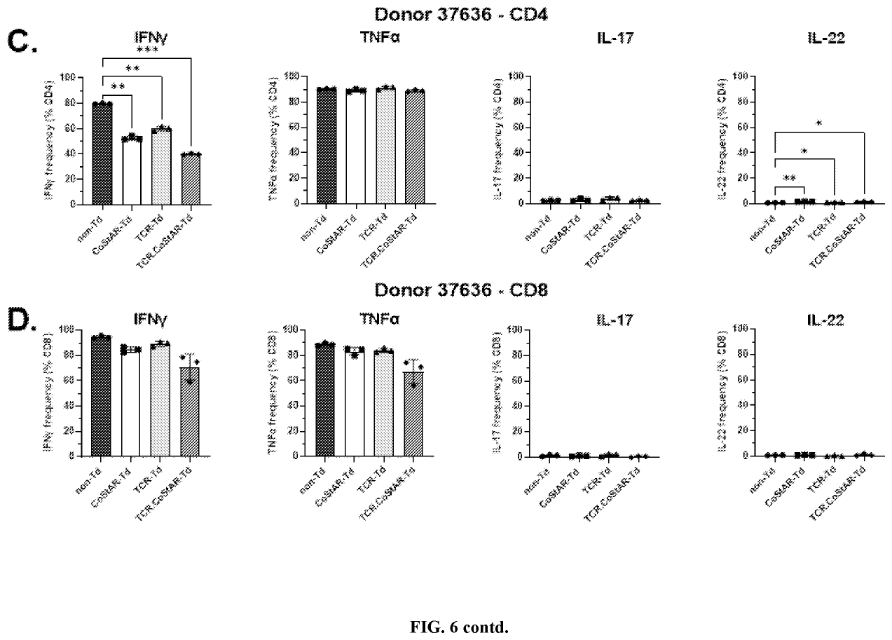

All T cell groups displayed robust intracellular cytokine levels of the proinflammatory cytokines IFNγ and TNFα with little to no production of IL-17 and IL-22 in response to PMA and ionomycin. Therefore, all T cell groups alike were able to functionally respond to T cell stimulation. Across all donors and status cell groups, the frequencies of CD4 T cells which produced IFNγ, TNFα, IL-17 and IL-22 were 28.5% to 79.8%, 74.5% to 91.5%, <4.5% and <3.7%. The frequencies of CD8 T cells which produced IFNγ, TNFα, IL-17 and IL-22 were 70.9% to 94.8%, 63.6% to 88.9%, <1.3% and <1.1%. No consistent trends were observed for cytokine expression in CD4 or CD8 T cells between all T cell groups for any cytokines measured, although some significant differences were observed when comparing Non-Td with the other T cell groups (FIG. 6).

For donor 41179, no significant differences in any cytokines produced by CD4 T cells were observed between Non-Td and the other T cell groups (FIG. 6 panel A). Similarly, no significant differences in the frequency of CD8 T cells producing IL-17 or IL-22 was observed across all T cell groups (FIG. 6 panel B). There were also no significant changes in the frequency of TCR-Td and TCR.CoStAR-Td CD8 T cells producing either IFNγ or TNFα (FIG. 6 panel B). The CD8 T cell production of IFNγ and TNFα was detected in 90% (SD±0.85%) and 63.6% (SD±1.9%) of Non-Td T cells, respectively (FIG. 6 panel B). A significant decrease in IFNγ expressing cells of 14.4% (**P<0.01) and an increase in TNFα producing cells of 7.3% (*P<0.05) was observed for CoStAR-Td CD8 T cells relative to Non-Td CD8 T cells, respectively (FIG. 6 panel B). This observation was not made for TCR-Td or TCR.CoStAR-Td CD4 or CD8 T cells.

For T cells from donor 37636, there were no significant differences in the frequency of CD4 T cells producing TNFα or IL-17 (FIG. 6 panel C). There were minor but significant differences between Non-Td and TCR-Td, CoStAR-Td and TCR.CoStAR-Td CD8 T cells of <1% for IL-22 production (FIG. 6 panel C). Conversely, the frequency of IFNγ production by CoStAR-Td (**P<0.01), TCR-Td (**P<0.01) and TCR.CoStAR-Td (***P<0.001) groups in CD4 T cells were 52.6% (SD±1.7%), 60.2% (SD±1.7%), 40.1% (SD±0.57%) which were significantly fewer T cells than the 79.7% (SD±0.3%) observed for the Non-Td condition (FIG. 6 panel C). Moreover, the lowest frequency of IFNγ producing CD4 T cells was in TCR.CoStAR-Td rather than in CoStAR-Td or TCR-Td CD4 T cells. Finally, there were no significant differences in the frequency of CD8 T cells producing IFNγ, TNFα, IL-17 or IL-22 for donor 37636. CoStAR-Td T cells showed sustained proliferation and persistence when stimulated multiple times. Expression of PD-1 was lower in CoStAR-Td T cells compared with non-Td control T-cells. The fraction of CD4+ and CD8+ T cells was not impacted by CoStAR. CoStAR transduction was associated with a lower fraction of terminally differentiated effector memory T cells (Temra cells). Thus, it appears that CoStAR can maintain a “younger” T-cell phenotype as demonstrated by lower PD-1 expression, lower fraction of Temra, and high proliferation potential.

### Example 5

### Functional Characterization of Non-Td, TCR-Td, CoStAR-Td, and TCR.CoStAR-Td Healthy Donor T Cells

### Materials and Methods

A 50 μL aliquot of fibronectin (10 μg/mL in PBS) was used to coat each well of an ACEA Bio E plate 96 (1 hour, 37° C., 5% CO2) before removal. Target H508.Luc.Puro.FRα cells were counted using the ViCell BLU automated cell counter and plated in TCM at a density of 2.5×105 cells per well and growth curves assessed by impedance (Cell index). At −30 hours, recovered T cells from all groups were added to added to target cells at an effector to target ratio of 10:1 in a final volume of 200 μL. A no treatment control using target cells and a full lysis control were included using a final concentration of triton-x-100 at 0.5%. The impact of all T cell groups upon target cell growth was assessed for 48 hours by normalized cell index.

The cytolytic activity of Non-Td and Td T cells was evaluated using an xCELLigence RTCA cytotoxicity assay prior to planned in vivo experimentation. The H508.Luc.Puro.FRα target cell line used was analogous to the line used for the in vivo efficacy study in NC014. The cell line is positive for the carcinoembryonic antigen (CEA), and the folate receptor and can engage both the transgenic anti-CEA TCR and anti-FRα CoStAR molecule. Here, the area under the curve (AUC) was used to compare treatment groups as a function of target cell growth over time.

### Results

The TCR-Td and TCR.CoStAR-Td was able to mediate target cell death in 41179 donor T cells (FIG. 7). The AUC values of the no treatment control and 41179 Non-Td T cell treatment group were 201.2 (SD±2.44) and 200.1 (SD±9.03), respectively. The AUC of the 41179 CoStAR-Td treatment group was 175.4 (SD±10.75), which was small but significantly reduced relative to the Non-Td treatment group (**P<0.01). The AUC of 41179 TCR-Td and TCR.CoStAR-Td groups were significantly lower (**P<0.01) at 30.4 (SD±0.48) and 32.7 (SD±1.8), respectively, demonstrating transgenic anti-CEA TCR is required for T cell cytotoxicity (FIG. 7).

In T cells derived from donor 37636, cytotoxicity of TCR-Td T cells from donor 37636 was not observed (FIG. 7), which can be a function of the reduced CD8 T cell frequency (90.4% [SD±0.49%] CD4 and 4.69% [SD±0.28%] CD8 T cells) relative to donor 41179 (11.6% [SD±0.43%] CD4 and 83.8% [SD±0.41%] CD8 T cells). However, cytotoxicity was observed for 37636 TCR.CoStAR-Td T cells, with a significantly reduced AUC of 91.2 (SD±0.96) relative to no treatment (**P<0.01; FIG. 7). Therefore, the lack of cytotoxicity due to low CD8 T cell frequency in the TCR-Td group can be overcome by anti-FRα CoStAR molecule enhancement of the CD8 cells that were present (FIG. 7).

Relative to the Non-Td control group, a small, but significant (**P<0.01), reduction in target cell growth in the 41179 CoStAR-Td group was measured, which had an AUC of 175.4 (SD±10.8; FIG. 7). Similar minor decreases were observed for donor 37636 CoStAR-Td and TCR-Td groups although they were not significantly different from Non-Td control conditions (FIG. 7).

### Example 6

### In Vitro Reconstitution of IL-2 and Preparation of all Groups of T-Cell Doses

### Materials and Methods

IL-2 was reconstituted in phosphate-buffered saline (PBS) to a concentration of 9×10⁵ IU/mL or 1 dose/50 μL. Reconstituted IL-2 was transferred immediately to Sygnature and stored at −20° C. until use.

Cryopreserved non-Td, TCR-Td, CoStAR-Td, and TCR.CoStAR-Td T cells were thawed in a 37° C. bead bath before transfer to a class II safety cabinet. Samples were immediately transferred into a 10× volume of complete T-cell media (TCM) (Roswell Park Memorial Institute [RPMI] 1640 Medium GlutaMAX™ Supplement, HEPES, 10% fetal bovine serum [FBS], 1× penicillin-streptomycin, and 50 μM β-mercaptoethanol). To remove residual cryopreservation medium, T cells were centrifuged (400 g, 5 min), the media was removed, and cells were resuspended in another 10× volume of TCM. To determine T-cell viability, a 50-4, aliquot was taken per sample for dead (DRAQ7+Annexin V+) and apoptotic (DRAQ7−Annexin V+) cell staining. The live (DRAQ7−Annevin V−) cell number was determined using a Novocyte 3005 Flow Cytometer System. The post-thaw anti-CEA TCR+ frequency of all groups of T cells was previously determined and used to normalize live T-cell dose in TCR-Td and TCR.CoStAR-Td for intravenous (IV) injection of 5×10⁶ TCR+ live cells. An additional dose level of 2.5×10⁶ TCR+ live cells was tested but not included in this analysis. The maximum number of total T cells in the TCR-Td or CoStAR.TCR-Td T cell doses was used as the live T-cell dose for non-Td and CoStAR-Td live cells. All T-cell groups were kept in TCM at 4° C. until transfer to Sygnature for injection. Immediately prior to transfer, T cells were centrifuged (400 g, 5 min), the media was removed, and cells were resuspended in an appropriate volume of PBS to provide 1 dose/100 μL.

### Example 7

### Staging, Dosing, Sample Collection, and Monitoring of In Vivo Tumor Efficacy Study

### Materials and Methods

Briefly, 1×10⁷ H508.Luc.GFP.FRα cells were subcutaneously injected on the left flank of 6-week-old female NSG mice. After 21 days engraftment, mice were randomized into treatment groups and given tail vein IV injections of 100 μL PBS (no treatment) or non-Td, TCR-Td, CoStAR-Td, or TCR.CoStAR-Td T cells in PBS the following day (day 0). In IL-2-designated groups, mice received 50 μL PBS containing 45,000 IU IL-2 subcutaneously on days 0 to 7. Tumor growth was assessed by digital caliper measurements, and mice were weighed at the same time. When tumor was undetectable, a caliper measurement of 0.001 cm³ was recorded. Mice were sacrificed at tumor volume limits (10% tumor volume by mouse body weight) or if clinical condition reached in accordance with the animal science procedures act (ASPA) 1986 and the project license of Sygnature Discovery. Live tail vein bleeds of up to 100 μL were collected on days 14 and 21 in ethylenediaminetetraacetic acid (EDTA)-containing capillary tubes before transfer to Instil Bio at room temperature. CoStAR-Td T cells did not show any cytotoxicty in vitro. For the TCR conditions, a transgenic, HLA-A*02-restricted, high-affinity CEA peptide-reactive TCR was introduced as a surrogate for polyclonal TCRs. Tumor growth, survival, and T-cell expansion in the periphery were assessed in 2 donors up to Day 99. Studies were performed with and without exogenous IL-2 support on Days 0-7 (FIG. 7).

### Example 8

### Flow Cytometric Characterization of Peripheral Murine Blood

### Materials and Methods

Tail vain bleeds on days 14 and 21 underwent RBC lysis using diluted 10× RBC lysis buffer (Biolegend) according to the manufacturer's protocol. Samples subsequently underwent fluorescent staining. The cytometric panel included a murine Fc receptor block, viability stain, and antibodies against CD3, CD4, CD8, and transgenic TCRβ. Anti-FRα-Fc and a fluorescent secondary was used to stain for anti-FRα CoStAR molecule. Following staining, cells were washed and resuspended in PEF (PBS, 0.4% EDTA, and 0.5% FBS) for analysis using a Novocyte 3005 Flow Cytometer System.

### Example 9

### Measurement of Post-Thaw Viability in all T-Cell Groups, Cell Dose Formulation, and Injection

### Results

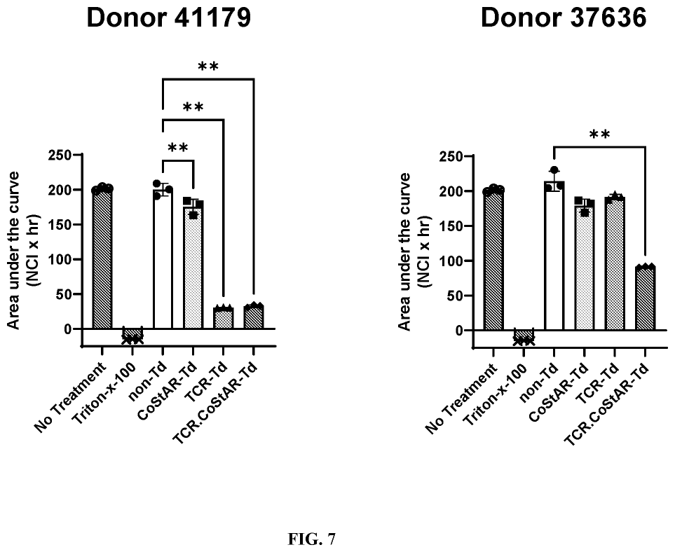

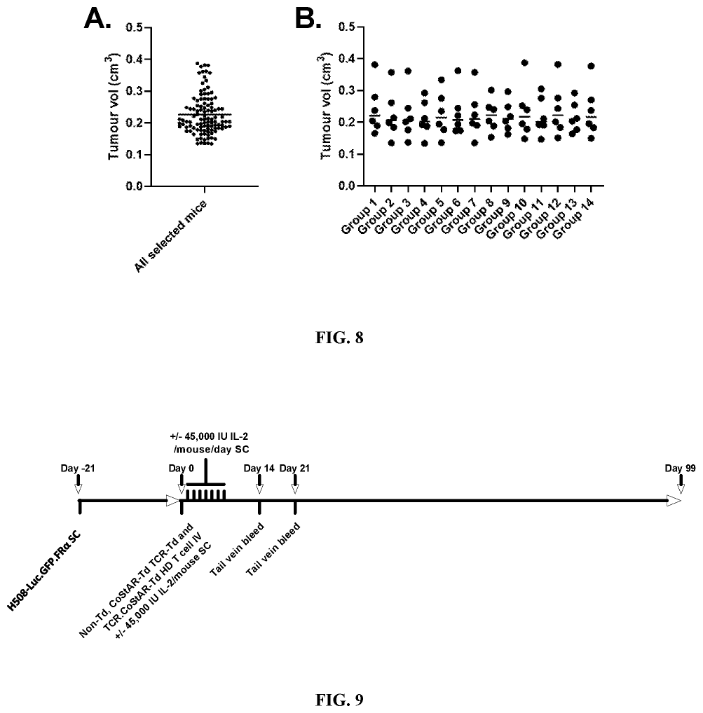

No differences were measured in the tumor size or mouse weight between tumor-bearing mice treatment groups the day prior to adoptive cell transfer. Subcutaneously administered H508.Luc.GFP. FRα cells established tumors in the flank of NSG mice with an average size of 0.23 (standard deviation [SD]±0.06) cm³ following 21 days of engraftment (FIG. 8 panel A). The successful tumor engraftment enabled randomization into treatment groups (Table 6) with comparable tumor sizes prior to adoptive cell transfer of T cells (FIG. 8 panel B). As a result, the observed differences among all the experimental groups in the in vivo study were a function of the respective treatment groups (TABLE 6 and FIG. 9).

TABLE 6
Tumor-Bearing Mice Treatment Groups
and Supportive IL-2 Regimens

| Supportive IL-2 IU/mouse/day | T-Cell Donor | Treatment Group |
|---|---|---|
| 45,000 | N/A | PBS (no Treatment) |
| 45,000 | 41179 | non-Td |
| | | CoStAR-Td |
| | | TCR-Td |
| | | TCR.CoStAR-Td |
| 45,000 | 37636 | non-Td |
| | | CoStAR-Td |
| | | TCR-Td |
| | | TCR.CoStAR-Td |
| 0 | N/A | PBS (no Treatment) |
| 0 | 37636 | non-Td |
| | | CoStAR-Td |
| | | TCR-Td |
| | | TCR.CoStAR-Td |

rhIL2 (supportive IL-2) was given subcutaneously on day 0 immediately following intravenous adoptive T-cell transfer and each day up to day 7.
Abbreviations: CoStAR, costimulatory antigen receptor; IL-2, interleukin-2; IU, international units; PBS; phosphate-buffered saline; rh, recombinant human; TCR, T-cell receptor; Td, transduced.

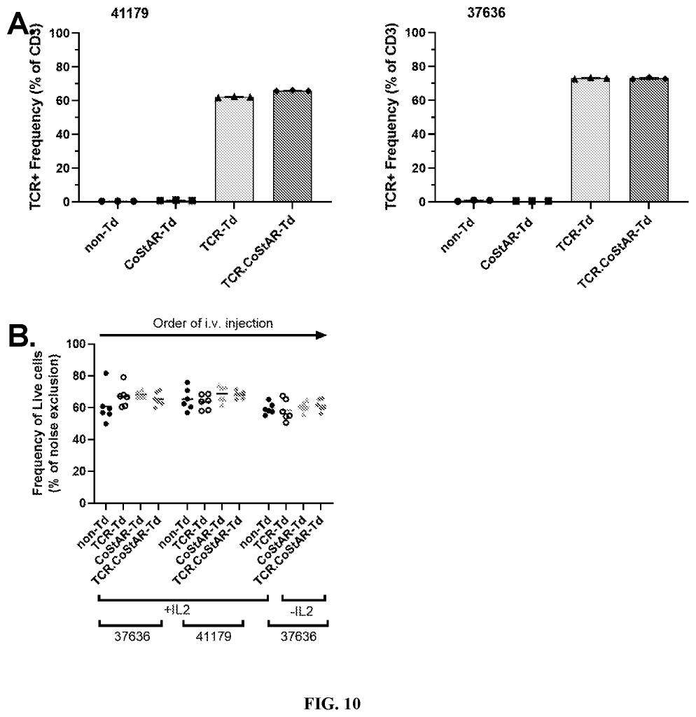

The T cells used in this study (derived from healthy donors 41179 and 37636) were successfully manufactured and characterized in Examples 1-7. TCR-Td and TCR.CoStAR-Td T cells were positive for the tumor-reactive transgenic anti-CEA TCR after cryopreservation (FIG. 10 panel A), and the equivalent dose of 5×10⁶ anti-CEA TCR+ T cells was successfully administered per group. TCR-Td and TCR.CoStAR-Td T cells from donors 37636 and 41179 were, on average, 72.96% (SD±0.442%) and 63.99% (SD±2.00%) anti-CEA TCR+, respectively. The number of administered non-Td and CoStAR-Td T cells was normalized to the maximum total T-cell dose in TCR-Td and TCR.CoStAR-Td groups, ensuring antitumor activity endowed by transgenic anti-CEA TCR was comparable between groups in vivo.

Recovered samples of T-cell doses were viable after passing through the injection needle, with no relationship observed between viability and time of injection. Adoptively transferred T cells were on average 63.63% (±SD 6.3%) viable with no differences in viability between treatment groups (FIG. 10 panel B).

### Example 10

### Quantification of Circulating T Cells in Mouse Peripheral Blood Via Flow Cytometry

### Results

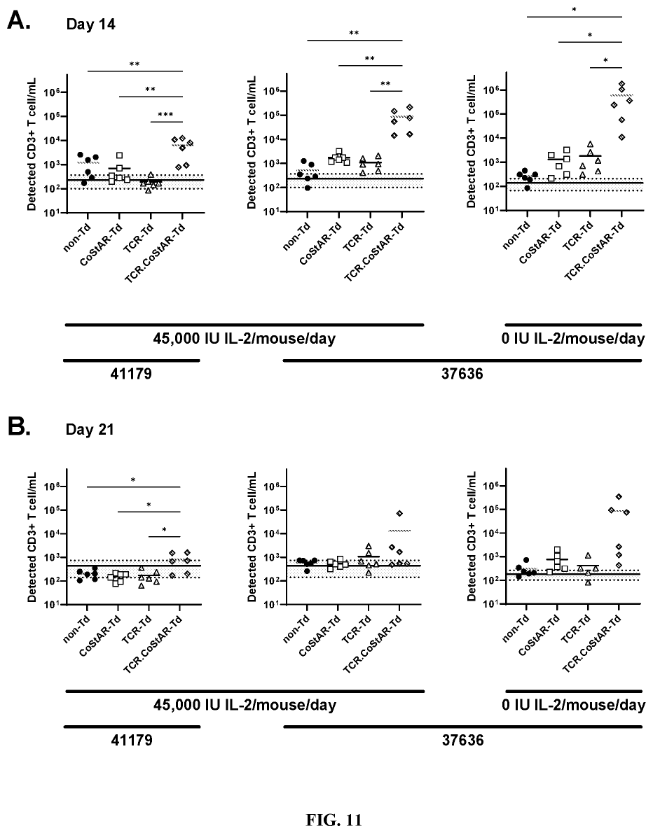

TCR.CoStAR-Td T cells showed improved in vivo T-cell expansion when compared with all other treatment groups. CoStAR-Td T cells did not confer improved expansion to T cells in the absence of anti-CEA TCR-mediated signal 1 (ie, TCR-Td group), demonstrating its functional dependency on signal 1 activation. Anti-human CD3 antibody was used to detect adoptively transferred human T cells in the murine peripheral blood. In mice treated with T cells from donor 41179, there was a 5.3-, 9.5-, and 33.3-fold increase in human T-cell concentration detected on day 14 in the TCR.CoStAR-Td treatment group relative to non-Td, CoStAR-Td, and TCR-Td treatment groups, respectively (FIG. 11 panel A, left panel). The detected concentration of 6.33 (±SD 4.96)×10³ CD3 T cells/mL in the TCR.CoStAR-Td group was significantly higher than in non-Td (** P<0.01), CoStAR-Td (** P<0.01), and TCR-Td (*** P<0.001) treatment groups, whose concentrations were 1.19 (±SD 0.41)×10³, 0.67 (±SD 0.86)×10³, and 0.19 (±SD 0.10)×10³ CD3 T cells/mL, respectively (FIG. 11 panel A, left panel). The detected T-cell blood concentration of non-Td, CoStAR-Td, and TCR-Td treatment groups was not statistically elevated above the detection limit, as determined by measurements in mice who received PBS. Similarly, observations in mice treated with T cells from donor 37636 showed increases of 166.6-, 50.2-, and 81.0-fold T cells/mL in TCR.CoStAR-Td recipient mice relative to non-Td, CoStAR-Td, and TCR-Td treatment groups (FIG. 11 panel A, middle panel). The detected concentration of 8.68 (±SD 7.86)×10⁴ CD3 T cells/mL in the TCR.CoStAR-Td group was significantly higher than for non-Td (** P<0.01), CoStAR-Td (** P<0.01), and TCR-Td (*** P<0.001) groups whose concentrations were 0.52 (±SD 0.46)×10³, 1.73 (±SD 0.78)×10³, and 1.07 (±SD 0.64)×10³ CD3 T cells/mL, respectively (FIG. 11 panel A, middle panel). This suggests that anti-FRα CoStAR molecule binding of FRα on tumor cells (signal 2) was conferring enhanced costimulation and persistence to CEA tumor antigen-specific anti-CEA TCR T cells.

In order to assess whether the enhanced costimulation requires exogenous IL-2 support, in vivo expansion and tumor efficacy of all T-cell groups was measured with and without supportive IL-2. In vivo proliferation was measured for donor 37636 in the absence of IL-2 support with 2,237.7-, 447.8-, and 319.7-fold increases of CD3 T cells/mL of murine peripheral blood in the TCR.CoStAR-Td treatment group relative to non-Td, CoStAR-Td, and TCR-Td treatment groups, respectively, on day 14 (FIG. 11 panel A, right panel). The detected concentration of 5.9 (±SD 7.03)×10⁵ CD3 T cells/mL in the TCR.CoStAR-Td group was significantly higher than in non-Td (* P<0.05), CoStAR-Td (* P<0.05), and TCR-Td (* P<0.05) treatment groups, whose concentrations were 0.26 (±SD 0.12)×10³, 1.31 (±SD 1.18)×10³, and 1.84 (±SD 2.10)×10³ CD3 T cells/mL, respectively (FIG. 11 panel A, right panel). These data indicate that supportive IL-2 administration was not required for the enhanced in vivo expansion observed in the TCR.CoStAR-Td group.

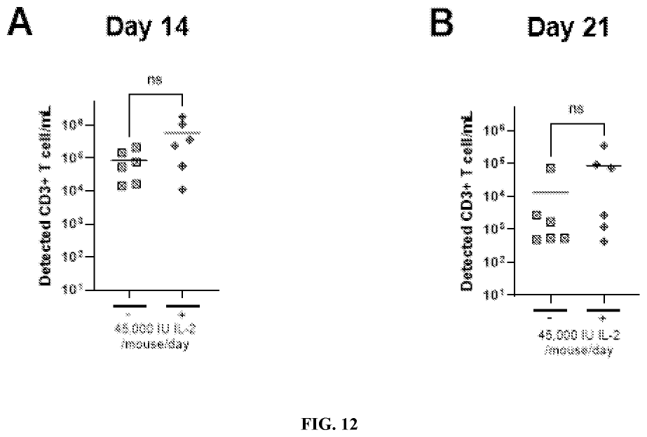

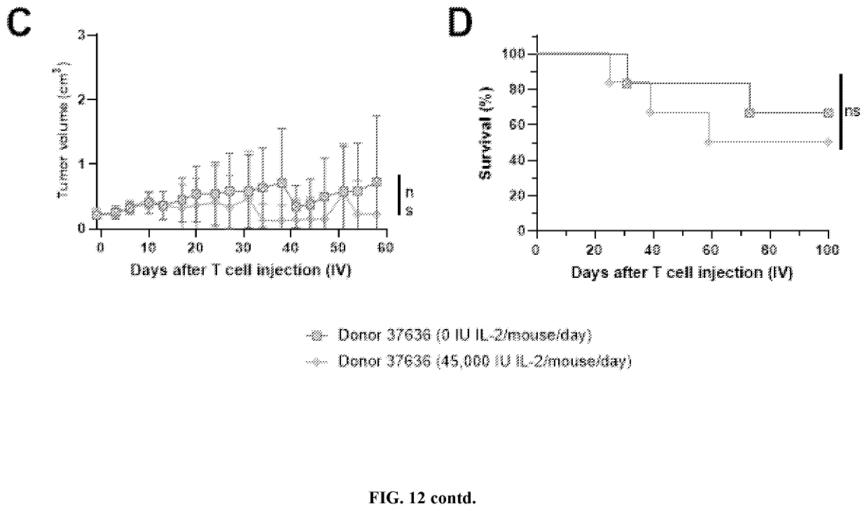

Elevated concentrations of TCR.CoStAR-Td T cells in the blood relative to other treatment groups were sustained up to day 21, albeit concentrations of transferred T cells were lower than at day 14. For all treatment groups from donor 41179, a significantly higher average concentration of 0.81 (±SD 0.62)×10³ CD3 T cells/mL was observed in the TCR.CoStAR-Td group relative to non-Td (* P<0.05), CoStAR-Td (* P<0.05), and TCR-Td (* P<0.05) treatment groups, whose concentrations were 0.20 (±SD 0.09)×10³, 0.15 (±SD 0.06)×10³, and 0.17 (±SD 0.11)×10³ CD3 T cells/mL, respectively (FIG. 11 panel B, left panel). In agreement with this observation, TCR.CoStAR-Td T cells from donor 37636 had an average increase of 22.4-, 24.5-, and 12.2-fold relative to non-Td, CoStAR-Td, and TCR-Td treatment groups, respectively (FIG. 11 panel B, middle panel). Furthermore, without supportive IL-2 administration, 279.5-, 112.4-, and 206.2-fold increases in T-cell peripheral blood concentrations were observed in TCR.CoStAR-Td treatment groups relative to non-Td, CoStAR-Td, and TCR-Td groups, respectively (FIG. 11 panel B, right panel). Across all donors, the detected CD3 T cells in the periphery of non-Td, CoStAR-Td, and TCR-Td treatment groups were not statistically elevated above detection limit as determined by measuring mice who received PBS (no treatment) on day 21. When the TCR.CoStAR-Td group from donor 37636 with exogenous IL-2 and without exogenous IL-2 were directly compared, no significant differences were detected (FIG. 12 panels A and B). Together, these data demonstrate that anti-FRα CoStAR molecule improves T-cell in vivo expansion in this model only in the presence TCR-pMHC-mediated signal 1, and that the improved T-cell expansion observed in TCR.CoStAR-Td groups does not require supportive IL-2.

### Example 11

### Caliper Tumor Volume Measurements Across all Treatment Groups

### Results

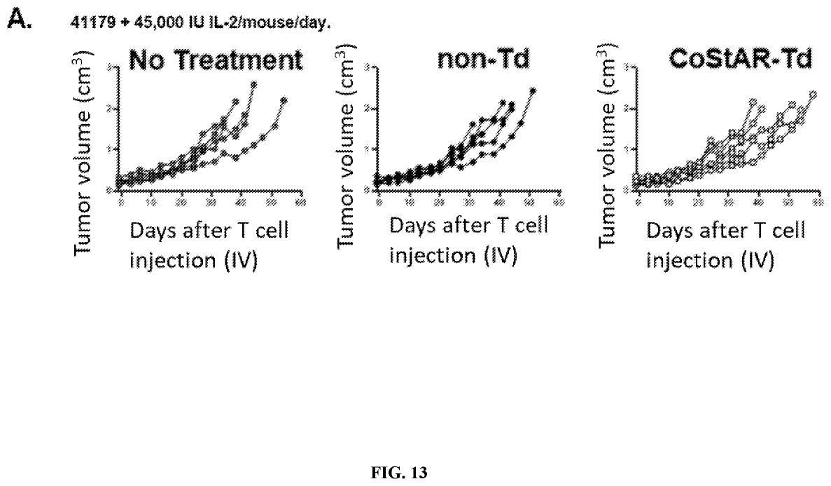

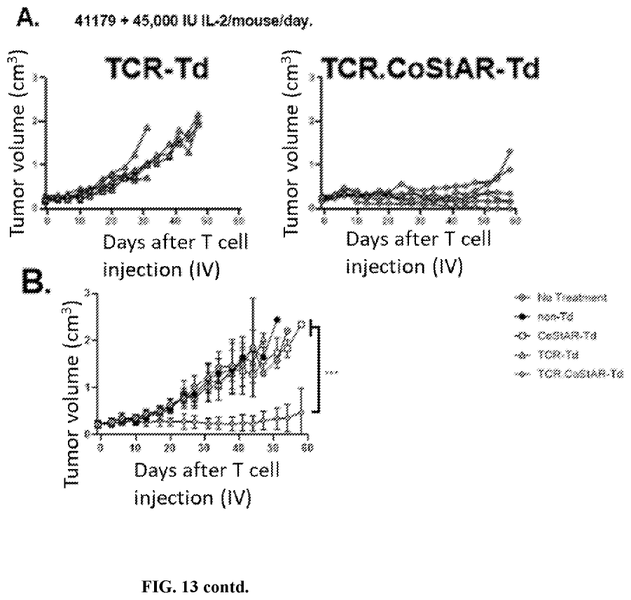

Administration of TCR.CoStAR-Td T cells led to dramatic control of tumor growth relative to non-Td, CoStAR-Td, and TCR-Td treatment groups. CoStAR-Td alone did not limit tumor growth, demonstrating that TCR engagement of pMHC is a requirement for anti-FRα CoStAR molecule activity. In mice receiving no treatment, the average tumor volume was 1.99 (SD±0.43) cm³ at experimental endpoints (FIG. 13 panel A). At day 58, the average tumor volume for the TCR.CoStAR-Td treatment group was 0.48 (SD±0.51) cm³ (FIG. 13 panel A and FIG. 13 panel A). When mice were administered TCR.CoStAR-Td T cells from donor 41179, their tumor volume was significantly smaller than non-Td, CoStAR-Td, and TCR-Td treatment groups from day 0 to 58 (*** P<0.001) (FIG. 13B panel). Conversely, there was no significant difference in tumor volumes between untreated mice and those that received non-Td, CoStAR-Td, or TCR-Td T cells, which had average tumor volumes of 1.95 (SD±0.38), 1.91 (SD±0.51), and 1.62 (SD±0.51) cm³, respectively, at experimental endpoints or day 58 (FIG. 13 panel B).

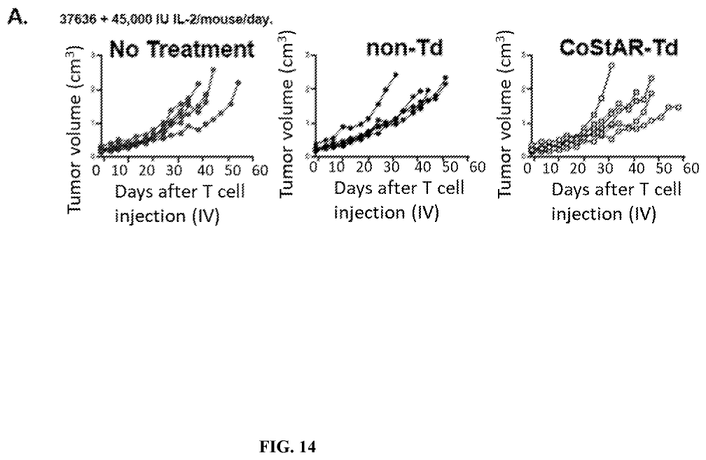

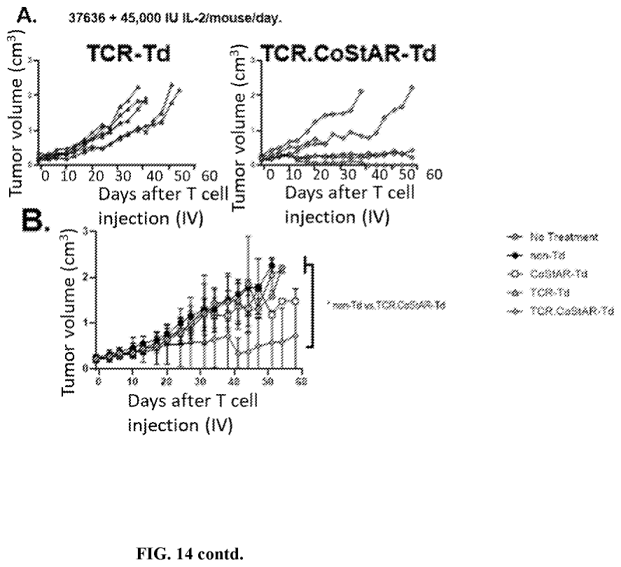

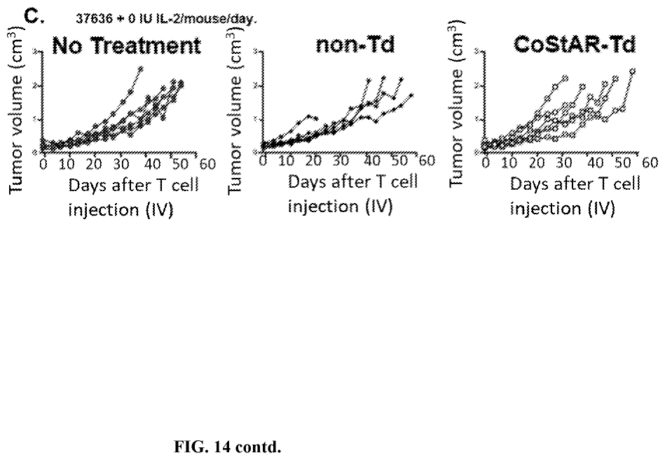

Similar control of tumor growth was observed in mice treated with TCR.CoStAR-Td T cells from donor 37636 (FIG. 14). This was irrespective of supportive IL-2 administration, suggesting that improved costimulation provided by anti-FRα CoStAR molecule may overcome the need for exogenous IL-2 support. For non-Td, CoStAR-Td, TCR-Td, and TCR.CoStAR-Td treatment groups, average tumor volumes were 1.98 (SD±0.52), 1.95 (SD±0.51), 1.95 (SD±0.40), and 0.94 (SD±0.99) cm³, respectively, at experimental endpoints or day 58 (FIG. 14 panel A). Of the mice in the TCR.CoStAR-Td treatment group, 3 out of 6 responded to treatment and responder mice in the TCR.CoStAR-Td treatment group had an average tumor volume of 0.22 (SD±0.22) cm³ at experimental endpoints or day 58 (FIG. 14 panel A.). When mice were administered TCR.CoStAR-Td T cells from donor 37636, their tumor volume was significantly smaller than the non-Td treatment group from days 0 to 58 (* P<0.05; FIG. 14 panel B.). Conversely, non-Td, TCR-Td, and CoStAR-Td treatment groups receiving T cells had no statistical differences from the no treatment group (FIG. 14 panel B.). Without supportive IL-2 administration, 4 out of 6 mice in the TCR.CoStAR-Td treatment group were responder mice. The terminal, or day 58, average tumor volume of mice in the TCR.CoStAR-Td group was 0.51 (SD±0.83) cm³ and 0.001 (SD±0.0) cm³ in responder mice (FIG. 14 panel C.). Further, the tumors of TCR.CoStAR-Td T cells were significantly smaller than non-Td (* P<0.05), CoStAR-Td (** P<0.01), and TCR-Td (** P<0.05) treatment groups from days 0 to 58 (FIG. 14 panel D). Further, when the TCR.CoStAR-Td groups from donor 37636 with exogenous IL-2 and without exogenous IL-2 were directly compared, no significant differences were found (FIG. 14 panel C). Therefore, anti-FRα CoStAR molecule improved control of tumor growth in this model only in the presence of anti-CEA TCR (signal 1), irrespective of exogenous IL-2 support.

### Example 12

### Measurement of Survival Across all T-Cell Treatment Groups

### Results

Adoptive cell transfer of TCR.CoStAR-Td T cells led to a significant improvement in survival of tumor-bearing mice, showing that enhanced in vivo expansion and tumor control or regression led to a survival benefit in vivo. This benefit was not observed in non-Td, CoStAR-Td, and TCR-Td treatment groups. These differences in survival highlight the dependency of anti-FRα CoStAR molecule on anti-CEA TCR signaling to confer a therapeutic benefit.

All mice in the no treatment group (PBS) reached experimental endpoints by day 55 (FIG. 15 panels A and B). Similarly, for T-cell treatment groups from donor 41179, non-Td, CoStAR-Td, and TCR-Td T cells all reached their tumor volume limits by days 52, 59, and 48, respectively (FIG. 15 panel A). Survival was significantly increased for the TCR.CoStAR-Td T-cell treatment group (** P<0.01) with 5 of 6 mice surviving until the study was terminated at day 99 (FIG. 15 panel A). For mice receiving T cells from donor 37636, non-Td, CoStAR-Td, and TCR-Td treatment groups all reached their tumor volume limits by days 52, 76, and 55, respectively (FIG. 15 panel B). In the CoStAR-Td treatment group, 5 of 6 mice had reached experimental endpoints by day 48, with 1 of 6 mice surviving until day 76. Conversely, in the TCR.CoStAR-Td T-cell group, 3 of 6 mice survived until the end of the study at day 99 (FIG. 15 panel B).

The improved survival of tumor-bearing mice following treatment with TCR.CoStAR T cells was independent of supportive IL-2 administration. All tumor-bearing mice in the PBS treatment group reached experimental endpoints by day 55. Treatment groups receiving non-Td, CoStAR-Td, and TCR-Td T cells from donor 37636 reached experimental endpoints by days 69, 59, and 55, respectively (FIG. 15 panel C). In the TCR.CoStAR-Td treatment group, survival was improved relative to TCR-Td treatment groups and significantly improved compared with non-Td and CoStAR-Td T-cell treatment groups (* P<0.05), with 4 of 6 mice surviving until the end of the study (FIG. 15 panel C). Furthermore, when the TCR.CoStAR-Td groups from donor 37636 with exogenous IL-2 and without exogenous IL-2 were directly compared, there were no significant differences (FIG. 15). This importantly demonstrates that anti-FRα CoStAR molecule enhancement of survival observed in this model was dependent on the presence of anti-CEA TCR but independent of exogenous IL-2 administration when combined with anti-CEA TCR.

### Example 13

### Characterization of Toxicities Observed in the In Vivo Study and Association with all T-Cell Treatment Groups

### Results

The toxicities associated with H508.Luc.GFP.FRα tumor engraftment were ulceration, hemorrhaging, and rupturing of the tumor. These incidences occurred equally across groups receiving PBS (no treatment), non-Td, CoStAR-Td, and TCR-Td T cells but at a lower frequency in those receiving TCR.CoStAR-Td T cells (TABLE 7). None of the other T-cell treatment groups was associated with additional toxicities, and those resulting from H508.Luc.GFP. FRα cell engraftment were equivalently evident in mice who did not receive adoptive cell therapy (TABLE 7). In TCR.CoStAR-Td groups, fewer incidences of toxicity were observed, likely due to the lower tumor volume in these treatment groups rather than a direct function of anti-CEA TCR and anti-FRα CoStAR molecule coexpression on the injected T cells (TABLE 7). The study was ended at day 99 due to the suspected onset of graft-versus-host disease from xenoreactivity. This was observed as mice appearing ungroomed, with persistent reddening around the eyes and the snout. Only mice in TCR.CoStAR-Td groups remained at day 99, and aberrant grooming and condition was observed in 2 recipient mice treated with TCR.CoStAR-Td T cells from donor 37636 that had received IL-2 support.

TABLE 7
Description and Incidence of Tumor-Associated Toxicities
Observed Across All T-Cell treatment Groups

| Supportive IL- | | | | |
|---|---|---|---|---|
| 2/Mouse/Day | Donor | Treatment Group | Toxicity | Incidence |
| 45,000 | N/A | PBS (no treatment) | Ulcerated tumor | 2/6 |
| | | | Internally hemorrhaged tumor | 2/6 |
| | | | Externally ruptured tumor | 0/6 |
| 45,000 | 41179 | non-Td | Ulcerated tumor | 1/6 |
| | | | Internally hemorrhaged tumor | 2/6 |
| | | | Externally ruptured tumor | 0/6 |
| | | CoStAR-Td | Ulcerated tumor | 0/6 |
| | | | Internally hemorrhaged tumor | 1/6 |
| | | | Externally ruptured tumor | 0/6 |
| | | TCR-Td | Ulcerated tumor | 1/6 |
| | | | Internally hemorrhaged tumor | 3/6 |
| | | | Externally ruptured tumor | 0/6 |
| | | TCR.CoStAR-Td | Ulcerated tumor | 0/6 |
| | | | Internally hemorrhaged tumor | 0/6 |
| | | | Externally ruptured tumor | 0/6 |
| 45,000 | 37636 | non-Td | Ulcerated tumor | 2/6 |
| | | | Internally hemorrhaged tumor | 2/6 |
| | | | Externally ruptured tumor | 0/6 |
| | | CoStAR-Td | Ulcerated tumor | 2/6 |
| | | | Internally hemorrhaged tumor | 3/6 |
| | | | Externally ruptured tumor | 0/6 |
| | | TCR-Td | Ulcerated tumor | 1/6 |
| | | | Internally hemorrhaged tumor | 1/6 |
| | | | Externally ruptured tumor | 0/6 |
| | | TCR.CoStAR-Td | Ulcerated tumor | 1/6 |
| | | | Internally hemorrhaged tumor | 0/6 |
| | | | Externally ruptured tumor | 1/6 |
| 0 | N/A | PBS (no treatment) | Ulcerated tumor | 0/6 |
| | | | Internally hemorrhaged tumor | 0/6 |
| | | | Externally ruptured tumor | 0/6 |
| 0 | 37636 | non-Td | Ulcerated tumor | 3/6 |
| | | | Internally hemorrhaged tumor | 0/6 |
| | | | Externally ruptured tumor | 1/6 |
| | | CoStAR-Td | Ulcerated tumor | 2/6 |
| | | | Internally hemorrhaged tumor | 2/6 |
| | | | Externally ruptured tumor | 0/6 |
| | | TCR-Td | Ulcerated tumor | 0/6 |
| | | | Internally hemorrhaged tumor | 2/6 |
| | | | Externally ruptured tumor | 0/6 |
| | | TCR.CoStAR-Td | Ulcerated tumor | 0/6 |
| | | | Internally hemorrhaged tumor | 0/6 |
| | | | Externally ruptured tumor | 0/6 |

Animal and tumor condition was assessed from day 0 until termination of the study.
Abbreviations: CoStAR, costimulatory antigen receptor; IL-2, interleukin-2; IU, international units; PBS, phosphate-buffered saline; TCR, T-cell receptor; Td, transduced.

### Example 14

### ITIL-306-201 Study

### Materials and Methods

Safety and feasibility of ITIL-306 will be evaluated in a multicenter, first in human, singlearm phase 1a/1b dose escalation and expansion study (FIG. 17) in adult patients with solid tumors whose disease has relapsed or is refractory to standard therapies. Phase 1a will comprise dose escalation and will include n=6-18 patients, where the primary endpoint is incidence of dose limiting toxicity. Phase 1b will comprise expansion and will include n=3 patients in 3 cohorts. In both phases, patients will comprise epithelial, ovarian, and fallopian tube, and peritoneal carcinomas, non-small cell lung cancer, and renal cell carcinoma patients, where the primary endpoint of is safety. The study Arms and Assigned Interventions, Outcome Measures, and Eligibility Criteria of ITIL 306-201 are included in TABLES 1-3.

Following initial screening, the study will proceed to enrollment/tumor resection. Patients will then undergo lymphodepleting therapy accomplished by cyclophosphamide 500 mg/m 2 IV and fludarabine 30 mg/m 2 IV, both provided on Days −5 to −3. Patients will then undergo ITIL-306 infusion of either a single IV fixed dose (3 dosage levels) on Day 0, or, patients will be infused with a single IV of dose selected in Phase 1a on Day 0. Patients will be assessed posttreatment on Days 14 and 28. Secondary endpoints include duration of response, objective response rate, progression free survival, and overall survival.

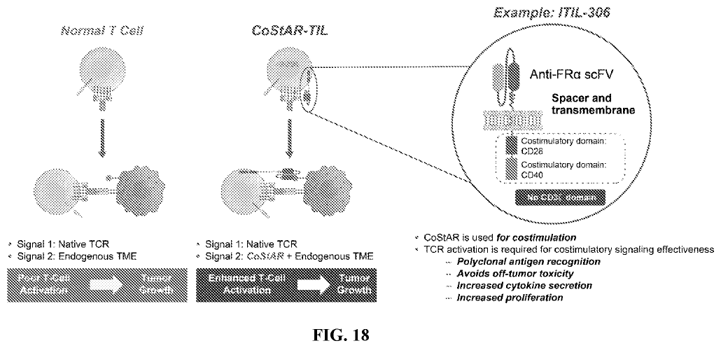

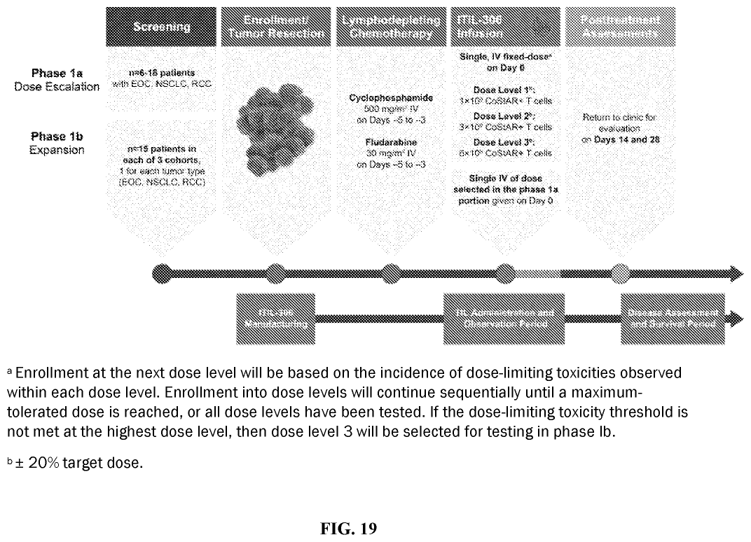

Eligible patients will be aged ≥18 years with histologically confirmed EOC, NSCLC, or RCC that has progressed during or after ≥1 prior line of systemic standard-of-care therapy, have ECOG performance status 0-1, and have viable tumor tissue that is suitable to resect with anticipated aggregate of ≥2 grams for TIL harvest. Patients will be enrolled in either phase 1a (dose escalation in a standard 3+3 design; n≈6-18) or 1b (expansion; n≈5 in each of 3 cohorts, 1 for each tumor type). Following tumor resection for TIL harvest, patients must have ≥1 remaining measurable lesion per RECIST v1.1. Patients will receive 3 days of intravenous lymphodepleting chemotherapy (cyclophosphamide×3 days overlapping with fludarabine×3 days) followed by a single, intravenous fixed-dose of ITIL-306 (FIG. 18) in phase 1a (1 of 3 dose levels) or 1b (dose selected in the phase 1a portion). The phase 1a primary endpoint is incidence of dose-limiting toxicities. The phase 1b primary endpoint is frequency and severity of treatment-emergent adverse events (AEs), serious AEs, and AEs of special interest. Secondary endpoints include manufacturing success rate, objective response rate per modified RECIST v1.1, disease control rate, best overall response, time to response, duration of response, progression-free survival, and overall survival (FIG. 19). The study is open (NCT05397093).

### Example 15

Schematics showing the structures of the FRα, anti-pembrolizumab, CEA, and MSLN CoStAR embodiments provided herein with associated sequences for each domain are illustrated in FIGS. 20-23.

### Example 16

ITIL-306 is a genetically engineered autologous TIL cell therapy that amplifies TCR-specific antigen recognition (Signal 1) with an FRα-specific CoStimulatory Antigen Receptor (CoStAR; Signal 2). ITIL-306 is depicted in FIG. 20D with its parts shown in FIG. 20A-20C. T-cell activation through the endogenous TCR is dependent on the concentration of cognate peptide-WIC antigen. This study examined T-cell activation across a range of physiologically relevant FRα expression levels and characterized whether functional T-cell avidity (response to cognate antigen concentration) was impacted with CoStAR engagement.

### Methods

In vitro cocultures were used to determine T-cell response to variations in strength of Signal 1 and Signal 2. To evaluate the role of FRα on amplification of T-cell responses, stable cell lines expressing membrane-anchored OKT3 and different FRα levels were established. Healthy donor (HD) T cells transduced with anti-FRα CoStAR or non-transduced (control) were used as effector cells. Cytolytic activity and cytokine levels (IL-2, IFN-γ, TNF-α) were assessed.

To assess the effect of CoStAR on TCR functional avidity, HD T cells were non-transduced or transduced with a defined TCR recognizing HLA-A*02/MART-1 antigen, anti-FRα CoStAR, or both. Parental T2 or FRα-transduced T2 cells were used as targets. Target cells were pulsed with titrated concentrations of 4 different MART-1-altered peptide ligands of varying antigenicity. Cytokine secretion after coculture was measured, and antigen half-maximal effective concentration (EC₅₀) was calculated.

### Results:

CoStAR amplified T-cell responses at all FRα expression levels. IL-2 secretion was significantly higher at any FRα expression level versus no FRα (P<0.0001). CoStAR-transduced and non-transduced T cells were not activated in coculture with cells expressing any level of FRα alone. Kinetic activation studies demonstrated that engaging CoStAR (Signal 2) followed by TCR activation (Signal 1) at a later time resulted in amplified T-cell activity.

Cytokine secretion was increased from MART-1-TCR+CoStAR T cells versus MART-1-TCR T cells when cocultured with T2-FRα cells pulsed with titrated concentrations of all cognate peptides evaluated. EC₅₀ was not impacted by CoStAR for cognate peptides with EC₅₀ between 10⁻¹⁰ to 10⁻⁷ M.

### Conclusions:

CoStAR augmented T-cell function across a range of physiologically relevant FRα expression levels and TCR/cognate peptide affinities. TCR/cognate antigen affinity (EC₅₀) was unchanged by CoStAR, suggesting that CoStAR TIL will have identical specificity as unmodified TIL. Further, CoStAR improved T-cell function at low FRα expression levels, supporting the evaluation of ITIL-306 activity across multiple tumors, including those with low FRα expression. These results are being explored in a first-in-human clinical study with ITIL-306 (NCT05397093).

### Example 17

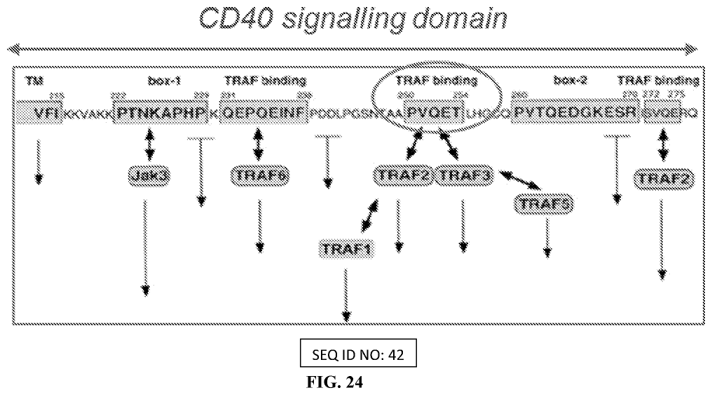

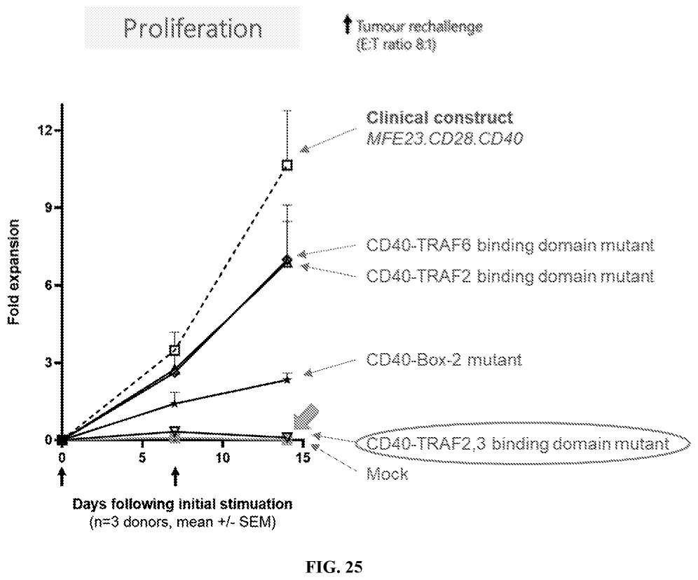

The biology of the CoStAR receptor was further explored to identify the signaling interactions taking place in the CD40 signaling domain that enable CoStAR function. FIG. 24 describes the CD40 signaling domain and identifies box-1, box-1, TRAF binding sites and which TRAFs interact with which binding sites. CD40 mutants were developed for the anti-CEA CoStAR (MFE23 scFv). Mutants included a TRAF6 binding domain mutant, TRAF2 binding domain mutant, TRAF2, 3 binding domain mutant, and Box-2 mutant. Upon tumor challenge (E:T 8:1) the CD40 TRAF2, 3 binding domain mutant failed to expand by day 15, suggesting a critical role for TRAF2, 3 binding in CoStAR signaling (FIG. 25).

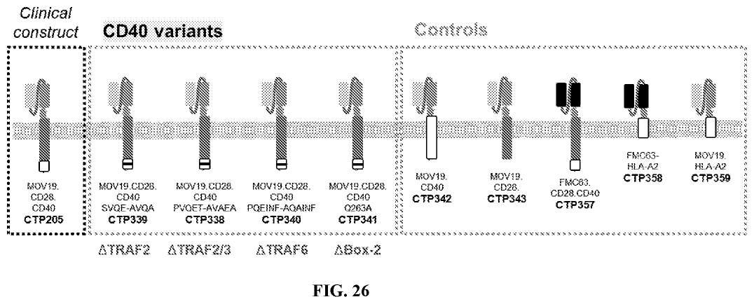

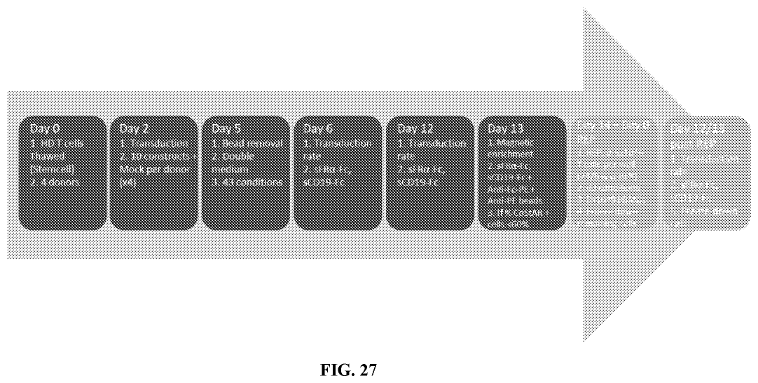

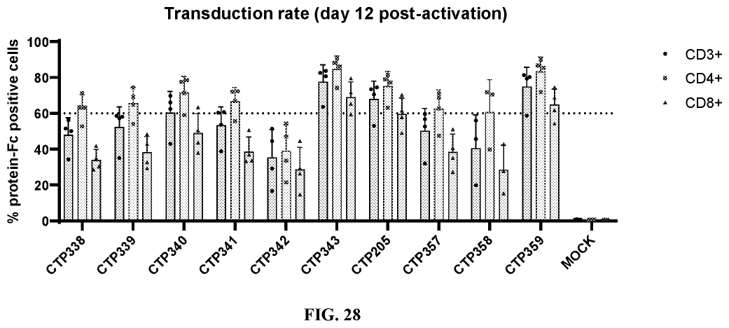

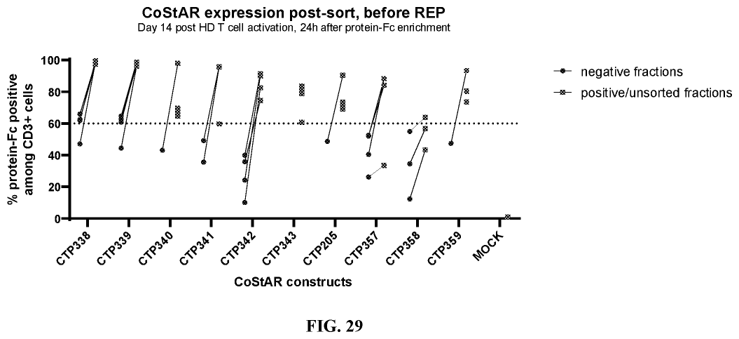

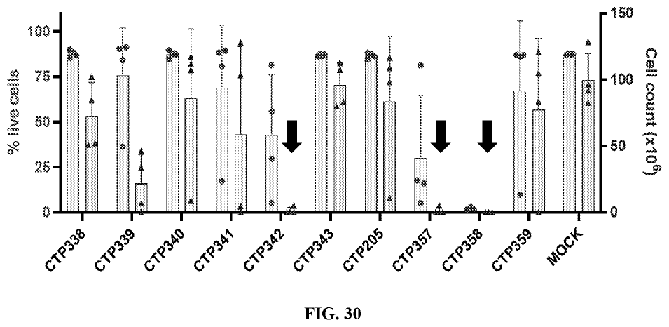

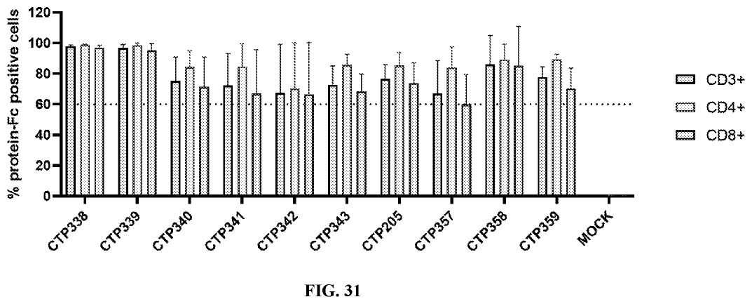

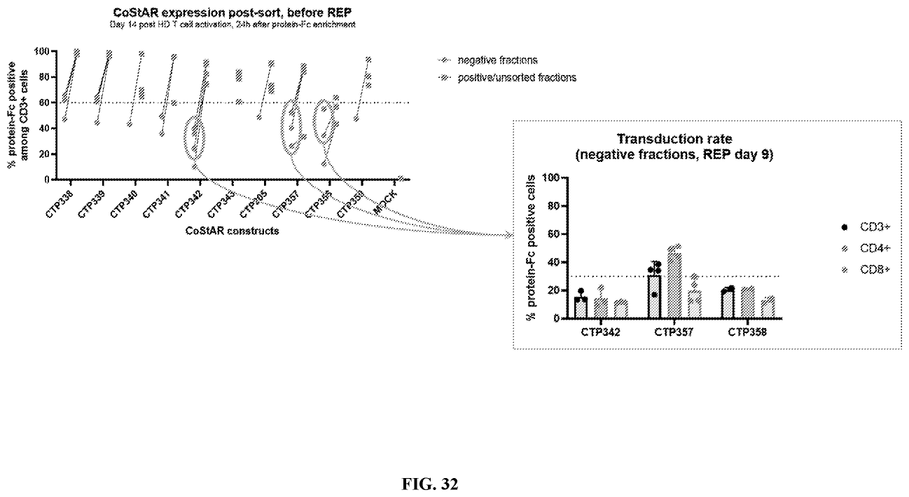

Additional studies were designed to investigate the TRAF6 binding domain mutant, TRAF2 binding domain mutant, TRAF2, 3 binding domain mutant, and Box-2 mutant in the context of the anti-FRα targeting CoStAR (MOV19 scFv) (FIG. 26). The CoStAR constructs described in FIG. 26 were developed according to the schematic of FIG. 27. Briefly, T cells from four healthy donors were thawed on day 0 and transduced on day 2 with the 10 constructs and mock conditions. On day 5, beads were removed and the media was doubled. sFRα-fc and sCD19-fc were added and transduction rate was assessed on Day 6 and again on Day 12. Magnetic enrichment was performed on Day 13 and if the % of CoStAR positive cells was less than 60%, the cells were subjected to a rapid expansion protocol (REP). Transduction rate on day 12 post activation, but prior to enrichment, is depicted in FIG. 28 for CD3, CD4, and CD8 T cells for each construct. CD4 transduction was over 60% for all constructs except CTP342 while CD8 T cell transduction was less than 60% for most constructs. As shown in FIG. 29, CoStAR expression increased in positive sorted fractions by day 14 post T cell activation, 24 hours after post fc enrichment. However, three control constructs CTP342, CTP357, CTP358 possessed low percentages of live cells and low cell count on day 12 post enrichment and REP (FIG. 30). FIG. 31 demonstrates that on day 12 after enrichment and REP, all constructs aside from CTP357 had a transduction rate above 60% for CD3, CD4, and CD8 T cells. As shown in FIG. 32, the negative fractions for CTP342, CTP357, CTP358 underwent a nine day REP to enhance the transduction rate. CTP357 and CTP358 attained a high enough transduction efficiency to be used in later experiments, however, CTP342 maintained low viability and poor transduction rate and was excluded from later experiments.

Serial stimulation assays were conducted to assess the impact of CD40 binding sites on CoStAR activity. 100,000 T cells were used per well in an 8:1 ratio with BAF3.0KT3.FRα target cells. No IL-2 was added to the assay and targets were added every 7-8 days. Viability, cell count, exhaustion profile, and % CoStAR positive cells were evaluated.

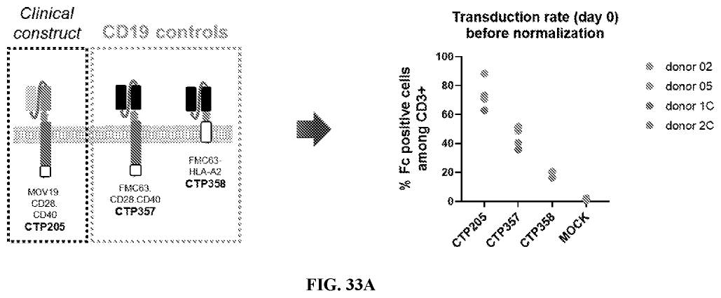

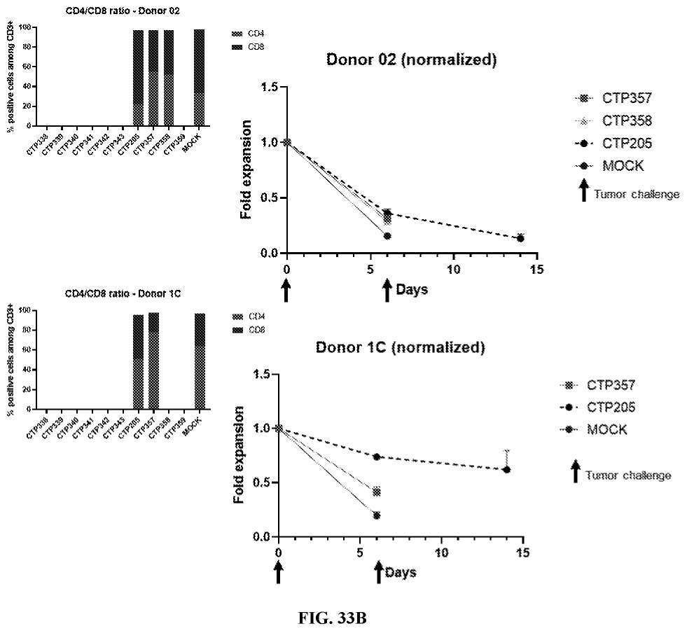

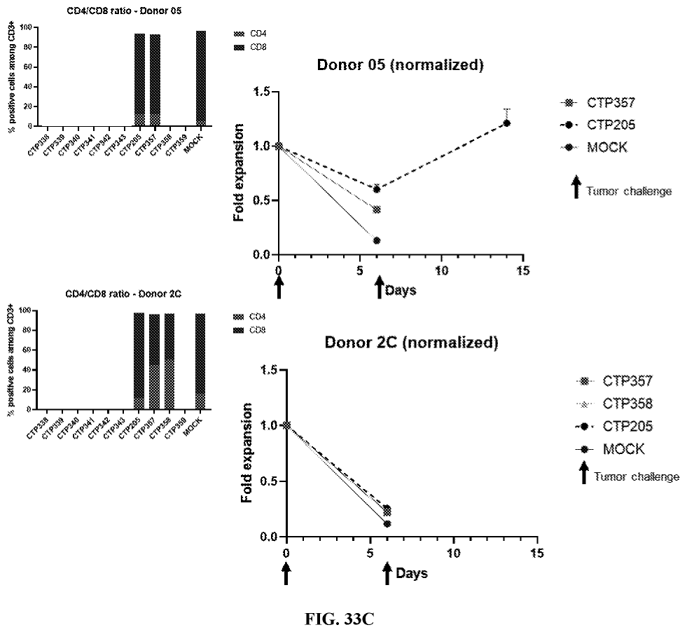

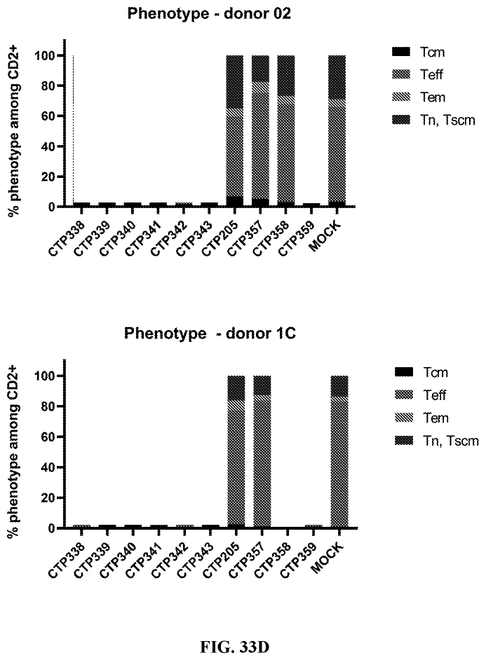

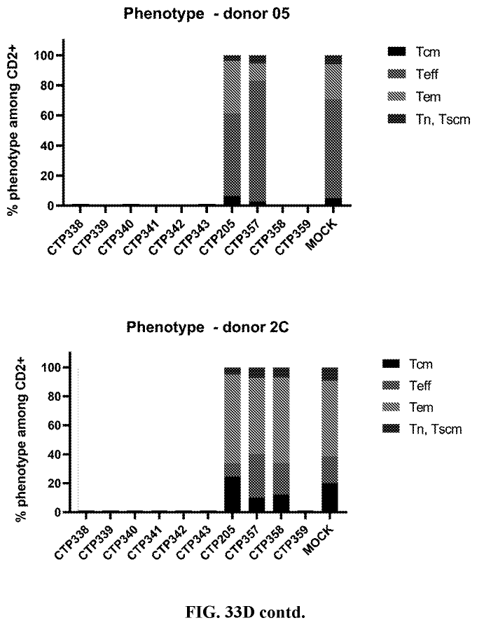

The first assay assessed MOV19 CoStAR fold expansion compared to CD19 target irrelevant controls. FIG. 33A shows the transduction rate of clinical construct CTP205 compared to the CD19 controls CTP357 and CTP358 on day 0. Fold expansion, CD4/CD8 ratios, and T cell phenotype for the three constructs for the four donors are shown in FIG. 33B-D. MOV19 CoStAR transduced cells were found to persist longer compared to CD19 irrelevant scFv controls when assessed with two tumor challenges over 14 days, with cells from three out of four donors still being detectable at day 14 (FIG. 33B, C).

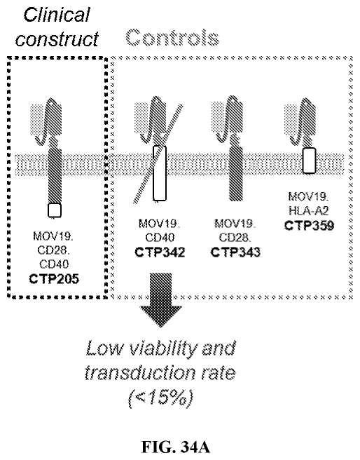

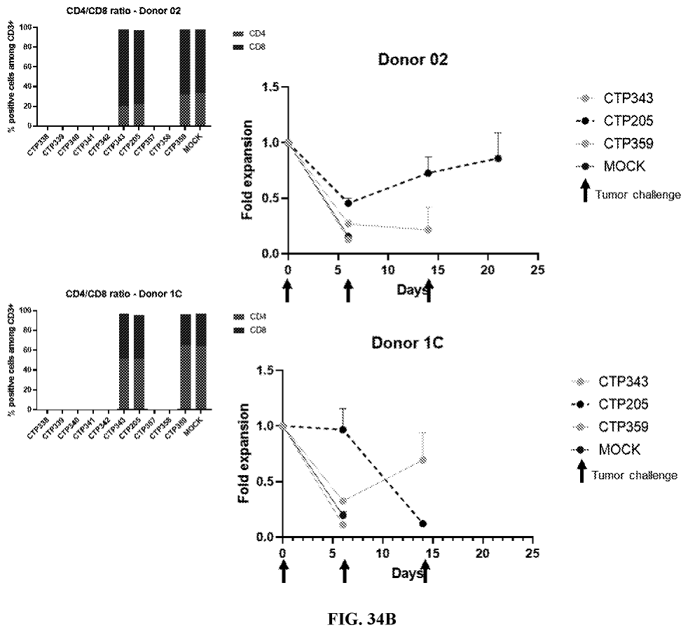

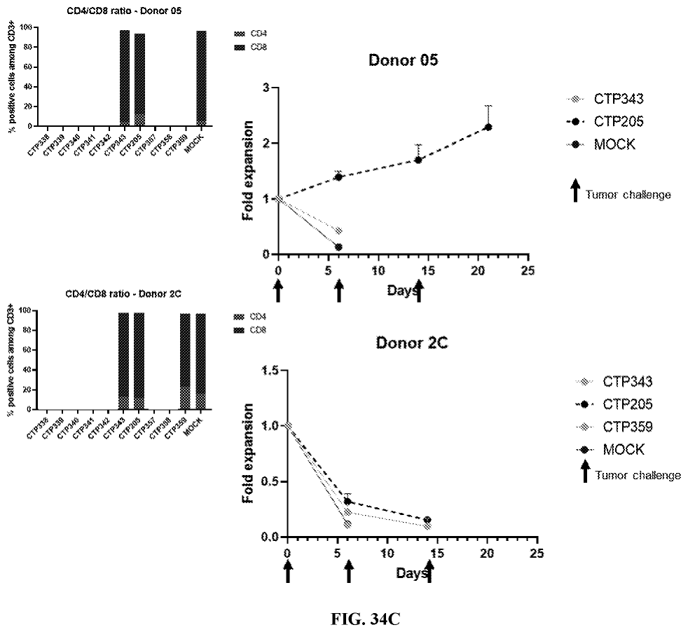

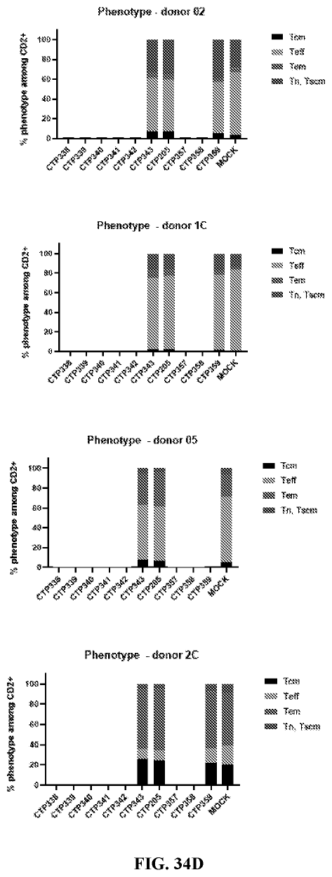

Next, cells expressing the CTP205 clinical construct were compared to cells expressing constructs comprising scFv+CD28 (CTP343), scFv+HLA-A2 (CTP359) (FIG. 34A). The serial stimulation assay was performed over 21 days and featured three tumor challenges (FIG. 34B-34D). As shown in FIG. 34B-34C, two out of four donors showed persistence of the CTP205 clinical construct out to 21 days, indicating the importance of CD40 signaling in maintaining CoStAR expansion and survival.

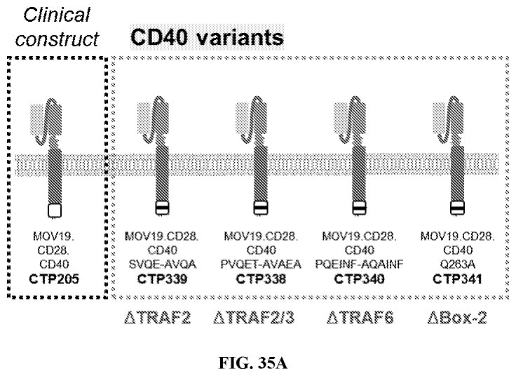

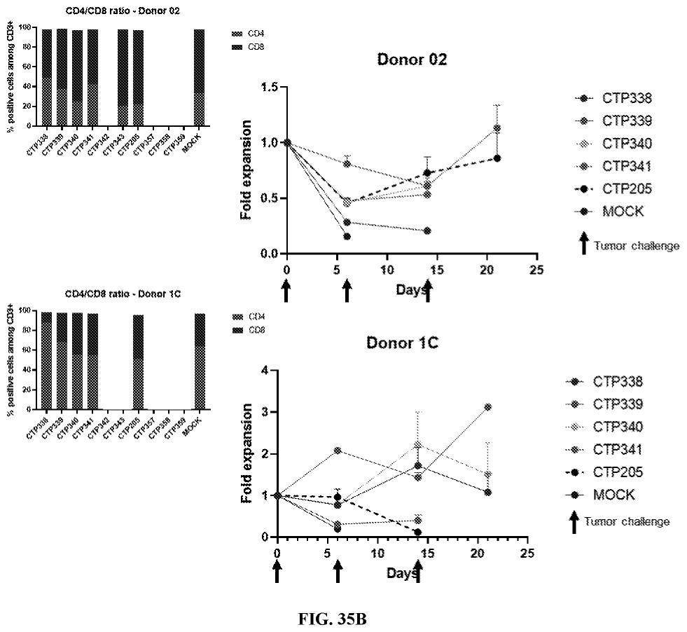

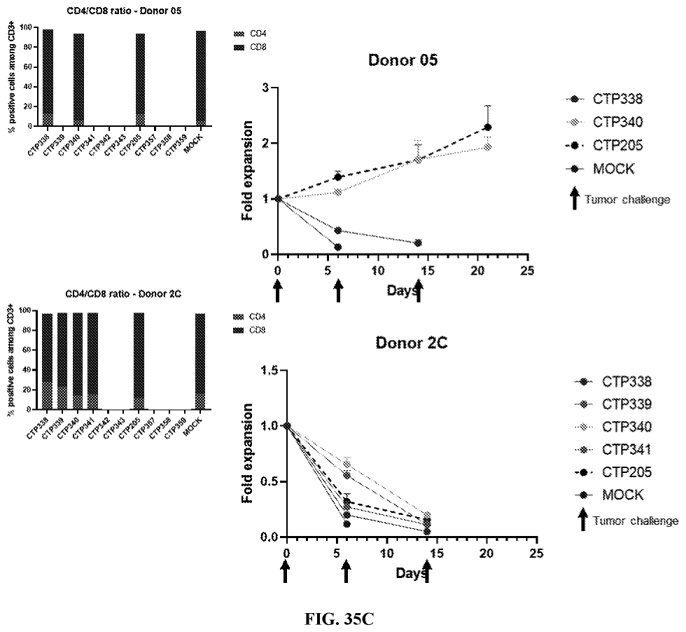

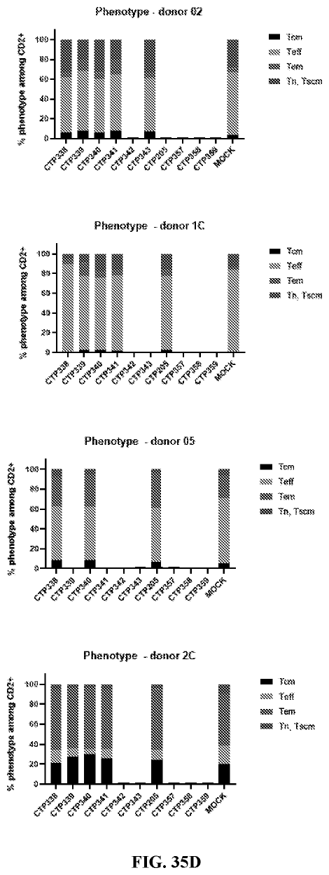

Cells expressing the CTP205 clinical construct were next compared to cells expressing four different CD40 mutations: ΔTRAF-2 (CTP339), ΔTRAF-2/3 (CTP338), ΔTRAF6 (CTP340), ΔBox-2 (CTP341) (FIG. 35A). As shown in FIG. 35B-35D, cells were assessed with three tumor challenges over a 21-day period and the TRAF2,3 binding site mutation impaired survival and proliferation of the CoStAR expressing cells compared to the clinical construct.

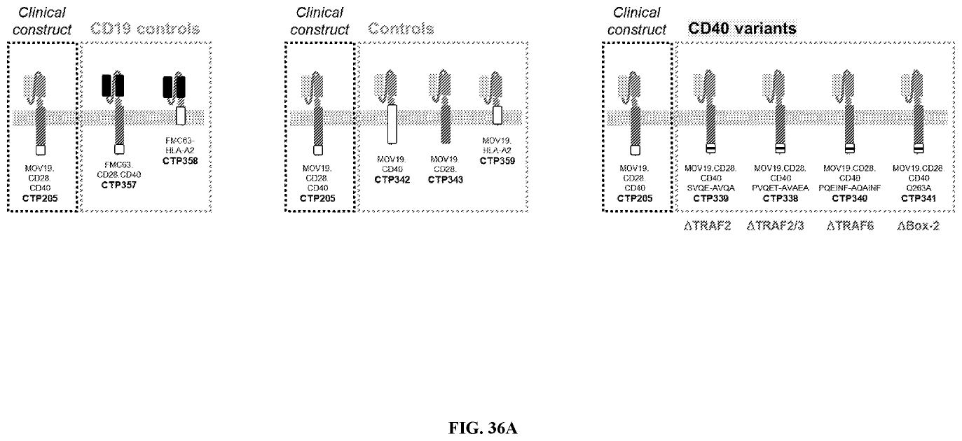

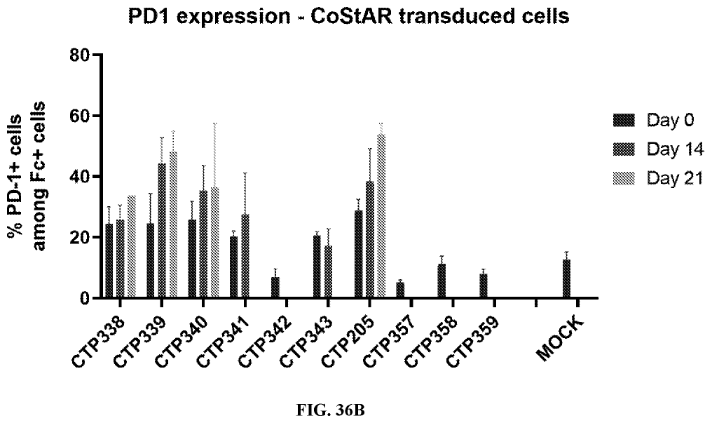

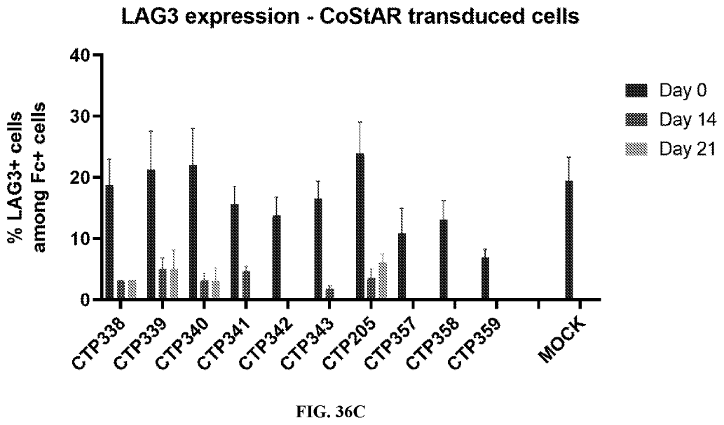

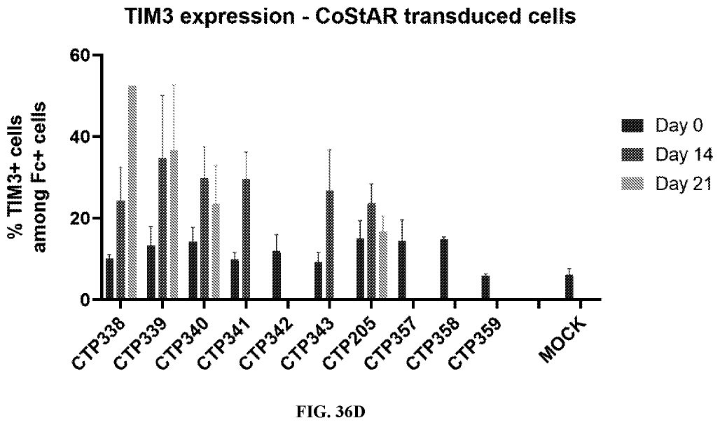

Lastly, cells expressing the ten constructs shown in FIG. 36A were evaluated for exhaustion profile. On days 0, 14, and 21 cells were assessed for expression of PD-1 (FIG. 36B), LAG3 (FIG. 36C), and TIM3 (FIG. 36D). Interestingly, TIM3 expression is much higher in cells with TRAF2, 3 or TRAF2 binding site mutations (FIG. 36D), suggesting that TRAF 2, 3 signaling is involved in maintaining the younger phenotype associated with CoStAR expression.

### Example 18

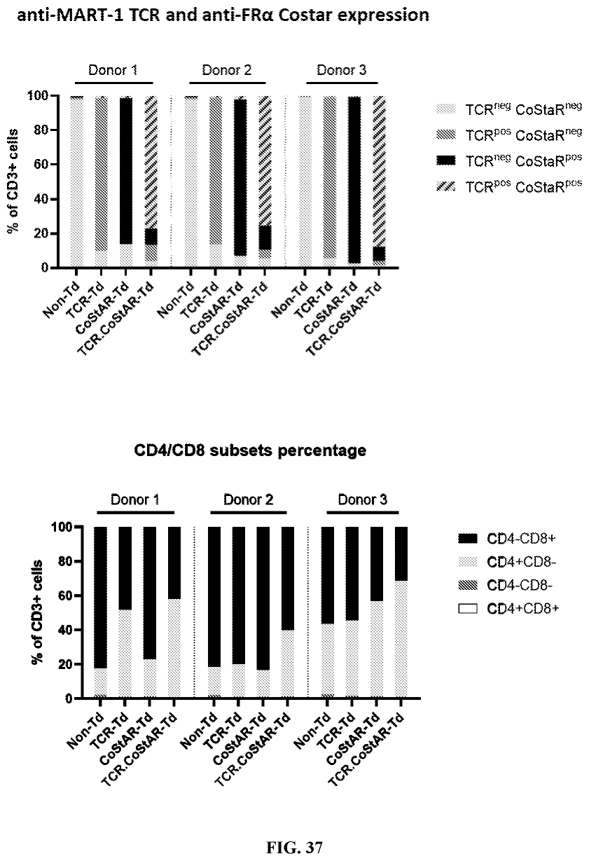

T cell activation can be mediated through a broad range of agonists, varying both in binding quality and concentration. In most situations these agonist interactions are likely to be less potent than the OKT3 signal used to demonstrate CoStAR activity thus far. We therefore developed a TCR/CoStAR co-transfer model to better understand the relationship between TCR agonism and CoStAR activity. We chose an HLA-A*02-restricted MelanA/MART-1 TCR model for which multiple agonist peptides with varying levels of activation have been identified and characterized. T cells from three donors were engineered with either CoStAR or TCR alone or in combination and sorted to achieve bulk populations enriched for expression (FIG. 37). In all three donors, over 85% of CD3+ cells expressed anti-MART1 TCR in TCR-Td condition, over 84% of CD3+ cells expressed anti-FRα CoStAR molecule in the CoStAR-Td condition, and over 75% of CD3+ cells expressed both anti-MART1 TCR and anti-FRα CoStAR molecule in the TCR.CoStAR-Td condition. T cells were then cocultured with WT or FRα-transduced T2 target cells pulsed with varying concentrations of four different agonist peptides (FATGIGILTV, ELAGIGILTV, ELTGIGILTV and ALGIGILTV) of decreasing agonist activity and cytokine secretion measured after 20 h coculture.

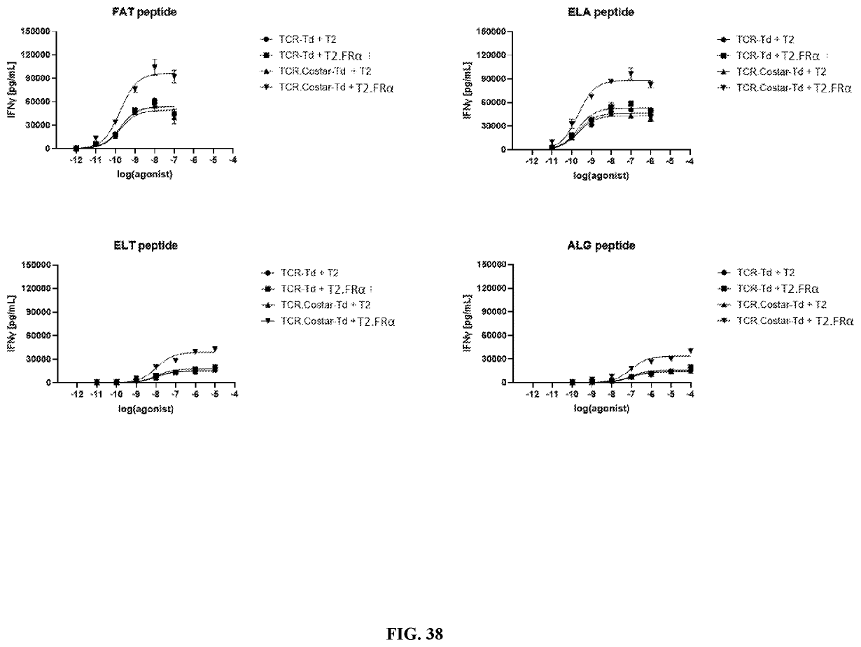

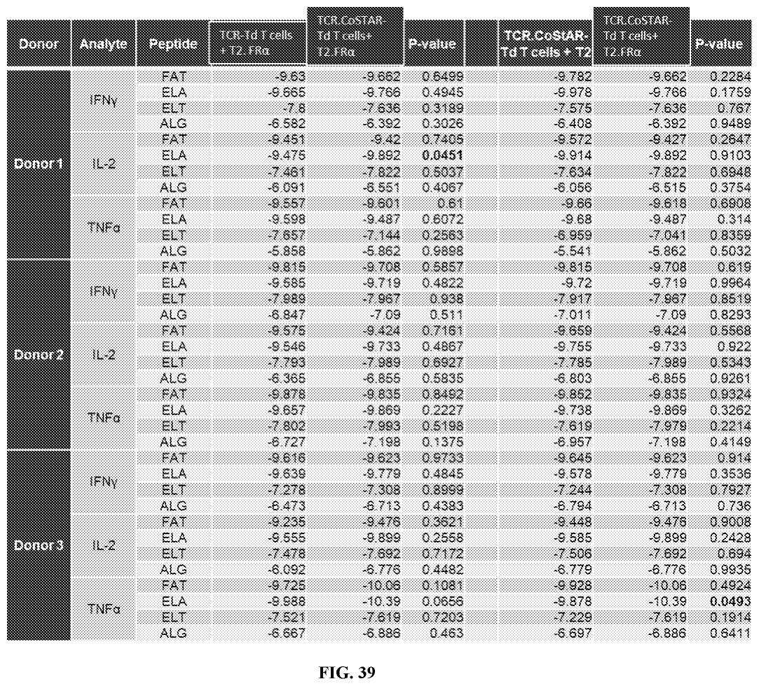

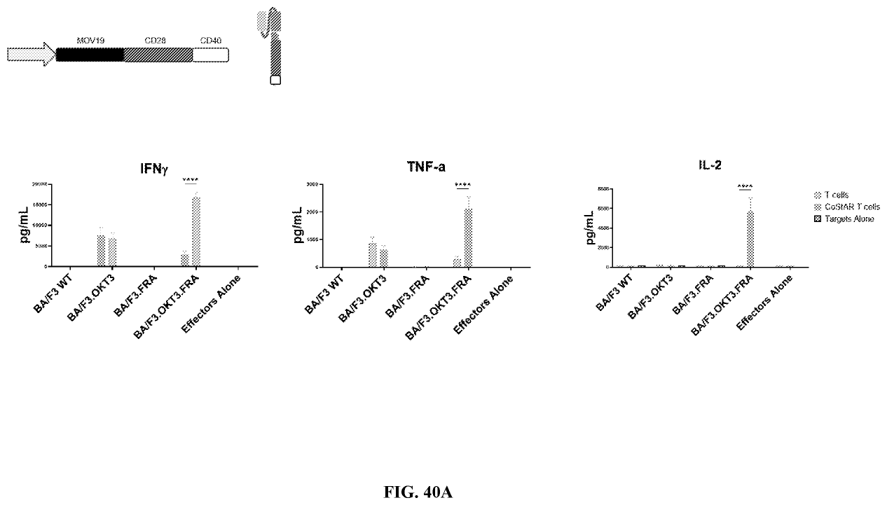

IFN-γ release by activated T cells (TCR-Td or TCR.CoStAR-Td conditions) showed a dose-dependent correlation with the peptide concentration used to load parental T2 and T2.FRα target cells. Also, the intensity of response correlated with described pMHC affinity towards anti-MART1-TCR (Clement et al, 2011; Bridgeman et al, 2012; Ekeruche-Makinde et al, 2012; Madura et al, 2015), with FAT peptide generating the strongest response, closely followed by ELA peptide, ELT generating weaker response, and ALG generating the weakest response from the 4 tested peptides (FIG. 38). Consistent with previous observations, CoStAR enhanced IL-2 secretion when combined with signal 1 agonists, compared with dose-response curves generated from cocultures with either TCR alone transduced cells responding to parental or FRα engineered T2, or with TCR.CoStAR-Td cells responding to parental T2 cells. Cocultures without a signal 1 element, between T2.WT or T2.FRα cells and CoStAR Td cells did not induce IFN-γ secretion above detectable limits (data not shown). We used dose response curves generated to calculate EC50 values for each condition (FIG. 39). Interestingly, we found that although overall CoStAR enhanced effector function elicited by a number of altered peptide ligands, there was no observable difference in the concentration of peptide required to elicit 50% maximal activation. Thus, CoStAR does not affect the EC50 of pMHC engagement mediated through the TCR.

### Example 19

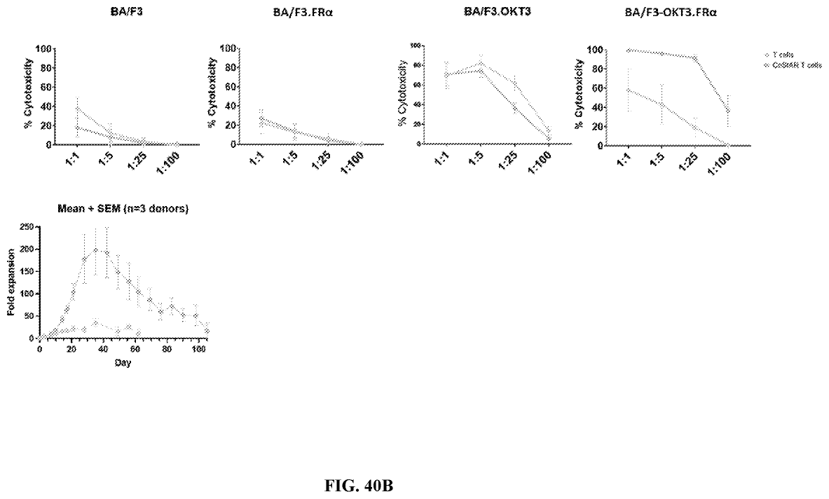

The anti-FRαCoStAR consists of a MOV19-derived scFv fused via a glycine-serine linker (×2 GSG) to the extracellular, transmembrane, and cytoplasmic domains of CD28 (amino acids 21-220) and CD40 (amino acids 216-277) (FIG. 40A). The nucleotide sequence was codon optimised for human expression and removal of cryptic splice sites to enhance expression. CoStAR was expressed from a third-generation lentiviral vector under control of an MND promoter. T cells isolated from three healthy donors were transduced at an MOI of 10, resulting in an average transduction rate of 81.43% (FIG. 40A). To test CoStAR response exclusively to FRα upon coengagement of the TCR we utilized the murine cell line Ba/F3 engineered to express either OKT3, to induce TCR stimulation (signal 1); FRα, to induce CoStAR stimulation alone (signal 2); or OKT3 and FRα to trigger TCR stimulation alongside co-stimulation through the CoStAR molecule (signal 1 and 2).

Non-transduced and CoStAR transduced T cells were cocultured for 24 hours with these individual target lines at an E:T of 1:1. Production of IL-2, TNFα, and IFNγ from cocultures was then measured (FIG. 40A). Production of all three cytokines from transduced and non-transduced cells alone, or in cocultures with WT Ba/F3 or Ba/F3. FRα was below the level of detection. Although all three cytokines could be detected in cocultures with Ba/F3.OKT3, there was no significant difference between non-transduced and CoStAR transduced cells. However, in cocultures with target cells providing both signal 1 and signal 2 (Ba/F3.0KT3.FRα) there was significantly increased production of all three cytokines in CoStAR-Td compared with non-Td cells. Similar results were seen with additional cytokines (data not shown).

Next, we assessed the ability of CoStAR to modulate T cell mediated cytotoxicity. To this end transduced and non-transduced cells were cocultured with the engineered target lines at varying effector:target ratios (1:1, 1:5, 1:25 & 1:100) and absolute counts of target cells were enumerated after 5 days (FIG. 40B). Although there was observed cytotoxicity against WT Ba/F3 at high E:T ratios, cytotoxicity did not exceed an average of 38%, and is potentially attributed to xenoreactivity. Importantly, no difference in cytotoxicity was observed between CoStAR and non-transduced T cells. Similar results were seen with Ba/F3.FRα target lines, with no enhanced killing mediated by CoStAR engineered cells. As expected, efficient killing of Ba/F3.0KT3 cells was observed, with maximal cytotoxicity of 82% and 74% seen at E:T of 1:5 by CoStAR and non-transduced cells respectively. A ratio dependent effect was observed with >20% cytotoxicity observed by either transduced or non-transduced cells. Importantly there was no significant difference in killing between transduced and non-transduced cells at any ratio tested. In co-cultures with Ba/F3.0KT3.FRα similar ratio dependent killing responses were seen with transduced and non-transduced cells, however there was significantly enhanced killing of target cells by CoStAR transduced cells at all E:T ratios tested.

To examine an impact on the ability of CoStAR to modulate T cell proliferation, transduced and non-transduced cells were cocultured with Ba/F3.0KT3.FRα at an E:T of 8:1 in the presence of 200 IU/mL IL-2, and fold expansion was measured by taking viable cell counts every 2-3 days (FIG. 40B). Non-Td cells expanded to an average of 36-fold by day 35 before undergoing population contraction, falling below detectable levels by day 62. In contrast, CoStAR transduced cells underwent significantly enhanced proliferation, reaching an average of 198-fold expansion by day 35, and surviving to day 105.

### Example 20

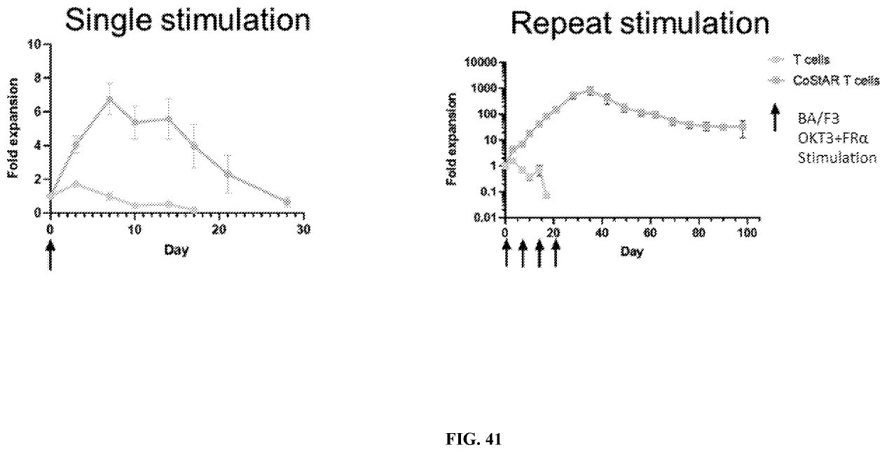

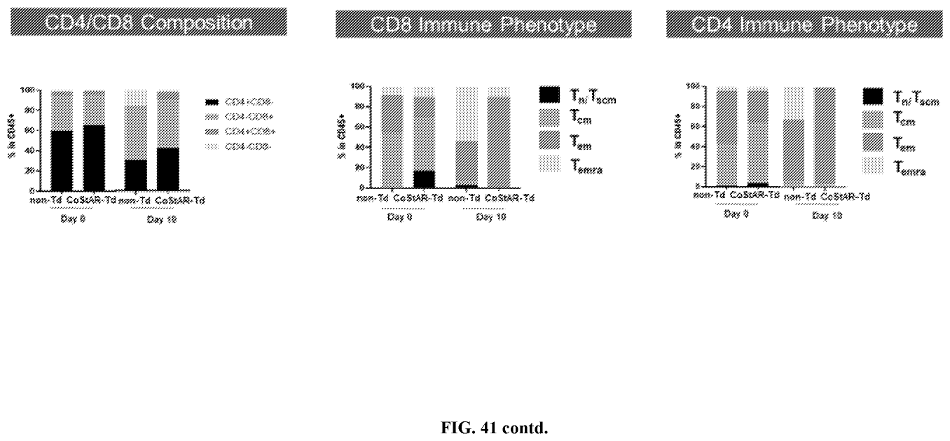

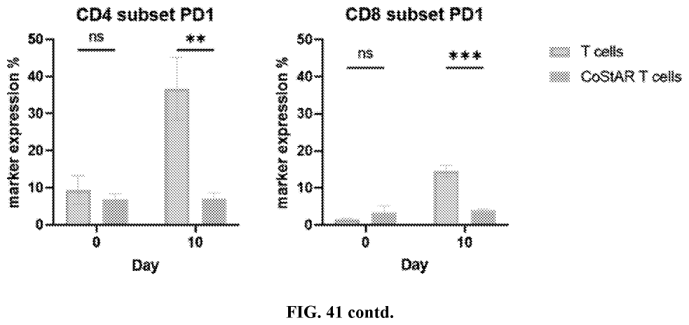

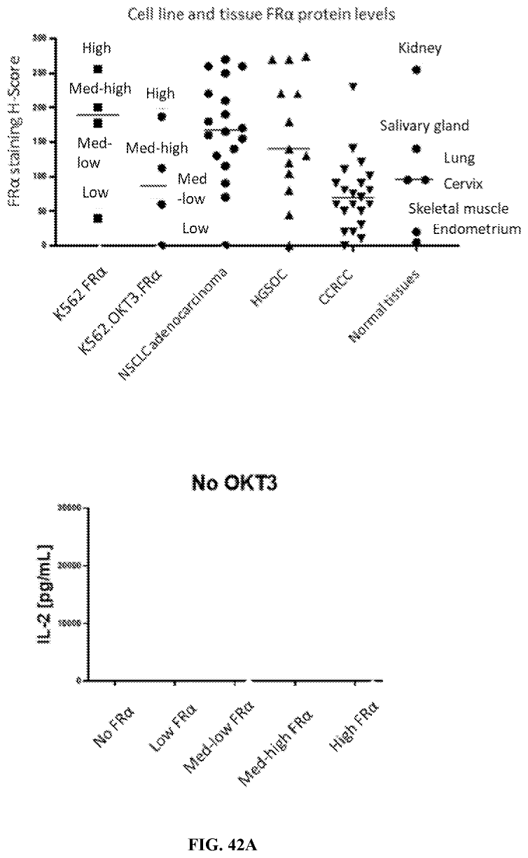

TIL therapy is currently limited by the dependence on treatment of patients with high dose IL-2 post-infusion. To determine whether CoStAR could mitigate this IL-2 dependence we performed an in vitro model of serial stimulation to mimic constitutive tumor engagement. To this end we performed stimulation with single or repeat additions of Ba/F3.0KT3.FRα cells at days 0, 7, 14 and 21 in the absence of exogenous IL-2; T-cell expansion was assessed by enumeration of total viable cells (FIG. 41). Following a single stimulation, non-Td cells expanded 1.7-fold by day 3 before contracting to undetectable levels by day 17, whereas CoStAR-Td cells expanded to 6.7-fold by day 7 and survived to day 28. With repeated stimulation, non-Td cells reached an average fold expansion of 1.5 at day 3 before cell numbers contracted and were undetectable by day 17. In contrast, CoStAR engineered cells were capable of proliferation upon each repeat stimulation with target cells, reaching an average expansion of 829-fold by day 35.

Phenotypic analysis of transduced and non-transduced cells at day 0 showed a similar CD4/CD8 split of approximately 60/40 in both transduced and non-transduced cells. By day 10, following two rounds of antigenic stimulation, PD1 expression in CD4+ cells were, on average, below 10% in both transduced and non-transduced cells at day 0. By day 10, following two rounds of stimulation, PD1 expression in transduced cells was unchanged, whereas non-transduced cells had significantly elevated levels at an average of 37% (FIG. 41). Similar results were observed in CD8+ T cells, with significantly elevated levels of PD1 in non-transduced vs transduced cells at day 10. These data demonstrate that CoStAR enhances proliferation of T cells in an exogenous IL-2 independent manner and keeps the resulting cells in a less exhausted state.

### Example 21

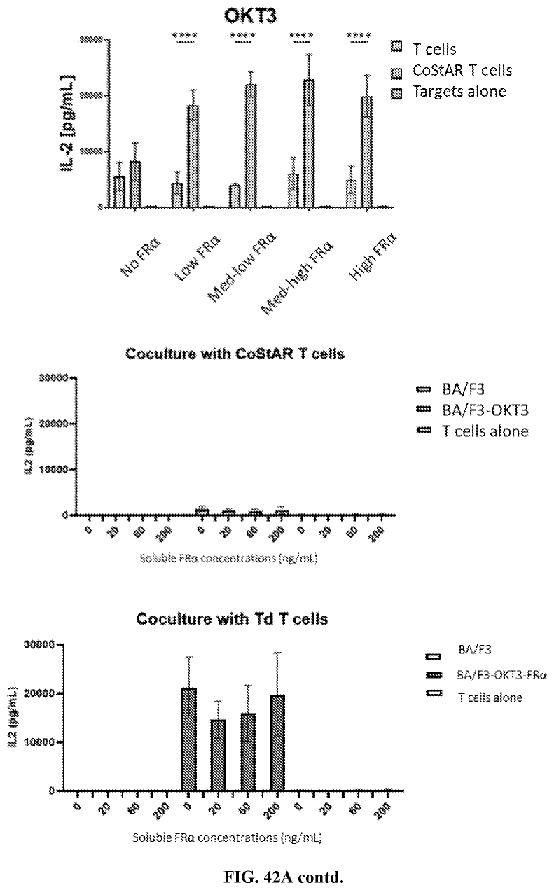

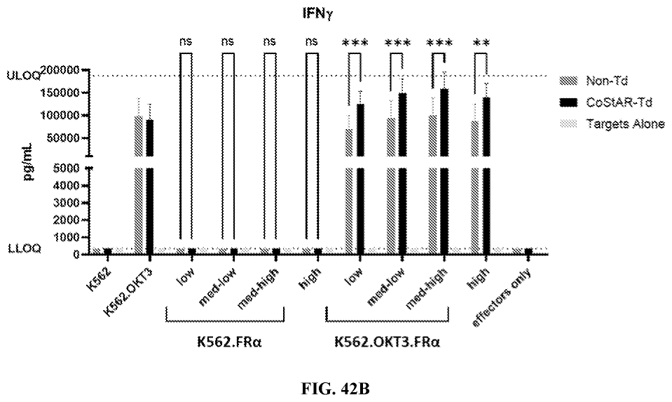

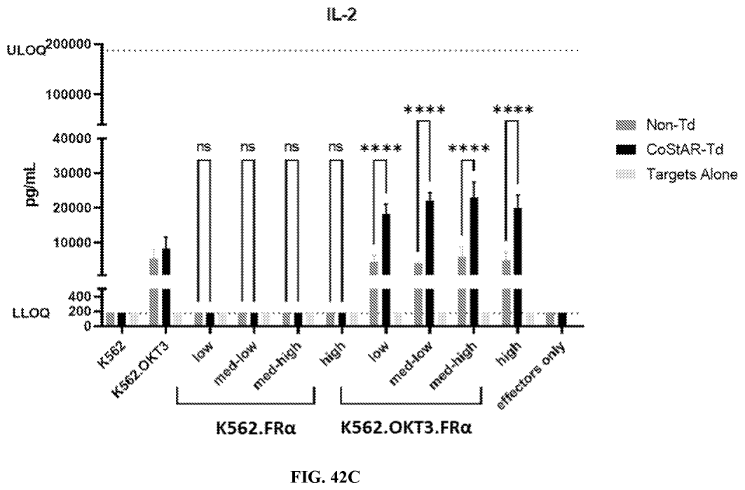

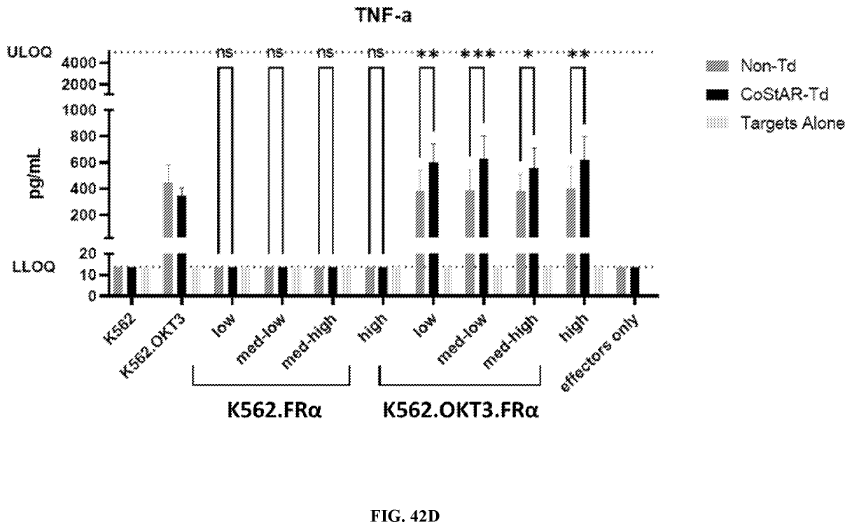

Although FRα is overexpressed in tumor, expression may vary from patient to patient, and across regions within the tumor itself; furthermore, it is known that there is restricted expression on some normal tissue. To explore how different levels of FRα in the presence and absence of signal 1 affect CoStAR activity, we developed an in vitro model system which could be used to further interrogate both off target toxicity and on-target efficacy. K562 cells were engineered to express varying levels of FRα, with or without membrane anchored OKT3. The engineered cell lines as well as tissue sections from both neoplastic (non-small cell lung cancer adenocarcinoma, high grade serous ovarian cancer and clear cell renal cell carcinoma) and normal tissue (kidney, salivary gland, lung, cervix, skeletal muscle and endometrium) were immunohistologically examined with an IVD approved FRα antibody, and the resulting H-scores calculated (FIG. 42). The widest range of FRα was observed in HGSOC (H-score range 0-275, median 180). NSCLC had a similar range of FRα expression, with ccRCC having a more restricted range of approximately half that of NSCLC and HGSOC. In normal tissue, the highest observed expression was in normal kidney, salivary gland, lung and cervix had intermediate H scores, with skeletal muscle and endometrium having low H scores. K562.FRα (high) had a similar H score to normal kidney, with K562.FRα (low) having similar expression to skeletal muscle. K562.0KT3.FRα (high) had similar FRα expression to an average NSCLC sample, with the K562.0KT3.FRα (med-high) and (med-low) being similar in FRα expression to an average ccRCC sample. These K562 lines were thus indicative of physiological samples for the purposes of the experiment.

To examine the effect of signal 2 alone on CoStAR bearing cells, transduced and non-transduced cells were incubated with K562.FRα target cells expressing different levels of FRα, and secreted IL-2 was measured after 24 h (FIG. 42). IL-2 was below the lower limit of detection for all conditions, regardless of FRα expression level or presence of CoStAR. These data demonstrate the safety of the CoStAR technology, highlighting the absolute requirement for Signal 1 to synergize with CoStAR.

Next, we assessed CoStAR T-cell activation by cells expressing varying levels of FRα, with OKT3 as an activating signal. To this end transduced and non-Td T cells were cocultured with K562.0KT3.FRα for 24 h and then IL-2 secretion measured. IL-2 production from transduced and non-Td T cells cocultured with K562.OKT3 (no FRα) was not significantly different. However, CoStAR transduced cells demonstrated significantly enhanced IL-2 production in response to K562.OKT3 expressing a range of FRα levels compared to non-transduced cells. Importantly, there was no obvious trend with regards to FRα level and IL-2 production suggesting that CoStAR cells are intimately tuned to respond to even low levels of target antigen.

FRα can be shed from the cell surface and is present at high levels in cancer patient serum (Kurosaki et al. 2016). We therefore questioned whether sFRα could: i) costimulate T cells in the presence of a TCR agonist; and ii) block costimulation mediated through membrane anchored FRα. To this end, we performed cocultures of transduced T cells with Ba/F3.OKT3 and Ba/F3.0KT3.FRα in the presence of sFRα concentrations reported in ovarian cancer patient serum as well as at supraphysiological levels (FIG. 42). Binding of solFRα to the CoStAR-Td T cells was confirmed by staining the cells post-incubation with a secondary PE anti-His antibody (data not shown). In cocultures with Ba/F3.OKT3, there was no increase in secreted IL-2 in the presence of increasing concentrations of sFRα. These data demonstrate that sFRα cannot costimulate CoStAR T cells even at supraphysiological concentrations. Next, to assess the potential impact of sFRα on blocking of CoStAR mediated costimulation through membrane bound FRα, Td T cells were cocultured with Ba/F3.OKT3. FRα targets in presence of increasing concentrations of sFOLR1α. Consistent with previous observations, Ba/F3.0KT3.FRα elicited a higher amount of secreted IL-2 from CoStAR T cells compared to Ba/F3.OKT3. Interestingly, we did not detect inhibition of CoStAR-mediated IL-2 secretion in the presence of Ba/F3.0KT3.FRα with increasing concentrations of sFRα, demonstrating that sFRα is not expected to block CoStAR activity at the physiological concentrations observed in cancer patients.

To assess the impact of FRα level on T cell functional avidity, K562 derived cell lines with low, med-low, and med-high expression of FRα and with or without OKT3 expression were cocultured with non-transduced T cells or t cells transduced with a FRα specific CoStAR. K562 cells lacking OKT3 expression failed to induce high levels of cytokine secretion from T cells. Target cell expression of OKT3 enhanced effector cell cytokine secretion, with the highest levels of secreted cytokines seen when K562.0KT3.FRα cells were incubated with CoStAR transduced T cells. For IFNγ, TNFα, and IL-2, CoStAR transduced cells exhibited higher levels of cytokine secretion than non-transduced cells when incubated with K562.0KT3.FRα cells. Critically, comparable levels of secreted cytokines were observed regardless of whether K562 cells with low, low-med, or med-high expression of FRα were used (FIG. 42B-42D). Therefore, this result indicates that the level of FRα expression does not affect functional avidity of T cell expressing cells.

### Example 22

Next, we sought to investigate the activity of CoStAR in vivo. To this end we developed a subcutaneous solid cancer xenograft cell line model in nonobese diabetic/severe combined immunodeficiency IL2γnull (NSG) mice, engrafted with FRα engineered NCI-H508 (H508.Luc.GFP.FRα) cell line which presents a CEA derived peptide (CEA:691-699 IMIGVLVGV) via HLA-A*02 to a high affinity CEA-specific TCR (Parkhurst et al. 2009).

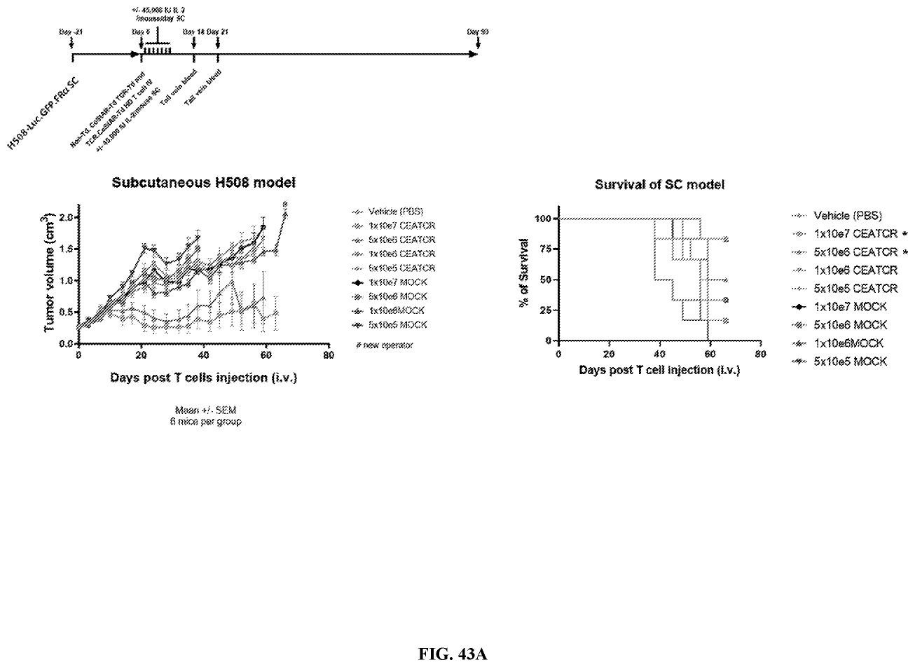

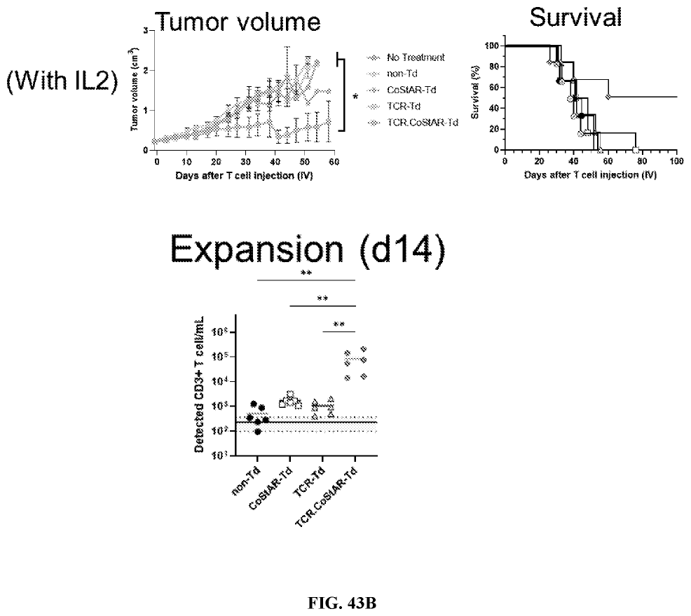

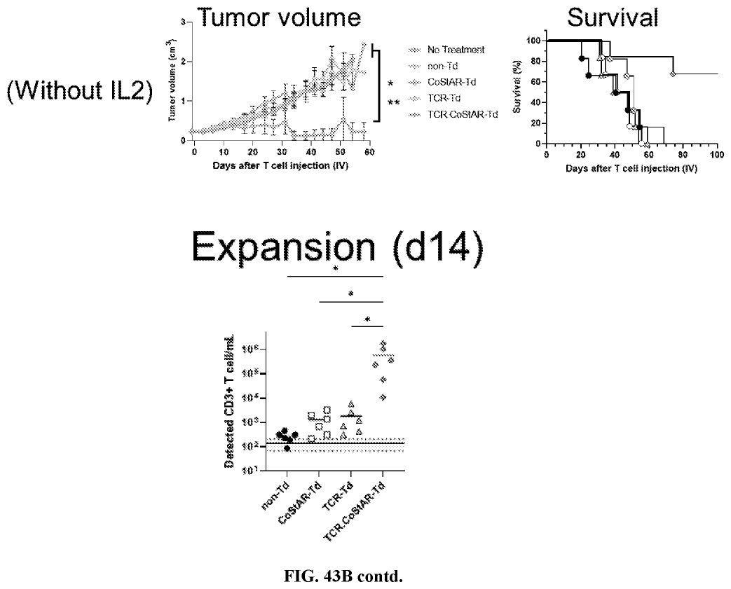

To determine the optimal TCR-Td cells dose in vivo, 1×107 H508.Luc.GFP.FRα cells were injected subcutaneously into mice at day −21. Mice were then randomized into groups of six animals and injected with either PBS or TCR-Td or non-Td T cells at doses of 1×10⁷, 5×10⁶ or 5×10⁵ cells on day 0. In mice receiving exogenous IL-2, doses of 45,000 IU were administered on day 0 to 7. Tumor volumes were then measured up to day 63. Doses of 1×10⁷ and 5×10⁶ TCR-Td cells were able to mediate tumor control compared to all other groups, as observed in average tumor volume, with 1×10⁷ and 5×10⁶ TCR-Td cells leading to 83 and 50% survival at day 63 (FIG. 43A).

Patients receiving TIL therapies to date also receive high-dose interleukin-2 (IL-2) to support T-cell engraftment (Dudley et al, 2005), which we recapitulate in our mouse model with administration of clinical-grade aldesleukin. Given the mechanism of action of the anti-FRα CoStAR molecule, which increases the secretion of proinflammatory cytokines such as IL-2 and supports in vitro expansion of T cells in an IL-2 independent manner, we utilized our in vivo model to examine whether the anti-FRα CoStAR molecule circumvented the requirement of exogenous administration of IL-2 for in vivo T-cell expansion, antitumor efficacy, and survival benefit in the present mouse model.

To test this, 1×10⁷ H508.Luc.GFP. FRα cells were injected subcutaneously into mice at day −21. Mice were randomized and injected with either PBS or TCR, CoStAR or TCR.CoStAR-Td cells on day 0. Mice were grouped either to not receive IL2 or were administered 45,000 IU of IL-2 subcutaneously on days 0-7.

Administration of TCR.CoStAR-Td T cells led to better control of tumor growth relative to non-Td, CoStAR-Td, and TCR-Td treatment groups. CoStAR-Td alone did not limit tumor growth, demonstrating that TCR engagement of pMHC is a requirement for anti-FRα CoStAR activity in vivo, and supports our in vitro studies. For non-Td, CoStAR-Td, TCR-Td, and TCR.CoStAR-Td treatment groups, average tumor volumes were 1.98 (SD±0.52), 1.95 (SD±0.51), 1.95 (SD±0.40), and 0.94 (SD±0.99) cm³, respectively, at experimental endpoints or day 58 (FIG. 43B). Of the mice in the TCR.CoStAR-Td treatment group, 3 out of 6 responded to treatment. Responder mice in the TCR.CoStAR-Td treatment group had an average tumor volume of 0.22 (SD±0.22) cm³ at experimental endpoints or day 58 (FIG. 43B). When mice were administered TCR.CoStAR-Td T cells from donor 1, their tumor volume was significantly smaller than the non-Td treatment group from days 0 to 58 (* P<0.05; FIG. 43B). Conversely, non-Td, TCR-Td, and CoStAR-Td treatment groups receiving T cells had no differences in tumor size from the no-treatment group (FIG. 43B).

Adoptive cell transfer of TCR.CoStAR-Td T cells led to a significant improvement in survival of tumor-bearing mice. This benefit was not observed in non-Td, CoStAR-Td, or TCR-Td treatment groups. These differences in survival highlight the dependency of anti-FRα CoStAR molecule on TCR signaling to confer a therapeutic benefit. All tumor-bearing mice in the PBS treatment group reached experimental endpoints by day 55. For mice receiving T cells from donor 1, non-Td, CoStAR-Td, and TCR-Td treatment groups all reached their tumor volume limits by days 52, 76, and 55, respectively (FIG. 43A). In the CoStAR-Td treatment group, 5 of 6 mice had reached experimental endpoints by day 48. Conversely, in the TCR.CoStAR-Td T-cell group, 3 of 6 mice survived until the end of the study at day 99 (FIG. 43A).

TCR.CoStAR-Td T cells showed improved in vivo T-cell expansion when compared with all other treatment groups. CoStAR-Td T cells did not confer improved expansion to T cells in the absence of anti-CEA TCR-mediated signal 1 (ie, TCR-Td group), demonstrating its functional dependency on signal 1 activation. In mice treated with T cells from donor 1 increases of 166.6-, 50.2-, and 81.0-fold T cells/mL in TCR.CoStAR-Td recipient mice relative to non-Td, CoStAR-Td, and TCR-Td treatment groups were observed at day 14 (FIG. 43B). The detected concentration of 8.68 (±SD 7.86)×104 CD3 T cells/mL in the TCR.CoStAR-Td group was significantly higher than for non-Td (** P<0.01), CoStAR-Td (** P<0.01), and TCR-Td (*** P<0.001) groups whose concentrations were 0.52 (±SD 0.46)×10³, 1.73 (±SD 0.78)×10³, and 1.07 (±SD 0.64)×10³ CD3 T cells/mL, respectively. This suggests that anti-FRα CoStAR molecule binding of FRα on tumor cells (signal 2) is conferring enhanced costimulation and persistence to CEA tumor antigen-specific anti-CEA TCR T cells.

Without supportive IL-2 administration, 4 out of 6 mice in the TCR.CoStAR-Td treatment group were responder mice. The terminal, or day 58, average tumor volume of mice in the TCR.CoStAR-Td group was 0.51 (SD±0.83) cm³ with 4/6 mice having undetectable tumors on day 58 (FIG. 43B). Furthermore, the tumors of TCR.CoStAR-Td T cells were significantly smaller than non-Td (* P<0.05), CoStAR-Td (** P<0.01), and TCR-Td (** P<0.05) treatment groups from days 0 to 58 (FIG. 43B). Therefore, anti-FRα CoStAR molecule improved control of tumor growth in this model only in the presence of anti-CEA TCR (signal 1), irrespective of exogenous IL-2 support.

The improved survival of tumor-bearing mice following treatment with TCR.CoStAR T cells was independent of supportive IL-2 administration. All tumor-bearing mice in the PBS treatment group reached experimental endpoints by day 55. Treatment groups receiving non-Td, CoStAR-Td, and TCR-Td T cells from donor 1 reached experimental endpoints by days 69, 59, and 55, respectively (FIG. 43B). In the TCR.CoStAR-Td treatment group, survival was improved relative to TCR-Td treatment groups and significantly improved compared with non-Td and CoStAR-Td T-cell treatment groups (* P<0.05), with 4 of 6 mice surviving until the end of the study (FIG. 43B). Furthermore, when the TCR.CoStAR-Td groups from donor 1 with exogenous IL-2 and without exogenous IL-2 were directly compared, there were no significant differences (FIG. 43B). Importantly, this demonstrates that anti-FRα CoStAR molecule enhancement of survival observed in this model is dependent on the presence of anti-CEA TCR but independent of exogenous IL-2 administration when combined with anti-CEA TCR.

In vivo proliferation was measured for donor 1 in the absence of IL-2 support with 2,237.7-, 447.8-, and 319.7-fold increases of CD3 T cells/mL of murine peripheral blood in the TCR.CoStAR-Td treatment group relative to non-Td, CoStAR-Td, and TCR-Td treatment groups, respectively, on day 14 (FIG. 43B). The detected concentration of 5.9 (±SD 7.03)×10⁵ CD3 T cells/mL in the TCR.CoStAR-Td group was significantly higher than in non-Td (* P<0.05), CoStAR-Td (* P<0.05), and TCR-Td (* P<0.05) treatment groups, whose concentrations were 0.26 (±SD 0.12)×10³, 1.31 (±SD 1.18)×10³, and 1.84 (±SD 2.10)×10³ CD3 T cells/mL, respectively (FIG. 43B). These data suggest that IL-2 administration is not required for the enhanced in vivo expansion observed in the TCR.CoStAR-Td group. Together, these data demonstrate that anti-FRα CoStAR molecule improves T-cell in vivo expansion in this model only in the presence TCR-pMHC-mediated signal 1, and that the improved T-cell expansion observed in TCR.CoStAR-Td groups does not require exogenous IL-2. Data presented herein reinforces the potential therapeutic benefit that could be endowed by CoStAR within the context of healthy donor T cells responding to a T cell (OKT3), or through an exogenously introduced TCR.

### Example 23

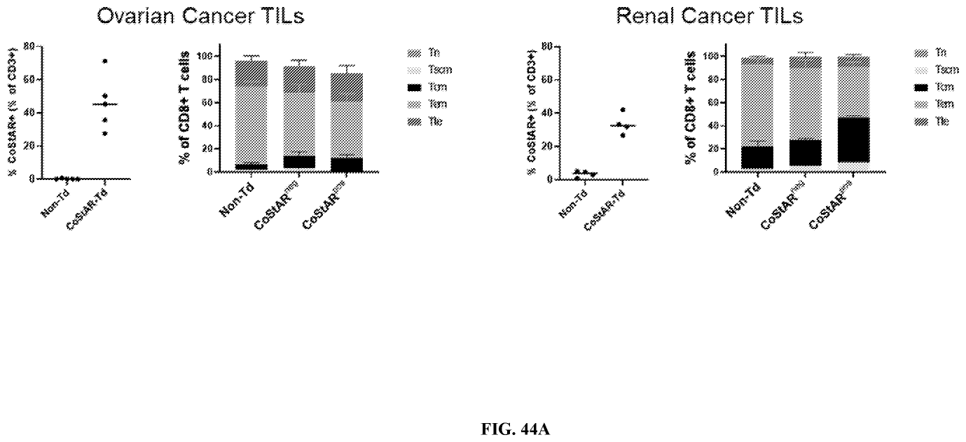

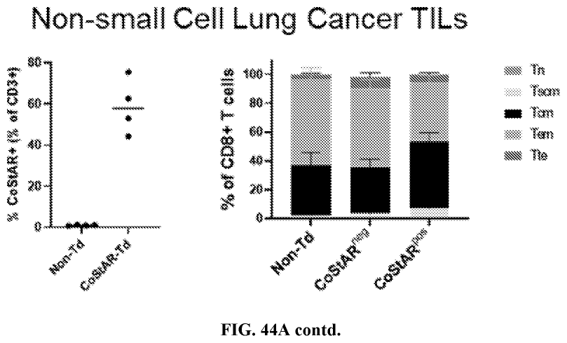

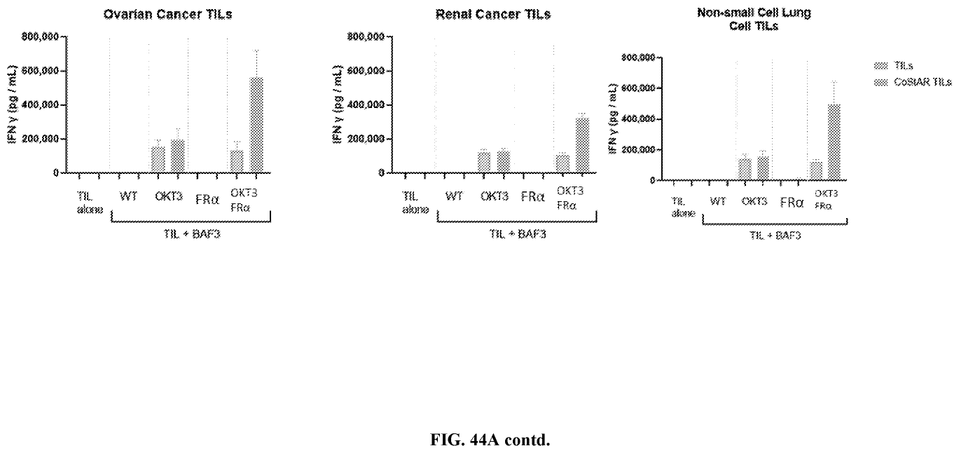

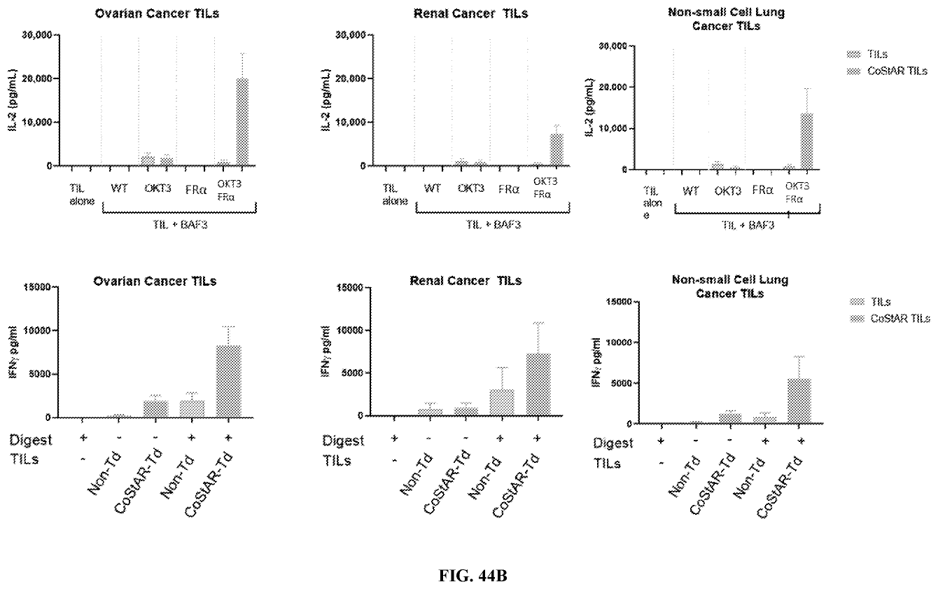

To verify the functional attributes of CoStAR in clinically relevant TIL, we developed a lentiviral transfer protocol to deliver CoStAR to patient derived TIL with high efficiency. TIL from five ovarian, four renal, and four lung tumor samples were successfully transduced with lentiviral particles to average efficiencies of 45, 34, and 59% respectively (FIG. 44A). The phenotype of TIL within the non-transduced populations as well as the CoStARneg and CoStARpos cells within the transduced populations were assessed to determine whether endowing CoStAR expression affected the phenotype of TIL (FIG. 44A). Ovarian TIL had a dominant Tem phenotype, followed by Tte, and a smaller proportion of Tcm, with no significant differences observed between the three populations analysed. Renal TIL tended towards a less Tte, and a more Tcm skewed phenotype than ovarian TIL, with CoStARpos cells harbouring a significantly lower frequency of Tem than Non-Td TIL. TIL derived from lung tumours on average had a propensity towards a more Tcm phenotype than either the renal or ovarian TIL, but retaining a more Tem phenotype overall. CoStARpos cells had significantly lower frequencies of Tem than Non-Td or CoStARneg cells and a higher frequency of Tcm than CoStARneg cells. Although differences were seen within some individual populations within indications, overall TIL phenotypes between Non-Td, CoStARneg and CoStARpos populations looked remarkably similar.

To assess the biological activity of CoStAR in TIL we cocultured transduced and non-transduced TIL from the three indications with WT parental Ba/F3 cells or Ba/F3 expressing OKT3, FRα or OKT3 and FRα, with cytokines measured after overnight culture (FIG. 44A-44B). TIL from the three indications, whether transduced or not, did not produce IFNγ nor IL-2 above the level of detection either alone or in response to WT BA/F3. Responses to Ba.F3.FRα were equally undetectable, with the exception of a very small amount of IFNγ production by CoStAR-Td lung TIL. Both IFNγ and IL-2 was detectable from cocultures with Ba/F3.OKT3, but did not significantly differ between transduced and non-Td TIL suggesting CoStAR does not negatively affect TIL natural ability to respond to TCR signaling. Importantly, we were able to demonstrate that in response to Ba/F3.0KT3.FRα CoStAR-Td produced significantly more IFNγ (ovarian: 4.2-fold; renal: 3.1-fold; lung 4.0-fold) and IL-2 (ovarian: 19.9-fold; renal: 15.0-fold; lung: 18.3-fold) than non-Td TIL.

Reactivity of non-Td and CoStAR-Td TIL towards autologous tumour was assessed to ascertain the potential enhancement of direct anti-tumor effects by the CoStAR molecule against ovarian, renal and lung tumor that express FRα (FIG. 44B). CoStAR-Td and non-Td TIL were cocultured with autologous digest overnight at an E:T of 1:1 before cytokine analysis. IFNγ secretion from digest alone was undetectable with varying amounts of background IFNγ observed from CoStAR-Td and non-Td TIL alone. In the presence of autologous digest ovarian CoStAR-Td TIL produced 4.3-fold more IFNγ; renal CoStAR-Td TIL produced 2.4-fold more IFNγ and lung CoStAR-Td TIL produced 6.4-fold more IFNγ than non-Td TIL. This demonstrates CoStAR enhanced anti-tumor activity across multiple FRα expressing tumors.

### Example 24

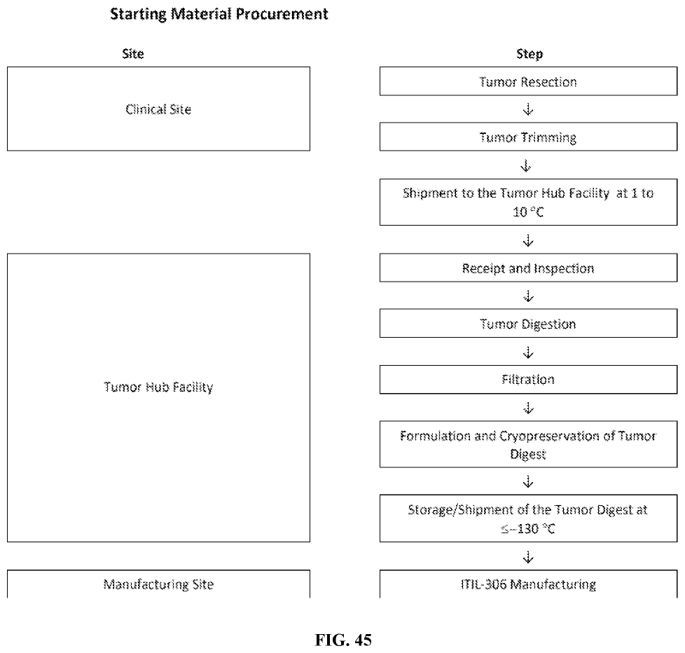

The starting material for ITIL-306 manufacturing is the digested cell suspension containing autologous TILs from resected tumor material. FIG. 45 provides the process flow diagram describing the procurement of starting material prior to the start of manufacturing. Following resection and trimming of the tumor at the clinical site, the starting material is shipped to the Tumor Hub Facility (also referred to as the Tumor Hub) for further processing. After receipt and inspection at the Tumor Hub, the starting material is digested, filtered, and cryopreserved prior to storage at −130° C.

At the clinical site, the tumor is surgically resected and then trimmed to remove visibly necrotic tissue, visibly heathy or noncancerous tissue, and excess blood. Each clinical subject lot is assigned a unique subject ID number, chain of identity number, and manufacturing batch number. These unique identification numbers are carried through the entire manufacturing process to ensure product custody and traceability.

The trimmed tumor is weighed, placed into a sterile bag, and then heat sealed. The trimmed tumor material is then prepared for transportation by introducing phosphate-buffered saline (PBS) containing 10% human serum albumin (HSA) with antimicrobial reagents, by gravity draining it through a closed tubing connection. The bag is then labeled and shipped to the tumor hub or manufacturing site at 1 to 10° C. (using the NanoCool™ shipper).

For the tumor digestion step, 15 mL of enzyme digest media (EDM) is added to the bag containing the tumor. The bag containing resected tumor and digest media is then subjected to controlled, mechanical compression at a target temperature of 35° C. for a minimum of 45 minutes using an automated device (VIA Extractor™ connected to the VIA Freeze™ from Cytiva LifeSciences), thereby facilitating mechanical and enzymatic digestion. The tumor is digested to generate a homogeneous cell suspension.

The tumor digest material is then filtered using a blood filtration set (not more than ˜200 μm pore size) in a closed system. The digested tumor is then formulated with BloodStor 55-5 to achieve a final concentration of 5% dimethyl sulfoxide (DMSO) and cryopreserved using a defined cryopreservation program. The cryopreserved cell suspension is stored in the vapor-phase of liquid nitrogen (LN2) at −130° C. and transported to the GMP manufacturing site in a qualified shipper that maintains the cryopreserved cell suspension at ≤−130° C.

### Example 25

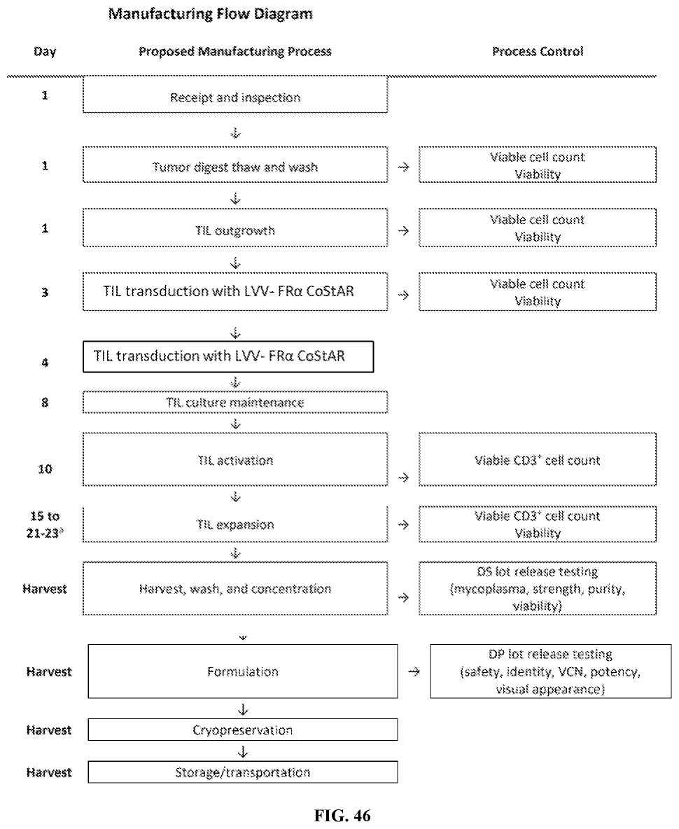

A flow diagram for the ITIL-306 drug substance manufacturing is provided in FIG. 46.

### Step 1: Receipt and Inspection

Cryopreserved tumor digest is received at Instil manufacturing facility and placed in an access controlled room whether it goes through the receipt and inspection process. As part of the inspection process, the tumor digest bag is removed from the exterior packaging and inspected to ensure bag integrity. The bag containing cryopreserved tumor digest is then thawed under controlled conditions.

### Step 2: Tumor Digest Thaw and Wash

The first step of the ITIL-306 manufacturing process is designed to transfer the cells out of EDM and DMSO. The cell suspension is first diluted to approximately 300±60 mL in T cell media (TCM) supplemented with 10% (v/v) irradiated FBS, 0.25 μg/mL amphotericin B, 10 μg/mL gentamicin, 50 μg/mL vancomycin, and 3000 IU/mL IL-2, then washed in the same media using an automated cell-processing system (Sefia™ from Cytiva LifeSciences). The cells are then concentrated and resuspended in 30 mL of TCM supplemented with 10% (v/v) irradiated FBS, 0.25 μg/mL amphotericin B, 10 μg/mL gentamicin, 50 μg/mL vancomycin, and 3000 IU/mL IL-2 in a single-use culture bag.

### Step 3: TIL Outgrowth

This process step enables the outgrowth of TILs from the tumor digest material to prepare for further processing. The TIL outgrowth process step includes cell seeding and incubation of the cell culture with media addition. This process step is carried out in functionally closed, single-use culture bags. If the total viable cell concentration from the tumor digest wash step is greater than 0.5×10⁶ viable cells/mL, the cell suspension is further diluted with TCM supplemented with a target of 10% (v/v) irradiated FBS, 0.25 μg/mL amphotericin B, 10 μg/mL gentamicin, 50 μg/mL vancomycin, and 3000 IU/mL IL-2 as needed to achieve a target concentration of 0.5×10⁶ viable cells/mL. At the beginning of the TIL outgrowth phase on day 1, the cell suspension is seeded at approximately 0.5×10⁶ viable cells/mL and incubated under standard cell culture conditions (37° C., 5% CO2).

### Step 4: TIL Transduction and Culture Maintenance

On days 3 and 4 of the ITIL-306 manufacturing process, the cells are counted and the appropriate amount of LVV (LVV-FRα CoStAR) is added to the cell culture to reach a target MOI of 5. The TIL outgrowth culture is monitored for T cell count and viability and diluted with TCM supplemented with a target of 10% (v/v) irradiated FBS, 0.50 μg/mL amphotericin B, 20 μg/mL gentamicin, 100 μg/mL vancomycin, and 6000 IU/mL IL-2 as needed. On the last day of TIL outgrowth (process day 10), the cell culture is counted and if the resulting viable total T cell count is between 1×10⁶ and 20×10⁶, the entire TIL outgrowth cell suspension is transferred to the subsequent process step. If the viable T cell count is >20×10⁶, the excess T cells may be cryopreserved in 10% DMSO and stored as reserve material, if needed for future processing.

### Step 5: TIL Activation

Following the TIL outgrowth phase, TIL activation is mediated using anti-CD3 antibodies to provide the primary signal and irradiated, allogeneic PBMCs to provide additional costimulation to support T-cell activation. For activation seeding, 1×10⁶ to 20×10⁶ viable T cells are added to a final ratio of 1:200 viable T cells: irradiated PBMCs (range of 1:100 to 1:200 viable T cells: irradiated PBMCs) in 2±0.6 L of TCM supplemented with a target of 30 ng/mL anti-CD3 antibody, 8% irradiated human AB serum, and 3000 IU/mL IL-2. The TIL activation culture is incubated for 5 to 6 days under standard cell culture conditions (37° C., 5% CO2) and monitored for viable T cell count and viability.

### Step 6: TIL Expansion

The activated TILs (2±0.6 L cell suspension) are transferred aseptically into a single-use culture bag in a functionally closed and regulated bioreactor and cultured under standard cell culture conditions (37° C., 5% CO2). The cell suspension is provided a semi-continuous feed of TCM supplemented with IL-2 at a target of 3000 IU/mL and is routinely monitored for viable T cell count and viability for 6 to 8 days. The TIL expansion step is designed to robustly achieve high cell densities. To meet the ITIL-306 final product dose requirements, a minimum number of anti-FRα CoStAR+ T cells required by the dose escalation phase is targeted by the end of the TIL expansion step to move forward to harvest. Cell growth rate varies from subject lot to subject lot; therefore, if the minimum viable T cell count of anti-FRα CoStAR+ cells is not achieved by Day 21, the batch manufacturing records allow for the TIL expansion step to extend an extra 2 days to ensure the final ITIL-306 dose requirements are met.

### Step 7: Harvest, Wash, and Concentration

Following the TIL expansion step, cells are harvested through a single-use disposable blood filtration set into a single-use culture bag, washed to reduce process-related impurities, and concentrated using PBS supplemented with 5% HAS using an automated cell-processing system (Sefia™ from Cytiva LifeSciences). The cells are then concentrated and resuspended in PBS supplemented with 5% HAS, in preparation for the formulation step.

### Step 8: Formulation

Following the wash and concentration process step, precooled CryoStor® CS10 (animal component-free medium containing 10% DMSO) is added to the cell suspension in PBS and 5% HSA at a 1:1 ratio to formulate ITIL-306 final product. Based on the amount of anti-FRα CoStAR+ cells required for dose, the final product is formulated to 170±20 mL in the final product bag resulting in a final formulation of 5% DMSO and 2.5% HSA.

### Step 9: Cryopreservation

Following the formulation step, the final product bag containing formulated ITIL-306 final product is visually inspected, labelled with the final product label, placed in a cryostorage cassette with the final product label, and transferred into a controlled-rate freezer. The final product is cryopreserved using a predefined program with a freezing rate of −1° C./minute to a final temperature of −80° C.

### Step 10: Storage and Transportation

Following the cryopreservation step, the ITIL-306 final product bag inside the cassette, is transferred to vapor-phase LN2 at ≤−130° C. for storage. ITIL-306 is maintained cryopreserved in storage (≤−130° C.) until release and transport to the treatment site. Once released, the cassette containing the final product bag is removed from LN2 storage, place into a validated LN2 shipper, and shipped to the treatment site at −130° C.

### Example 26

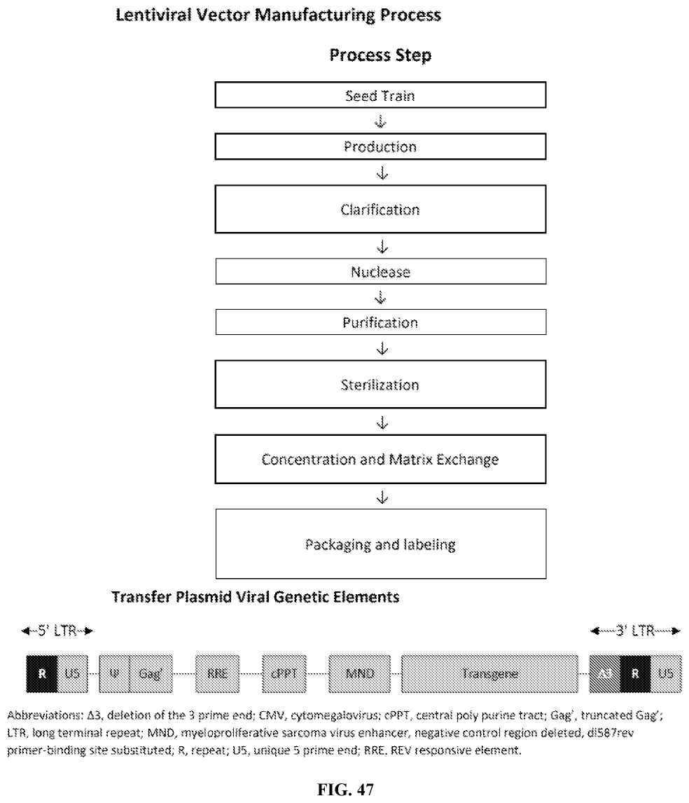

A flow diagram for the lentivirus genetic elements and manufacturing is provided in FIG. 47.

A third-generation self-inactivating replication-deficient LVV will be used to introduce aN anti-FRα CoStAR into TILs from each subject enrolled in the clinical study. The LVV, LV34, will be manufactured by VIVEbiotech. An adherent, serum-based process will be used by VIVEbiotech to manufacture the LVV lot. A HEK293T master cell bank (MCB) produced under cGMP will be used for manufacture of the LV34 LVV. The HEK293T cell line was obtained by VIVEbiotech from the American Type Culture Collection (ATCC) with reference number CRL-3216. HEK293T cells were expanded within the VIVEbiotech classified area following documented procedures to product the MCB. The MCB is stored at VIVEbiotech as well as at a separate location (Clean Cells, Bouffere, France) under controlled conditions with continuous monitoring. To increase the safety of the LVV system, components necessary for viral production are split among 4 plasmids (1 transfer plasmid encoding the anti-FRα CoStAR and 3 helper plasmids encoding REV, gag-pol, and the VSV-G envelope protein).

The manufacturing process is based on the transient polyethylenimine transfection of HEK293T cells in one single-use 10-m2 bioreactor or four 2.4-m2 bioreactors capable of producing up to 20 L of viral supernatant per batch, followed by a purification process consisting of several filtration and chromatographic steps, including ultrafiltration/diafiltration and ion exchange chromatography. A flow diagram of the LVV manufacturing process is shown in FIG. 47. The total batch volume is approximately 60 mL of final product filled into vials and stored at ≤−65° C.

The LVV utilized in ITIL-306 is a third generation self-inactivating vector. Lentiviral gene transfer vectors are based on the HIV-1 virus with a number of essential genes deleted that make them replication incompetent and non-immunogenic while retaining high efficiency of gene transfer into target cell genomes for long term stable expression of anti-FRα CoStAR. Third generation lentiviruses utilize the separation of genes required for virus packaging across four plasmids. This ensures that there is minimal possibility of recombination events leading to replication competent virus. In addition, modification to the 3 prime (3′) long terminal repeat (LTR) region prevents packaging of integrated genomes even if relevant packaging machinery is present, making the virus self-inactivating. Combined, these modifications render the virus incapable of replicating or mobilizing within the transduced cell.

### Example 27

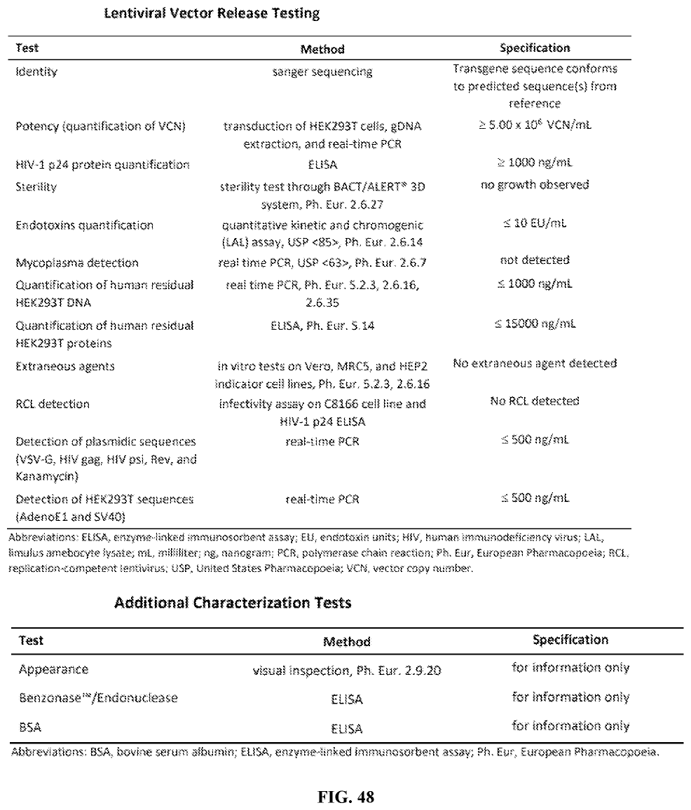

After completion of cGMP manufacture and testing, VIVEbiotech Quality Assurance and Qualified Person will release the lot based upon a panel of release tests shown in FIG. 48. Additional characterization tests are performed for information only purposes as shown in FIG. 48. Instil will also perform a ddPCR based identity test upon receipt of each lot of LVV prior to release for ITIL-306 manufacturing.

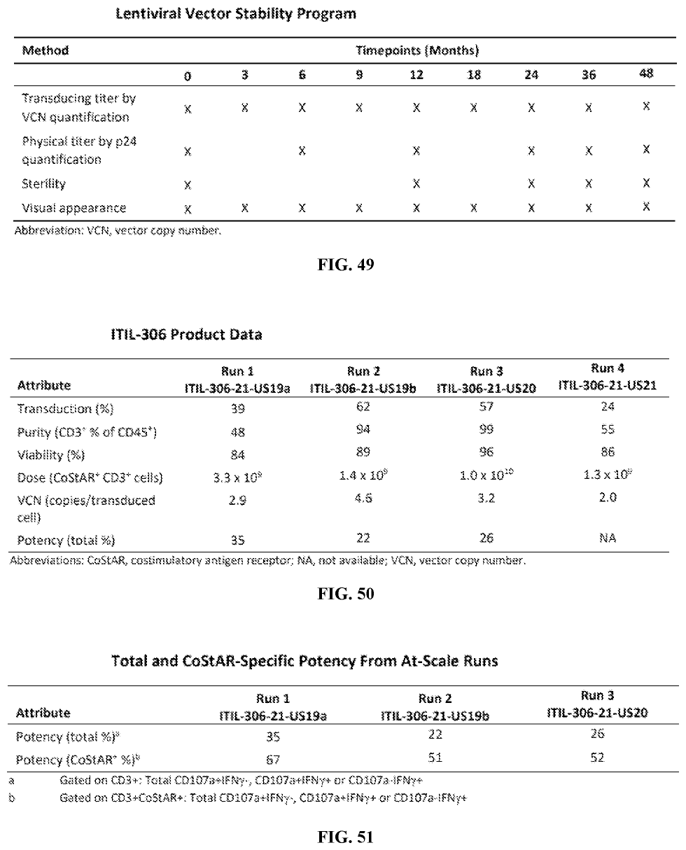

A stability program for the LVV is outlined in FIG. 49. Stability studies will be executed at VIVEBiotech for up to 48 months at ° C. The vector will be tested for sterility, transducing titer by VCN quantification, and physical titer by p24 quantification at defined timepoints as in FIG. 49.

### Example 28

This section will cover the ITIL-306 development studies performed with ovarian tumor. The manufacturing process consists of several distinct process steps that are carried out over a period of 21 to 23 days. Four process development runs with ovarian tumor starting material were performed at manufacturing scale per the process intended for the clinical trial. The in-process data demonstrates that the TILs expanded as intended. Note that the expansion cell growth plot utilized only the TILs seeded as the starting cell count and did not include the PBMCs. The batch analysis data for the runs are summarized in FIG. 50. Two runs (ITIL-306-21-US19b and ITIL-306-21-US20) met all criteria for final product release. A lower T cell purity was observed for runs ITIL-306-21-US19a and ITIL-306-21-US21, but met all other release criteria.

### Example 29

An in vitro coculture-based potency assay has been developed as a surrogate measurement for the in vivo biological activity of ITIL-306. Additional information about potency assays and uses thereof can be found in PCT App. PCT/US2022/034606, filed on Jun. 22, 2022 with the title “Methods Of Isolating Of Tumor Infiltrating Lymphocytes And Use Thereof”, which is hereby expressly incorporated by reference in its entirety. The method is a bioassay performed on thawed final, formulated, transduced T cells with a polychromatic flow cytometry endpoint for quantitation of ITIL-306 potency. The potency assay quantitates functional T cells in response to coculture with target cell lines engineered to express OKT3 anti-CD3 scFv and FRα. These target cells provide TCR stimulation to all T cells via OKT3 engagement of CD3 and enables CoStAR engagement of FRα in CoStAR-transduced cells. The potency method detects the differential activation and downstream function of T cells from individual transduced and nontransduced populations of ITIL-306 product upon antigen recognition. Since CoStAR transduction is expected to provide only a costimulatory signal, both the transduced and nontransduced populations are expected to be potent. Following coculture of TILs and target cells, each well is stained with a cocktail of antibodies which allows for the discrimination of total TILs and CoStAR-transduced TILs. T cell functionality is measured by detection of a degranulation marker, CD107a, and activation marker, interferon-gamma (IFN-γ), by flow cytometry.

CD107a is expressed on the surface of the T cell only during secretion of cytotoxic molecules in response to activation, and thus directly quantifies the percentage of activated TILs, which are capable of killing target cells. Likewise, IFN-γ staining provides quantitative analysis on the percentage of TILs that express this important cytokine known to be involved in antitumor responses. The method will report potency of total ITIL-306 product as shown in FIG. 51. Preliminary data from CoStAR specific potency from transduced cells is shown in FIG. 51. Due to challenges with the detection method in the coculture system, there are ongoing method development activities to improve accuracy of the reportable from CoStAR-transduced cells.

### Example 30

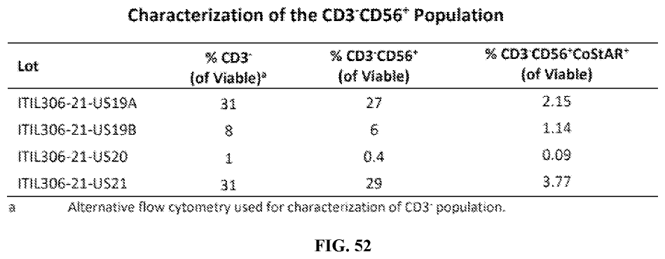

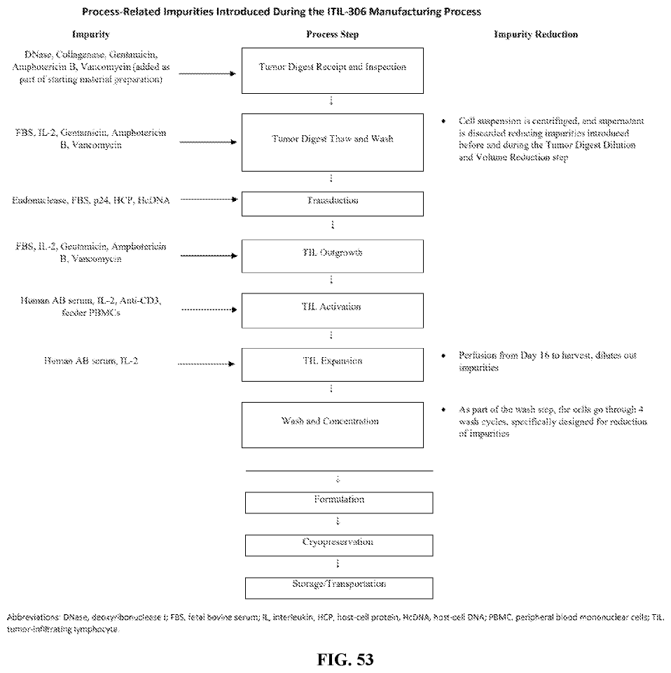

The starting material contains TILs and may have residual normal tissue cells such as macrophages, B cells and monocytes. Residual autologous lymphocytes pose a low safety risk to the patient. During process development with EOC tumor samples, a population of transduced and nontransduced CD3-CD56+ cells were observed in 2 of 4 final product lots. These cells have a surface phenotype consistent with that of NK cells as shown in FIG. 52. NK cells are innate immune cells with strong antitumor and antiviral responses (Herberman et al, 1975; Lim et al, 2015).

A flow cytometry assay will be performed to characterize the CD3-CD56+ population and evaluate the risk of these transduced cells in the ITIL-306 final product.

Like the T cells in ITIL-306, the NK population observed in the runs using ovarian tumors are derived from the patient and thus are already ‘self-tolerized’ (Yokoyama et al, 2010). Post infusion, NK cells are generally short-lived in vivo (7-10 days) in the absence of systemic IL-2 administration (Benyunes et al, 1995; Miller et al, 2005). Additionally, it has been reported that NK functionality is impacted after cryopreservation without overnight recovery with cytokines prior to infusion into the patient (Berg et al, 2009; Lapteva et al, 2014; Pittari et al, 2015). The levels of transduction observed in the NK cell population, as shown in FIG. 52, are consistent with literature describing low transduction efficiencies of lentiviral vector mediate gene modification of NK cells (Mehta and Rezvani, 2018; Pfefferle and Huntington, 2020). While either the transduced or nontransduced NK cells in ITIL-306 are not expected to be a safety risk nor impact the T cell function in ITIL-306.

### Example 31

Process-related impurities include reagents and suspensions used in the tumor material preparation and manufacturing process such as antimicrobial reagents (gentamicin, amphotericin B, and vancomycin), tumor-digest media components (DNAse and collagenase), TIL outgrowth media components (FBS and IL-2), LVV (endonuclease, FBS, p24, host-cell DNA, host-cell protein [HCP]), TIL activation (anti-CD3 and feeder PBMCs), and TIL expansion components (human AB serum and IL-2).

The ITIL-306 manufacturing process shown in FIG. 53 contains 2 impurity-reduction steps. First, the TIL expansion step includes perfusion from day 16 to harvest, which dilutes and removes impurities. Second, the ITIL-306 manufacturing process has a final wash step that consists of 4 wash cycles specifically designed for impurity reduction. During each automated wash cycle, the cell suspension is centrifuged, resulting in retention of T cells and removal of process-related impurities in the supernatant fraction.

### Example 32

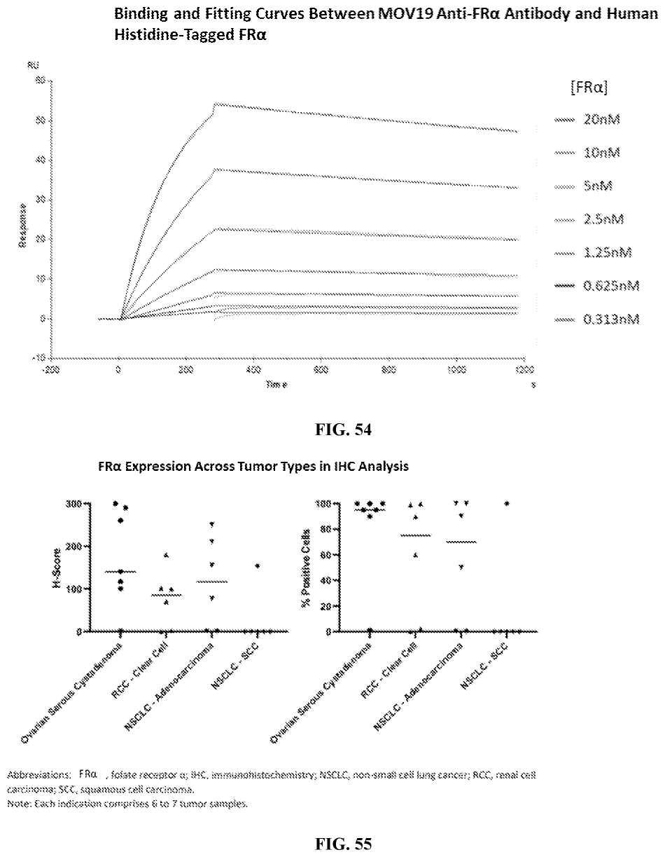

The scFv contained in the anti-FRα CoStAR protein was derived from the MOV19 monoclonal antibody originally isolated and purified from a mouse hybridoma derived from mice immunized against a protein extract from the ovarian cancer cell line OvCa4343/83 (Miotti et al, 1987). Surface plasma resonance studies demonstrated that the MOV19 antibody has high affinity against FRα with a dissociation constant (KD) around 0.46 nM (FIG. 54).

### Example 33

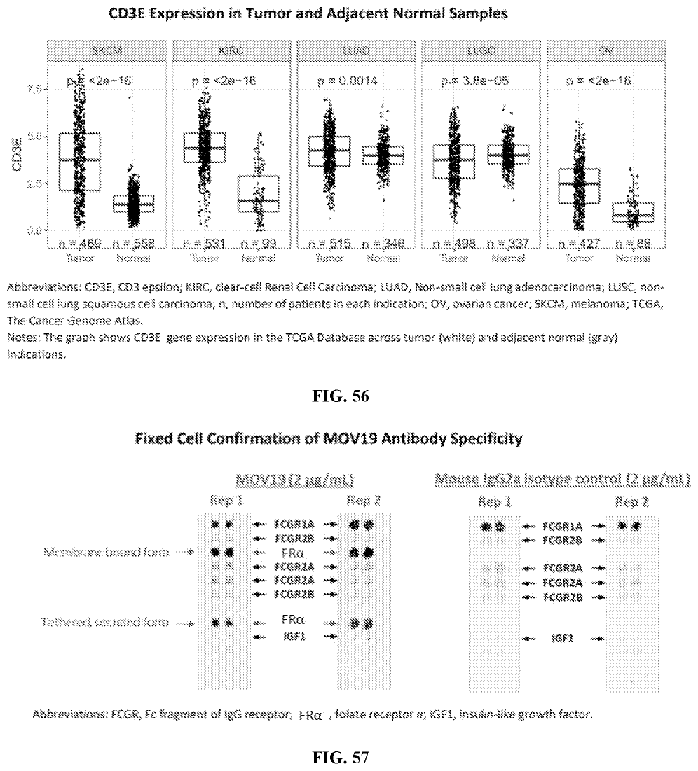

IHC staining analyses were performed in solid tumors across 9 different tumor indications (ovarian, NSCLC, triple-negative breast, pancreatic, RCC, and uterine carcinosarcomas) with 6 to 7 patient samples in each indication (FIG. 55). These analyses show that across the 7 ovarian cancer tissues, expression of FRα ranged from 1% to 100%, with an average of 83% and a median of 95% by pathologist tumor cell score. Positive FRα expression was observed in all ovarian cancer tissues (7/7; frequency of 100%). The pathologist tumor H-score ranged from 1 to 300, with an average of 173 and a median of 140. Across the 6 clear-cell RCC tissues, expression of FRα ranged from 0% to 100%, with an average of 59% and a median of 75% by pathologist tumor cell score. Positive FRα expression was observed in 5 clear-cell RCC tissues (5/6; frequency of 83%). The pathologist tumor H-score ranged from 0 to 180 with an average of 76 and a median of 85. Positive FRα expression was observed in 4 NSCLC, adenocarcinoma tissues (4/6; frequency of 67%). The pathologist tumor H-score ranged from 0 to 250, with an average of 115 and a median of 116 (FIG. 55). In addition, FRα expression shows a broad range of distribution across 32 distinct tumor types in The Cancer Genome Atlas (TCGA) (data on file). These data also validate the highest expressing tumors being ovarian, non-small cell lung and renal cancers in much larger sample size cohorts. In addition, these tumor types also showed similar expression levels of CD3 epsilon (CD3E), a surrogate marker for T-cell infiltration (FIG. 56). Together these data suggest that these indications would potentially benefit from anti-FRα CoStAR TIL therapy.

### Example 34

In vitro studies were conducted to characterize the specificity profile of the MOV19 parental antibody from which the binder in anti-FRα CoStAR protein was derived. A screen was performed to evaluate its binding capacity against a library of 5475 full-length human plasma membrane proteins and cell surface-tethered human secreted proteins plus 371 human heterodimers, representing over 90% of all membrane bound and secreted proteins. This assay was conducted by Retrogenix, Ltd and the results demonstrated that MOV19 antibody is highly selective against its target FRα. The study was divided into 3 phases. First, a prescreen was undertaken to determine the level of background binding of the test antibody to nontransfected and FRα overexpressing HEK293 cells. These data were used to assess the suitability and optimal concentrations for onward screening. Second, in the library screen, the test antibody was screened for binding against fixed HEK293 cells overexpressing the protein library to identify hits. Finally, in the confirmation/specificity screens, all library hits were re-expressed, and probed with the test antibody or control treatments, to determine which hit(s), if any, were repeatable and specific to the test antibody. This was performed both on fixed and live cells (FIG. 57).

These assays confirmed that the MOV19 antibody specifically interacted only with plasma membrane and tethered secreted forms of FRα, the primary target, with medium/strong to strong intensity on both fixed- and live-cell microarrays. No further specific interactions were identified. These data indicate a high degree of specificity of MOV19 for its primary target FRα.

### Example 35

Anti-FRα CoStAR expression levels were measured via flow cytometry utilizing soluble FRα fused to Fc tag (sFRα-Fc) followed by a secondary antibody staining. Vector copy number was measured via ddPCR using primers specific against the anti-FRα CoStAR transgene.

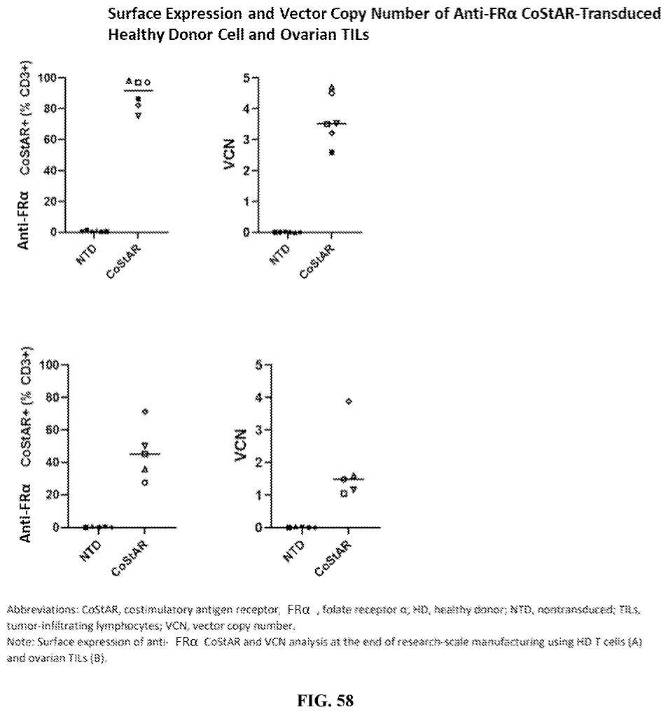

Transduction ranged from 75.5% to 86.5% with a VCN of 2.5 to 3.5 for the unsorted batch of healthy donor T cells and 97% to 98.3% with a VCN of 3.5 to 4.5 for the sorted batch at day 23 (FIG. 58). For the ovarian TILs, transduction efficiency in CD3+ T cells ranged from 27.65% to 71.28% with a VCN between 1 and 3.8 (FIG. 58).

These data demonstrate that both healthy donor T cells and ovarian TILs can be efficiently transduced and the anti-FRα CoStAR molecule can be detected by flow cytometry. In all samples tested the VCN was under 5.

### Example 36

FRα is often released from the cells via membrane-associated protease or glycosylphosphatidylinositol (GPI)-specific phospholipase in soluble form and has been proposed as a biomarker in serum for early detection and monitoring of ovarian cancer (Farran et al, 2019). Soluble FRα in serum is significantly higher in malignant (median 2059 pg/ml, range 1487-2812 pg/ml) compared to early stage (median 807.0 pg/ml; 95% CI: 720.0-980.0 pg/ml) ovarian cancer patients (Kurosaki et al, 2016). Recombinant sFRα binds anti-FRα CoStAR expressed on the T cell surface, as demonstrated in the present studies. Since sFRα is commonly found in circulation of ovarian cancer patients at significant levels, there is a potential that interactions between sFRα and anti-FRα CoStAR molecule will result in either activation of the costimulatory signal off-tumor or, conversely, may result in the inhibition of the costimulatory signal within the tumor microenvironment. Experiments were performed to elucidate whether the binding of sFRα to CoStAR interferes with its intended activity by either inducing cell contact-independent costimulation or blocking normal CoStAR-triggered costimulation.

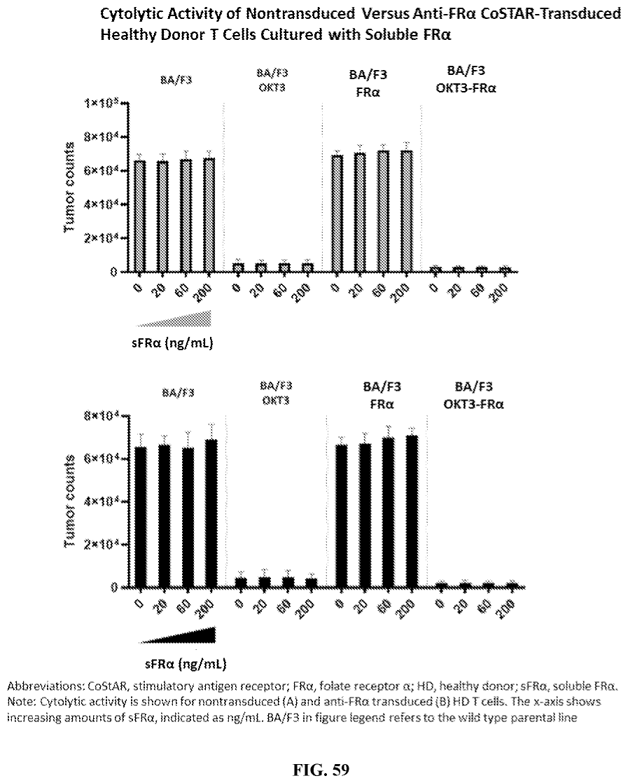

Following a similar experimental paradigm as previously described, nontransduced and anti-FRα CoStAR-transduced T cells were cultured with different target cell lines expressing OKT3, FRα, neither or both (FIG. 59). T cells in both groups were pre-incubated with increasing concentrations of sFRα and binding was confirmed via flow cytometry (data on file). Readouts included measurements of cytolytic activity and cytokine secretion.

Analysis of the cytolytic activity demonstrated the expected increase in the presence of both OKT3 (signal 1 alone) and OKT3+FRα (signal 1+2) target cell lines for both nontransduced and anti-FRα CoStAR-transduced T cells compared to the control cell line not containing OKT3 (signal 1). No statistically significant increase or decrease in the cytolytic activity was observed in either group in the presence of increasing concentrations of sFRα as expected.

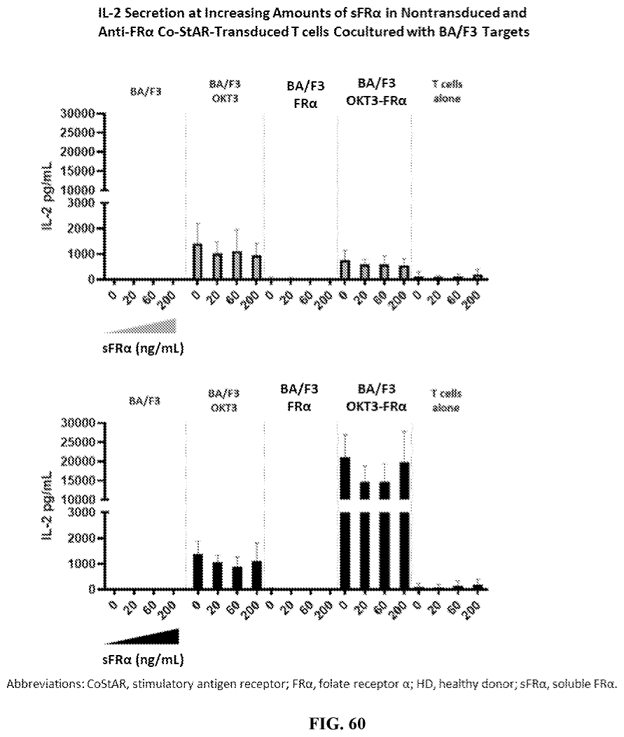

Cytokine secretion confirmed an increase in the levels of IL-2 secretion in the anti-FRα CoStAR T cells when cocultured with target cell lines expressing both OKT3+FRα (signal 1+2) compared to OKT3 (signal 1) alone or the nontransduced reference control (FIG. 60). Importantly, no statistically significant increase in the levels of IL-2 were observed in anti-FRα CoStAR T cells when exposed to OKT3 target cell line (signal 1 alone) at increasing amounts of sFRα, indicating that CoStAR does not induce costimulation when triggered by FRα in its soluble form. Similarly, no statistically significant decrease in the levels of IL-2 were observed in anti-FRα CoStAR T cells cocultured with the OKT3+FRα target cell lines (signal 1+2), suggesting sFRα cannot inhibit the costimulatory signal. Together these data suggest sFRα does not modulate or interfere with the activity of anti-FRα CoStAR-transduced T cells.

### Example 37

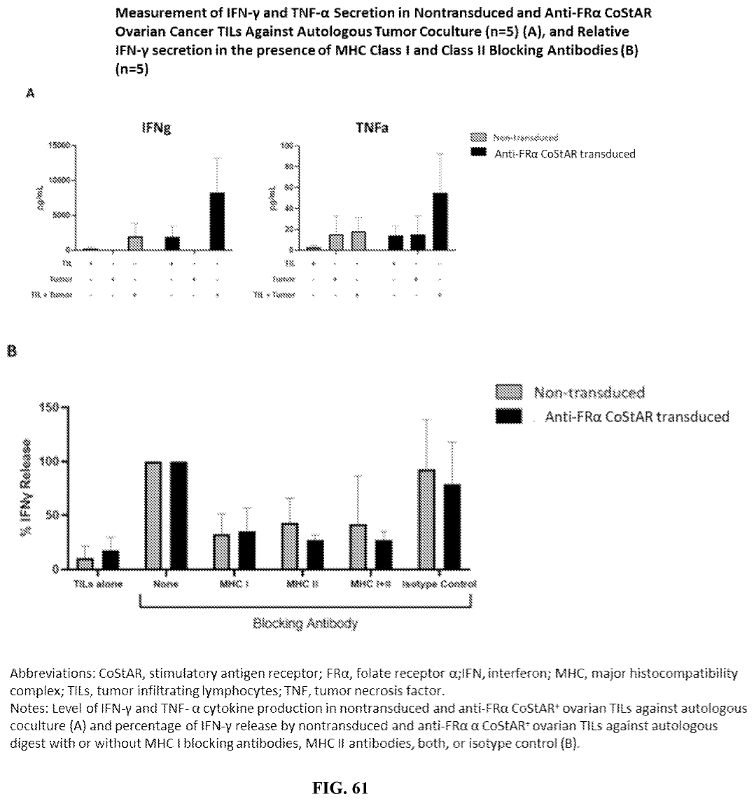

As previously demonstrated, the anti-FRα CoStAR molecule is designed to exclusively provide costimulation and is not expected to induce cytolytic activity in the absence of TCR activation ie, signal 1. The T cells still express the thymically selected, self-tolerant TCRs which continue to be the gatekeepers for activation through normal pMHC engagement. To confirm this, experiments were performed by setting up cocultures of anti-FRα CoStAR+ ovarian TILs against autologous tumor in the presence of blocking antibodies against MEW class I and class II. As predicted, the levels of cytokine were reduced in both nontransduced and anti-FRα CoStAR+ ovarian TILs when the TCR-pMHC recognition was blocked via MEW blocking antibodies (FIG. 61). These data indicate that tumor recognition is exclusively gated on TCR-pMHC recognition and not the anti-FRα CoStAR molecule, which only provides costimulatory signal in the presence of FRα.

### Example 38

The TCR repertoire in TILs is polyclonal by nature, containing a diverse TCR population with varying degrees of reactivity against tumor antigens. It is important to note that anti-FRα CoStAR molecule is designed to exclusively provide a costimulatory signal to the transduced T cells. Combined with the polyclonal reactivity of the TILs, this results in only a fraction of the T cells that could potentially benefit from anti-FRα CoStAR. More specifically, only tumor-reactive T cells that are transduced and actively engaging in TCR-pMHC and FRα/anti-FRα CoStAR interactions are expected to experience an improvement in T cell effector function. In the absence of TCR stimulation, either due to lack of TCR reactivity or absence of active engagement of the TCR-pMHC complex, the anti-FRα CoStAR molecule does not activate T cells, regardless of the presence of its ligand FRα.

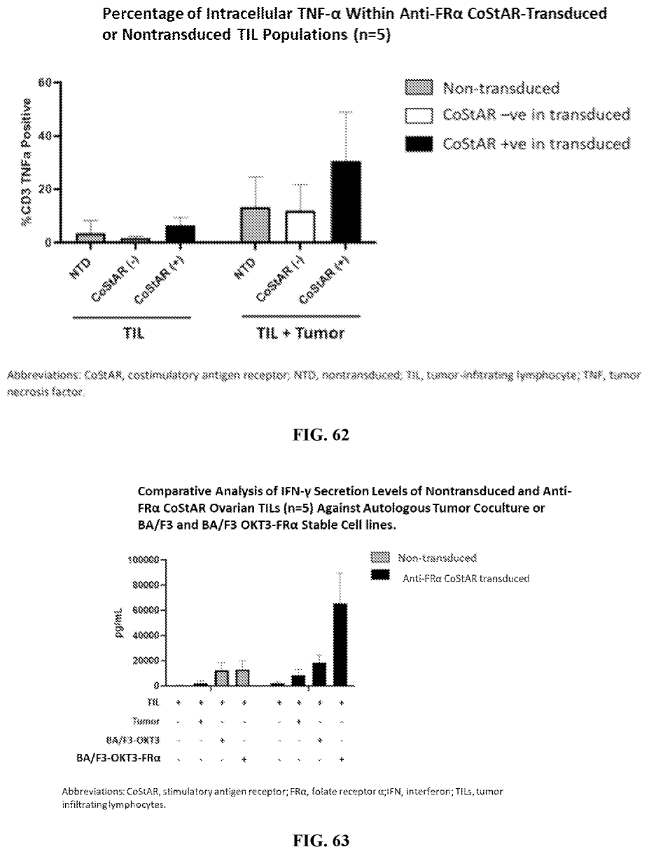

To understand the relative contribution of the anti-FRα CoStAR+ and CoStAR− populations within the anti-FRα CoStAR ovarian TILs, intracellular staining of TNFα was performed in a coculture setting between TILs and autologous tumor to measure the amount of tumor-reactive T cells within the TIL population. This analysis showed no statistically significant difference in the proportions of cytokine positive CD3 TILs when comparing a nontransduced ovarian TIL (NTD) and a nontransduced fraction (CoStAR−) of the anti-FRα CoStAR ovarian TIL product. In contrast, the same analysis performed in the anti-FRα CoStAR+ transduced fraction showed a significant increase in the relative percentage of cytokine positive TILs (FIG. 62), suggesting that the overall increase in the cytokine levels previously described in the supernatant media are driven mostly by CoStAR+ TILs. This supports the rationale of dosing solely based on total amount of anti-FRα CoStAR+ T cells.

Tumor reactive (ie, TNF-α positive) TIL ultimately drive activity of ITIL-306, since these cells can generate signal 1. That said, not all CoStAR+ TIL are tumor reactive; in fact, only about 20% to 30% of CoStAR+ TIL appear to be tumor reactive as measured by intracellular TNF-α (FIG. 62), while published reports indicated a median frequency of 3.2% anti-tumor reactivity in ovarian cancer (Westergaard et al, 2019). Therefore, at the starting clinical dose of 1×109 CoStAR+ viable T cells, it is expected that only approximately 2×108 to 3×108 transduced T cells are tumor reactive. This dose range has been found to be safe with approved CD19 and BCMA targeting CAR T therapies, and well below the number of tumor-reactive cells administered in transgenic TCR-T trials (Robbins et al, 2011; Nagarsheth et al, 2021). As opposed to hematologic CAR T-cell products, ITIL-306 CoStAR+ cells will only be fully activated once encountering and recognizing specific pMHC, a highly stochastic process, and FRα within the tumor microenvironment. Hence the compared CAR T cells carry a higher toxicity risk per cell, since they target densely expressed surface antigens found in the blood and simultaneously activate signal 1 and 2. Furthermore, based on the demonstrated mechanism of action of ITIL-306, no off-tumor toxicities against FRα positive cells are anticipated, regardless of the ability of the transduced TILs to recognize and eliminate tumor cells.

In addition, preliminary data generated using peptide pulse experiments suggests that total cytokine levels produced by anti-FRα CoStAR T cells are a function of the amount of peptide being presented as well as FRα expression levels in the target cell line (data on file). Studies comparing the levels of cytokine production in anti-FRα CoStAR TILs using autologous tumor (ie, containing physiologically relevant amounts of peptide and FRα) and stable cell lines (engineered to provide maximal stimulation from the TCR and FRα), demonstrate that TILs engineered with anti-FRα CoStAR produce approximately 8-fold less cytokine against autologous tumor than against the stable cell line (FIG. 63).

### Example 39

Methods

Tumour and normal tissue was analyzed for FRα expression, and K562 cells with or without OKT3 (Signal 1) generated with physiological levels of FRα. Healthy donor T cells were engineered with CoStAR and cocultured with the target lines before assessment of IL2, TNFα and IFNγ in the supernatant and counts of remaining target cells. A serial stimulation assay was established using Ba/F3 cells engineered with either OKT3 (Signal 1) and/or FRα (Signal 2), to recapitulate scenarios in which CoStAR cells may encounter tumour and normal tissue in sequence. Healthy donor T cells engineered with CoStAR were cocultured with the indicated Ba/F3 cells presenting either signal 1, alone, signal 2 alone, both or neither. After 7 days the T cells were restimulated with additional Ba/F3 cells before analysis of cytokines. Healthy donor T cells were singly or co-transduced with an HLA-A*02 Melan-A/MART-1 specific TCR and FRα specific CoStAR. HLA-A*02+T2 were transduced with FRα or left non-transduced. T2-FRα were then pulsed with Melan-A/MART-1 heteroclitic (ELAGIGILTV 17 μM) or altered peptide ligands of varying antigenicity FATGIGIITV (3 μM), ELTGIGILTV (82 μM) and ALGIGILTV (very low affinity) (10,11) and cytokine secretion measured after 20 h coculture. Tumour and normal tissue was analyzed for FRα expression, and K562 cells with or without OKT3 (Signal 1).

### Results

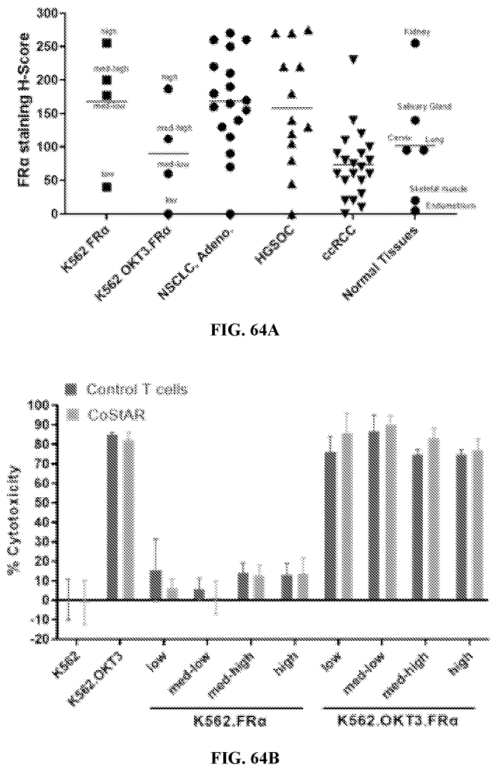

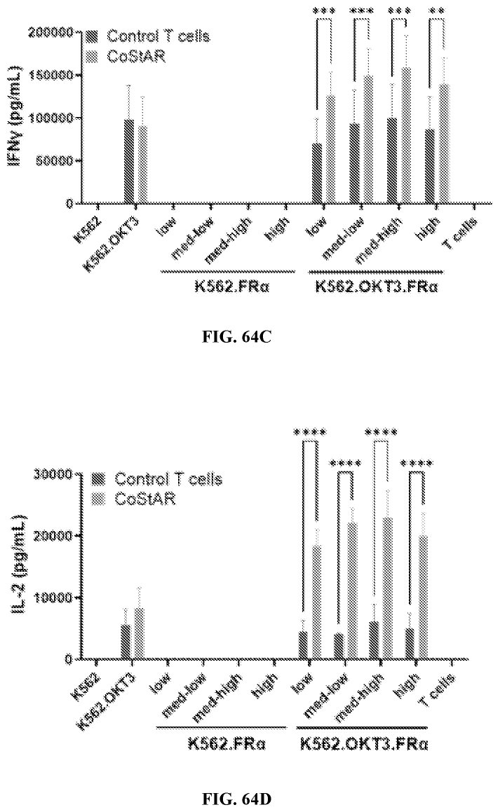

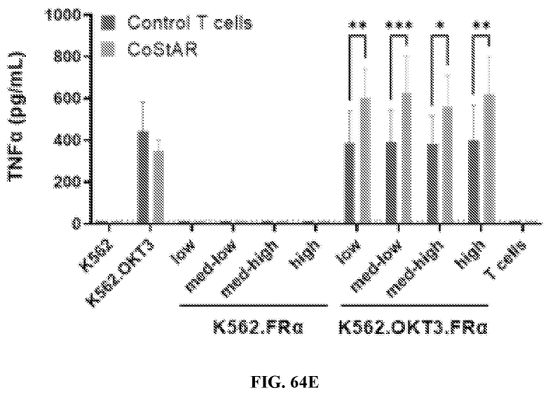

Lysis of K562 target lines (FIG. 64A-64B) and cytokine release was not observed (FIG. 64C-64E) in the absence of signal 1, regardless of the level of FRα present, indicating that FRα alone is insufficient to elicit effector function. In conditions with OKT3 expressing lines CoStAR demonstrated FRα dependent enhancement in activity, with all levels of FRα significantly enhancing IL-2, IFNg and TNFa release (FIG. 64C-64E) Enhancements in cytokine production at low FRα levels was observed, an expression level equivalent to that observed at the lower end of the range seen in lung, ovarian and renal cancer.

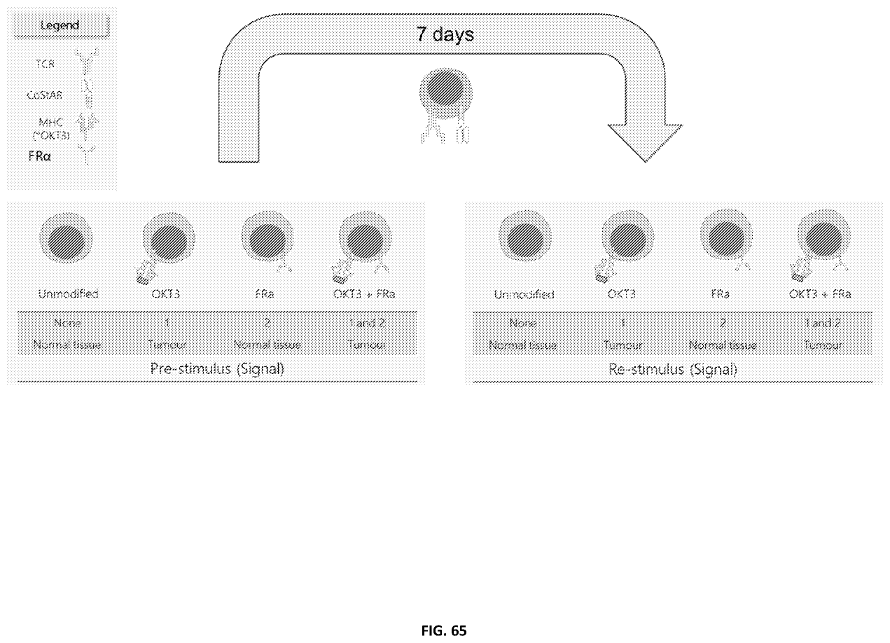

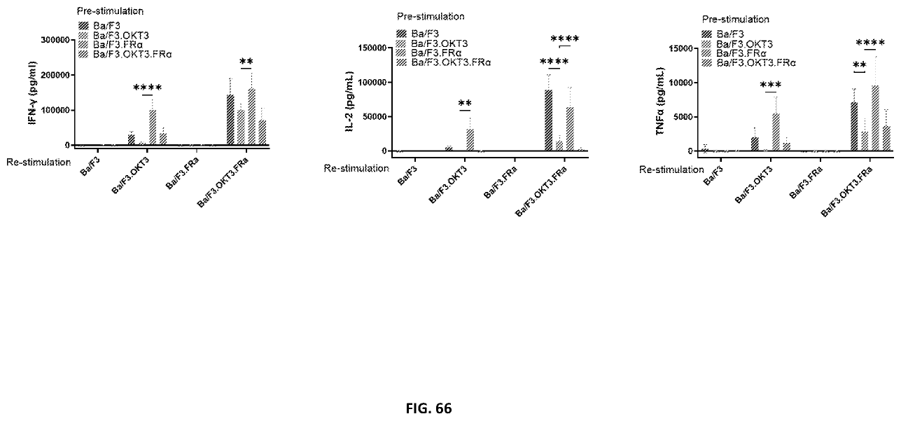

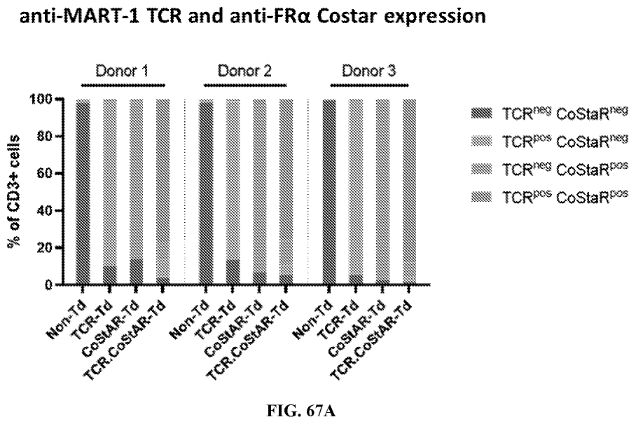

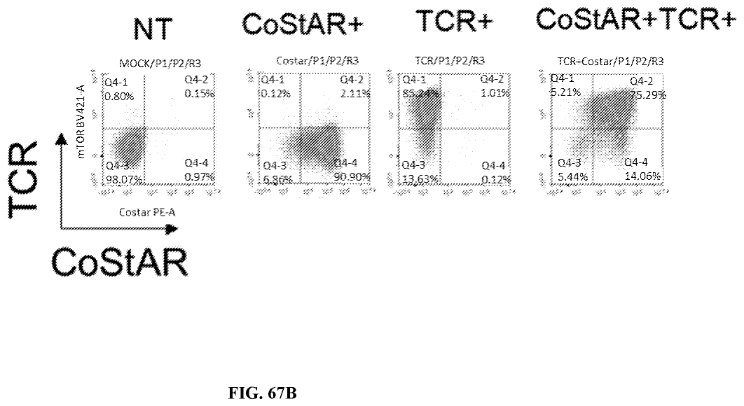

Restimulated T cells showed no response to non-transduced Ba/F3 cells or Ba/F3 cells expressing FRα alone, regardless of the primary stimulation (FIG. 65-66). T cells pre-stimulated with Ba/F3 expressing OKT3 with or without FRα responded less potently to Ba/F3 expressing OKT3 and Fra. T-cells pre-stimulated with FRα alone and then restimulated with OKT3 produced significantly more IL-2, IFNγ and TNFα than T-cells pre-stimulated on target cells without FRα. These data indicate that CoStAR pre-stimulation with FRα primes T cells to enhanced responsiveness to subsequent stimulation in the absence of CoStAR engagement.

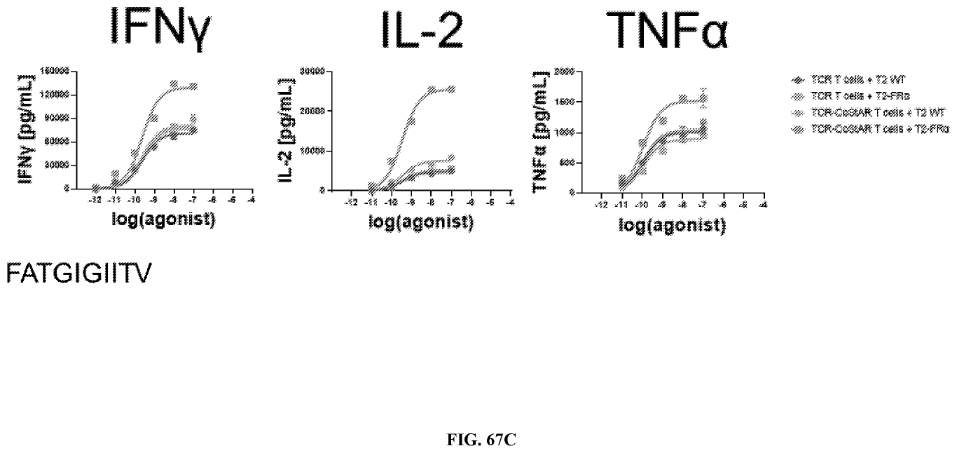

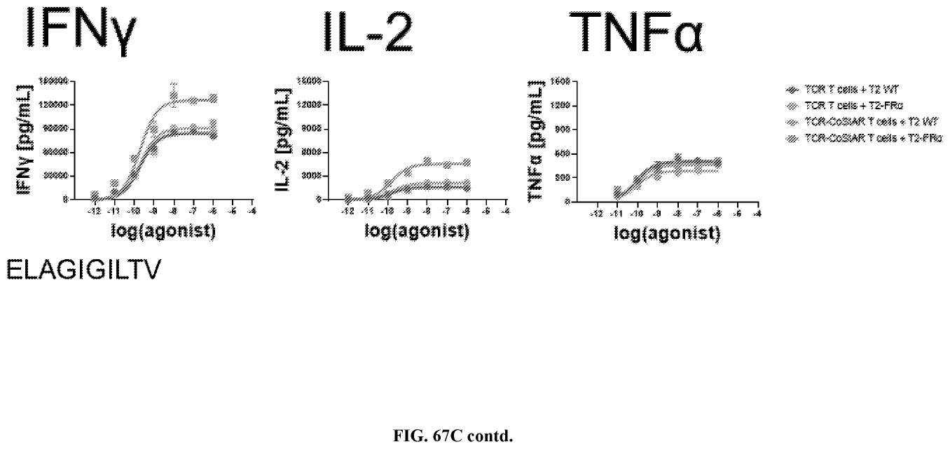

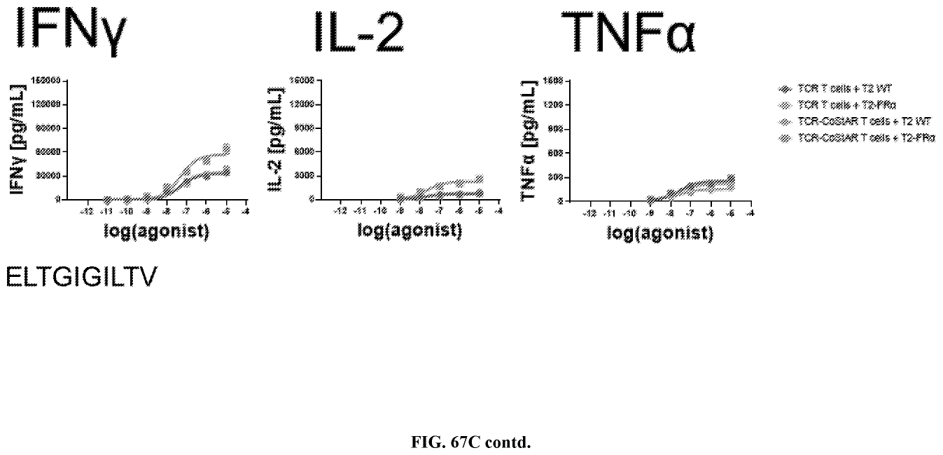

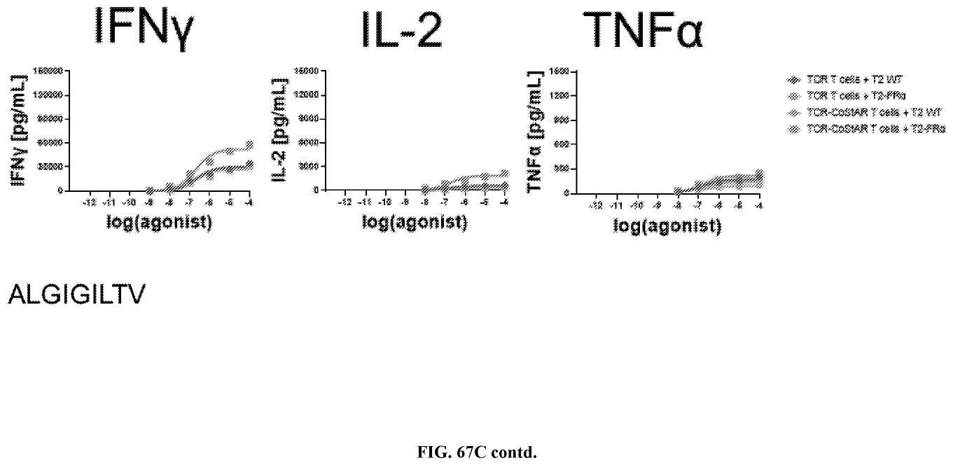

T cells from three healthy donors were engineered with a Melan-A/MART-1 specific TCR and CoStAR. Over 85% of CD3+ cells expressed anti-MART1 TCR in TCR-Td condition, over 84% of CD3+ cells expressed anti-FRα CoStAR molecule in the CoStAR-Td condition, and over 75% of CD3+ cells expressed both anti-MART1 TCR and anti-FRα CoStAR molecule in the TCR.CoStAR-Td condition (FIG. 67A-67B). TCR and TCR.CoStAR T-cells responded in a dose dependent manner to peptide loaded T2 or T2.FRα cells via production of IFNγ, IL-2 and TNFα. Differences were seen with the peptides tested, with the strongest HLA binder, FAT, eliciting a greater degree of activity followed in the order: ELA, ELT and ALG eliciting the lowest level of cytokine secretion. TCR engineered cells responded to peptide loaded T2 or T2.Fra cells and TCR.CoStAR cells responded to peptide loaded T2 cells with similar levels of activity. TCR.CoStAR cells responded more potently to peptide loaded T2 cells against all peptides tested (FIG. 67C).

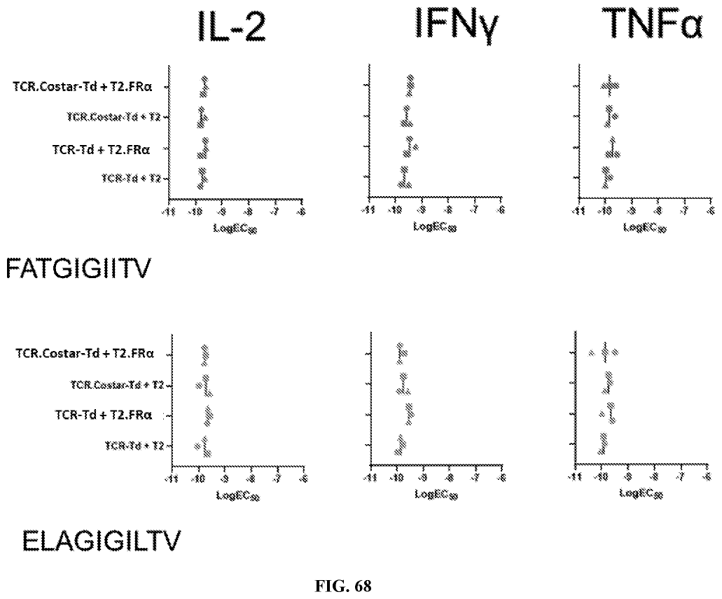

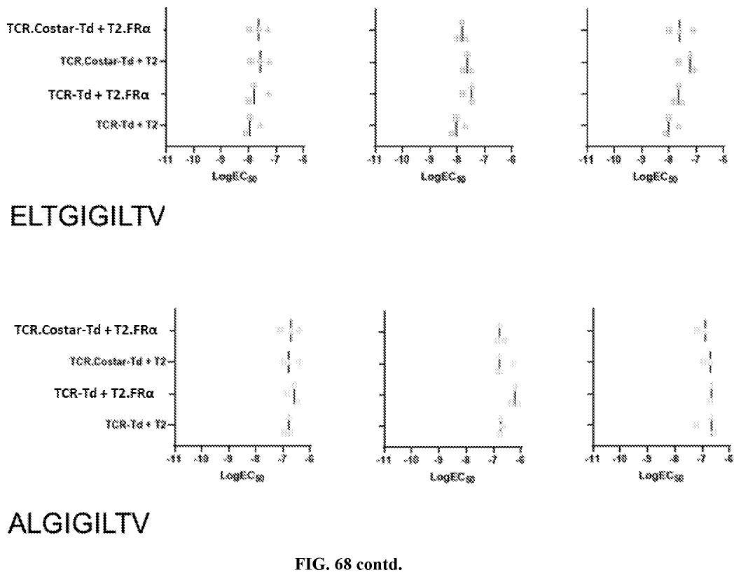

CoStAR did not impact the affinity of peptide antigen recognition (FIG. 68). EC50 values were calculated for each scenario (+/−CoStAR and +/−FRα) and plotted for each peptide and each effector function.

### Conclusions

Anti-FRα CoStAR enhances T-cell function in response to target antigen regardless of the degree of FRα expression. CoStAR does not respond to FRα in the absence of TCR stimulation (cytokine production or cytotoxicity), even at physiologically high levels of FRα. Stimulation of CoStAR with FRα alone primes subsequent responses to TCR agonism, suggesting that priming of CoStAR with FRα may enhance subsequent activity towards any tumour targets lacking FRα expression. CoStAR enhances T cell activity for pMHC ligands across a range of avidity, but does not change the EC50. This suggests that TCR avidity or promiscuity will not change with CoStAR engagement and enhanced T cell activation. These results support the clinical exploration of anti-FRα CoStAR in tumor indications expressing variable FRα levels.

### Example 40

Next, ITIL-306 was evaluated for demonstration of enhanced activity towards autologous FRα expressing tumor types. ITIL-306 expressing NSCLC, RCC, and renal TILs were evaluated for anti-tumor activity. Matching autologous tumor from NSCLC, RCC, and ovarian patients were used as target cells. It was observed that CoStAR-TILs demonstrate increased anti-tumor reactivity against matching autologous tumor from NSCLC, RCC, and ovarian patients in comparison to non-transduced TILs, as evidenced by increased secretion of IFNγ.

### Example 41

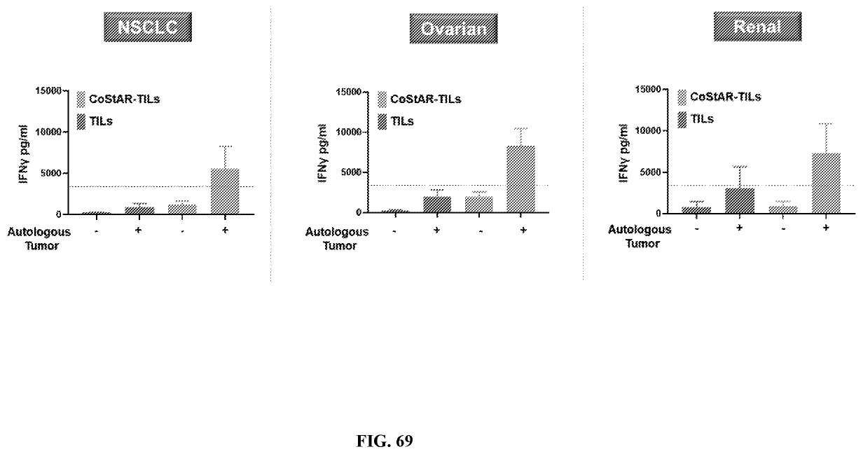

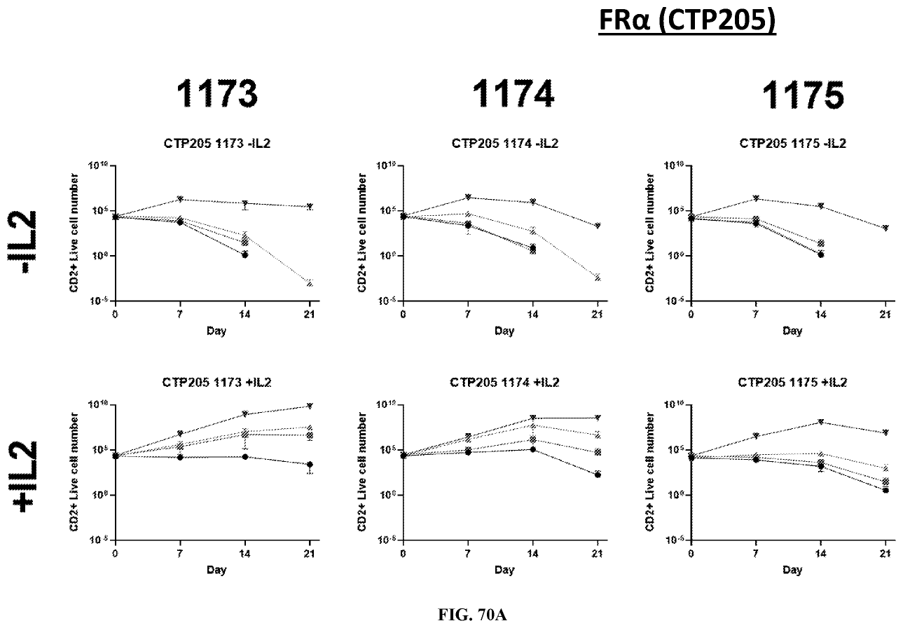

CoStAR enhancement of antitumor activity was evaluated by transducing TILs with ITIL-306 and incubating the CoStAR-TILs with matching autologous tumors from NSCLC, RCC, and ovarian patients. Anti-tumor activity was evaluated by assessing IFNγ secretion. CoStAR-TILs demonstrated enhanced anti-tumor reactivity over TILs (FIG. 69). Notably, enhanced anti-tumor activity was consistent against tumor cells with varying levels of FRα.

### Example 42

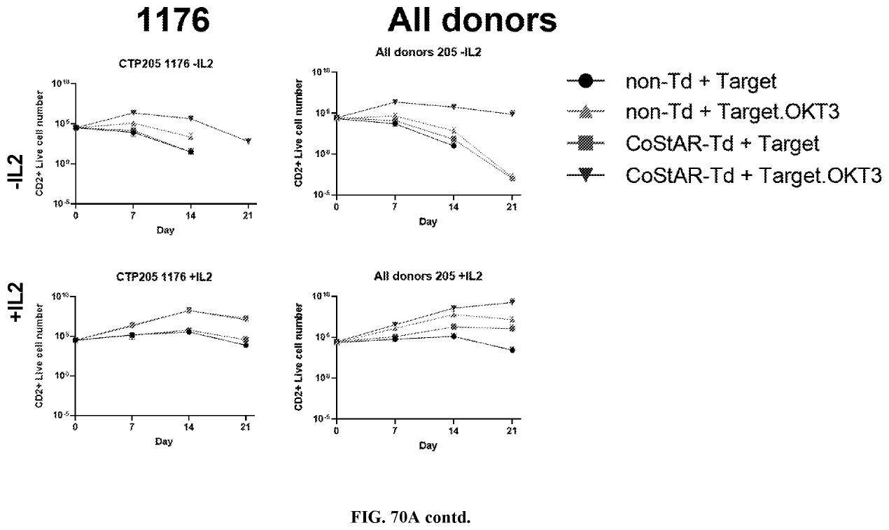

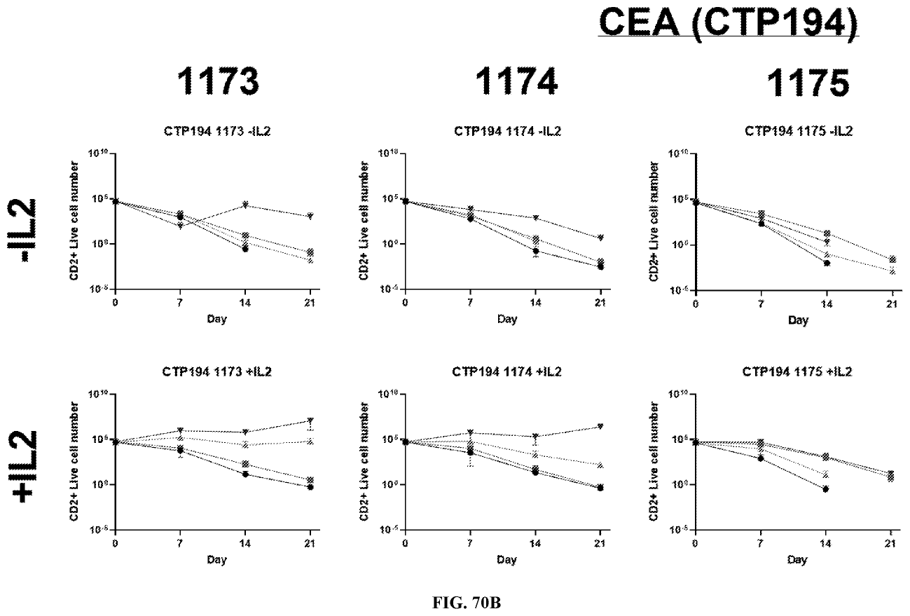

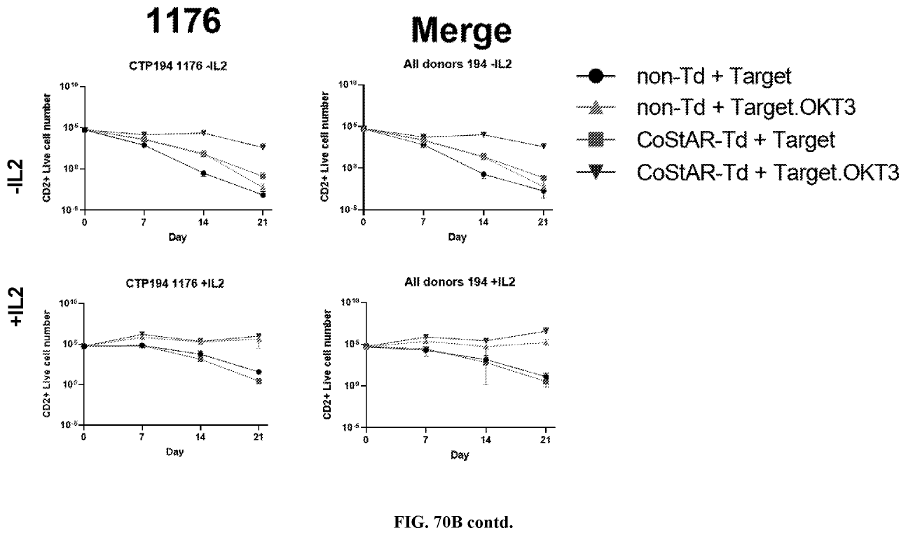

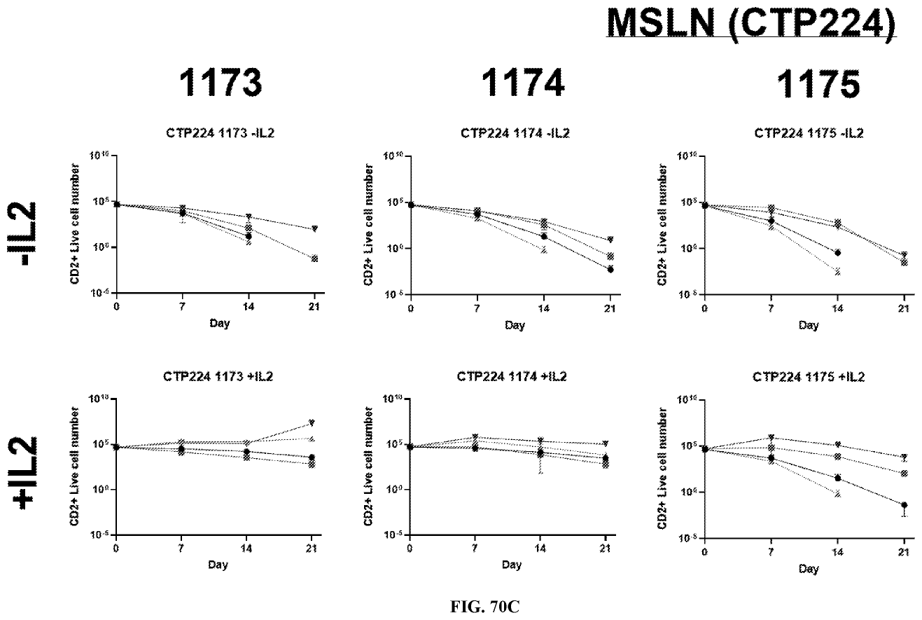

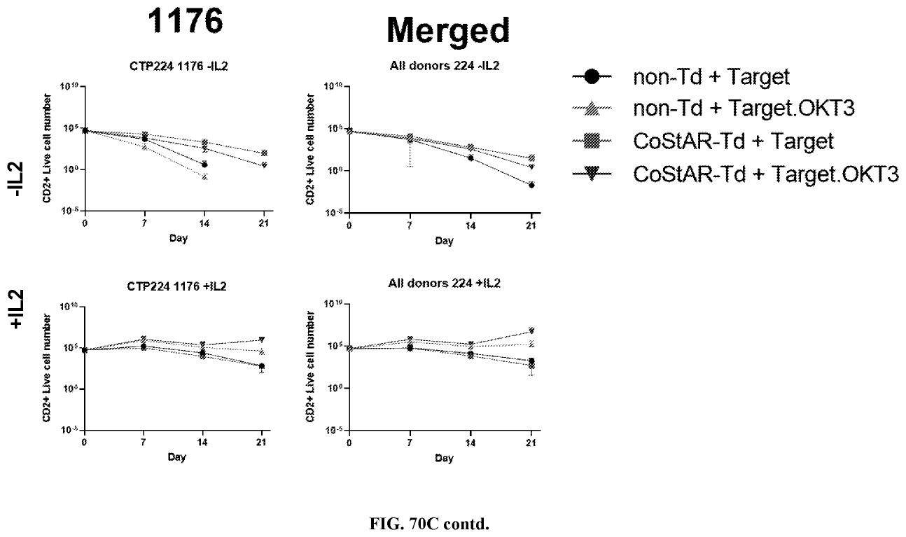

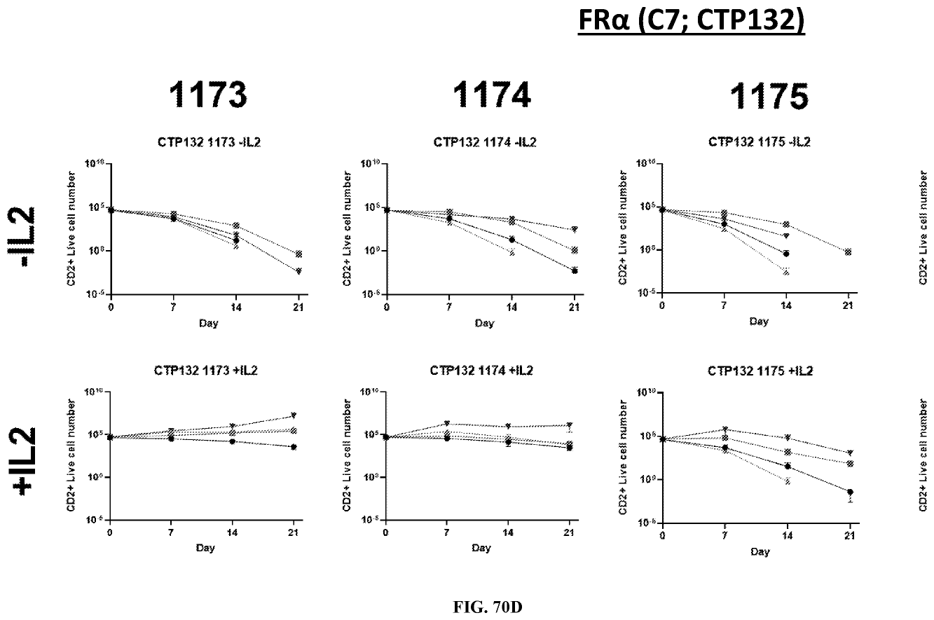

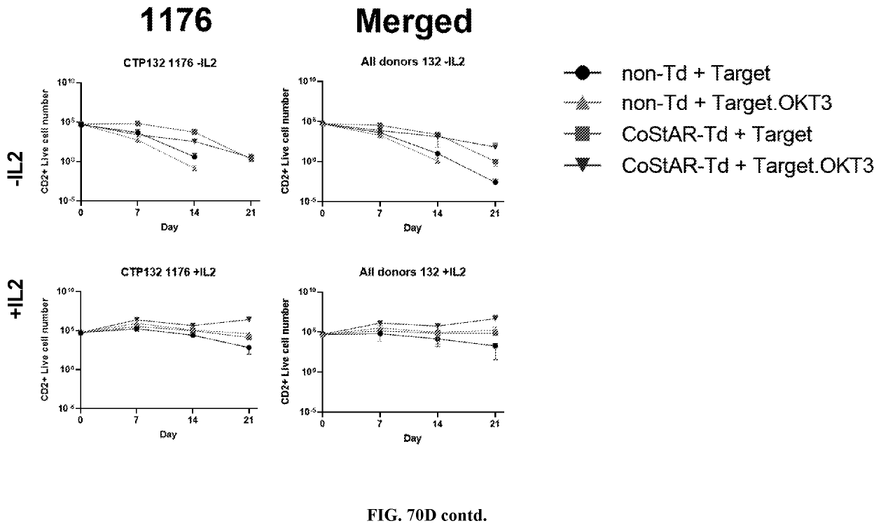

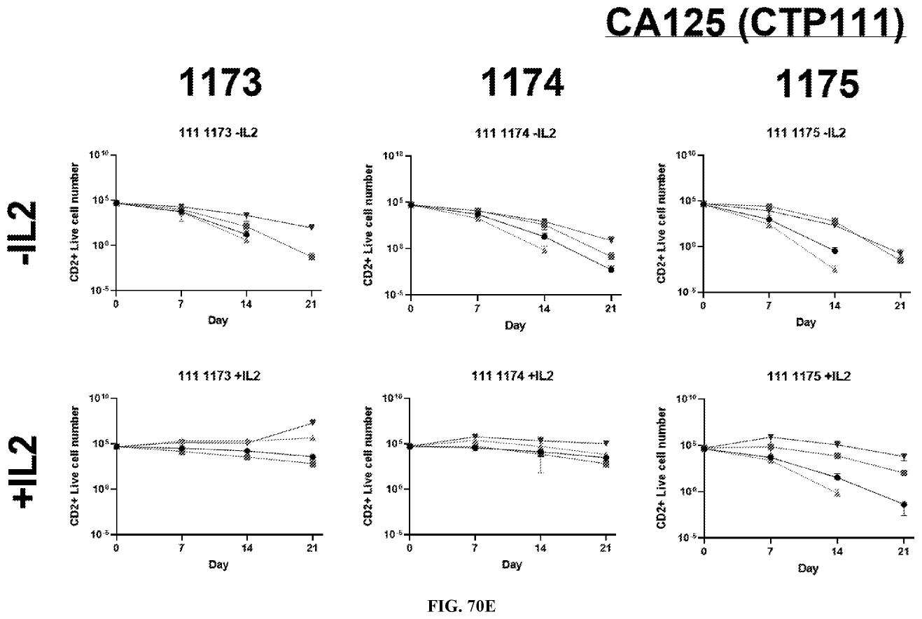

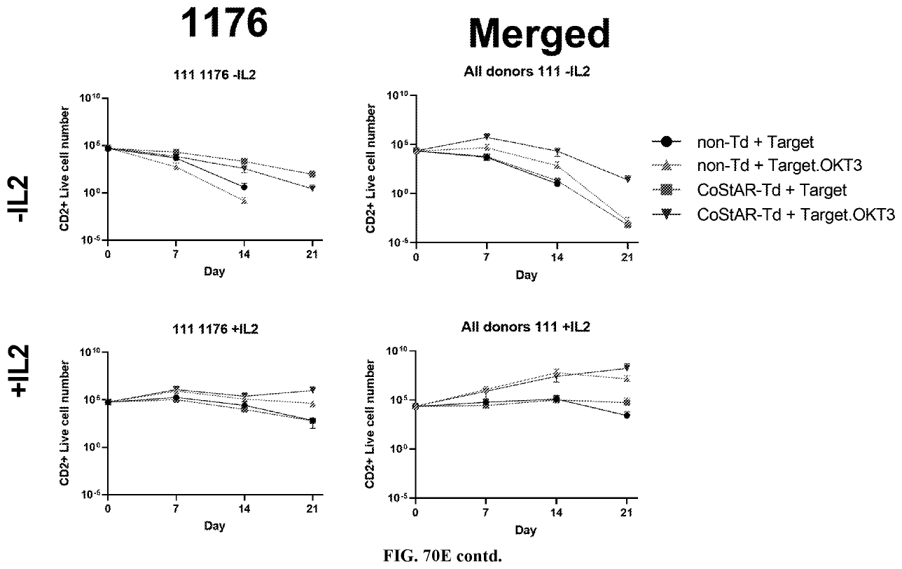

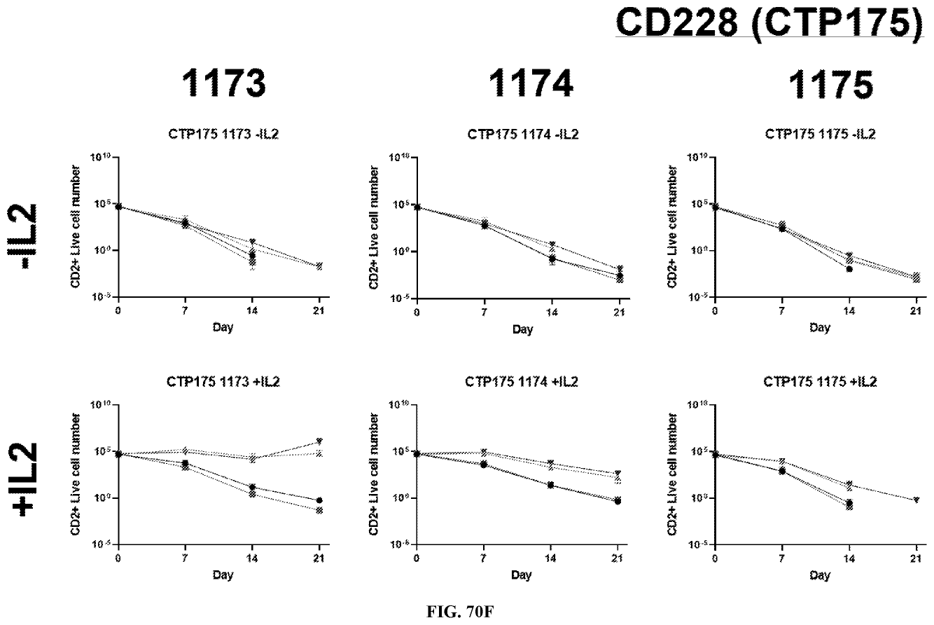

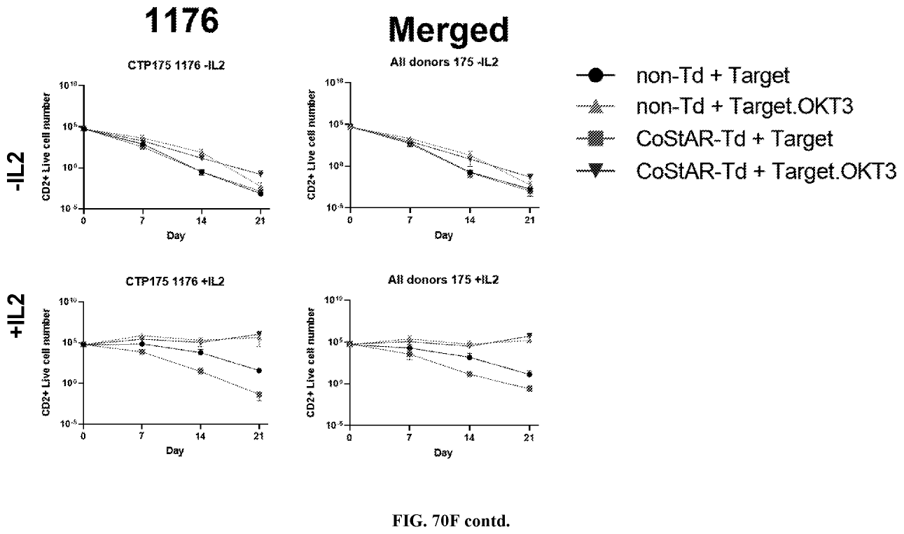

Next, an experiment was conducted to assess the effect of six CoStAR constructs on proliferation of transduced healthy donor T cells. CoStAR transduced cells expressed CoStAR targeted to FRα, CEA, MSLN, CA125, and CD228, an additional CoStAR featured the high affinity FRα binding peptide FRα C7. Healthy donor (HD) T-cells from four different donors were modified with the CoStAR constructs and cocultured with target cells+/−OKT3, and an E:T ratio of less than 8 was maintained. Cells were cultured+/−IL-2 for a period of 21 days and proliferation was assessed by measuring CD2 live cell counts at days 0, 7, 14, and 21 compared to nontransduced controls. FIG. 70A depicts the results for FRα CoStAR (CTP205). FIG. 70B depicts the results for CEA CoStAR (CTP194). FIG. 70C depicts the results for MSLN CoStAR (CTP224). FIG. 70D depicts the results for FRα CoStAR (C7, CTP132). FIG. 70E depicts the results for CA125 CoStAR (CTP111). FIG. 70F depicts the results for CD228 CoStAR (CTP175).

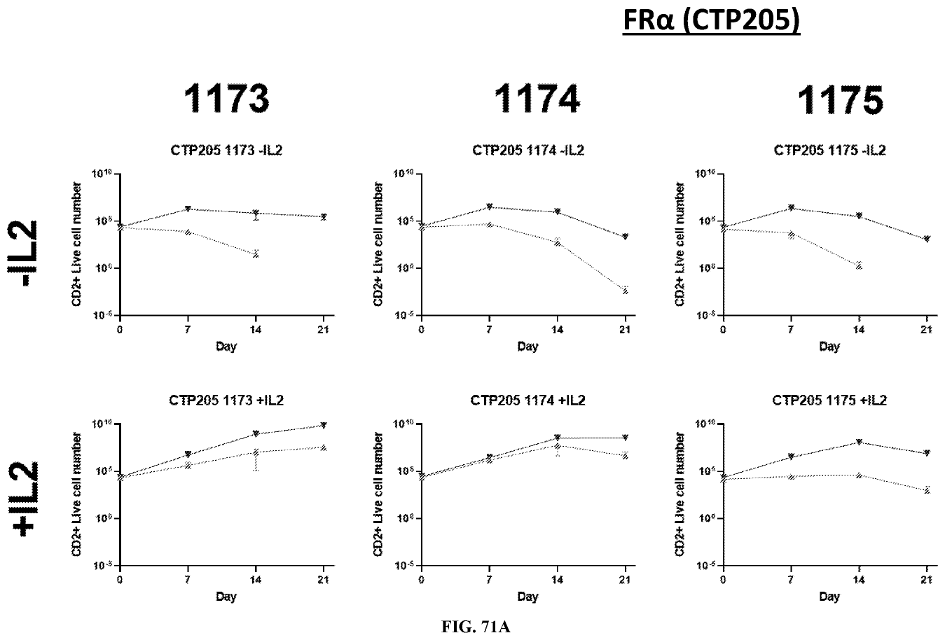

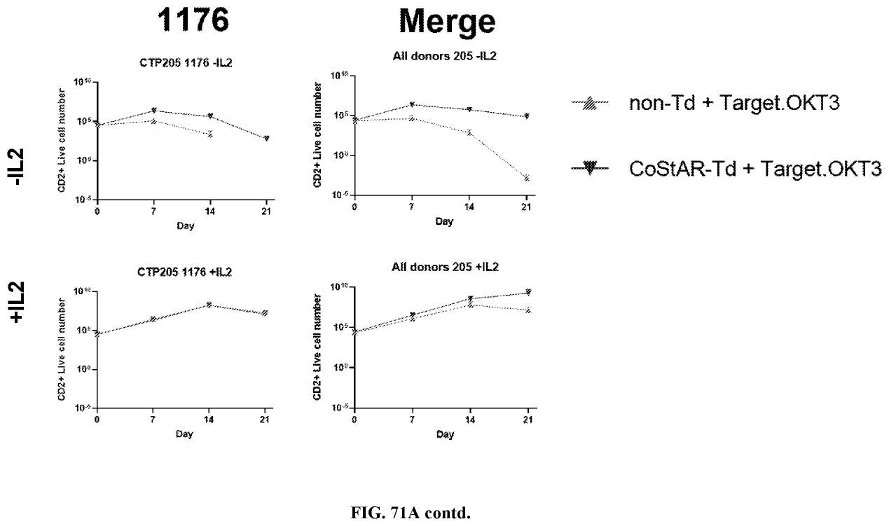

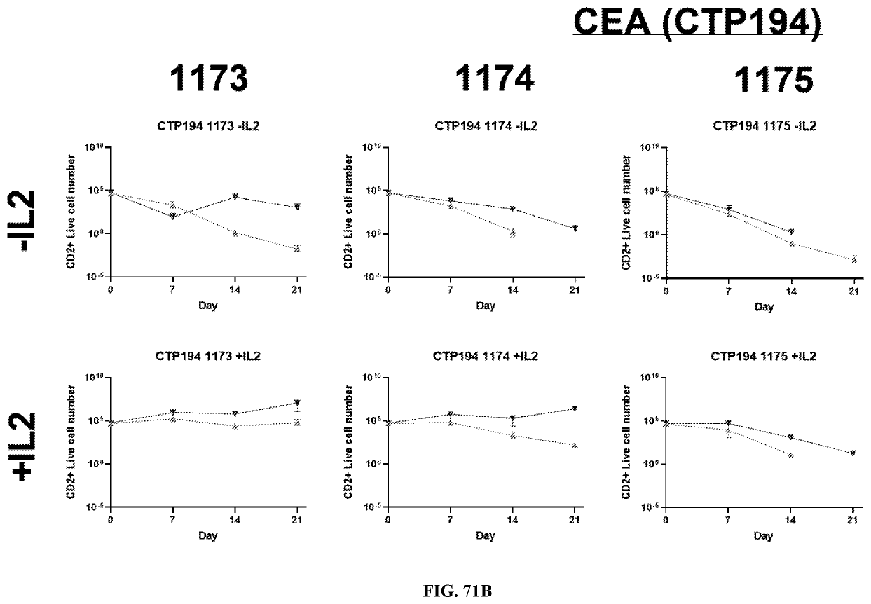

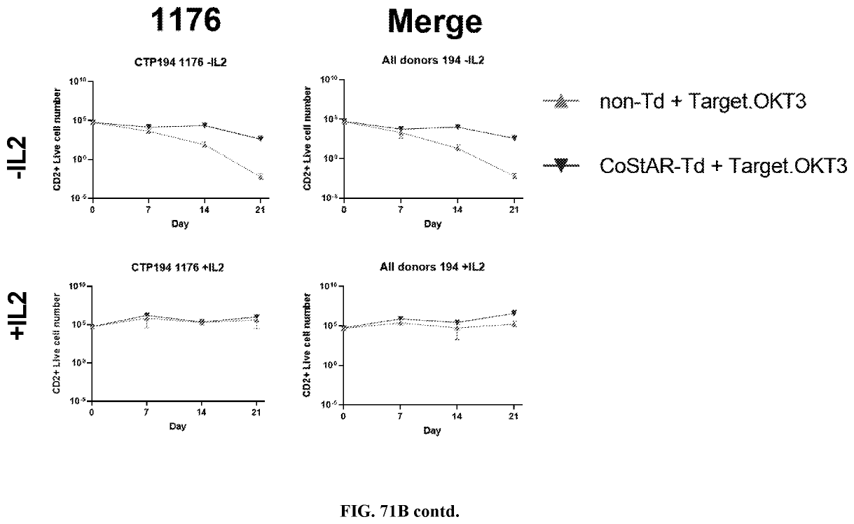

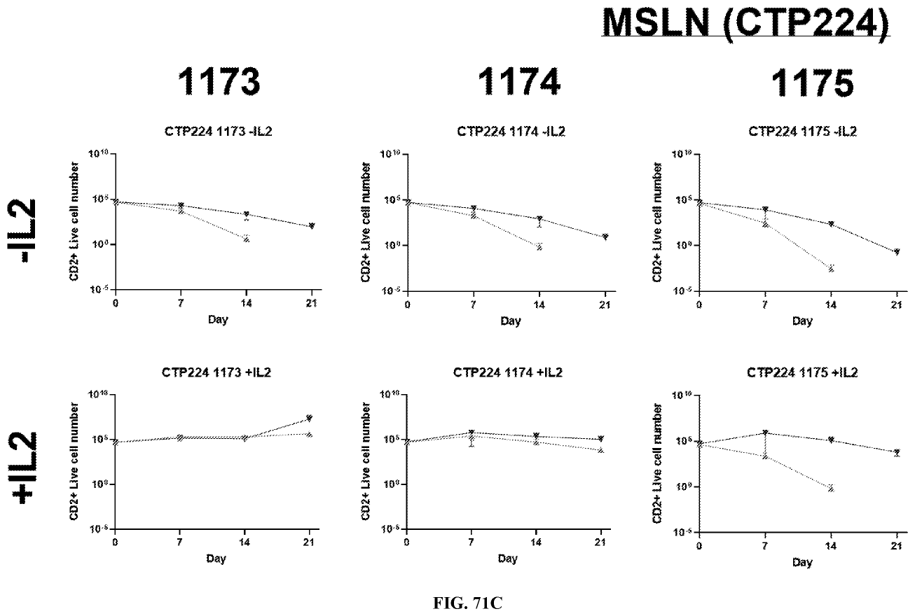

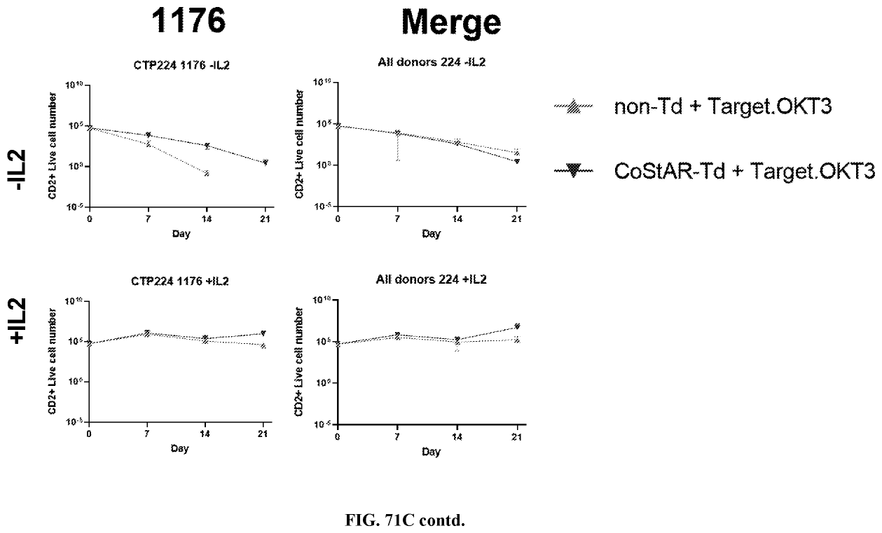

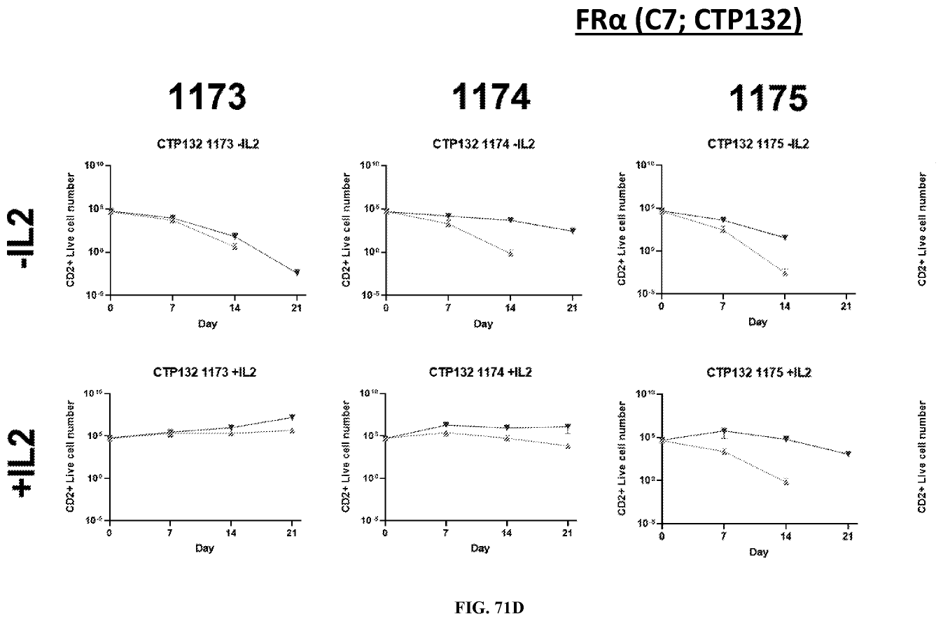

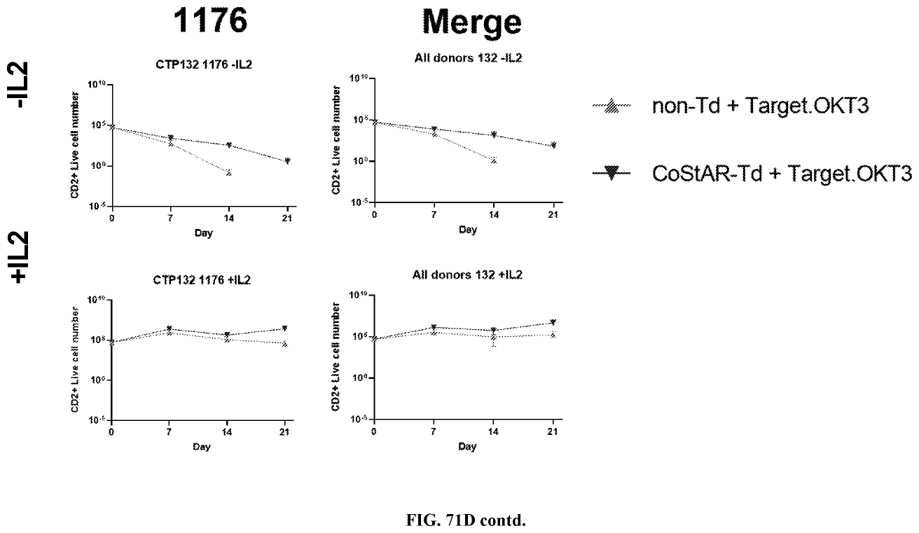

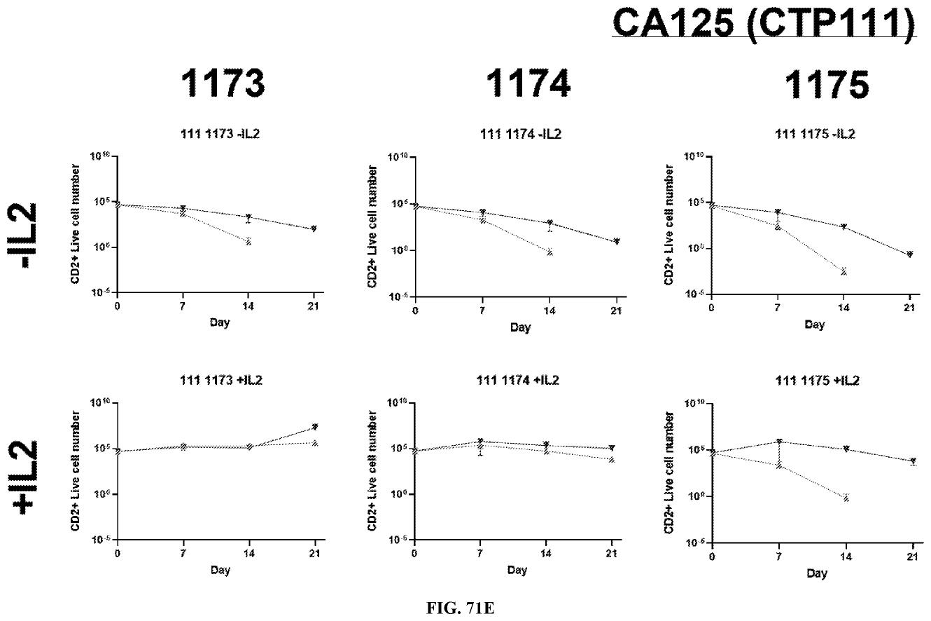

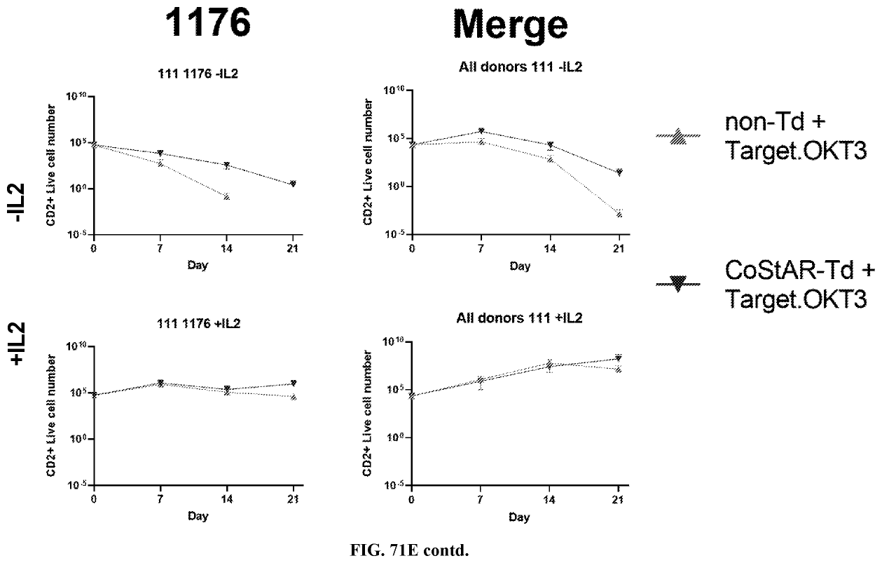

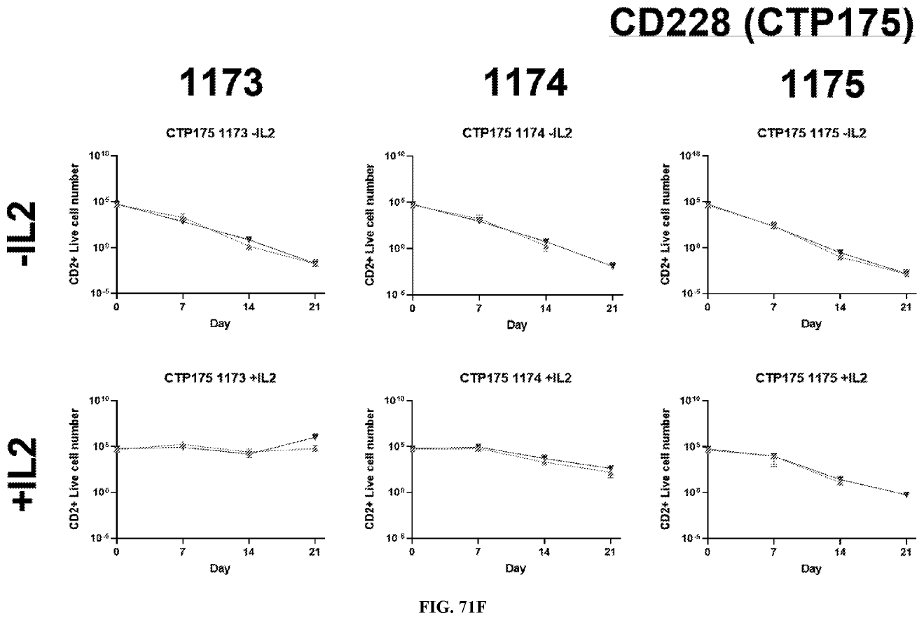

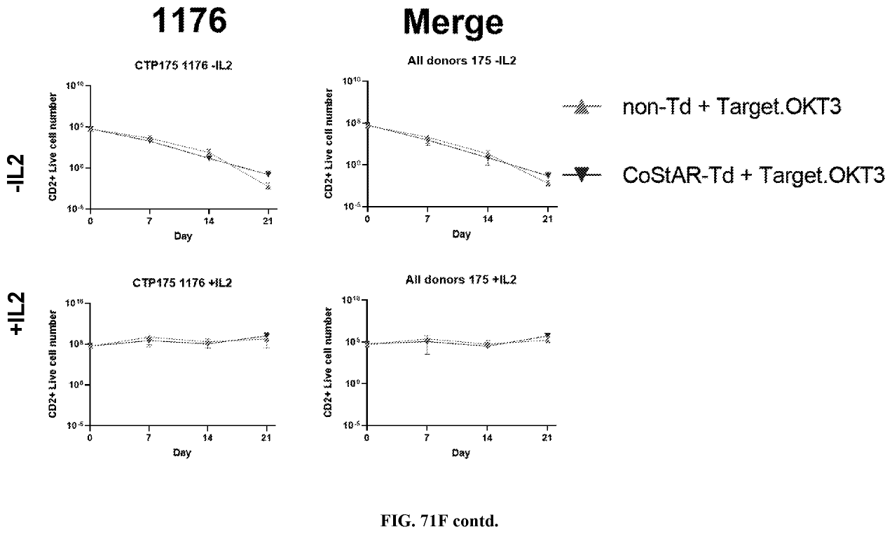

Simplified graphs for this experiment are shown in FIG. 71, comparing nontransduced and CoStAR transduced cells incubated with target.OKT3 cells.

FIG. 71A depicts the results for FRα CoStAR (CTP205). FIG. 71B depicts the results for CEA CoStAR (CTP194). FIG. 71C depicts the results for MSLN CoStAR (CTP224). FIG. 71D depicts the results for FRα CoStAR (C7, CTP132). FIG. 71E depicts the results for CA125 CoStAR (CTP111). FIG. 71F depicts the results for CD228 CoStAR (CTP175). Together, these results demonstrate that the use of CoStAR with no IL2 supplementation is a strategy that is applicable to the CoStAR platform, and is not limited to the examples with MOV19-FOLR1 or MFE23-CEA discussed above.

### Example 43

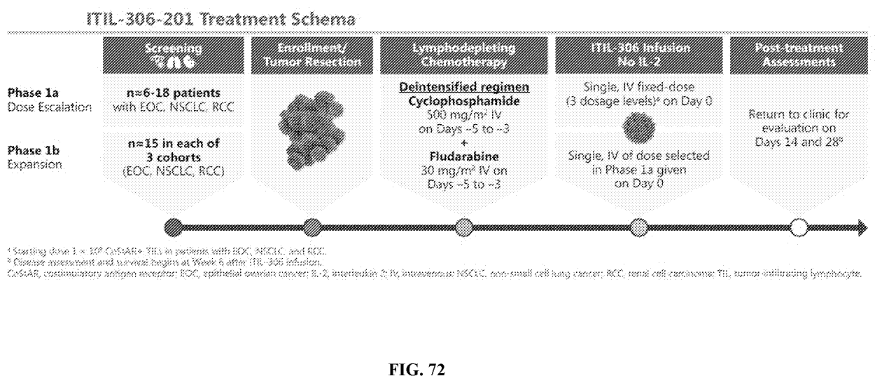

This example provides the first clinical and translational results for clinical trial ITIL-306-201. The schematic for ITIL-306-201 for some embodiments of administering some FRα CoStARs is illustrated in FIG. 72. As shown in FIG. 72, the ITIL 306-201 study, includes Dose Escalation and Expansion phases and Screening, Enrollment/Tumor Resection, Lymphodepleting Chemotherapy, ITIL-306 Infusion without IL-2, and Post Treatment Assessment steps. The lymphodepleting chemotherapy may include a deintensified regimen of cyclophosphamide 500 mg/m 2 IV on days −5 to −3, and fludarabine 30 mg/m 2 IV on days −5 to −3. 6-18 patients are part of the dose escalation phase and receive a single, IV fixed-dose on Day 0, where the dose is one of three dosage levels of ITIL-306-201 infusion with no IL-2. In the Expansion phase, approximately 15 patients may receive a single, IV of dose of ITIL-306-201 infusion on Day 0, where the dose is selected in the dose escalation phase. Patients return to clinic for evaluation on days 14 and 28.

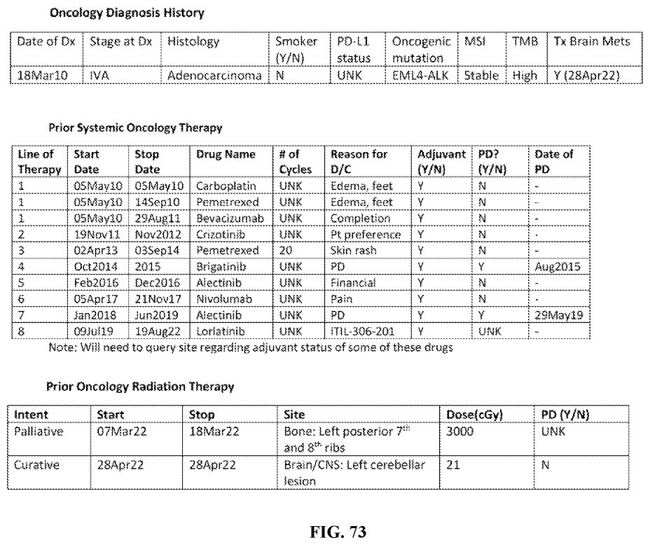

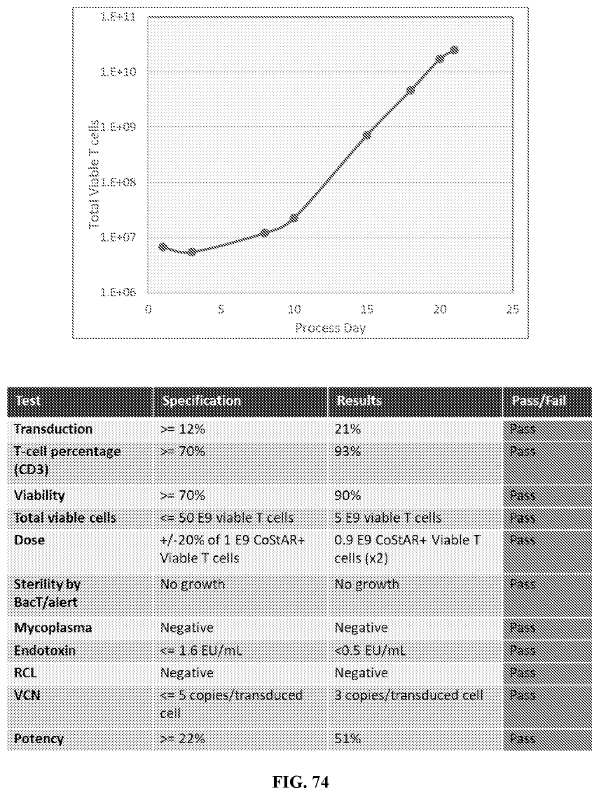

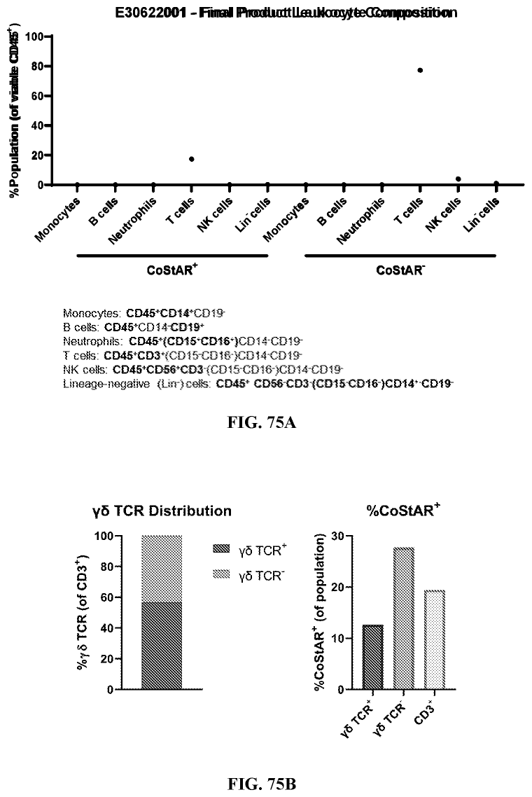

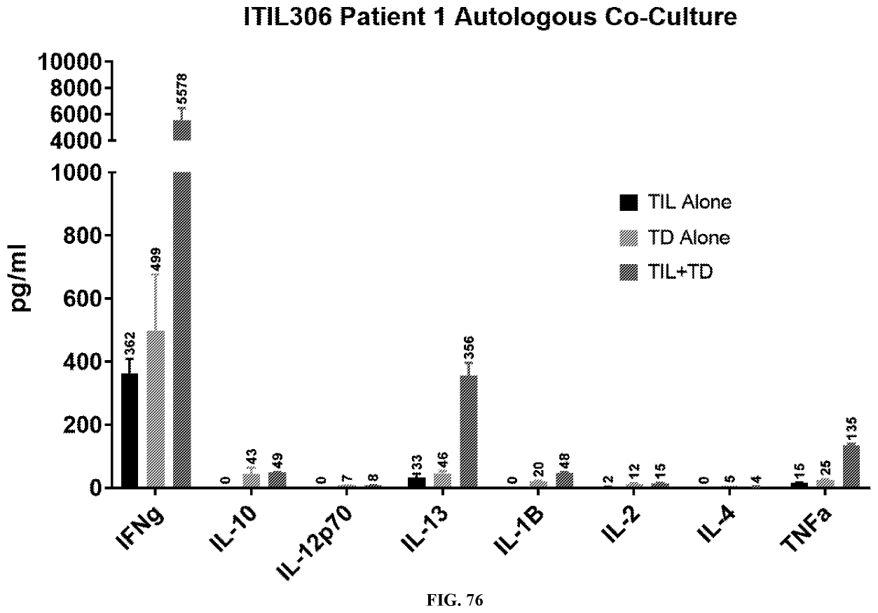

The patient history for Patient 1 enrolled in the ITIL-306-201 trial is included in FIG. 73 indicating past diagnosis, and oncology and radiation therapies. An overview of the CoStAR transduced TIL product (30622001) generated from the TILs of Patient 1 is summarized in FIG. 74. The count of total viable T cells was found to increase as as the days of the process advanced (FIG. 74). 30622001 showed results within specifications for transduction %, T cell % (CD3), viability %, total viable cell number, sterility by BacT/alert, mycoplasma status, endotoxin levels, replication competent lentivirus (RCL), viral copy number (VCN), and potency. 30622001 consisted of approximately 77% nontransduced T cells and 18% transduced T cells, with only about 0.2% of cells belonging to contaminating subsets (transduced NK cells) (FIG. 75A). Unexpectedly, approximately 50% of CD3+ cells were δγ TCR+ (FIG. 75B). Furthermore, approximately 12% of CoStAR transduced cells were δγ TCR+ (FIG. 75B). Cytokine production from CoStAR transduced TIL product 30622001 generated from the TILs of Patient 1 was analyzed following autologous coculture by V-PLEX Proinflammatory Panel 1 Human Kit from MesoScale Discovery (MSD) for TILs alone, transduced TILs alone (TD), and TIL+TD (FIG. 76). As shown in FIG. 76, the TIL+TD condition demonstrated enhanced IFNγ, IL-13, and TNFα levels compared to the other two conditions.

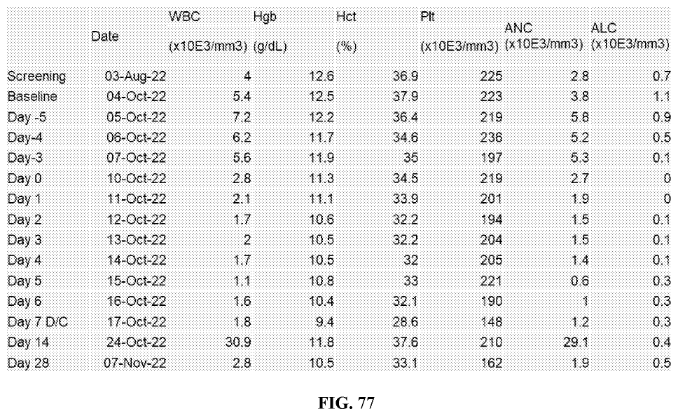

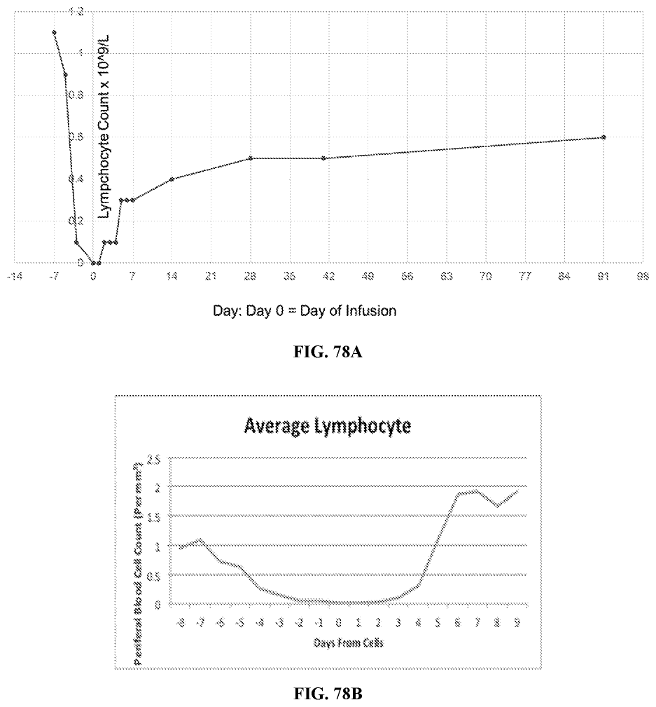

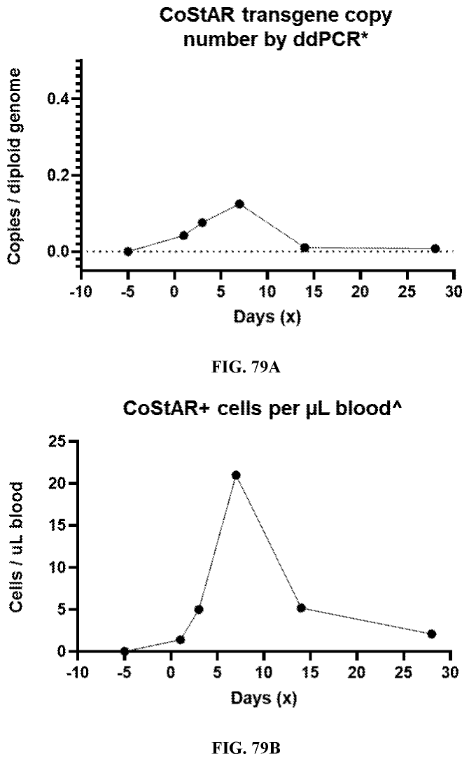

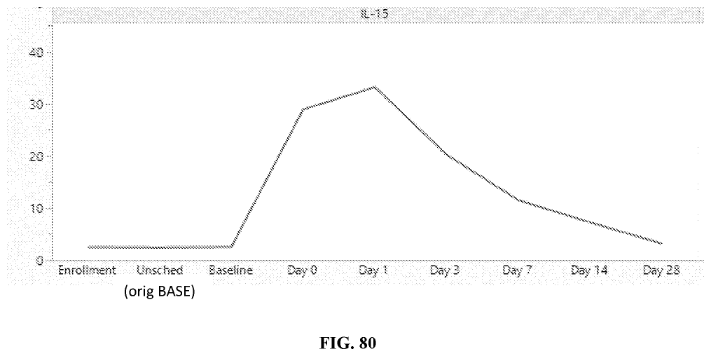

Blood results from Patient 1 taken from patient screening until Day 28 indicated no significant neutropenia or thrombocytopenia, and desirable levels of lymphopenia (FIG. 77). depicts results from blood testing of Patient 1 the ITIL-306-201 study from initial screening to Day 28. Lymphocyte counts during treatment indicated reasonable levels of engraftment, where the speed of engraftment may have been influenced by the lack of an IL-2 dose (FIGS. 78A-78B). FRα-CoStAR transgene was detectable out to Day 28 post-ITIL-306 infusion by pharmacokinetics droplet digital (dd)PCR, where the method was developed and qualified by analytical sciences/quality control for ITIL product VCN release (FIG. 79A). Additionally, CoStAR+ cells continued to be detected in blood out to Day 28 post-ITIL-306 infusion, where preliminary calculations factored in product VCN and lymphocyte+monocyte counts from site-reported CBC (complete blood count) data (FIG. 79B). Furthermore, IL-15 levels were shown to peak at Day 0 and Day 1 of treatment (FIG. 80).

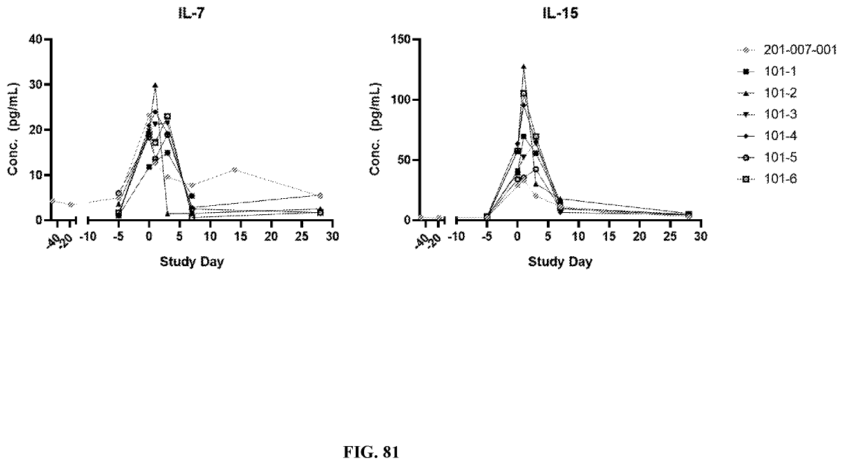

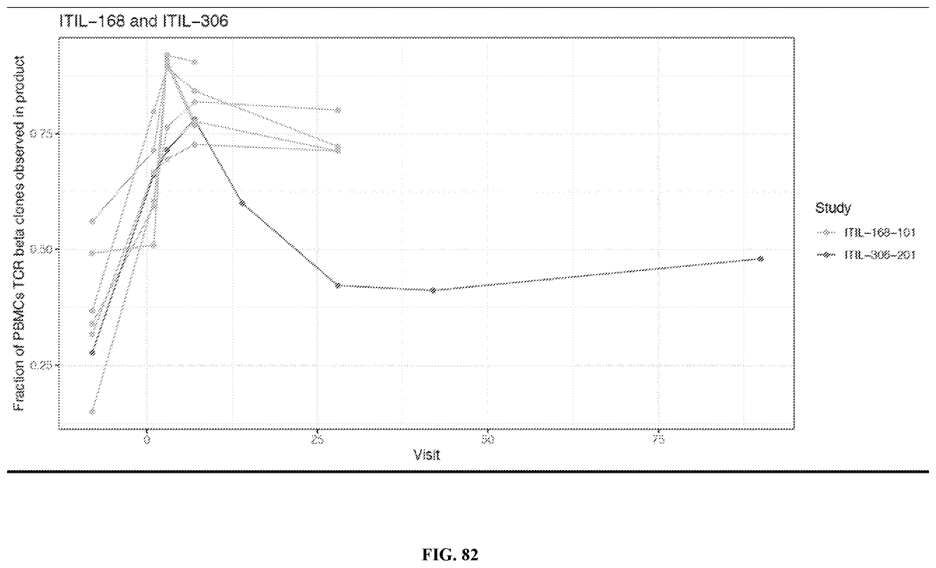

Next, IL-7 and IL-15 levels from Patient 1 undergoing treatment in ITIL-306-201 were compared to IL-7 and IL-15 levels in 6 patients undergoing treatment in ITIL-168-101 (FIG. 81). As indicated in the left panel of FIG. 81, levels of IL-7 were similar across time period measured for both patient 1 in ITIL-306-201 and the 6 patients evaluated in ITIL-168-101. As indicated in the right panel of FIG. 81, levels of IL-15 from Patient 1 in ITIL-306-201 aligned with the lowest range of IL-15 levels from ITIL-168-101. Additionally, while ITIL-168-101 products demonstrated persistence of product related clones out to approximately 28 days, additional testing conducted in ITIL-306-201 demonstrated persistence of product related clones beyond 75 days (FIG. 82).

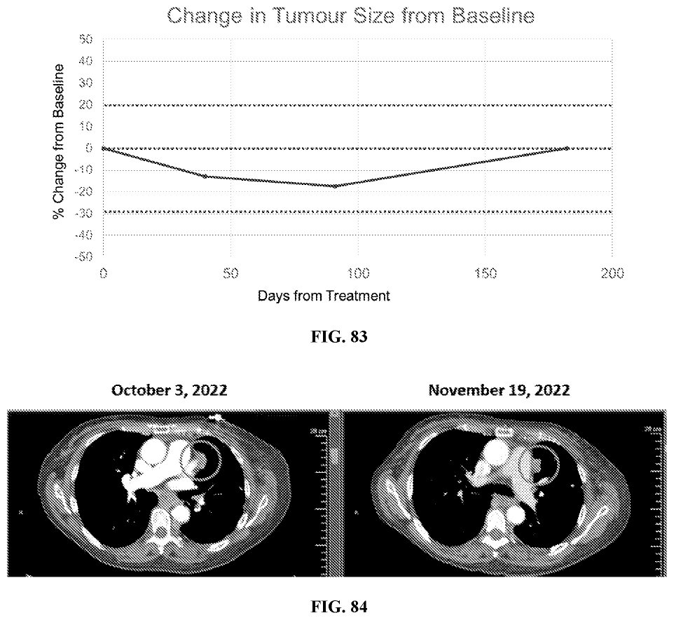

Finally, as shown in FIG. 83, tumor size in Patient 1 was reduced from baseline by approximately 12% prior to Day 50 from ITIL-306-201 treatment, and was reduced by approximately 17% before Day 100 from ITIL-306-201 treatment. The reduction in tumor size is further demonstrated in the CT scan images of the mediastinal lymph node from Patient 1 in FIG. 84. Together, the results achieved with Patient 1 in ITIL-306-201 indicated minimal toxicity, good lymphopenia from the preconditioning chemotherapy, reasonable lymphocyte engraftment and an encouraging clinical outcome marked by stable disease for at least 6 months.

### Example 44

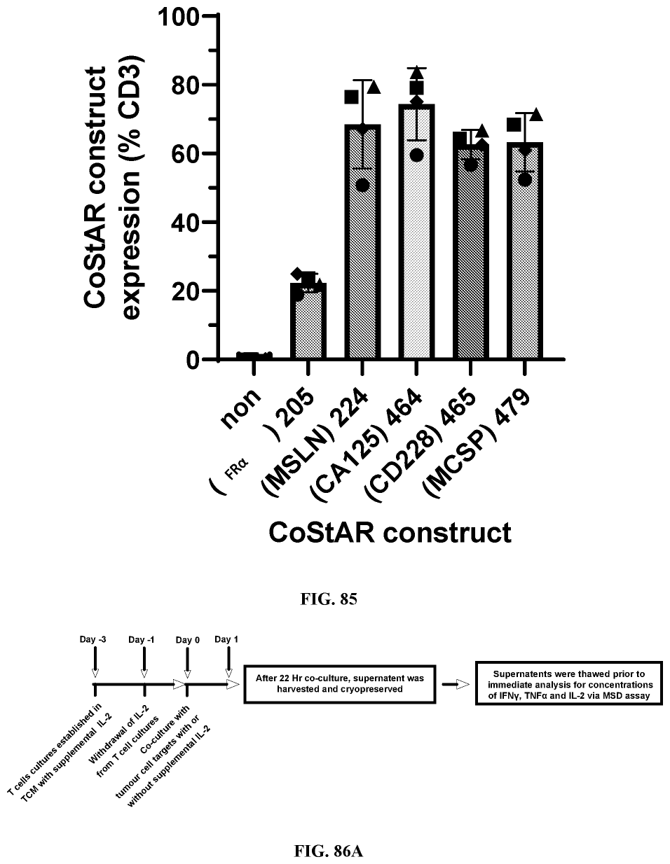

Next, the dependence on intracellular signalling domains, rather than scFv region and tumour associated antigen target, of CoStAR enhancement of cytokine secretion and proliferation by T cells was evaluated. Healthy donor T cells were isolated and activated prior to transduction with lentivirus encoding CoStAR constructs. CoStAR constructs designated 224, 464, 465 and 479 encoded CoStAR linked via 2A sequence to a CD34 marker, and transduced healthy donor T cells were positively sorted using CD34 magnetic isolation beads. The CoStAR construct designated 205 encoded for a CoStAR alone, thus non-transduced and 205-transduced T cells did not undergo positive CD34 magnetic isolation. Non and CoStAR transduced T cells then underwent a rapid-expansion protocol prior to assessment of CoStAR (CoStAR designation 205) or a marker gene expression (CoStAR designation 224, 462, 463, 464, 465 & 479). CoStAR construct expression levels by flow cytometry are shown in FIG. 85.

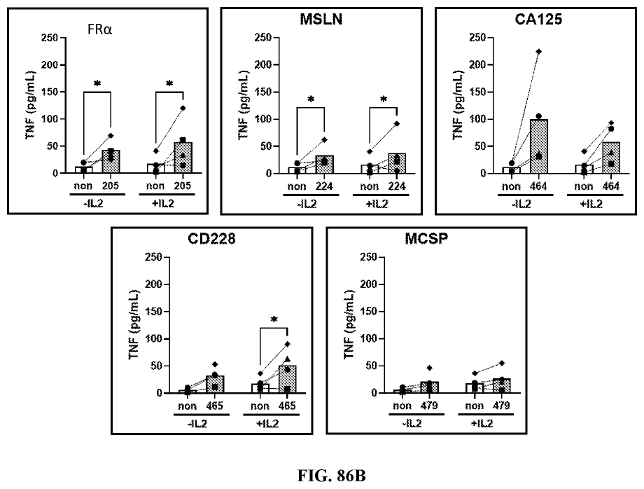

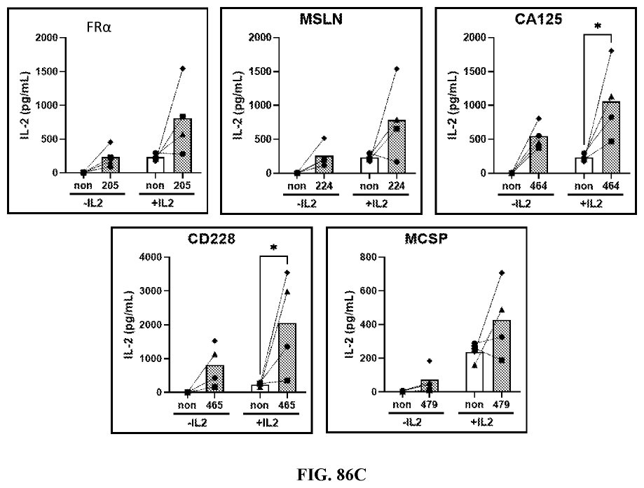

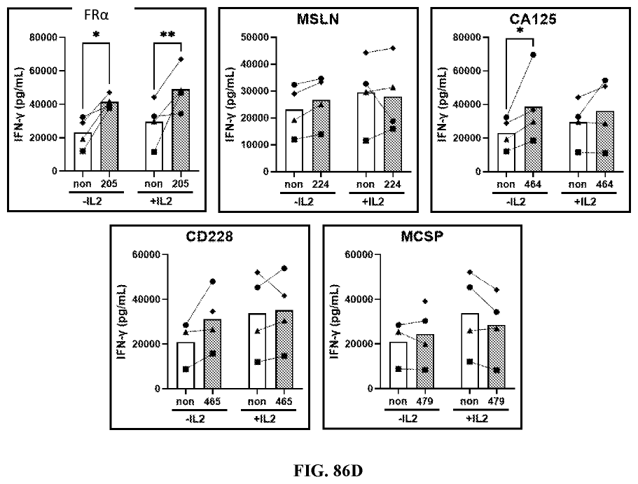

Next, as shown in the experimental schema of FIG. 86A, an assay to evaluate dependence on intracellular signalling domains, rather than scFv region and tumour associated antigen target, of CoStAR enhancement of cytokine secretion by T cells was performed. T cell cultures were established at 1×10\hat{ }6 live cells/mL and allowed to recover from cryopreservation in the presence of 200 IU/mL supplemental IL-2 for 48 hours. Supplemental IL-2 was withdrawn 16 hours prior to co-culture of T cells and tumour targets. In the presence or absence of supplemental IL-2, 50,000 nontransduced or CoStAR transduced T cells from four donors were seeded at a 8:1 effector-to-target ratio with tumour target cells which co-expressed surface-bound OKT3, and CoStAR antigen. For CoStAR designations 465 & 479 the target cells were OVCAR-3.GFP.OKT3 (ovarian cancer), and for 205, 224 & 464 the target cells were SK-MEL-5 GFP.OKT3 (melanoma). Following 22 hours co-culture, the cell-free supernatant was harvested and cryopreserved. Thawed cell-free supernatant was assessed for effector cytokines IFNγ, TNFα and IL-2 by MSD immunoassay according to the manufacturer's protocol.

The secretion of TNFα (FIG. 86B), IL-2 (FIG. 86C), and IFNγ (FIG. 86D) by nontransduced or CoStAR transduced T cells following co-culture with either OVCAR-3 (CoStAR designation 205, 224 and 464) or SK-MEL-5 (CoStAR designation 465 and 479) tumour target lines co-expressing surface-bound OKT3 and CoStAR-antigen (205, FOLR1; 224, MSLN; 464, CA125; 465, CD228; 479, MCSP) was assessed by MSD immunoassay. It was observed that CoStAR enhancement of T cell TNFα, IL-2, and IFNγ secretion was dependent on intracellular signalling domains. Enhancement of secretion was observed against several distinct tumour associated antigen targets and was not dependent on IL-2 supplementation (FIG. 86B-86D).

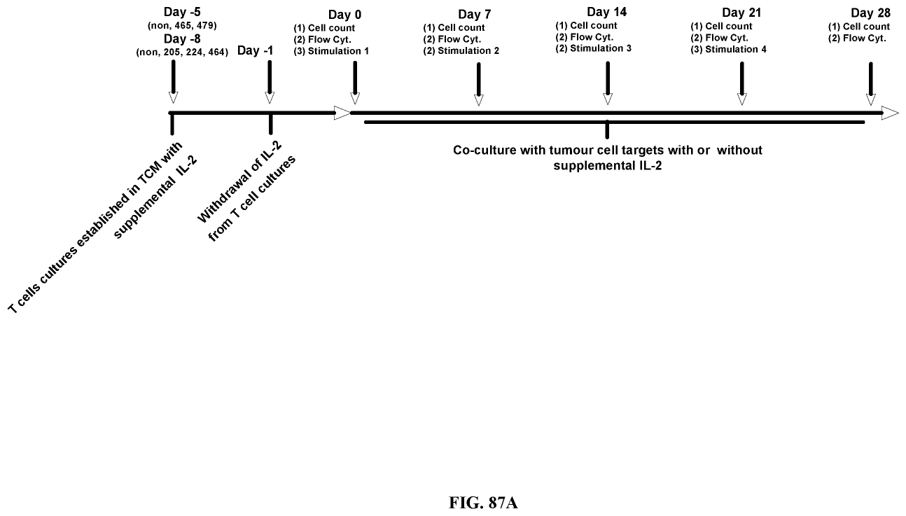

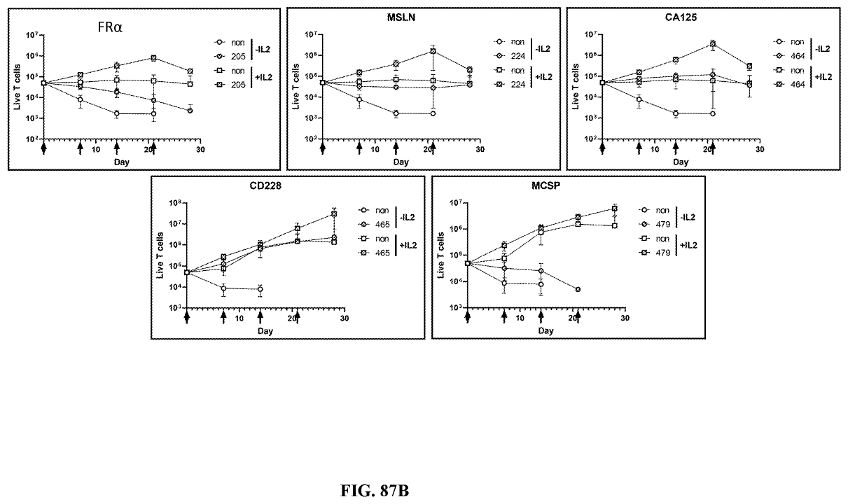

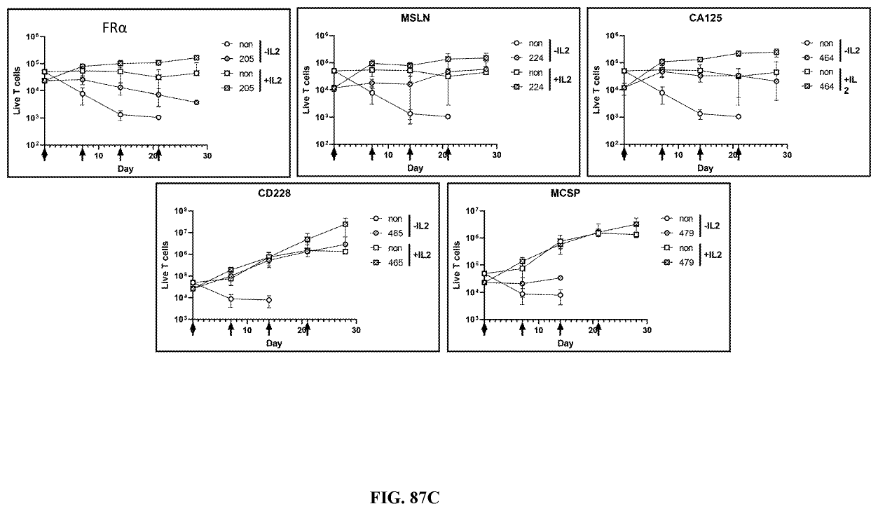

Next, as shown in the experimental schema of FIG. 87A, an assay to evaluate dependence on intracellular signalling domains, rather than scFv region and tumour associated antigen target, of CoStAR enhancement of T cell proliferation was performed. T cell cultures were established at 1×10\hat{ }6 live cells/mL and allowed to recover from cryopreservation in the presence of 200 IU/mL from day −5 or −8 until Day 0 with IL-2 being supplemented every 2-3 days. Supplemental IL-2 was withdrawn 17.5-19 hours prior to co-culture of T cells and tumour targets. Prior to stimulation with target cells, aliquots were taken for flow cytometric determination of (1) the number of live CD2+ T cells per well, (2) the frequency of live CD3+ T cells expressing the CoStAR construct, and (3) in the presence or absence of supplemental IL-2, 50,000 nontransduced or CoStAR transduced T cells from four donors were seeded at a 8:1 effector-to-target ratio with tumour target cells which co-expressed surface-bound OKT3, and CoStAR antigen. The frequency of CoStAR construct expression by live CD3+ T cells was determined either on the day of stimulation, or the following day. In the presence or absence of supplemental IL-2, 50,000 nontransduced or CoStAR transduced T cells from four donors were seeded at a 8:1 effector-to-target ratio with tumour target cells which co-expressed surface-bound OKT3, and CoStAR antigen. For CoStAR designations 465 & 479 the target cells were OVCAR-3.GFP.OKT3 (ovarian cancer), and for 205, 224 & 464 the target cells were SK-MEL-5.GFP.OKT3 (melanoma). T cells were re-stimulated with targets at an 8:1 effector-to-target ratio every 7 days up to stimulation 4.

The proliferation of nontransduced or CoStAR transduced T cells following co-culture with either OVCAR-3 (CoStAR designation 205, 224 and 464) or SK-MEL-5 (CoStAR designation 465 and 479) tumour target lines co-expressing surface-bound OKT3 and CoStAR-antigen (205, FOLR1; 224, MSLN; 464, CA125; 465, CD228; 479, MCSP) was assessed by flow cytometric cell counting of live CD2+ cell counts. As shown by FIGS. 87B-87C, CoStAR enhancement of T cell proliferation was dependent on intracellular signalling domains. Enhancement of proliferation was observed against several distinct tumour associated antigen targets and not dependent on IL-2 supplementation.

1. A method of treating cancer in a subject that expresses a tumor associated antigen (TAA), the method comprising:
a. identifying a subject, wherein the subject has cancer that expresses a TAA; and
b. administering to the subject a cell comprising a fusion protein, wherein the fusion protein comprises:
i) a binding domain specific for the TAA linked to;
ii) a transmembrane domain that is linked to;
iii) a CD28 signaling domain that is linked to;
iv) a CD40 signaling domain;
c. wherein the subject does not receive exogenous IL-2 in a manner that is adequate for cell stimulation of TILs in vivo.

2-81. (canceled)

82. The method of claim 1, wherein the TAA is folate receptor α (FRα), mesothelin (MSLN), cancer antigen 125 (CA125), CD228, melanoma chondroitin sulfate proteoglycan (MCSP), or carcinoembryonic antigen (CEA).

83. The method of claim 1, wherein the fusion protein comprises any one or more of the constructs in FIG. 1, FIG. 16 (individually or combined), FIGS. 20A-20D (individually or combined and/or directed to FRα), FIGS. 21A-21D (individually or combined and/or directed to anti-pembrolizumab), FIGS. 22A-22D (individually or combined and/or directed to anti-CEA), FIGS. 23A-23D (individually or combined and/or directed to anti-MSLN).

84. The method of claim 1, wherein the fusion protein comprises the CDRs as depicted in any one of FIG. 20C, 21C, 22C, or 23D.

85. The method of claim 1, wherein the fusion protein comprises the VH as depicted in any one of FIG. 20C, 21C, 22C, or 23D or a binding fragment thereof.

86. The method of claim 1, wherein the fusion protein comprises the VL as depicted in any one of FIG. 20C, 21C, 22C, or 23D or a binding fragment thereof.

87. The method of claim 1, wherein the fusion protein comprises the sequence as depicted in any one of FIG. 20D, 21D, 22D, or 23D or a sequence at least 85, 86, 87, 88, 89, 90, 91, 92, 93, 94, 95, 96, 97, 98, 99 or 100% identical thereto.

88. The method of claim 1, wherein the fusion protein lacks the signal peptide sequence.

89. The method of claim 1, wherein the fusion protein lacks one of SEQ ID NO: 36, 34, or 2.

90. The method of claim 1, wherein the administered cells comprise a dosage of 5×10\hat{ }8 fusion protein-positive (CoStAR+) T cells, 1×10\hat{ }9 CoStAR+ viable T cells, 3×10\hat{ }9 CoStAR+ viable T cells, or 6×10\hat{ }9 CoStAR+ viable T cells.

91. The method of claim 1, wherein the cell comprises a human tumor infiltrating lymphocyte (TIL), an αβ T cell, a γδ T cell, or an NK T cell.

92. The method of claim 1, wherein the cancer comprises at least one of: solid tumors, renal cancer, lung cancer, or ovarian cancer.

93. A method of treating cancer in a subject that expresses folate receptor α (FRα), the method comprising:
a. identifying a subject, wherein the subject has cancer that expresses FRα; and
b. administering to the subject a cell comprising a fusion protein, wherein the fusion protein comprises:
i) a binding domain specific for FRα linked to;
ii) a transmembrane domain that is linked to;
iii) a CD28 signaling domain that is linked to;
iv) a CD40 signaling domain;
c. wherein the subject does not receive exogenous IL-2 in a manner that is adequate for cell stimulation of TILs in vivo.

94. A method of cell therapy comprising:
a. identifying a subject in need of tumor infiltrating lymphocyte (“TIL”) cell therapy; and
b. administering to the subject a TIL cell therapy, wherein the TIL cell therapy:
i. comprises a fusion protein that comprises:
a) a binding domain specific for folate receptor α (FRα) linked to;
b) a transmembrane domain that is linked to;
c) a CD28 signaling domain that is linked to;
d) a CD40 signaling domain; and
ii. wherein the TIL cell therapy does not include a level of IL-2 administered to the subject, wherein the level is one that is sufficient to provide for IL-2 stimulated TIL cell therapy.

95. A population of genetically engineered immune cells, wherein each immune cell comprises a fusion protein that comprises:
a) a binding domain specific for folate receptor α (FRα) linked to;
b) a transmembrane domain linked to;
c) a CD28 signaling domain linked to;
d) a CD40 signaling domain; and
wherein the population of genetically engineered immune cells has been administered to a subject who has not received an amount of IL-2 that is adequate to promote proliferation in vivo without the fusion protein, and wherein the population of immune cells has been expanded in the absence of IL-2 in vivo.

96. A method of providing treatment to a subject that expresses CEA, MSLN or has pembrolizumab in their system, the method comprising:
a. identifying a subject, wherein the subject has cancer that expresses CEA, MSLN, or has pembrolizumab in their system; and
b. administering to the subject a cell comprising a fusion protein, wherein the fusion protein comprises:
i) a binding domain specific for the corresponding CEA, MSLN or pembrolizumab linked to;
ii) a transmembrane domain that is linked to;
iii) a CD28 signaling domain for the MSLN or pembrolizumab binding domain or ICOS for the CEA binding domain that is linked to;
iv) a CD40 signaling domain;
c. wherein the subject does not receive exogenous IL-2 in a manner that is adequate for cell stimulation of TILs in vivo.

97. A method of cell therapy comprising:
a. identifying a subject in need of tumor infiltrating lymphocyte (“TIL”) cell therapy; and
b. administering to the subject a TIL cell therapy, wherein the TIL cell therapy:
i. comprises a fusion protein that comprises:
a) a binding domain specific for CEA, MSLN or pembrolizumab linked to;
b) a transmembrane domain that is linked to;
c) a CD28 signaling domain (for the MSLN or pembrolizumab binding domain) or an ICOS domain (for the CEA binding domain) that is linked to;
d) a CD40 signaling domain; and
ii. wherein the TIL cell therapy does not include a level of IL-2 administered to the subject, wherein the level is one that is sufficient to provide for IL-2 stimulated TIL cell therapy.

98. A method of in vivo T cell expansion, the method comprising administering a T cell comprising a fusion protein to a subject, wherein IL-2 is not used to promote TIL stimulation, and wherein the fusion protein comprises:
a) a binding domain specific for CEA, MSLN or pembrolizumab linked to;
b) a transmembrane domain that is linked to;
c) a CD28 signaling domain (for the MSLN or pembrolizumab binding domain) or an ICOS domain (for the CEA binding domain) that is linked to;
d) a CD40 signaling domain.
